(19) United States
(12) Patent Application Publication
(10) Pub. No.: US 2026/0125702 A1
(43) Pub. Date: May 7, 2026

(54) COMPOSITIONS AND METHODS FOR ADENO-ASSOCIATED VIRAL PRODUCTION

(71) Applicant: Asimov Inc., Boston, MA (US)

(72) Inventors: Jeremy J. Gam, Somerville, MA (US); Alec A.K. Nielsen, Brookline, MA (US)

(73) Assignee: Asimov Inc., Boston, MA (US)

(21) Appl. No.: 19/119,143

(22) PCT Filed: Oct. 12, 2023
(86) PCT No.: PCT/US2023/076644

§ 371 Date: Apr. 8, 2025

Related U.S. Application Data
(60) Provisional application No. 63/379,158, filed on Oct. 12, 2022.

(51) Int. Cl. C12N 15/86; C12N 9/12

(52) U.S. Cl. CPC C12N 15/86; C12N 9/1241; C12N 2750/14143; C12N 2750/14152

(57) ABSTRACT

Disclosed herein are genetically engineered cells for AAV production. The genetically engineered cells comprise molecular systems for temporal control of expression of genes required for AAV production. Also disclosed herein are methods of using genetically engineered cells for AAV production.

### RELATED APPLICATION

This application claims the benefit under 35 U.S.C. § 119 of U.S. provisional application Ser. No. 63/379,158, filed Oct. 12, 2022, the entire contents of which are incorporated by reference herein.

### REFERENCE TO AN ELECTRONIC SEQUENCE LISTING

The contents of the electronic sequence listing (A121070009WO00-SEQ-JRV.xml; Size: 344,150 bytes; and Date of Creation: Oct. 11, 2023) is herein incorporated by reference in its entirety.

### FIELD

Described herein are Adeno-Associated Virus (AAV) production systems. Also described herein are engineered cells and kits comprising an AAV production system and methods of using the same for AAV production.

### BACKGROUND

AAV is a promising gene delivery modality for cell and gene therapy. AAV can be modified to carry therapeutic genetic payloads to cells within a subject. The production of AAV normally entails transient transfection of plasmids containing genes required for viral vector production into cell culture. However, transient transfection has several shortfalls. Large quantities of DNA and transfection reagent must be procured for the transfection process, which is costly. Also, poor transfection efficiency can result in minimal numbers of “transfected” cells and increased variation associated with transfection steps and viral production.

### SUMMARY

Described herein are AAV production systems that introduce inducible control of gene products required for AAV production including cytostatic or cytotoxic gene products. This inducible control can be mediated by recombinases. Each of the described AAV production systems can be integrated into the genome using random integration, targeted integration, or transposon-mediated integration.

In some aspects, the disclosure relates to Adeno-Associated Virus (AAV) production systems. In some embodiments, an AAV production system comprises an expression control component and an AAV production component, wherein: (a) the expression control component comprises a polynucleic acid molecule encoding for a recombinase; and (b) the AAV production component comprises one or more polynucleic acid molecules collectively encoding for: Rep52 or Rep40; Rep78 or Rep68; E2A; E4Orf6; VARNA; VP1; VP2; VP3; and AAP; at least one of which comprises a nucleic acid sequence of an excisable element, wherein the excisable element comprises, from 5′ to 3′: (i) a first intron splice donor; (ii) a first recombinase site; (iii) a first intron splice acceptor; (iv) an exon comprising a stop codon; (v) a second intron splice donor; (vi) a second recombinase site; (vii) a second intron splice acceptor; wherein the first recombinase site and the second recombinase site correspond to the recombinase of (a).

In some embodiments, the first recombinase attachment site comprises a nucleic acid sequence having at least 80% identity with the nucleic acid sequence of any one of SEQ ID NOs: 5-96. In some embodiments, the second recombinase attachment site comprises a nucleic acid sequence having at least 80% identity with the nucleic acid sequence of any one of SEQ ID NOs: 5-96.

In some embodiments, the first intron splice donor comprises a nucleic acid sequence having at least 80% identity with the nucleic acid sequence of any one of SEQ ID NOs: 1-2. In some embodiments, the second intron splice donor comprises a nucleic acid sequence having at least 80% identity with the nucleic acid sequence of any one of SEQ ID NOs: 1-2.

In some embodiments, the first intron splice acceptor comprises a nucleic acid sequence having at least 80% identity with the nucleic acid sequence of any one of SEQ ID NOs: 3-4. In some embodiments, the second intron splice acceptor comprises a nucleic acid sequence having at least 80% identity with the nucleic acid sequence of any one of SEQ ID NOs: 3-4.

In some embodiments, the first intron splice acceptor of the excisable element is stronger than the second intron splice acceptor of the excisable element.

In some embodiments, the exon of the excisable element comprises a stop codon in all coding frames.

In some embodiments, the exon of the excisable element comprises a nucleic acid sequence having at least 80% identity with the nucleic acid sequence of any one of SEQ ID NOs: 130-131.

In some embodiments, the nucleic acid sequence encoding for Rep52 or Rep40 and/or the nucleic acid sequence encoding for Rep78 or Rep68 comprises the nucleic acid sequence of the excisable element. In some embodiments, the AAV production component comprises the nucleic acid sequence having at least 80% identity with the nucleic acid sequence of any one of SEQ ID NOs: 137-139.

In some embodiments, the nucleic acid sequence encoding for VP1, VP2, and/or VP3 comprises the nucleic acid sequence of the excisable element.

In some embodiments, the nucleic acid sequence encoding for E2A and/or the nucleic acid sequence encoding for E4Orf6 comprises the nucleic acid sequence of the excisable element. In some embodiments, the AAV production component comprises the nucleic acid sequence having at least 80% identity with the nucleic acid sequence of any one of SEQ ID NOs: 134-136.

In some embodiments, an AAV production system further comprises a helper component, wherein the helper component comprises one or more polynucleic acid molecules collectively encoding for: UL5, UL8, UL29, UL30, UL42, UL52, UL12, ICP10, ICP4, and ICP22.

In some embodiments, the recombinase is a PhiC31 recombinase, a Cre recombinase, a VCre recombinase, a Flp recombinase, a Bxb1 recombinase, or a TP901 recombinase. In some embodiments, the recombinase comprises an amino acid sequence having at least 80% identity with the amino acid sequence of any one of SEQ ID NOs: 140-181.

In some aspects, the disclosure relates to engineered cells comprising an Adeno-Associated Virus (AAV) production system described herein. In some embodiments, one or more polynucleic acid molecules of the AAV production component are stably integrated into the genome of the engineered cell.

IN some embodiments, an engineered cell comprises an Adeno-Associated Virus (AAV) production system having an AAV production component comprising one or more polynucleic acid molecules collectively encoding for: Rep52 or Rep40; Rep78 or Rep68; E2A; E40rf6; VARNA; VP1; VP2; VP3; and AAP; at least one of which comprises a nucleic acid sequence of an excisable element, wherein the excisable element comprises, from 5′ to 3′: (i) a first intron splice donor; (ii) a first recombinase site; (iii) a first intron splice acceptor; (iv) an exon comprising a stop codon; (v) a second intron splice donor; (vi) a second recombinase site; (vii) a second intron splice acceptor. In some embodiments, one or more of the polynucleic acid molecules of the AAV production component are stably integrated into the genome of the engineered cell.

In some embodiments, the first recombinase attachment site comprises a nucleic acid sequence having at least 80% identity with the nucleic acid sequence of any one of SEQ ID NOs: 5-96. In some embodiments, the second recombinase attachment site comprises a nucleic acid sequence having at least 80% identity with the nucleic acid sequence of any one of SEQ ID NOs: 5-96.

In some embodiments, the first intron splice donor comprises a nucleic acid sequence having at least 80% identity with the nucleic acid sequence of any one of SEQ ID NOs: 1-2. In some embodiments, the second intron splice donor comprises a nucleic acid sequence having at least 80% identity with the nucleic acid sequence of any one of SEQ ID NOs: 1-2.

In some embodiments, the first intron splice acceptor comprises a nucleic acid sequence having at least 80% identity with the nucleic acid sequence of any one of SEQ ID NOs: 3-4. In some embodiments, the second intron splice acceptor comprises a nucleic acid sequence having at least 80% identity with the nucleic acid sequence of any one of SEQ ID NOs: 3-4.

In some embodiments, the first intron splice acceptor of the excisable element is stronger than the second intron splice acceptor of the excisable element.

In some embodiments, the exon of the excisable element comprises a stop codon in all coding frames.

In some embodiments, the exon of the excisable element comprises a nucleic acid sequence of any one of SEQ ID NOs: 130-131.

In some embodiments, the nucleic acid sequence encoding for Rep52 or Rep40 and/or the nucleic acid sequence encoding for Rep78 or Rep68 comprises the nucleic acid sequence of the excisable element. In some embodiments, the AAV production component comprises a nucleic acid sequence having at least 80% identity with the nucleic acid sequence of any one of SEQ ID NOs: 137-139.

In some embodiments, the nucleic acid sequence encoding for VP1, VP2, and/or VP3 comprises the nucleic acid sequence of the excisable element.

In some embodiments, the nucleic acid sequence encoding for E2A and/or the nucleic acid sequence encoding for E4Orf6 comprises the nucleic acid sequence of the excisable element. In some embodiments, the AAV production component comprises the nucleic acid sequence having at least 80% identity with the nucleic acid sequence of any one of SEQ ID NOs: 134-136.

In some embodiments, the AAV production system further comprises a helper component further comprises a helper component, wherein the helper component comprises one or more polynucleic acid molecules collectively encoding for: UL5, UL8, UL29, UL30, UL42, UL52, UL12, ICP10, ICP4, and ICP22.

In some embodiments, the AAV production system further comprises an expression control component, wherein the expression control component comprises a polynucleic acid molecule encoding for a recombinase, wherein the first recombinase site of the excisable element and the second recombinase site of the excisable element correspond to the recombinase. In some embodiments, the polynucleic acid molecule of the expression control component is stably integrated into the genome of the engineered cell.

In some embodiments, the recombinase is a PhiC31 recombinase, a Cre recombinase, a VCre recombinase, a Flp recombinase, a Bxb1 recombinase, or a TP901 recombinase. In some embodiments, the recombinase comprises an amino acid sequence having at least 80% identity with the amino acid sequence of any one of SEQ ID NOs: 140-181.

In some embodiments, an engineered cell further comprises a transfer polynucleic acid molecule having a sequence encoding, from 5′ to 3′: (i) a nucleic acid sequence of a 5′ inverted tandem repeat; (ii) a multiple cloning site; and (iii) a nucleic acid sequence of a 3′ inverted tandem repeat. In some embodiments, the transfer polynucleic acid molecule is stably integrated into the genome of the engineered cell.

In some aspects, the disclosure relates to kits comprising an Adeno-Associated Virus (AAV) production system described herein, an engineered cell described herein, or a combination thereof. In some embodiments, a kit comprises a transfer polynucleic acid molecule encoding, from 5′ to 3′: (i) a nucleic acid sequence of a first inverted tandem repeat; (ii) a multiple cloning site; and (iii) a nucleic acid sequence of a second inverted tandem repeat. In some embodiments, the transfer polynucleic acid molecule is a plasmid or a vector.

In some aspects, the disclosure relates to methods of producing an AAV vector. In some embodiments a method of producing an AAV vector comprises expressing, in an engineered cell comprising an AAV production system described herein, the recombinase of the expression control component and Rep52 or Rep40, Rep78 or Rep68, E2A, E4Orf6, VARNA, VP1, VP2, VP3, and AAP of the AAV production component.

In some embodiments, the method further comprises introducing a transfer polynucleic acid molecule into the engineered cell. In some embodiments, the transfer polynucleic acid molecule comprises a sequence encoding, from 5′ to 3′: (i) a nucleic acid sequence of a first inverted tandem repeat; (ii) a protein and/or an RNA of interest; and (iii) a nucleic acid sequence of a second inverted tandem repeat. In some embodiments, the transfer polynucleic acid molecule is a plasmid or a vector.

In some aspects, the disclosure relates to a polynucleic acid molecule comprising a nucleic acid sequence having at least 80% identity with the nucleic acid sequence of any one of SEQ ID NOs: 134-139.

In some aspects, the disclosure relates to an engineered cell comprising a nucleic acid sequence having at least 80% identity with the nucleic acid sequence of any one of SEQ ID NOs: 134-139.

### BRIEF DESCRIPTION OF DRAWINGS

FIG. 1 show a plasmid schematic for control of AAV gene product expression (e.g., E4 expression) using an excisable intron containing one or more stop codons. A stop codon-containing exon is flanked by introns (raised lines) defined by consensus splice donor (SD) and splice acceptor (SA) sequences. Recombinase sites (triangles) are located within the flanking introns. Upon recombinase addition, excision of the sequence between the recombinase sites removes the stop codon-containing exon, enabling expression of the full-length coding sequence.

FIG. 2 shows infectious titers generated by exemplary plasmids of an AAV production system. In the absence of a recombinase (e.g., a Cre recombinase), the inclusion of the excisable exon in either pHelper genes E2A and E4orf6 or in AAV Rep resulted in substantially decreased titers (“−recombinase”) compared to standard plasmid titers (left bar of “wt”). Co-transfection with a recombinase increased titers (“+recombinase”) but to reduced values compared to the standard plasmid system. Designs in which helper genes contained an excisable exon showed higher leaky production of AAV in the absence of recombinase, but higher titers in the presence of recombinase, compared to titers from designs in which Rep/Cap genes contained an excisable exon. However, designs in which Rep/Cap genes contained an excisable exon exhibited near background (left bar of “pAAV only”) production of AAV in absence of Cre recombinase. The right bar of “pAAV only” and the right bar of “wt” indicate negative and positive controls, respectively, in the presence of recombinase. Three insertion sites for the excisable intron were tested for each of the pHelper and pRC plasmids.

### DETAILED DESCRIPTION

Viral vectors are a promising gene delivery modality for cell and gene therapy. The production of viral vectors normally entails transient transfection of plasmids into cell culture. However, stable integration of genes necessary to produce therapeutic viral vectors into the genome offers several advantages compared to traditional production via transient transfection. Since cells amplify the viral genes during their own cell division, large quantities of DNA and transfection reagent no longer need to be procured for the transfection process, reducing costs. Also, since the DNA is already within the nucleus, viral titers may be higher and more consistent due to minimal numbers of “untransfected” cells and reduced variation associated with transfection steps. The simpler production process also saves scientist time.

However, several genes required for adeno-associated viral (AAV) vector production have been demonstrated by others to be cytostatic or cytotoxic, namely Rep, E2A and E4. The cytotoxic and cytostatic nature of these proteins has hampered the development of stable AAV producer cell lines in the widely used HEK293 cell line, since the native expression of adenovirus E1 genes in HEK293 cells upregulates expression of these toxic genes. Cells stably transfected with these genes fail to survive selection steps or have silenced expression, resulting in an inability to produce relevant quantities of AAV.

### I. Adeno-Associated Virus Production Systems

In some aspects, the disclosure relates to adeno-associated virus (AAV) production systems. In some embodiments, AAV production systems allow for inducible control of a gene product(s) required for AAV production, including a product(s) that is cytotoxic or cytostatic to a cell. This inducible control can be mediated by recombinases. Because recombinases remove or invert sequences to produce a close to wild type final sequence, expression of viral genes can be controlled by native viral promoters and other native sequences, resulting in near-native expression levels. For some designs, part of the viral coding sequences themselves include an excisable element that includes a stop codon, resulting in near zero expression of the full coding sequence in the absence of excision. Excisable elements have been used by others previously for conditional gene expression in other contexts (see e.g., Shaffer et al., Proc Natl Acad Sci U.S.A. 2022 Jan. 18; 119 (3): e2117451119; Caron et al., Mol Metab. 2016 February; 5 (2): 102-112; Pechisker A., Science Creative Quarterly, August 2004; Wang et al., PLOS One. 2012; 7 (5): €37308). The possibility for low background expression in the absence of recombinase and near-native expression in presence of recombinase makes recombinases a promising technology for viral platforms which have complex and poorly characterized regulation. In contrast, systems that directly regulate viral genes with synthetic promoters (e.g., Tet-On or cumate) require significant tuning and may result in leaky expression in the off state.

An AAV production system, as described herein, may comprise one or more nucleic acid molecules collectively comprising: (a) an AAV production component; (b) an expression control component; (c) a transcriptional activator; (d) a transfer polynucleic acid molecule; (c) a selection marker; or (f) a combination thereof.

a. AAV Production Component

In some embodiments, an AAV production system comprises an AAV production component. An AAV production component, may comprise one or more nucleic acid molecules that collectively encode gene products required for generation of an AAV in a recombinant host cell (or an “engineered cell” as described herein). Exemplary AAV gene products include Rep52, Rep40, Rep78, Rep68, E1, E2A, E4Orf6, VARNA, CAP (VP1, VP2, VP3), AAP, and MAAP or functional variants thereof. The Rep gene products (comprising Rep52, Rep40, Rep78 and Rep68) are involved in AAV genome replication and packaging. The E1 genes upregulate transcription of several adenovirus and AAV genes. The E2A gene product is involved in aiding DNA synthesis processivity during AAV replication. The E4Orf6 gene product supports AAV replication. The VARNA gene product plays a role in regulating translation. The CAP gene products (comprising VP1, VP2, VP3) encode viral capsid proteins. The AAP gene product plays a role in capsid assembly. MAAP is a protein residing in an alternate reading from of VP1 and appears to play a role in the viral capsid as described in Ogden et al. Science 366.6469 (2019): 1139-1143, which is incorporated by reference in its entirety.

In some embodiments, a nucleic acid sequence encoding an AAV gene product is operably linked to a promoter. As used herein, the term “promoter” refers to a nucleic acid sequence that is bound by proteins to initiate transcription of RNA from DNA. A promoter may be a constitutive promoter (i.e., an unregulated promoter that allows for continual transcription). Examples of constitutive promoters are known in the art and include, but are not limited to, cytomegalovirus (CMV) promoters, elongation factor 1 α (EF1α) promoters, simian vacuolating virus 40 (SV40) promoters, ubiquitin-C (UBC) promoters, U6 promoters, p5 promoters, p19 promoters, p40 promoters, E2A promoters, E4 promoters and phosphoglycerate kinase (PGK) promoters. See e.g., Ferreira et al. Proc. Natl. Acad. Sci. U.S.A. 2013 July; 110(28): 11284-89; Pub. No.: US 2014/377861 A1; Qin et al. PloS one 5.5 (2010): e10611.—the entireties of which are incorporated herein by reference. Alternatively, a promoter may be an inducible promoter (i.e., only activates transcription under specific circumstances). An inducible promoter may be a chemically inducible promoter, a temperature inducible promoter, or a light inducible promoter. Additional types of inducible promoters are known to those having ordinary skill in the art. Examples of inducible promoters are known in the art and include, but are not limited to, tetracycline/doxycycline inducible promoters, cumate inducible promoters, ABA inducible promoters, CRY2-CIB1 inducible promoters, DAPG inducible promoters, pTRE3G promoters, pTREtight promoters, the Gal4 UAS operator sequences and mifepristone inducible promoters, and promoters containing at least one of VanR, TtgR, Ph1F, or CymR operator sequences. See e.g., Stanton et al., ACS Synth. Biol. 2014 Dec. 19; 3(12): 880-91; Liang et al., Sci. Signal. 2011 Mar. 15; 4(164): rs2; U.S. Pat. Nos. 7,745,592 B2; 7,935,788 B2—the entireties of which are incorporated herein by reference.

In some embodiments, an AAV production component comprises a nucleic acid sequence encoding a functional variant of Rep52 comprising at least 80% (e.g., at least 80%, at least 85%, at least 90%, at least 95%, or at least 99%) identity to the amino acid sequence of SEQ ID NO: 97, wherein the functional variant is capable of functioning in AAV genome replication. In some embodiments, the AAV production component comprises a nucleic acid sequence encoding a Rep52 polypeptide comprising the amino acid sequence of SEQ ID NO: 97 operably linked to a promoter (as described herein).

In some embodiments, an AAV production component comprises a nucleic acid sequence encoding a functional variant of Rep40 comprising at least 80% (e.g., at least 80%, at least 85%, at least 90%, at least 95%, or at least 99%) identity to the amino acid sequence of SEQ ID NO: 98, wherein the functional variant is capable of functioning in AAV genome replication. In some embodiments, the AAV production component comprises a nucleic acid sequence encoding a Rep40 polypeptide comprising the amino acid sequence of SEQ ID NO: 98 operably linked to a promoter (as described herein).

In some embodiments, an AAV production component comprises a nucleic acid sequence encoding a functional variant of Rep78 comprising at least 80% (e.g., at least 80%, at least 85%, at least 90%, at least 95%, or at least 99%) identity to the amino acid sequence of SEQ ID NO: 99, wherein the functional variant is capable of functioning in AAV genome replication. In some embodiments, the AAV production component comprises a nucleic acid sequence encoding a Rep78 polypeptide comprising the amino acid sequence of SEQ ID NO: 99 operably linked to a promoter (as described herein).

In some embodiments, an AAV production component comprises a nucleic acid sequence encoding a functional variant of Rep68 comprising at least 80% (e.g., at least 80%, at least 85%, at least 90%, at least 95%, or at least 99%) identity to the amino acid sequence of SEQ ID NO: 100, wherein the functional variant is capable of functioning in AAV genome replication. In some embodiments, an AAV production component comprises a nucleic acid sequence encoding a Rep68 polypeptide comprising the amino acid sequence of SEQ ID NO: 100 operably linked to a promoter (as described herein).

In some embodiments, an AAV production component comprises a nucleic acid sequence encoding a functional variant of E2A comprising at least 80% (e.g., at least 80%, at least 85%, at least 90%, at least 95%, or at least 99%) identity to the amino acid sequence of SEQ ID NO: 101, wherein the functional variant is capable of aiding DNA synthesis processivity during AAV replication. In some embodiments, the AAV production component comprises a nucleic acid sequence encoding a E2A polypeptide comprising the amino acid sequence of SEQ ID NO: 101 operably linked to a promoter (as described herein).

In some embodiments, an AAV production component comprises a nucleic acid sequence encoding a functional variant of E4ORF6 comprising at least 80% (e.g., at least 80%, at least 85%, at least 90%, at least 95%, or at least 99%) identity to the amino acid sequence of SEQ ID NO: 102, wherein the functional variant is capable of supporting AAV replication. In some embodiments, the AAV production component comprises a nucleic acid sequence encoding a functional variant of E4ORF6 comprising at least 80% (e.g., at least 80%, at least 85%, at least 90%, at least 95%, or at least 99%) identity to the nucleic acid sequence of SEQ ID NO: 103, wherein the functional variant is capable of supporting AAV replication. In some embodiments, the AAV production component comprises a nucleic acid sequence encoding a E4ORF6 polypeptide comprising the amino acid sequence of SEQ ID NO: 102 operably linked to a promoter (as described herein). In some embodiments, the AAV production component comprises a nucleic acid sequence encoding SEQ ID NO: 103 operably linked to a promoter (as described herein).

In some embodiments, an AAV production component comprises a nucleic acid sequence encoding a functional variant of VARNA comprising at least 80% (e.g., at least 80%, at least 85%, at least 90%, at least 95%, or at least 99%) identity to the nucleic acid sequence of SEQ ID NO: 104, wherein the functional variant is capable regulating translation. In some embodiments, the AAV production component comprises a nucleic acid sequence encoding a VARNA of SEQ ID NO: 104 operably linked to a promoter (as described herein).

In some embodiments, an AAV production component comprises a nucleic acid sequence encoding a functional variant of VP1 comprising at least 80% (e.g., at least 80%, at least 85%, at least 90%, at least 95%, or at least 99%) identity to the amino acid sequence of SEQ ID NO: 105, wherein the functional variant is capable of being incorporated into the AAV capsid. In some embodiments, the AAV production component comprises a nucleic acid sequence encoding a VP1 polypeptide comprising the amino acid sequence of SEQ ID NO: 105 operably linked to a promoter (as described herein).

In some embodiments, an AAV production component comprises a nucleic acid sequence encoding a functional variant of VP2 comprising at least 80% (e.g., at least 80%, at least 85%, at least 90%, at least 95%, or at least 99%) identity to the amino acid sequence of SEQ ID NO: 106, wherein the functional variant is capable of being incorporated into the AAV capsid. In some embodiments, the AAV production component comprises a nucleic acid sequence encoding a VP2 polypeptide comprising the amino acid sequence of SEQ ID NO: 106 operably linked to a promoter (as described herein).

In some embodiments, an AAV production component comprises a nucleic acid sequence encoding a functional variant of VP3 comprising at least 80% (e.g., at least 80%, at least 85%, at least 90%, at least 95%, or at least 99%) identity to the amino acid sequence of SEQ ID NO: 107, wherein the functional variant is capable of being incorporated into the AAV capsid. In some embodiments, the AAV production component comprises a nucleic acid sequence encoding a VP3 polypeptide comprising the amino acid sequence of SEQ ID NO: 107 operably linked to a promoter (as described herein).

In some embodiments, an AAV production component comprises a nucleic acid sequence encoding a functional variant of AAP comprising at least 80% (e.g., at least 80%, at least 85%, at least 90%, at least 95%, or at least 99%) identity to the amino acid sequence of SEQ ID NO: 108, wherein the functional variant is capable of regulating AAV capsid assembly. In some embodiments, the AAV production component comprises a nucleic acid sequence encoding an AAP polypeptide comprising the amino acid sequence of SEQ ID NO: 108 operably linked to a promoter (as described herein).

In some embodiments, an AAV production component comprises a nucleic acid sequence encoding a functional variant of MAAP comprising at least 80% (e.g., at least 80%, at least 85%, at least 90%, at least 95%, or at least 99%) identity to the amino acid sequence of SEQ ID NO: 109, wherein the functional variant is capable of regulating AAV capsid assembly. In some embodiments, the AAV production component comprises a nucleic acid sequence encoding an MAAP polypeptide comprising the amino acid sequence of SEQ ID NO: 109 operably linked to a promoter (as described herein).

In some embodiments, an AAV production component is (i.e., the gene products of the AAV component are) encoded on a single nucleic acid molecule. In other embodiments, multiple nucleic acid molecules collectively comprise the AAV production component (i.e., at least two of the gene products of the AAV production component are encoded on different nucleic acid molecules). For example, an AAV production component may comprise at least 2, at least 3, at least 4, at least 5, at least 6, at least 7, at least 8, at least 9, at least 10, or at least 11 nucleic acid molecules. In some embodiments, an AAV production component comprises 2, 3, 4, 5, 6, 7, 8, 9, 10, or 11 nucleic acid molecules.

In some embodiments, an AAV production system comprises one or more nucleic acid sequences that collectively encode the gene products: Rep52 or Rep40; Rep78 or Rep68; E2A; E4Orf6; VARNA; VP1; VP2; VP3; and AAP. In some embodiments, an AAV production system comprises one or more nucleic acid sequences that collectively encode the gene products: Rep52, Rep40, Rep78, Rep68, E2A, E4Orf6, VARNA, VP1, VP2, VP3, and AAP. In some embodiments, the one or more nucleic acid molecules that collectively encode the gene products required for generation of an AAV are each operably linked to a promoter as described herein.

In some embodiments, an AAV production component comprises a polynucleic acid encoding an excisable element. As used herein, the term “excisable element” refers to a nucleic acid sequence comprising, from 5′ to 3′: (i) a first recombinase site; (ii) an internal sequence; and (iii) a second recombinase site; wherein the first recombinase site and the second recombinase site are capable of being bound by the same recombinase, wherein recombinase binding results in excision of the sequence between the first and the second recombinase attachment sites.

In some embodiments, the internal sequence of an excisable element comprises an exon having a stop codon (or a combination of stop codons). In some embodiments, an excisable element comprises more than one stop codon. For example, in some embodiments the internal sequence comprises 2 or more, 3 or more, 4 or more, 5 or more, 6 or more, 7 or more, 8 or more, 9 or more, or 10 or more stop codons. In some embodiments the internal sequence (e.g., exon) comprises 1-2, 1-3, 1-4, 1-5, 1-6, 1-7, 1-8, 1-9, 1-10, 2-3, 2-4, 2-5, 2-6, 2-7, 2-8, 2-9, 2-10, 3-4, 3-5, 3-6, 3-7, 3-8, 3-9, 3-10, 4-5, 4-6, 4-7, 4-8, 4-9, or 4-10 stop codons. In some embodiments the internal sequence (e.g., exon) comprises 1, 2, 3, 4, 5, 6, 7, 8, 9, or 10 stop codons. In some embodiments, the internal sequence (e.g., exon) of an excisable element comprises a stop codon in every reading frame.

In some embodiments, a polynucleic acid molecule of an AAV production component comprises, from 5′ to 3′: a promoter; an excisable element; and a nucleic acid sequence encoding an AAV gene product (or a combination of AAV gene products); wherein the excisable element comprises, from 5′ to 3′: (i) a first recombinase site; (ii) an internal sequence comprising a protein translation initiation site and a start codon, optionally wherein the start codon is out of frame with nucleic acid sequence encoding the AAV gene product (or the combination of AAV gene products); and (iii) a second recombinase site; wherein the first recombinase site and the second recombinase site are capable of being bound by the same recombinase, and wherein recombinase binding results in excision of the sequence between the first and the second recombinase attachment sites. In some embodiments, the nucleic acid sequence of the AAV gene product comprises a nucleic acid sequence encoding for Rep52 (or a functional variant thereof), Rep40 (or a functional variant thereof), Rep78 (or a functional variant thereof), Rep68 (or a functional variant thereof), E1 (or a functional variant thereof), E2A (or a functional variant thereof), E4Orf6 (or a functional variant thereof), VP1 (or a functional variant thereof), VP2 (or a functional variant thereof), VP3 (or a functional variant thereof), AAP (or a functional variant thereof), and/or MAAP (or a functional variant thereof).

In some embodiments, a polynucleic acid molecule of an AAV production component comprises, from 5′ to 3′: a promoter; and a nucleic acid sequence encoding for an AAV gene product (or a combination of AAV gene products); wherein the nucleic acid sequence encoding for the AAV gene product (or the combination of AAV gene products) comprises an excisable element having, from 5′ to 3′: (i) a first recombinase site; (ii) an internal sequence (e.g., exon) comprising a stop codon (or a combination of stop codons), wherein the stop codon is in frame with the start codon of the AAV gene production (or the combination of AAV gene products); and (iii) a second recombinase site; wherein the first recombinase site and the second recombinase site are capable of being bound by the same recombinase, and wherein recombinase binding results in excision of the sequence between the first and the second recombinase attachment sites. In some embodiments, the nucleic acid sequence of the AAV gene product comprises a nucleic acid sequence encoding for Rep52 (or a functional variant thereof), Rep40 (or a functional variant thereof), Rep78 (or a functional variant thereof), Rep68 (or a functional variant thereof), E1 (or a functional variant thereof), E2A (or a functional variant thereof), E4Orf6 (or a functional variant thereof), VP1 (or a functional variant thereof), VP2 (or a functional variant thereof), VP3 (or a functional variant thereof), AAP (or a functional variant thereof), and/or MAAP (or a functional variant thereof).

In some embodiments, an excisable element comprises, from 5′ to 3′: (i) a first intron splice donor; (ii) a first recombinase site; (iii) a first intron splice acceptor; (iv) an exon comprising a stop codon; (v) a second intron splice donor; (vi) a second recombinase site; (vii) a second intron splice acceptor; wherein the first recombinase site and the second recombinase site correspond to the same recombinase (i.e., are capable of being bound by the same recombinase, wherein the binding results in excision of the exon of the excisable element).

In some embodiments, an exon of an excisable element comprises a nucleic acid sequence having at least 80% (e.g., at least 80%, at least 85%, at least 90%, at least 95%, or at least 99%) identity to the nucleic acid sequence of any one of SEQ ID NOs: 130-131. In some embodiments, an exon of an excisable element comprises a polynucleic acid having the nucleic acid sequence of any one of SEQ ID NOs: 130-131.

In some embodiments, an excisable element comprises a nucleic acid sequence having at least 80% (e.g., at least 80%, at least 85%, at least 90%, at least 95%, or at least 99%) identity to the nucleic acid sequence of any one of SEQ ID NOs: 132-133. In some embodiments, an excisable element comprises the nucleic acid sequence of any one of SEQ ID NOs: 132-133.

### 1. Recombinase Attachment Sites

The excisable elements described herein comprise an internal sequence flanked by a pair of recombinase attachment sites that are capable of being bound and recombined by a recombinase. Numerous recombinase attachment sites have been described previously and are known to those having ordinary skill in the art.

In some embodiments, an exon of an excisable element is flanked by a recombinase attachment site that comprises a nucleic acid sequence having at least 80% (e.g., at least 80%, at least 85%, at least 90%, at least 95%, or at least 99%) identity to the nucleic acid sequence of any one of SEQ ID NOs: 5-96 (or the reverse complement of any one of SEQ ID NOs: 5-96). In some embodiments, a recombinase attachment site comprises the nucleic acid sequence of any one of SEQ ID NOs: 5-96 (or the reverse complement of any one of SEQ ID NOs: 5-96).

In some embodiments, an exon of an excisable element is flanked by recombinase attachment sites that are capable of being bound and recombined by a PhiC31 recombinase.

In some embodiments, an exon of an excisable element is flanked on one end by a first recombinase attachment site having at least 80% (e.g., at least 80%, at least 85%, at least 90%, at least 95%, or at least 99%) identity to the nucleic acid sequence of SEQ ID NO: 5 (or the reverse complement of SEQ ID NO: 5) and on the other end by a second recombinase attachment site having at least 80% (e.g., at least 80%, at least 85%, at least 90%, at least 95%, or at least 99%) identity to the nucleic acid sequence of SEQ ID NO: 21 (or the reverse complement of SEQ ID NO: 21), wherein the first and the second recombinase attachment sites are capable of being bound and recombined by a PhiC31 recombinase. In some embodiments, an exon of an excisable element is flanked on one end by a first recombinase attachment site having nucleic acid sequence of SEQ ID NO: 5 (or the reverse complement of SEQ ID NO: 5) and on the other end by a second recombinase attachment site having the nucleic acid sequence of SEQ ID NO: 21 (or the reverse complement of SEQ ID NO: 21).

In some embodiments, an exon of an excisable element is flanked on one end by a first recombinase attachment site having at least 80% (e.g., at least 80%, at least 85%, at least 90%, at least 95%, or at least 99%) identity to the nucleic acid sequence of SEQ ID NO: 6 (or the reverse complement of SEQ ID NO: 6) and on the other end by a second recombinase attachment site having at least 80% (e.g., at least 80%, at least 85%, at least 90%, at least 95%, or at least 99%) identity to the nucleic acid sequence of SEQ ID NO: 22 (or the reverse complement of SEQ ID NO: 22), wherein the first and the second recombinase attachment sites are capable of being bound and recombined by a PhiC31 recombinase. In some embodiments, an exon of an excisable element is flanked on one end by a first recombinase attachment site having nucleic acid sequence of SEQ ID NO: 6 (or the reverse complement of SEQ ID NO: 6) and on the other end by a second recombinase attachment site having the nucleic acid sequence of SEQ ID NO: 22 (or the reverse complement of SEQ ID NO: 22).

In some embodiments, an exon of an excisable element is flanked on one end by a first recombinase attachment site having at least 80% (e.g., at least 80%, at least 85%, at least 90%, at least 95%, or at least 99%) identity to the nucleic acid sequence of SEQ ID NO: 7 (or the reverse complement of SEQ ID NO: 7) and on the other end by a second recombinase attachment site having at least 80% (e.g., at least 80%, at least 85%, at least 90%, at least 95%, or at least 99%) identity to the nucleic acid sequence of SEQ ID NO: 23 (or the reverse complement of SEQ ID NO: 23), wherein the first and the second recombinase attachment sites are capable of being bound and recombined by a PhiC31 recombinase. In some embodiments, an exon of an excisable element is flanked on one end by a first recombinase attachment site having nucleic acid sequence of SEQ ID NO: 7 (or the reverse complement of SEQ ID NO: 7) and on the other end by a second recombinase attachment site having the nucleic acid sequence of SEQ ID NO: 23 (or the reverse complement of SEQ ID NO: 23).

In some embodiments, an exon of an excisable element is flanked on one end by a first recombinase attachment site having at least 80% (e.g., at least 80%, at least 85%, at least 90%, at least 95%, or at least 99%) identity to the nucleic acid sequence of SEQ ID NO: 8 (or the reverse complement of SEQ ID NO: 8) and on the other end by a second recombinase attachment site having at least 80% (e.g., at least 80%, at least 85%, at least 90%, at least 95%, or at least 99%) identity to the nucleic acid sequence of SEQ ID NO: 24 (or the reverse complement of SEQ ID NO: 24), wherein the first and the second recombinase attachment sites are capable of being bound and recombined by a PhiC31 recombinase. In some embodiments, an exon of an excisable element is flanked on one end by a first recombinase attachment site having nucleic acid sequence of SEQ ID NO: 8 (or the reverse complement of SEQ ID NO: 8) and on the other end by a second recombinase attachment site having the nucleic acid sequence of SEQ ID NO: 24 (or the reverse complement of SEQ ID NO: 24).

In some embodiments, an exon of an excisable element is flanked on one end by a first recombinase attachment site having at least 80% (e.g., at least 80%, at least 85%, at least 90%, at least 95%, or at least 99%) identity to the nucleic acid sequence of SEQ ID NO: 9 (or the reverse complement of SEQ ID NO: 9) and on the other end by a second recombinase attachment site having at least 80% (e.g., at least 80%, at least 85%, at least 90%, at least 95%, or at least 99%) identity to the nucleic acid sequence of SEQ ID NO: 25 (or the reverse complement of SEQ ID NO: 25), wherein the first and the second recombinase attachment sites are capable of being bound and recombined by a PhiC31 recombinase. In some embodiments, an exon of an excisable element is flanked on one end by a first recombinase attachment site having nucleic acid sequence of SEQ ID NO: 9 (or the reverse complement of SEQ ID NO: 9) and on the other end by a second recombinase attachment site having the nucleic acid sequence of SEQ ID NO: 25 (or the reverse complement of SEQ ID NO: 25).

In some embodiments, an exon of an excisable element is flanked on one end by a first recombinase attachment site having at least 80% (e.g., at least 80%, at least 85%, at least 90%, at least 95%, or at least 99%) identity to the nucleic acid sequence of SEQ ID NO: 10 (or the reverse complement of SEQ ID NO: 10) and on the other end by a second recombinase attachment site having at least 80% (e.g., at least 80%, at least 85%, at least 90%, at least 95%, or at least 99%) identity to the nucleic acid sequence of SEQ ID NO: 26 (or the reverse complement of SEQ ID NO: 26), wherein the first and the second recombinase attachment sites are capable of being bound and recombined by a PhiC31 recombinase. In some embodiments, an exon of an excisable element is flanked on one end by a first recombinase attachment site having nucleic acid sequence of SEQ ID NO: 10 (or the reverse complement of SEQ ID NO: 10) and on the other end by a second recombinase attachment site having the nucleic acid sequence of SEQ ID NO: 26 (or the reverse complement of SEQ ID NO: 26).

In some embodiments, an exon of an excisable element is flanked on one end by a first recombinase attachment site having at least 80% (e.g., at least 80%, at least 85%, at least 90%, at least 95%, or at least 99%) identity to the nucleic acid sequence of SEQ ID NO: 11 (or the reverse complement of SEQ ID NO: 11) and on the other end by a second recombinase attachment site having at least 80% (e.g., at least 80%, at least 85%, at least 90%, at least 95%, or at least 99%) identity to the nucleic acid sequence of SEQ ID NO: 27 (or the reverse complement of SEQ ID NO: 27), wherein the first and the second recombinase attachment sites are capable of being bound and recombined by a PhiC31 recombinase. In some embodiments, an exon of an excisable element is flanked on one end by a first recombinase attachment site having nucleic acid sequence of SEQ ID NO: 11 (or the reverse complement of SEQ ID NO: 11) and on the other end by a second recombinase attachment site having the nucleic acid sequence of SEQ ID NO: 27 (or the reverse complement of SEQ ID NO: 27).

In some embodiments, an exon of an excisable element is flanked on one end by a first recombinase attachment site having at least 80% (e.g., at least 80%, at least 85%, at least 90%, at least 95%, or at least 99%) identity to the nucleic acid sequence of SEQ ID NO: 12 (or the reverse complement of SEQ ID NO: 12) and on the other end by a second recombinase attachment site having at least 80% (e.g., at least 80%, at least 85%, at least 90%, at least 95%, or at least 99%) identity to the nucleic acid sequence of SEQ ID NO: 28 (or the reverse complement of SEQ ID NO: 28), wherein the first and the second recombinase attachment sites are capable of being bound and recombined by a PhiC31 recombinase. In some embodiments, an exon of an excisable element is flanked on one end by a first recombinase attachment site having nucleic acid sequence of SEQ ID NO: 12 (or the reverse complement of SEQ ID NO: 12) and on the other end by a second recombinase attachment site having the nucleic acid sequence of SEQ ID NO: 28 (or the reverse complement of SEQ ID NO: 28).

In some embodiments, an exon of an excisable element is flanked on one end by a first recombinase attachment site having at least 80% (e.g., at least 80%, at least 85%, at least 90%, at least 95%, or at least 99%) identity to the nucleic acid sequence of SEQ ID NO: 13 (or the reverse complement of SEQ ID NO: 13) and on the other end by a second recombinase attachment site having at least 80% (e.g., at least 80%, at least 85%, at least 90%, at least 95%, or at least 99%) identity to the nucleic acid sequence of SEQ ID NO: 29 (or the reverse complement of SEQ ID NO: 29), wherein the first and the second recombinase attachment sites are capable of being bound and recombined by a PhiC31 recombinase. In some embodiments, an exon of an excisable element is flanked on one end by a first recombinase attachment site having nucleic acid sequence of SEQ ID NO: 13 (or the reverse complement of SEQ ID NO: 13) and on the other end by a second recombinase attachment site having the nucleic acid sequence of SEQ ID NO: 29 (or the reverse complement of SEQ ID NO: 29).

In some embodiments, an exon of an excisable element is flanked on one end by a first recombinase attachment site having at least 80% (e.g., at least 80%, at least 85%, at least 90%, at least 95%, or at least 99%) identity to the nucleic acid sequence of SEQ ID NO: 14 (or the reverse complement of SEQ ID NO: 14) and on the other end by a second recombinase attachment site having at least 80% (e.g., at least 80%, at least 85%, at least 90%, at least 95%, or at least 99%) identity to the nucleic acid sequence of SEQ ID NO: 30 (or the reverse complement of SEQ ID NO: 30), wherein the first and the second recombinase attachment sites are capable of being bound and recombined by a PhiC31 recombinase. In some embodiments, an exon of an excisable element is flanked on one end by a first recombinase attachment site having nucleic acid sequence of SEQ ID NO: 14 (or the reverse complement of SEQ ID NO: 14) and on the other end by a second recombinase attachment site having the nucleic acid sequence of SEQ ID NO: 30 (or the reverse complement of SEQ ID NO: 30).

In some embodiments, an exon of an excisable element is flanked on one end by a first recombinase attachment site having at least 80% (e.g., at least 80%, at least 85%, at least 90%, at least 95%, or at least 99%) identity to the nucleic acid sequence of SEQ ID NO: 15 (or the reverse complement of SEQ ID NO: 15) and on the other end by a second recombinase attachment site having at least 80% (e.g., at least 80%, at least 85%, at least 90%, at least 95%, or at least 99%) identity to the nucleic acid sequence of SEQ ID NO: 31 (or the reverse complement of SEQ ID NO: 31), wherein the first and the second recombinase attachment sites are capable of being bound and recombined by a PhiC31 recombinase. In some embodiments, an exon of an excisable element is flanked on one end by a first recombinase attachment site having nucleic acid sequence of SEQ ID NO: 15 (or the reverse complement of SEQ ID NO: 15) and on the other end by a second recombinase attachment site having the nucleic acid sequence of SEQ ID NO: 31 (or the reverse complement of SEQ ID NO: 31).

In some embodiments, an exon of an excisable element is flanked on one end by a first recombinase attachment site having at least 80% (e.g., at least 80%, at least 85%, at least 90%, at least 95%, or at least 99%) identity to the nucleic acid sequence of SEQ ID NO: 16 (or the reverse complement of SEQ ID NO: 16) and on the other end by a second recombinase attachment site having at least 80% (e.g., at least 80%, at least 85%, at least 90%, at least 95%, or at least 99%) identity to the nucleic acid sequence of SEQ ID NO: 32 (or the reverse complement of SEQ ID NO: 32), wherein the first and the second recombinase attachment sites are capable of being bound and recombined by a PhiC31 recombinase. In some embodiments, an exon of an excisable element is flanked on one end by a first recombinase attachment site having nucleic acid sequence of SEQ ID NO: 16 (or the reverse complement of SEQ ID NO: 16) and on the other end by a second recombinase attachment site having the nucleic acid sequence of SEQ ID NO: 32 (or the reverse complement of SEQ ID NO: 32).

In some embodiments, an exon of an excisable element is flanked on one end by a first recombinase attachment site having at least 80% (e.g., at least 80%, at least 85%, at least 90%, at least 95%, or at least 99%) identity to the nucleic acid sequence of SEQ ID NO: 17 (or the reverse complement of SEQ ID NO: 17) and on the other end by a second recombinase attachment site having at least 80% (e.g., at least 80%, at least 85%, at least 90%, at least 95%, or at least 99%) identity to the nucleic acid sequence of SEQ ID NO: 33 (or the reverse complement of SEQ ID NO: 33), wherein the first and the second recombinase attachment sites are capable of being bound and recombined by a PhiC31 recombinase. In some embodiments, an exon of an excisable element is flanked on one end by a first recombinase attachment site having nucleic acid sequence of SEQ ID NO: 17 (or the reverse complement of SEQ ID NO: 17) and on the other end by a second recombinase attachment site having the nucleic acid sequence of SEQ ID NO: 33 (or the reverse complement of SEQ ID NO: 33).

In some embodiments, an exon of an excisable element is flanked on one end by a first recombinase attachment site having at least 80% (e.g., at least 80%, at least 85%, at least 90%, at least 95%, or at least 99%) identity to the nucleic acid sequence of SEQ ID NO: 18 (or the reverse complement of SEQ ID NO: 18) and on the other end by a second recombinase attachment site having at least 80% (e.g., at least 80%, at least 85%, at least 90%, at least 95%, or at least 99%) identity to the nucleic acid sequence of SEQ ID NO: 34 (or the reverse complement of SEQ ID NO: 34), wherein the first and the second recombinase attachment sites are capable of being bound and recombined by a PhiC31 recombinase. In some embodiments, an exon of an excisable element is flanked on one end by a first recombinase attachment site having nucleic acid sequence of SEQ ID NO: 18 (or the reverse complement of SEQ ID NO: 18) and on the other end by a second recombinase attachment site having the nucleic acid sequence of SEQ ID NO: 34 (or the reverse complement of SEQ ID NO: 34).

In some embodiments, an exon of an excisable element is flanked on one end by a first recombinase attachment site having at least 80% (e.g., at least 80%, at least 85%, at least 90%, at least 95%, or at least 99%) identity to the nucleic acid sequence of SEQ ID NO: 19 (or the reverse complement of SEQ ID NO: 19) and on the other end by a second recombinase attachment site having at least 80% (e.g., at least 80%, at least 85%, at least 90%, at least 95%, or at least 99%) identity to the nucleic acid sequence of SEQ ID NO: 35 (or the reverse complement of SEQ ID NO: 35), wherein the first and the second recombinase attachment sites are capable of being bound and recombined by a PhiC31 recombinase. In some embodiments, an exon of an excisable element is flanked on one end by a first recombinase attachment site having nucleic acid sequence of SEQ ID NO: 19 (or the reverse complement of SEQ ID NO: 19) and on the other end by a second recombinase attachment site having the nucleic acid sequence of SEQ ID NO: 35 (or the reverse complement of SEQ ID NO: 35).

In some embodiments, an exon of an excisable element is flanked on one end by a first recombinase attachment site having at least 80% (e.g., at least 80%, at least 85%, at least 90%, at least 95%, or at least 99%) identity to the nucleic acid sequence of SEQ ID NO: 20 (or the reverse complement of SEQ ID NO: 20) and on the other end by a second recombinase attachment site having at least 80% (e.g., at least 80%, at least 85%, at least 90%, at least 95%, or at least 99%) identity to the nucleic acid sequence of SEQ ID NO: 36 (or the reverse complement of SEQ ID NO: 36), wherein the first and the second recombinase attachment sites are capable of being bound and recombined by a PhiC31 recombinase. In some embodiments, an exon of an excisable element is flanked on one end by a first recombinase attachment site having nucleic acid sequence of SEQ ID NO: 20 (or the reverse complement of SEQ ID NO: 20) and on the other end by a second recombinase attachment site having the nucleic acid sequence of SEQ ID NO: 36 (or the reverse complement of SEQ ID NO: 36).

In some embodiments, an exon of an excisable element is flanked by recombinase attachment sites that are capable of being bound and recombined by a Bxb1 recombinase.

In some embodiments, an exon of an excisable element is flanked on one end by a first recombinase attachment site having at least 80% (e.g., at least 80%, at least 85%, at least 90%, at least 95%, or at least 99%) identity to the nucleic acid sequence of SEQ ID NO: 37 (or the reverse complement of SEQ ID NO: 37) and on the other end by a second recombinase attachment site having at least 80% (e.g., at least 80%, at least 85%, at least 90%, at least 95%, or at least 99%) identity to the nucleic acid sequence of SEQ ID NO: 53 (or the reverse complement of SEQ ID NO: 53), wherein the first and the second recombinase attachment sites are capable of being bound and recombined by a Bxb1 recombinase. In some embodiments, an exon of an excisable element is flanked on one end by a first recombinase attachment site having nucleic acid sequence of SEQ ID NO: 37 (or the reverse complement of SEQ ID NO: 37) and on the other end by a second recombinase attachment site having the nucleic acid sequence of SEQ ID NO: 53 (or the reverse complement of SEQ ID NO: 53).

In some embodiments, an exon of an excisable element is flanked on one end by a first recombinase attachment site having at least 80% (e.g., at least 80%, at least 85%, at least 90%, at least 95%, or at least 99%) identity to the nucleic acid sequence of SEQ ID NO: 38 (or the reverse complement of SEQ ID NO: 38) and on the other end by a second recombinase attachment site having at least 80% (e.g., at least 80%, at least 85%, at least 90%, at least 95%, or at least 99%) identity to the nucleic acid sequence of SEQ ID NO: 54 (or the reverse complement of SEQ ID NO: 54), wherein the first and the second recombinase attachment sites are capable of being bound and recombined by a Bxb1 recombinase. In some embodiments, an exon of an excisable element is flanked on one end by a first recombinase attachment site having nucleic acid sequence of SEQ ID NO: 38 (or the reverse complement of SEQ ID NO: 38) and on the other end by a second recombinase attachment site having the nucleic acid sequence of SEQ ID NO: 54 (or the reverse complement of SEQ ID NO: 54).

In some embodiments, an exon of an excisable element is flanked on one end by a first recombinase attachment site having at least 80% (e.g., at least 80%, at least 85%, at least 90%, at least 95%, or at least 99%) identity to the nucleic acid sequence of SEQ ID NO: 39 (or the reverse complement of SEQ ID NO: 39) and on the other end by a second recombinase attachment site having at least 80% (e.g., at least 80%, at least 85%, at least 90%, at least 95%, or at least 99%) identity to the nucleic acid sequence of SEQ ID NO: 55 (or the reverse complement of SEQ ID NO: 55), wherein the first and the second recombinase attachment sites are capable of being bound and recombined by a Bxb1 recombinase. In some embodiments, an exon of an excisable element is flanked on one end by a first recombinase attachment site having nucleic acid sequence of SEQ ID NO: 39 (or the reverse complement of SEQ ID NO: 39) and on the other end by a second recombinase attachment site having the nucleic acid sequence of SEQ ID NO: 55 (or the reverse complement of SEQ ID NO: 55).

In some embodiments, an exon of an excisable element is flanked on one end by a first recombinase attachment site having at least 80% (e.g., at least 80%, at least 85%, at least 90%, at least 95%, or at least 99%) identity to the nucleic acid sequence of SEQ ID NO: 40 (or the reverse complement of SEQ ID NO: 40) and on the other end by a second recombinase attachment site having at least 80% (e.g., at least 80%, at least 85%, at least 90%, at least 95%, or at least 99%) identity to the nucleic acid sequence of SEQ ID NO: 56 (or the reverse complement of SEQ ID NO: 56), wherein the first and the second recombinase attachment sites are capable of being bound and recombined by a Bxb1 recombinase. In some embodiments, an exon of an excisable element is flanked on one end by a first recombinase attachment site having nucleic acid sequence of SEQ ID NO: 40 (or the reverse complement of SEQ ID NO: 40) and on the other end by a second recombinase attachment site having the nucleic acid sequence of SEQ ID NO: 56 (or the reverse complement of SEQ ID NO: 56).

In some embodiments, an exon of an excisable element is flanked on one end by a first recombinase attachment site having at least 80% (e.g., at least 80%, at least 85%, at least 90%, at least 95%, or at least 99%) identity to the nucleic acid sequence of SEQ ID NO: 41 (or the reverse complement of SEQ ID NO: 41) and on the other end by a second recombinase attachment site having at least 80% (e.g., at least 80%, at least 85%, at least 90%, at least 95%, or at least 99%) identity to the nucleic acid sequence of SEQ ID NO: 57 (or the reverse complement of SEQ ID NO: 57), wherein the first and the second recombinase attachment sites are capable of being bound and recombined by a Bxb1 recombinase. In some embodiments, an exon of an excisable element is flanked on one end by a first recombinase attachment site having nucleic acid sequence of SEQ ID NO: 41 (or the reverse complement of SEQ ID NO: 41) and on the other end by a second recombinase attachment site having the nucleic acid sequence of SEQ ID NO: 57 (or the reverse complement of SEQ ID NO: 57).

In some embodiments, an exon of an excisable element is flanked on one end by a first recombinase attachment site having at least 80% (e.g., at least 80%, at least 85%, at least 90%, at least 95%, or at least 99%) identity to the nucleic acid sequence of SEQ ID NO: 42 (or the reverse complement of SEQ ID NO: 42) and on the other end by a second recombinase attachment site having at least 80% (e.g., at least 80%, at least 85%, at least 90%, at least 95%, or at least 99%) identity to the nucleic acid sequence of SEQ ID NO: 58 (or the reverse complement of SEQ ID NO: 58), wherein the first and the second recombinase attachment sites are capable of being bound and recombined by a Bxb1 recombinase. In some embodiments, an exon of an excisable element is flanked on one end by a first recombinase attachment site having nucleic acid sequence of SEQ ID NO: 42 (or the reverse complement of SEQ ID NO: 42) and on the other end by a second recombinase attachment site having the nucleic acid sequence of SEQ ID NO: 58 (or the reverse complement of SEQ ID NO: 58).

In some embodiments, an exon of an excisable element is flanked on one end by a first recombinase attachment site having at least 80% (e.g., at least 80%, at least 85%, at least 90%, at least 95%, or at least 99%) identity to the nucleic acid sequence of SEQ ID NO: 43 (or the reverse complement of SEQ ID NO: 43) and on the other end by a second recombinase attachment site having at least 80% (e.g., at least 80%, at least 85%, at least 90%, at least 95%, or at least 99%) identity to the nucleic acid sequence of SEQ ID NO: 59 (or the reverse complement of SEQ ID NO: 59), wherein the first and the second recombinase attachment sites are capable of being bound and recombined by a Bxb1 recombinase. In some embodiments, an exon of an excisable element is flanked on one end by a first recombinase attachment site having nucleic acid sequence of SEQ ID NO: 43 (or the reverse complement of SEQ ID NO: 43) and on the other end by a second recombinase attachment site having the nucleic acid sequence of SEQ ID NO: 59 (or the reverse complement of SEQ ID NO: 59).

In some embodiments, an exon of an excisable element is flanked on one end by a first recombinase attachment site having at least 80% (e.g., at least 80%, at least 85%, at least 90%, at least 95%, or at least 99%) identity to the nucleic acid sequence of SEQ ID NO: 44 (or the reverse complement of SEQ ID NO: 44) and on the other end by a second recombinase attachment site having at least 80% (e.g., at least 80%, at least 85%, at least 90%, at least 95%, or at least 99%) identity to the nucleic acid sequence of SEQ ID NO: 60 (or the reverse complement of SEQ ID NO: 60), wherein the first and the second recombinase attachment sites are capable of being bound and recombined by a Bxb1 recombinase. In some embodiments, an exon of an excisable element is flanked on one end by a first recombinase attachment site having nucleic acid sequence of SEQ ID NO: 44 (or the reverse complement of SEQ ID NO: 44) and on the other end by a second recombinase attachment site having the nucleic acid sequence of SEQ ID NO: 60 (or the reverse complement of SEQ ID NO: 60).

In some embodiments, an exon of an excisable element is flanked on one end by a first recombinase attachment site having at least 80% (e.g., at least 80%, at least 85%, at least 90%, at least 95%, or at least 99%) identity to the nucleic acid sequence of SEQ ID NO: 45 (or the reverse complement of SEQ ID NO: 45) and on the other end by a second recombinase attachment site having at least 80% (e.g., at least 80%, at least 85%, at least 90%, at least 95%, or at least 99%) identity to the nucleic acid sequence of SEQ ID NO: 61 (or the reverse complement of SEQ ID NO: 61), wherein the first and the second recombinase attachment sites are capable of being bound and recombined by a Bxb1 recombinase. In some embodiments, an exon of an excisable element is flanked on one end by a first recombinase attachment site having nucleic acid sequence of SEQ ID NO: 45 (or the reverse complement of SEQ ID NO: 45) and on the other end by a second recombinase attachment site having the nucleic acid sequence of SEQ ID NO: 61 (or the reverse complement of SEQ ID NO: 61).

In some embodiments, an exon of an excisable element is flanked on one end by a first recombinase attachment site having at least 80% (e.g., at least 80%, at least 85%, at least 90%, at least 95%, or at least 99%) identity to the nucleic acid sequence of SEQ ID NO: 46 (or the reverse complement of SEQ ID NO: 46) and on the other end by a second recombinase attachment site having at least 80% (e.g., at least 80%, at least 85%, at least 90%, at least 95%, or at least 99%) identity to the nucleic acid sequence of SEQ ID NO: 62 (or the reverse complement of SEQ ID NO: 62), wherein the first and the second recombinase attachment sites are capable of being bound and recombined by a Bxb1 recombinase. In some embodiments, an exon of an excisable element is flanked on one end by a first recombinase attachment site having nucleic acid sequence of SEQ ID NO: 46 (or the reverse complement of SEQ ID NO: 46) and on the other end by a second recombinase attachment site having the nucleic acid sequence of SEQ ID NO: 62 (or the reverse complement of SEQ ID NO: 62).

In some embodiments, an exon of an excisable element is flanked on one end by a first recombinase attachment site having at least 80% (e.g., at least 80%, at least 85%, at least 90%, at least 95%, or at least 99%) identity to the nucleic acid sequence of SEQ ID NO: 47 (or the reverse complement of SEQ ID NO: 47) and on the other end by a second recombinase attachment site having at least 80% (e.g., at least 80%, at least 85%, at least 90%, at least 95%, or at least 99%) identity to the nucleic acid sequence of SEQ ID NO: 63 (or the reverse complement of SEQ ID NO: 63), wherein the first and the second recombinase attachment sites are capable of being bound and recombined by a Bxb1 recombinase. In some embodiments, an exon of an excisable element is flanked on one end by a first recombinase attachment site having nucleic acid sequence of SEQ ID NO: 47 (or the reverse complement of SEQ ID NO: 47) and on the other end by a second recombinase attachment site having the nucleic acid sequence of SEQ ID NO: 63 (or the reverse complement of SEQ ID NO: 63).

In some embodiments, an exon of an excisable element is flanked on one end by a first recombinase attachment site having at least 80% (e.g., at least 80%, at least 85%, at least 90%, at least 95%, or at least 99%) identity to the nucleic acid sequence of SEQ ID NO: 48 (or the reverse complement of SEQ ID NO: 48) and on the other end by a second recombinase attachment site having at least 80% (e.g., at least 80%, at least 85%, at least 90%, at least 95%, or at least 99%) identity to the nucleic acid sequence of SEQ ID NO: 64 (or the reverse complement of SEQ ID NO: 64), wherein the first and the second recombinase attachment sites are capable of being bound and recombined by a Bxb1 recombinase. In some embodiments, an exon of an excisable element is flanked on one end by a first recombinase attachment site having nucleic acid sequence of SEQ ID NO: 48 (or the reverse complement of SEQ ID NO: 48) and on the other end by a second recombinase attachment site having the nucleic acid sequence of SEQ ID NO: 64 (or the reverse complement of SEQ ID NO: 64).

In some embodiments, an exon of an excisable element is flanked on one end by a first recombinase attachment site having at least 80% (e.g., at least 80%, at least 85%, at least 90%, at least 95%, or at least 99%) identity to the nucleic acid sequence of SEQ ID NO: 49 (or the reverse complement of SEQ ID NO: 49) and on the other end by a second recombinase attachment site having at least 80% (e.g., at least 80%, at least 85%, at least 90%, at least 95%, or at least 99%) identity to the nucleic acid sequence of SEQ ID NO: 65 (or the reverse complement of SEQ ID NO: 65), wherein the first and the second recombinase attachment sites are capable of being bound and recombined by a Bxb1 recombinase. In some embodiments, an exon of an excisable element is flanked on one end by a first recombinase attachment site having nucleic acid sequence of SEQ ID NO: 49 (or the reverse complement of SEQ ID NO: 49) and on the other end by a second recombinase attachment site having the nucleic acid sequence of SEQ ID NO: 65 (or the reverse complement of SEQ ID NO: 65).

In some embodiments, an exon of an excisable element is flanked on one end by a first recombinase attachment site having at least 80% (e.g., at least 80%, at least 85%, at least 90%, at least 95%, or at least 99%) identity to the nucleic acid sequence of SEQ ID NO: 50 (or the reverse complement of SEQ ID NO: 50) and on the other end by a second recombinase attachment site having at least 80% (e.g., at least 80%, at least 85%, at least 90%, at least 95%, or at least 99%) identity to the nucleic acid sequence of SEQ ID NO: 66 (or the reverse complement of SEQ ID NO: 66), wherein the first and the second recombinase attachment sites are capable of being bound and recombined by a Bxb1 recombinase. In some embodiments, an exon of an excisable element is flanked on one end by a first recombinase attachment site having nucleic acid sequence of SEQ ID NO: 50 (or the reverse complement of SEQ ID NO: 50) and on the other end by a second recombinase attachment site having the nucleic acid sequence of SEQ ID NO: 66 (or the reverse complement of SEQ ID NO: 66).

In some embodiments, an exon of an excisable element is flanked on one end by a first recombinase attachment site having at least 80% (e.g., at least 80%, at least 85%, at least 90%, at least 95%, or at least 99%) identity to the nucleic acid sequence of SEQ ID NO: 51 (or the reverse complement of SEQ ID NO: 51) and on the other end by a second recombinase attachment site having at least 80% (e.g., at least 80%, at least 85%, at least 90%, at least 95%, or at least 99%) identity to the nucleic acid sequence of SEQ ID NO: 67 (or the reverse complement of SEQ ID NO: 67), wherein the first and the second recombinase attachment sites are capable of being bound and recombined by a Bxb1 recombinase. In some embodiments, an exon of an excisable element is flanked on one end by a first recombinase attachment site having nucleic acid sequence of SEQ ID NO: 51 (or the reverse complement of SEQ ID NO: 51) and on the other end by a second recombinase attachment site having the nucleic acid sequence of SEQ ID NO: 67 (or the reverse complement of SEQ ID NO: 67).

In some embodiments, an exon of an excisable element is flanked on one end by a first recombinase attachment site having at least 80% (e.g., at least 80%, at least 85%, at least 90%, at least 95%, or at least 99%) identity to the nucleic acid sequence of SEQ ID NO: 52 (or the reverse complement of SEQ ID NO: 52) and on the other end by a second recombinase attachment site having at least 80% (e.g., at least 80%, at least 85%, at least 90%, at least 95%, or at least 99%) identity to the nucleic acid sequence of SEQ ID NO: 68 (or the reverse complement of SEQ ID NO: 68), wherein the first and the second recombinase attachment sites are capable of being bound and recombined by a Bxb1 recombinase. In some embodiments, an exon of an excisable element is flanked on one end by a first recombinase attachment site having nucleic acid sequence of SEQ ID NO: 52 (or the reverse complement of SEQ ID NO: 52) and on the other end by a second recombinase attachment site having the nucleic acid sequence of SEQ ID NO: 68 (or the reverse complement of SEQ ID NO: 68).

In some embodiments, an exon of an excisable element is flanked by recombinase attachment sites that are capable of being bound and recombined by a Cre recombinase.

In some embodiments, an exon of an excisable element is flanked on one end by a first recombinase attachment site having at least 80% (e.g., at least 80%, at least 85%, at least 90%, at least 95%, or at least 99%) identity to the nucleic acid sequence of any one of SEQ ID NOs: 69-77 (or the reverse complement of any one of SEQ ID NOs: 69-77) and on the other end by a second recombinase attachment site that is substantially identical to the first recombinase attachment site, wherein the first and second recombinase attachments sites are capable of being bound and recombined by a Cre recombinase. In some embodiments, the first recombinase attachment site has the nucleic acid sequence of any one of SEQ ID NOS: 69-77.

In some embodiments, an exon of an excisable element is flanked by recombinase attachment sites that are capable of being bound and recombined by a VCre recombinase. In some embodiments, an exon of an excisable element is flanked on one end by a first recombinase attachment site having at least 80% (e.g., at least 80%, at least 85%, at least 90%, at least 95%, or at least 99%) identity to the nucleic acid sequence of any one of SEQ ID NOs: 78-83 (or the reverse complement of any one of SEQ ID NOs: 78-83) and on the other end by a second recombinase attachment site that is substantially identical to the first recombinase attachment, wherein the first and the second recombinase attachment sites are capable of being bound and recombined by a VCre recombinase. In some embodiments, the first recombinase attachment site has the nucleic acid sequence of any one of SEQ ID NOs: 78-83.

In some embodiments, an exon of an excisable element is flanked by recombinase attachment sites that are capable of being bound and recombined by a F1p recombinase.

In some embodiments, an exon of an excisable element is flanked on one end by a first recombinase attachment site having at least 80% (e.g., at least 80%, at least 85%, at least 90%, at least 95%, or at least 99%) identity to the nucleic acid sequence of any one of SEQ ID NOs: 84-96 (or the reverse complement of any one of SEQ ID NOs: 84-96) and on the other end by a second recombinase attachment site that is substantially identical to the first recombinase attachment site, wherein the first and the second recombinase attachment sites are capable of being bound and recombined by a F1p recombinase. In some embodiments, the first recombinase attachment site has the nucleic acid sequence of any one of SEQ ID NOs: 84-96.

### 2. Intron Splice Donors/Acceptors

The excisable element described herein may comprise a nucleic acid sequence of an intron splice donor, a nucleic acid sequence of an intron splice acceptor, or a combination thereof. Numerous intron splice donor and intron splice acceptors sequences are known to those having ordinary skill in the art.

In some embodiments, an intron splice donor comprises a nucleic acid sequence having at least 80% (e.g., at least 80%, at least 85%, at least 90%, at least 95%, or at least 99%) identity to the amino acid sequence of any one of SEQ ID NOs: 1-2.

In some embodiments, an intron splice acceptor comprises a nucleic acid sequence having at least 80% (e.g., at least 80%, at least 85%, at least 90%, at least 95%, or at least 99%) identity to the amino acid sequence of any one of SEQ ID NOs: 3-4.

In some embodiments, an excisable element comprises a nucleic acid sequence of an intron splice acceptor. In some embodiments, an excisable element comprises a nucleic acid sequence of an intron splice donor.

In some embodiments, an excisable element comprises a nucleic acid sequence of an intron splice acceptor and a nucleic acid sequence of an intron splice donor.

In some embodiments, an excisable element comprises, from 5′ to 3′: (i) a first intron splice donor; (ii) a first intron splice acceptor; (iii) a second intron splice donor; and (iv) a second intron splice acceptor. In some embodiments, the first intron splice acceptor of the excisable element is stronger than the second intron splice acceptor of the excisable element. As used herein, a first intron splice acceptor is said to be “stronger” than a second intron splice acceptor, when the first intron splice donor preferentially selects the first intron splice acceptor over the second intron splice acceptor in a splicing reaction. Pairs of strong and weak intron splice acceptors, as well as methods of determining the strength of an intron splice acceptor, are known to those having ordinary skill in the art. See e.g., Dent et al., NAR Genom Bioinform. 2021 May 14; 3(2):lqab041; Mikl et al., Nat Commum. 2019 Oct. 8; 10(1):4572.

### 3. Exemplary Architecture 1

In some embodiments, a polynucleic acid molecule of an AAV production component comprises, from 5′ to 3′: a promoter; an excisable element; and a nucleic acid sequence encoding an AAV gene product (or a combination of AAV gene products); wherein the excisable element comprises, from 5′ to 3′: (i) a first recombinase site; (ii) an internal sequence comprising a protein translation initiation site (e.g., a Kozak sequence) and a start codon, wherein the start codon is out of frame with the nucleic acid sequence encoding the AAV gene product (or the combination of AAV gene products); and (iii) a second recombinase site; wherein the first recombinase site and the second recombinase site are capable of being bound by the same recombinase, and wherein recombinase binding results in excision of the sequence between the first and the second recombinase attachment sites.

In some embodiments, a polynucleic acid molecule of an AAV production component comprises, from 5′ to 3′: a promoter; an excisable element; and a nucleic acid sequence encoding Rep52 (or a functional variant thereof); wherein the excisable element comprises, from 5′ to 3′: (i) a first recombinase site; (ii) an internal sequence comprising a protein translation initiation site and a start codon, wherein the start codon is out of frame with the nucleic acid sequence encoding Rep52 (or the functional variant thereof); and (iii) a second recombinase site; wherein the first recombinase site and the second recombinase site are capable of being bound by the same recombinase, and wherein recombinase binding results in excision of the sequence between the first and the second recombinase attachment sites.

In some embodiments, a polynucleic acid molecule of an AAV production component comprises, from 5′ to 3′: a promoter; an excisable element; and a nucleic acid sequence encoding Rep40 (or a functional variant thereof); wherein the excisable element comprises, from 5′ to 3′: (i) a first recombinase site; (ii) an internal sequence comprising a protein translation initiation site and a start codon, wherein the start codon is out of frame with the nucleic acid sequence encoding Rep40 (or the functional variant thereof); and (iii) a second recombinase site; wherein the first recombinase site and the second recombinase site are capable of being bound by the same recombinase, and wherein recombinase binding results in excision of the sequence between the first and the second recombinase attachment sites.

In some embodiments, a polynucleic acid molecule of an AAV production component comprises, from 5′ to 3′: a promoter; an excisable element; and a nucleic acid sequence encoding Rep78 (or a functional variant thereof); wherein the excisable element comprises, from 5′ to 3′: (i) a first recombinase site; (ii) an internal sequence comprising a protein translation initiation site and a start codon, wherein the start codon is out of frame with the nucleic acid sequence encoding Rep78 (or the functional variant thereof); and (iii) a second recombinase site; wherein the first recombinase site and the second recombinase site are capable of being bound by the same recombinase, and wherein recombinase binding results in excision of the sequence between the first and the second recombinase attachment sites.

In some embodiments, a polynucleic acid molecule of an AAV production component comprises, from 5′ to 3′: a promoter; an excisable element; and a nucleic acid sequence encoding Rep68 (or a functional variant thereof); wherein the excisable element comprises, from 5′ to 3′: (i) a first recombinase site; (ii) an internal sequence comprising a protein translation initiation site and a start codon, wherein the start codon is out of frame with the nucleic acid sequence encoding Rep68 (or the functional variant thereof); and (iii) a second recombinase site; wherein the first recombinase site and the second recombinase site are capable of being bound by the same recombinase, and wherein recombinase binding results in excision of the sequence between the first and the second recombinase attachment sites.

In some embodiments, a polynucleic acid molecule of an AAV production component comprises, from 5′ to 3′: a promoter; an excisable element; and a nucleic acid sequence encoding E2A (or a functional variant thereof); wherein the excisable element comprises, from 5′ to 3′: (i) a first recombinase site; (ii) an internal sequence comprising a protein translation initiation site and a start codon, wherein the start codon is out of frame with the nucleic acid sequence encoding E2A (or the functional variant thereof); and (iii) a second recombinase site; wherein the first recombinase site and the second recombinase site are capable of being bound by the same recombinase, and wherein recombinase binding results in excision of the sequence between the first and the second recombinase attachment sites.

In some embodiments, a polynucleic acid molecule of an AAV production component comprises, from 5′ to 3′: a promoter; an excisable element; and a nucleic acid sequence encoding E4ORF6 (or a functional variant thereof); wherein the excisable element comprises, from 5′ to 3′: (i) a first recombinase site; (ii) an internal sequence comprising a protein translation initiation site and a start codon, wherein the start codon is out of frame with the nucleic acid sequence encoding E4ORF6 (or the functional variant thereof); and (iii) a second recombinase site; wherein the first recombinase site and the second recombinase site are capable of being bound by the same recombinase, and wherein recombinase binding results in excision of the sequence between the first and the second recombinase attachment sites.

In some embodiments, a polynucleic acid molecule of an AAV production component comprises, from 5′ to 3′: a promoter; an excisable element; and a nucleic acid sequence encoding VP1 (or a functional variant thereof); wherein the excisable element comprises, from 5′ to 3′: (i) a first recombinase site; (ii) an internal sequence comprising a protein translation initiation site and a start codon, wherein the start codon is out of frame with the nucleic acid sequence encoding VP1 (or the functional variant thereof); and (iii) a second recombinase site; wherein the first recombinase site and the second recombinase site are capable of being bound by the same recombinase, and wherein recombinase binding results in excision of the sequence between the first and the second recombinase attachment sites.

In some embodiments, a polynucleic acid molecule of an AAV production component comprises, from 5′ to 3′: a promoter; an excisable element; and a nucleic acid sequence encoding VP2 (or a functional variant thereof); wherein the excisable element comprises, from 5′ to 3′: (i) a first recombinase site; (ii) an internal sequence comprising a protein translation initiation site and a start codon, wherein the start codon is out of frame with the nucleic acid sequence encoding VP2 (or the functional variant thereof); and (iii) a second recombinase site; wherein the first recombinase site and the second recombinase site are capable of being bound by the same recombinase, and wherein recombinase binding results in excision of the sequence between the first and the second recombinase attachment sites.

In some embodiments, a polynucleic acid molecule of an AAV production component comprises, from 5′ to 3′: a promoter; an excisable element; and a nucleic acid sequence encoding VP3 (or a functional variant thereof); wherein the excisable element comprises, from 5′ to 3′: (i) a first recombinase site; (ii) an internal sequence comprising a protein translation initiation site and a start codon, wherein the start codon is out of frame with the nucleic acid sequence encoding VP3 (or the functional variant thereof); and (iii) a second recombinase site; wherein the first recombinase site and the second recombinase site are capable of being bound by the same recombinase, and wherein recombinase binding results in excision of the sequence between the first and the second recombinase attachment sites.

In some embodiments, a polynucleic acid molecule of an AAV production component comprises, from 5′ to 3′: a promoter; an excisable element; and a nucleic acid sequence encoding AAP (or a functional variant thereof); wherein the excisable element comprises, from 5′ to 3′: (i) a first recombinase site; (ii) an internal sequence comprising a protein translation initiation site and a start codon, wherein the start codon is out of frame with the nucleic acid sequence encoding AAP (or the functional variant thereof); and (iii) a second recombinase site; wherein the first recombinase site and the second recombinase site are capable of being bound by the same recombinase, and wherein recombinase binding results in excision of the sequence between the first and the second recombinase attachment sites.

In some embodiments, a polynucleic acid molecule of an AAV production component comprises, from 5′ to 3′: a promoter; an excisable element; and a nucleic acid sequence encoding MAAP (or a functional variant thereof); wherein the excisable element comprises, from 5′ to 3′: (i) a first recombinase site; (ii) an internal sequence comprising a protein translation initiation site and a start codon, wherein the start codon is in frame with the nucleic acid sequence encoding MAAP (or the functional variant thereof); and (iii) a second recombinase site; wherein the first recombinase site and the second recombinase site are capable of being bound by the same recombinase, and wherein recombinase binding results in excision of the sequence between the first and the second recombinase attachment sites.

### 4. Exemplary Architecture 2

In some embodiments, a polynucleic acid molecule of an AAV production component comprises, from 5′ to 3′: a promoter; and a nucleic acid sequence encoding for an AAV gene product (or a combination of AAV gene products); wherein the nucleic acid sequence encoding for the AAV gene product (or the combination of AAV gene products) comprises an excisable element having, from 5′ to 3′: (i) a first recombinase site; (ii) an internal sequence (e.g., exon) comprising a stop codon (or a combination of stop codons), wherein the stop codon is in frame with the start codon of the AAV gene production (or the combination of AAV gene products); and (iii) a second recombinase site; wherein the first recombinase site and the second recombinase site are capable of being bound by the same recombinase, and wherein recombinase binding results in excision of the sequence between the first and the second recombinase attachment sites.

In some embodiments, an AAV production system comprises a promoter operably linked to a nucleic acid sequence encoding for Rep52 (or a functional variant thereof), wherein the nucleic acid sequence encoding for Rep52 (or the functional variant thereof) comprises, from 5′ to 3′: (i) a nucleic acid sequence encoding a first portion of Rep52 (or functional variant thereof), wherein the nucleic acid sequence encoding the first portion of Rep52 comprises a start codon; (ii) a first intron splice donor; (iii) a first recombinase site; (iv) a first intron splice acceptor; (v) an exon comprising a stop codon (or combination of stop codons), wherein the stop codon is in frame with the start codon of the nucleic acid sequence encoding for the first portion of Rep52 (or the functional variant thereof); (vi) a second intron splice donor; (vii) a second recombinase site; (viii) a second intron splice acceptor; and (xi) a nucleic acid sequence encoding a second portion of Rep52 (or functional variant thereof); wherein the first recombinase site and the second recombinase site correspond to the same recombinase.

In some embodiments, an AAV production system comprises a promoter operably linked to a nucleic acid sequence encoding for Rep40 (or a functional variant thereof), wherein the nucleic acid sequence encoding for Rep40 (or the functional variant thereof) comprises, from 5′ to 3′: (i) a nucleic acid sequence encoding a first portion of Rep40 (or functional variant thereof), wherein the nucleic acid sequence encoding the first portion of Rep40 comprises a start codon; (ii) a first intron splice donor; (iii) a first recombinase site; (iv) a first intron splice acceptor; (v) an exon comprising a stop codon (or combination of stop codons), wherein the stop codon is in frame with the start codon of the nucleic acid sequence encoding for the first portion of Rep40 (or the functional variant thereof); (vi) a second intron splice donor; (vii) a second recombinase site; (viii) a second intron splice acceptor; and (xi) a nucleic acid sequence encoding a second portion of Rep40 (or functional variant thereof); wherein the first recombinase site and the second recombinase site correspond to the same recombinase.

In some embodiments, an AAV production system comprises a promoter operably linked to a nucleic acid sequence encoding for Rep78 (or a functional variant thereof), wherein the nucleic acid sequence encoding for Rep78 (or the functional variant thereof) comprises, from 5′ to 3′: (i) a nucleic acid sequence encoding a first portion of Rep78 (or functional variant thereof), wherein the nucleic acid sequence encoding the first portion of Rep78 comprises a start codon; (ii) a first intron splice donor; (iii) a first recombinase site; (iv) a first intron splice acceptor; (v) an exon comprising a stop codon (or combination of stop codons), wherein the stop codon is in frame with the start codon of the nucleic acid sequence encoding the first portion of Rep78 (or the functional variant thereof); (vi) a second intron splice donor; (vii) a second recombinase site; (viii) a second intron splice acceptor; and (xi) a nucleic acid sequence encoding a second portion of Rep78 (or functional variant thereof); wherein the first recombinase site and the second recombinase site correspond to the same recombinase.

In some embodiments, an AAV production system comprises a promoter operably linked to a nucleic acid sequence encoding for Rep68 (or a functional variant thereof), wherein the nucleic acid sequence encoding for Rep68 (or the functional variant thereof) comprises, from 5′ to 3′: (i) a nucleic acid sequence encoding a first portion of Rep68 (or functional variant thereof), wherein the nucleic acid sequence encoding the first portion of Rep68 comprises a start codon; (ii) a first intron splice donor; (iii) a first recombinase site; (iv) a first intron splice acceptor; (v) an exon comprising a stop codon (or combination of stop codons), wherein the stop codon is in frame with the start codon of the nucleic acid sequence encoding for the first portion of Rep68 (or the functional variant thereof); (vi) a second intron splice donor; (vii) a second recombinase site; (viii) a second intron splice acceptor; and (xi) a nucleic acid sequence encoding a second portion of Rep68 (or functional variant thereof); wherein the first recombinase site and the second recombinase site correspond to the same recombinase.

In some embodiments, an AAV production system comprises a promoter operably linked to a nucleic acid sequence encoding for Rep (or a functional variant thereof), wherein the nucleic acid sequence encoding for Rep (or the functional variant thereof) comprises, from 5′ to 3′: (i) a nucleic acid sequence encoding a first portion of Rep (or functional variant thereof), wherein the nucleic acid sequence encoding the first portion of Rep comprises a start codon; (ii) a first intron splice donor; (iii) a first recombinase site; (iv) a first intron splice acceptor; (v) an exon comprising a stop codon (or combination of stop codons), wherein the stop codon is in frame with the start codon of the nucleic acid sequence encoding for the first portion of Rep (or the functional variant thereof); (vi) a second intron splice donor; (vii) a second recombinase site; (viii) a second intron splice acceptor; and (xi) a nucleic acid sequence encoding a second portion of Rep (or functional variant thereof); wherein the first recombinase site and the second recombinase site correspond to the same recombinase.

In some embodiments, the first portion of Rep comprises an amino acid sequence having at least 80% identity (e.g., at least 80%, at least 85%, at least 90%, at least 95%, or at least 99%) to the amino acid sequence of SEQ ID NO: 112, the second portion of Rep comprises an amino acid sequence having at least 80% (e.g., at least 80%, at least 85%, at least 90%, at least 95%, or at least 99%) identity to the amino acid sequence of SEQ ID NO: 113, or a combination thereof. In some embodiments, the first portion of Rep consists of the amino acid sequence of SEQ ID NO: 112, and the second portion of Rep consists of the amino acid sequence of SEQ ID NO: 113. In some embodiments, the first portion of Rep comprises an amino acid sequence having at least 80% identity (e.g., at least 80%, at least 85%, at least 90%, at least 95%, or at least 99%) to the amino acid sequence of SEQ ID NO: 114, the second portion of Rep comprises an amino acid sequence having at least 80% (e.g., at least 80%, at least 85%, at least 90%, at least 95%, or at least 99%) identity to the amino acid sequence of SEQ ID NO: 115, or a combination thereof. In some embodiments, the first portion of Rep consists of the amino acid sequence of SEQ ID NO: 114, and the second portion of Rep consists of the amino acid sequence of SEQ ID NO: 115. In some embodiments, the first portion of Rep comprises a amino acid sequence having at least 80% identity (e.g., at least 80%, at least 85%, at least 90%, at least 95%, or at least 99%) to the amino acid sequence of SEQ ID NO: 116, the second portion of Rep comprises an amino acid sequence having at least 80% (e.g., at least 80%, at least 85%, at least 90%, at least 95%, or at least 99%) identity to the amino acid sequence of SEQ ID NO: 117, or a combination thereof. In some embodiments, the first portion of Rep consists of the amino acid sequence of SEQ ID NO: 116, and the second portion of Rep consists of the amino acid sequence of SEQ ID NO: 117.

In some embodiments, an AAV production system comprises a promoter operably linked to a nucleic acid sequence encoding for E2A (or a functional variant thereof), wherein the nucleic acid sequence encoding for E2A (or the functional variant thereof) comprises, from 5′ to 3′: (i) a nucleic acid sequence encoding a first portion of E2A (or functional variant thereof), wherein the nucleic acid sequence encoding the first portion of E2A comprises a start codon; (ii) a first intron splice donor; (iii) a first recombinase site; (iv) a first intron splice acceptor; (v) an exon comprising a stop codon (or combination of stop codons), wherein the stop codon is in frame with the start codon of the nucleic acid sequence encoding for the first portion of E2A (or the functional variant thereof); (vi) a second intron splice donor; (vii) a second recombinase site; (viii) a second intron splice acceptor; and (xi) a nucleic acid sequence encoding a second portion of E2A (or functional variant thereof); wherein the first recombinase site and the second recombinase site correspond to the same recombinase.

In some embodiments, the first portion of E2A comprises an amino acid sequence having at least 80% identity (e.g., at least 80%, at least 85%, at least 90%, at least 95%, or at least 99%) to the amino acid sequence of SEQ ID NO: 118, the second portion of E2A comprises an amino acid sequence having at least 80% (e.g., at least 80%, at least 85%, at least 90%, at least 95%, or at least 99%) identity to the amino acid sequence of SEQ ID NO: 119, or a combination thereof. In some embodiments, the first portion of E2A consists of the amino acid sequence of SEQ ID NO: 118, and the second portion of E2A consists of the amino acid sequence of SEQ ID NO: 119. In some embodiments, the first portion of E2A comprises an amino acid sequence having at least 80% identity (e.g., at least 80%, at least 85%, at least 90%, at least 95%, or at least 99%) to the amino acid sequence of SEQ ID NO: 120, the second portion of E2A comprises an amino acid sequence having at least 80% (e.g., at least 80%, at least 85%, at least 90%, at least 95%, or at least 99%) identity to the amino acid sequence of SEQ ID NO: 121, or a combination thereof. In some embodiments, the first portion of E2A consists of the amino acid sequence of SEQ ID NO: 120, and the second portion of E2A consists of the amino acid sequence of SEQ ID NO: 121. In some embodiments, the first portion of E2A comprises an amino acid sequence having at least 80% identity (e.g., at least 80%, at least 85%, at least 90%, at least 95%, or at least 99%) to the amino acid sequence of SEQ ID NO: 122, the second portion of E2A comprises an amino acid sequence having at least 80% (e.g., at least 80%, at least 85%, at least 90%, at least 95%, or at least 99%) identity to the amino acid sequence of SEQ ID NO: 123, or a combination thereof. In some embodiments, the first portion of E2A consists of the amino acid sequence of SEQ ID NO: 122, and the second portion of E2A consists of the amino acid sequence of SEQ ID NO: 123.

In some embodiments, an AAV production system comprises a promoter operably linked to a nucleic acid sequence encoding for E4ORF6 (or a functional variant thereof), wherein the nucleic acid sequence encoding for E4ORF6 (or the functional variant thereof) comprises, from 5′ to 3′: (i) a nucleic acid sequence encoding a first portion of E4ORF6 (or functional variant thereof), wherein the nucleic acid sequence encoding the first portion of E4ORF6 comprises a start codon; (ii) a first intron splice donor; (iii) a first recombinase site; (iv) a first intron splice acceptor; (v) an exon comprising a stop codon (or combination of stop codons), wherein the stop codon is in frame with the start codon of the nucleic acid sequence encoding the first portion of E4ORF6 (or the functional variant thereof); (vi) a second intron splice donor; (vii) a second recombinase site; (viii) a second intron splice acceptor; and (xi) a nucleic acid sequence encoding a second portion of E4ORF6 (or functional variant thereof); wherein the first recombinase site and the second recombinase site correspond to the same recombinase.

In some embodiments, the first portion of E4ORF6 comprises an amino acid sequence having at least 80% identity (e.g., at least 80%, at least 85%, at least 90%, at least 95%, or at least 99%) to the amino acid sequence of SEQ ID NO: 124, the second portion of E4ORF6 comprises an amino acid sequence having at least 80% (e.g., at least 80%, at least 85%, at least 90%, at least 95%, or at least 99%) identity to the amino acid sequence of SEQ ID NO: 125, or a combination thereof. In some embodiments, the first portion of E4ORF6 consists of the amino acid sequence of SEQ ID NO: 124, and the second portion of E4ORF6 consists of the amino acid sequence of SEQ ID NO: 125. In some embodiments, the first portion of E4ORF6 comprises an amino acid sequence having at least 80% identity (e.g., at least 80%, at least 85%, at least 90%, at least 95%, or at least 99%) to the amino acid sequence of SEQ ID NO: 126, the second portion of E4ORF6 comprises a amino acid sequence having at least 80% (e.g., at least 80%, at least 85%, at least 90%, at least 95%, or at least 99%) identity to the amino acid sequence of SEQ ID NO: 127, or a combination thereof. In some embodiments, the first portion of E4ORF6 consists of the amino acid sequence of SEQ ID NO: 126, and the second portion of E4ORF6 consists of the amino acid sequence of SEQ ID NO: 127. In some embodiments, the first portion of E4ORF6 comprises an amino acid sequence having at least 80% identity (e.g., at least 80%, at least 85%, at least 90%, at least 95%, or at least 99%) to the amino acid sequence of SEQ ID NO: 128, the second portion of E4ORF6 comprises an amino acid sequence having at least 80% (e.g., at least 80%, at least 85%, at least 90%, at least 95%, or at least 99%) identity to the amino acid sequence of SEQ ID NO: 129, or a combination thereof. In some embodiments, the first portion of E4ORF6 consists of the amino acid sequence of SEQ ID NO: 128, and the second portion of E4ORF6 consists of the amino acid sequence of SEQ ID NO: 129.

In some embodiments, an AAV production system comprises a promoter operably linked to a nucleic acid sequence encoding for VP1 (or a functional variant thereof), wherein the nucleic acid sequence encoding for VP1 (or the functional variant thereof) comprises, from 5′ to 3′: (i) a nucleic acid sequence encoding a first portion of VP1 (or functional variant thereof), wherein the nucleic acid sequence encoding the first portion of VP1 comprises a start codon; (ii) a first intron splice donor; (iii) a first recombinase site; (iv) a first intron splice acceptor; (v) an exon comprising a stop codon (or combination of stop codons), wherein the stop codon is in frame with the start codon of the nucleic acid sequence encoding the first portion of VP1 (or the functional variant thereof); (vi) a second intron splice donor; (vii) a second recombinase site; (viii) a second intron splice acceptor; and (xi) a nucleic acid sequence encoding a second portion of VP1 (or functional variant thereof); wherein the first recombinase site and the second recombinase site correspond to the same recombinase.

In some embodiments, an AAV production system comprises a promoter operably linked to a nucleic acid sequence encoding for VP2 (or a functional variant thereof), wherein the nucleic acid sequence encoding for VP2 (or the functional variant thereof) comprises, from 5′ to 3′: (i) a nucleic acid sequence encoding a first portion of VP2 (or functional variant thereof), wherein the nucleic acid sequence encoding the first portion of VP2 comprises a start codon; (ii) a first intron splice donor; (iii) a first recombinase site; (iv) a first intron splice acceptor; (v) an exon comprising a stop codon (or combination of stop codons), wherein the stop codon is in frame with the start codon of the nucleic acid sequence encoding the first portion of VP2 (or the functional variant thereof); (vi) a second intron splice donor; (vii) a second recombinase site; (viii) a second intron splice acceptor; and (xi) a nucleic acid sequence encoding a second portion of VP2 (or functional variant thereof); wherein the first recombinase site and the second recombinase site correspond to the same recombinase.

In some embodiments, an AAV production system comprises a promoter operably linked to a nucleic acid sequence encoding for VP3 (or a functional variant thereof), wherein the nucleic acid sequence encoding for VP3 (or the functional variant thereof) comprises, from 5′ to 3′: (i) a nucleic acid sequence encoding a first portion of VP3 (or functional variant thereof), wherein the nucleic acid sequence encoding the first portion of VP3 comprises a start codon; (ii) a first intron splice donor; (iii) a first recombinase site; (iv) a first intron splice acceptor; (v) an exon comprising a stop codon (or combination of stop codons), wherein the stop codon is in frame with the start codon of the nucleic acid sequence encoding the first portion of VP3 (or the functional variant thereof); (vi) a second intron splice donor; (vii) a second recombinase site; (viii) a second intron splice acceptor; and (xi) a nucleic acid sequence encoding a second portion of VP3 (or functional variant thereof); wherein the first recombinase site and the second recombinase site correspond to the same recombinase. In some embodiments, an AAV production system comprises a promoter operably linked to a nucleic acid sequence encoding for AAP (or a functional variant thereof), wherein the nucleic acid sequence encoding for AAP (or the functional variant thereof) comprises, from 5′ to 3′: (i) a nucleic acid sequence encoding a first portion of AAP (or functional variant thereof), wherein the nucleic acid sequence encoding the first portion of AAP comprises a start codon; (ii) a first intron splice donor; (iii) a first recombinase site; (iv) a first intron splice acceptor; (v) an exon comprising a stop codon (or combination of stop codons), wherein the stop codon is in frame with the start codon of the nucleic acid sequence encoding the first portion of AAP (or the functional variant thereof); (vi) a second intron splice donor; (vii) a second recombinase site; (viii) a second intron splice acceptor; and (xi) a nucleic acid sequence encoding a second portion of AAP (or functional variant thereof); wherein the first recombinase site and the second recombinase site correspond to the same recombinase.

In some embodiments, an AAV production system comprises a promoter operably linked to a nucleic acid sequence encoding for MAAP (or a functional variant thereof), wherein the nucleic acid sequence encoding for MAAP (or the functional variant thereof) comprises, from 5′ to 3′: (i) a nucleic acid sequence encoding a first portion of MAAP (or functional variant thereof), wherein the nucleic acid sequence encoding the first portion of MAAP comprises a start codon; (ii) a first intron splice donor; (iii) a first recombinase site; (iv) a first intron splice acceptor; (v) an exon comprising a stop codon (or combination of stop codons), wherein the stop codon is in frame with the start codon of the nucleic acid sequence encoding the first portion of MAAP (or the functional variant thereof); (vi) a second intron splice donor; (vii) a second recombinase site; (viii) a second intron splice acceptor; and (xi) a nucleic acid sequence encoding a second portion of MAAP (or functional variant thereof); wherein the first recombinase site and the second recombinase site correspond to the same recombinase.

b. The Expression Control Component

In some embodiments, an AAV production system comprises one or more polynucleic acid molecules collectively comprising an expression control component. An expression control component is used to control expression of one or more genes required for AAV production. In some embodiments, the expression control component comprises a recombinase (or a polynucleic acid molecule encoding the same). Exemplary recombinases, and their corresponding recombinase attachment sites, are known to those having skill in the art and include, but are not limited to, PhiC31 recombinases, Bxb1 recombinases, Cre recombinases, VCre recombinases, F1p recombinases, TP901 recombinases, R4 recombinases, and Dre recombinases.

In some embodiments, an expression control component comprises two or more recombinases (or polynucleic acid molecules encoding the same).

In some embodiments, an expression control component comprises a polynucleic acid molecule comprising a nucleic acid sequence encoding for a recombinase operably linked to a promoter (as described herein), such as an inducible promoter.

In some embodiments, an expression control component comprises a PhiC31 recombinase (or a polynucleic acid molecule encoding the same). In some embodiments, a PhiC31 recombinase comprises an amino acid sequence having at least 80% (e.g., at least 80%, at least 85%, at least 90%, at least 95%, or at least 99%) identity to the amino acid sequence of SEQ ID NO: 140. In some embodiments, a Phi3C1 recombinase comprises the amino acid sequence SEQ ID NO: 140. In some embodiments, a nucleic acid sequence encoding for a PhiC31 recombinase comprises a nucleic acid sequence having at least 80% (e.g., at least 80%, at least 85%, at least 90%, at least 95%, or at least 99%) identity to the nucleic acid sequence of SEQ ID NO: 182. In some embodiments, a nucleic acid sequence encoding for a PhiC31 recombinase comprises the nucleic acid sequence of SEQ ID NO: 182. In some embodiments, an expression control component comprises a polynucleic acid encoding for a PhiC31 recombinase operably linked to a promoter (as described herein).

In some embodiments, an expression control component comprises a Cre recombinase (or a polynucleic acid molecule encoding the same). In some embodiments, a Cre recombinase comprises an amino acid sequence having at least 80% (e.g., at least 80%, at least 85%, at least 90%, at least 95%, or at least 99%) identity to the amino acid sequence of SEQ ID NO: 141. In some embodiments, a Cre recombinase comprises the amino acid sequence SEQ ID NO: 141. In some embodiments, a nucleic acid sequence encoding for a Cre recombinase comprises a nucleic acid sequence having at least 80% (e.g., at least 80%, at least 85%, at least 90%, at least 95%, or at least 99%) identity to the nucleic acid sequence of SEQ ID NO: 183. In some embodiments, a nucleic acid sequence encoding for a Cre recombinase comprises the nucleic acid sequence of SEQ ID NO: 183. In some embodiments, an expression control component comprises a polynucleic acid encoding for a Cre recombinase operably linked to a promoter (as described herein).

In some embodiments, an expression control component comprises a VCre recombinase (or a polynucleic acid molecule encoding the same). In some embodiments, a VCre recombinase comprises an amino acid sequence having at least 80% (e.g., at least 80%, at least 85%, at least 90%, at least 95%, or at least 99%) identity to the amino acid sequence of SEQ ID NO: 142. In some embodiments, a VCre recombinase comprises the amino acid sequence SEQ ID NO: 142. In some embodiments, a nucleic acid sequence encoding for a VCre recombinase comprises a nucleic acid sequence having at least 80% (e.g., at least 80%, at least 85%, at least 90%, at least 95%, or at least 99%) identity to the nucleic acid sequence of SEQ ID NO: 184. In some embodiments, a nucleic acid sequence encoding for a VCre recombinase comprises the nucleic acid sequence of SEQ ID NO: 184. In some embodiments, an expression control component comprises a polynucleic acid encoding for a VCre recombinase operably linked to a promoter (as described herein).

In some embodiments, an expression control component comprises a F1p recombinase (or a polynucleic acid molecule encoding the same). In some embodiments, a F1p recombinase comprises an amino acid sequence having at least 80% (e.g., at least 80%, at least 85%, at least 90%, at least 95%, or at least 99%) identity to the amino acid sequence of SEQ ID NO: 143. In some embodiments, a F1p recombinase comprises the amino acid sequence SEQ ID NO: 143. In some embodiments, a nucleic acid sequence encoding for a F1p recombinase comprises a nucleic acid sequence having at least 80% (e.g., at least 80%, at least 85%, at least 90%, at least 95%, or at least 99%) identity to the nucleic acid sequence of SEQ ID NO: 185. In some embodiments, a nucleic acid sequence encoding for a F1p recombinase comprises the nucleic acid sequence of SEQ ID NO: 185. In some embodiments, an expression control component comprises a polynucleic acid encoding for a F1p recombinase operably linked to a promoter (as described herein).

In some embodiments, an expression control component comprises a Bxb1 recombinase (or a polynucleic acid molecule encoding the same). In some embodiments, a Bxb1 recombinase comprises an amino acid sequence having at least 80% (e.g., at least 80%, at least 85%, at least 90%, at least 95%, or at least 99%) identity to the amino acid sequence of SEQ ID NO: 144. In some embodiments, a Bxb1 recombinase comprises the amino acid sequence SEQ ID NO: 144. In some embodiments, a nucleic acid sequence encoding for a Bxb1 recombinase comprises a nucleic acid sequence having at least 80% (e.g., at least 80%, at least 85%, at least 90%, at least 95%, or at least 99%) identity to the nucleic acid sequence of SEQ ID NO: 186. In some embodiments, a nucleic acid sequence encoding for a Bxb1 recombinase comprises the nucleic acid sequence of SEQ ID NO: 186. In some embodiments, an expression control component comprises a polynucleic acid encoding for a Bxb1 recombinase operably linked to a promoter (as described herein).

In some embodiments, an expression control component comprises a TP901 recombinase (or a polynucleic acid molecule encoding the same). In some embodiments, a TP901 recombinase comprises an amino acid sequence having at least 80% (e.g., at least 80%, at least 85%, at least 90%, at least 95%, or at least 99%) identity to the amino acid sequence of SEQ ID NO: 145. In some embodiments, a TP901 recombinase comprises the amino acid sequence SEQ ID NO: 145. In some embodiments, a nucleic acid sequence encoding for a TP901 recombinase comprises a nucleic acid sequence having at least 80% (e.g., at least 80%, at least 85%, at least 90%, at least 95%, or at least 99%) identity to the nucleic acid sequence of SEQ ID NO: 187. In some embodiments, a nucleic acid sequence encoding for a TP901 recombinase comprises the nucleic acid sequence of SEQ ID NO: 187. In some embodiments, an expression control component comprises a polynucleic acid encoding for a TP901 recombinase operably linked to a promoter (as described herein).

In some embodiments, an expression control component comprises a R4 recombinase (or a polynucleic acid molecule encoding the same). In some embodiments, a R4 recombinase comprises an amino acid sequence having at least 80% (e.g., at least 80%, at least 85%, at least 90%, at least 95%, or at least 99%) identity to the amino acid sequence of SEQ ID NO: 146. In some embodiments, a R4 recombinase comprises the amino acid sequence SEQ ID NO: 146. In some embodiments, a nucleic acid sequence encoding for a R4 recombinase comprises a nucleic acid sequence having at least 80% (e.g., at least 80%, at least 85%, at least 90%, at least 95%, or at least 99%) identity to the nucleic acid sequence of SEQ ID NO: 188. In some embodiments, a nucleic acid sequence encoding for a R4 recombinase comprises the nucleic acid sequence of SEQ ID NO: 188. In some embodiments, an expression control component comprises a polynucleic acid encoding for a R4 recombinase operably linked to a promoter (as described herein).

In some embodiments, an expression control component comprises a Dre recombinase (or a polynucleic acid molecule encoding the same). In some embodiments, a Dre recombinase comprises an amino acid sequence having at least 80% (e.g., at least 80%, at least 85%, at least 90%, at least 95%, or at least 99%) identity to the amino acid sequence of SEQ ID NO: 147. In some embodiments, a Dre recombinase comprises the amino acid sequence SEQ ID NO: 147. In some embodiments, a nucleic acid sequence encoding for a Dre recombinase comprises a nucleic acid sequence having at least 80% (e.g., at least 80%, at least 85%, at least 90%, at least 95%, or at least 99%) identity to the nucleic acid sequence of SEQ ID NO: 189. In some embodiments, a nucleic acid sequence encoding for a Dre recombinase comprises the nucleic acid sequence of SEQ ID NO: 189. In some embodiments, an expression control component comprises a polynucleic acid encoding for a Dre recombinase operably linked to a promoter (as described herein).

In some embodiments, an expression control component comprises an Int1 recombinase (or a polynucleic acid molecule encoding the same). In some embodiments, an Int1 recombinase comprises an amino acid sequence having at least 80% (e.g., at least 80%, at least 85%, at least 90%, at least 95%, or at least 99%) identity to the amino acid sequence of SEQ ID NO: 148. In some embodiments, an Int1 recombinase comprises the amino acid sequence SEQ ID NO: 148. In some embodiments, a nucleic acid sequence encoding for an Int 1 recombinase comprises a nucleic acid sequence having at least 80% (e.g., at least 80%, at least 85%, at least 90%, at least 95%, or at least 99%) identity to the nucleic acid sequence of SEQ ID NO: 190. In some embodiments, a nucleic acid sequence encoding for an Int1 recombinase comprises the nucleic acid sequence of SEQ ID NO: 190. In some embodiments, an expression control component comprises a polynucleic acid encoding for an Int1 recombinase operably linked to a promoter (as described herein).

In some embodiments, an expression control component comprises an Int2 recombinase (or a polynucleic acid molecule encoding the same). In some embodiments, an Int2 recombinase comprises an amino acid sequence having at least 80% (e.g., at least 80%, at least 85%, at least 90%, at least 95%, or at least 99%) identity to the amino acid sequence of SEQ ID NO: 149. In some embodiments, an Int2 recombinase comprises the amino acid sequence SEQ ID NO: 149. In some embodiments, a nucleic acid sequence encoding for an Int2 recombinase comprises a nucleic acid sequence having at least 80% (e.g., at least 80%, at least 85%, at least 90%, at least 95%, or at least 99%) identity to the nucleic acid sequence of SEQ ID NO: 191. In some embodiments, a nucleic acid sequence encoding for an Int2 recombinase comprises the nucleic acid sequence of SEQ ID NO: 191. In some embodiments, an expression control component comprises a polynucleic acid encoding for an Int2 recombinase operably linked to a promoter (as described herein).

In some embodiments, an expression control component comprises an Int3 recombinase (or a polynucleic acid molecule encoding the same). In some embodiments, an Int3 recombinase comprises an amino acid sequence having at least 80% (e.g., at least 80%, at least 85%, at least 90%, at least 95%, or at least 99%) identity to the amino acid sequence of SEQ ID NO: 150. In some embodiments, an Int3 recombinase comprises the amino acid sequence SEQ ID NO: 150. In some embodiments, a nucleic acid sequence encoding for an Int3 recombinase comprises a nucleic acid sequence having at least 80% (e.g., at least 80%, at least 85%, at least 90%, at least 95%, or at least 99%) identity to the nucleic acid sequence of SEQ ID NO: 192. In some embodiments, a nucleic acid sequence encoding for an Int3 recombinase comprises the nucleic acid sequence of SEQ ID NO: 192. In some embodiments, an expression control component comprises a polynucleic acid encoding for an Int3 recombinase operably linked to a promoter (as described herein).

In some embodiments, an expression control component comprises an Int4 recombinase (or a polynucleic acid molecule encoding the same). In some embodiments, an Int4 recombinase comprises an amino acid sequence having at least 80% (e.g., at least 80%, at least 85%, at least 90%, at least 95%, or at least 99%) identity to the amino acid sequence of SEQ ID NO: 151. In some embodiments, an Int4 recombinase comprises the amino acid sequence SEQ ID NO: 151. In some embodiments, a nucleic acid sequence encoding for an Int4 recombinase comprises a nucleic acid sequence having at least 80% (e.g., at least 80%, at least 85%, at least 90%, at least 95%, or at least 99%) identity to the nucleic acid sequence of SEQ ID NO: 193. In some embodiments, a nucleic acid sequence encoding for an Int4 recombinase comprises the nucleic acid sequence of SEQ ID NO: 193. In some embodiments, an expression control component comprises a polynucleic acid encoding for an Int4 recombinase operably linked to a promoter (as described herein).

In some embodiments, an expression control component comprises an Int5 recombinase (or a polynucleic acid molecule encoding the same). In some embodiments, an Int5 recombinase comprises an amino acid sequence having at least 80% (e.g., at least 80%, at least 85%, at least 90%, at least 95%, or at least 99%) identity to the amino acid sequence of SEQ ID NO: 152. In some embodiments, an Int5 recombinase comprises the amino acid sequence SEQ ID NO: 152. In some embodiments, a nucleic acid sequence encoding for an Int5 recombinase comprises a nucleic acid sequence having at least 80% (e.g., at least 80%, at least 85%, at least 90%, at least 95%, or at least 99%) identity to the nucleic acid sequence of SEQ ID NO: 194. In some embodiments, a nucleic acid sequence encoding for an Int5 recombinase comprises the nucleic acid sequence of SEQ ID NO: 194. In some embodiments, an expression control component comprises a polynucleic acid encoding for an Int5 recombinase operably linked to a promoter (as described herein).

In some embodiments, an expression control component comprises an Int6 recombinase (or a polynucleic acid molecule encoding the same). In some embodiments, an Int6 recombinase comprises an amino acid sequence having at least 80% (e.g., at least 80%, at least 85%, at least 90%, at least 95%, or at least 99%) identity to the amino acid sequence of SEQ ID NO: 153. In some embodiments, an Int6 recombinase comprises the amino acid sequence SEQ ID NO: 153. In some embodiments, a nucleic acid sequence encoding for an Int6 recombinase comprises a nucleic acid sequence having at least 80% (e.g., at least 80%, at least 85%, at least 90%, at least 95%, or at least 99%) identity to the nucleic acid sequence of SEQ ID NO: 195. In some embodiments, a nucleic acid sequence encoding for an Int6 recombinase comprises the nucleic acid sequence of SEQ ID NO: 195. In some embodiments, an expression control component comprises a polynucleic acid encoding for an Int6 recombinase operably linked to a promoter (as described herein).

In some embodiments, an expression control component comprises an Int7 recombinase (or a polynucleic acid molecule encoding the same). In some embodiments, an Int7 recombinase comprises an amino acid sequence having at least 80% (e.g., at least 80%, at least 85%, at least 90%, at least 95%, or at least 99%) identity to the amino acid sequence of SEQ ID NO: 154. In some embodiments, an Int7 recombinase comprises the amino acid sequence SEQ ID NO: 154. In some embodiments, a nucleic acid sequence encoding for an Int7 recombinase comprises a nucleic acid sequence having at least 80% (e.g., at least 80%, at least 85%, at least 90%, at least 95%, or at least 99%) identity to the nucleic acid sequence of SEQ ID NO: 196. In some embodiments, a nucleic acid sequence encoding for an Int7 recombinase comprises the nucleic acid sequence of SEQ ID NO: 196. In some embodiments, an expression control component comprises a polynucleic acid encoding for an Int7 recombinase operably linked to a promoter (as described herein).

In some embodiments, an expression control component comprises an Int8 recombinase (or a polynucleic acid molecule encoding the same). In some embodiments, an Int8 recombinase comprises an amino acid sequence having at least 80% (e.g., at least 80%, at least 85%, at least 90%, at least 95%, or at least 99%) identity to the amino acid sequence of SEQ ID NO: 155. In some embodiments, an Int8 recombinase comprises the amino acid sequence SEQ ID NO: 155. In some embodiments, a nucleic acid sequence encoding for an Int8 recombinase comprises a nucleic acid sequence having at least 80% (e.g., at least 80%, at least 85%, at least 90%, at least 95%, or at least 99%) identity to the nucleic acid sequence of SEQ ID NO: 197. In some embodiments, a nucleic acid sequence encoding for an Int8 recombinase comprises the nucleic acid sequence of SEQ ID NO: 197. In some embodiments, an expression control component comprises a polynucleic acid encoding for an Int8 recombinase operably linked to a promoter (as described herein).

In some embodiments, an expression control component comprises an Int9 recombinase (or a polynucleic acid molecule encoding the same). In some embodiments, an Int9 recombinase comprises an amino acid sequence having at least 80% (e.g., at least 80%, at least 85%, at least 90%, at least 95%, or at least 99%) identity to the amino acid sequence of SEQ ID NO: 156. In some embodiments, an Int9 recombinase comprises the amino acid sequence SEQ ID NO: 156. In some embodiments, a nucleic acid sequence encoding for an Int9 recombinase comprises a nucleic acid sequence having at least 80% (e.g., at least 80%, at least 85%, at least 90%, at least 95%, or at least 99%) identity to the nucleic acid sequence of SEQ ID NO: 198. In some embodiments, a nucleic acid sequence encoding for an Int9 recombinase comprises the nucleic acid sequence of SEQ ID NO: 198. In some embodiments, an expression control component comprises a polynucleic acid encoding for an Int9 recombinase operably linked to a promoter (as described herein).

In some embodiments, an expression control component comprises an Int10 recombinase (or a polynucleic acid molecule encoding the same). In some embodiments, an Int 10 recombinase comprises an amino acid sequence having at least 80% (e.g., at least 80%, at least 85%, at least 90%, at least 95%, or at least 99%) identity to the amino acid sequence of SEQ ID NO: 157. In some embodiments, an Int10 recombinase comprises the amino acid sequence SEQ ID NO: 157. In some embodiments, a nucleic acid sequence encoding for an Int 10 recombinase comprises a nucleic acid sequence having at least 80% (e.g., at least 80%, at least 85%, at least 90%, at least 95%, or at least 99%) identity to the nucleic acid sequence of SEQ ID NO: 199. In some embodiments, a nucleic acid sequence encoding for an Int10 recombinase comprises the nucleic acid sequence of SEQ ID NO: 199. In some embodiments, an expression control component comprises a polynucleic acid encoding for an Int 10 recombinase operably linked to a promoter (as described herein).

In some embodiments, an expression control component comprises an Int11 recombinase (or a polynucleic acid molecule encoding the same). In some embodiments, an Int11 recombinase comprises an amino acid sequence having at least 80% (e.g., at least 80%, at least 85%, at least 90%, at least 95%, or at least 99%) identity to the amino acid sequence of SEQ ID NO: 158. In some embodiments, an Int11 recombinase comprises the amino acid sequence SEQ ID NO: 158. In some embodiments, a nucleic acid sequence encoding for an Int11 recombinase comprises a nucleic acid sequence having at least 80% (e.g., at least 80%, at least 85%, at least 90%, at least 95%, or at least 99%) identity to the nucleic acid sequence of SEQ ID NO: 200. In some embodiments, a nucleic acid sequence encoding for an Int11 recombinase comprises the nucleic acid sequence of SEQ ID NO: 200. In some embodiments, an expression control component comprises a polynucleic acid encoding for an Int11 recombinase operably linked to a promoter (as described herein).

In some embodiments, an expression control component comprises an Int12 recombinase (or a polynucleic acid molecule encoding the same). In some embodiments, an Int 12 recombinase comprises an amino acid sequence having at least 80% (e.g., at least 80%, at least 85%, at least 90%, at least 95%, or at least 99%) identity to the amino acid sequence of SEQ ID NO: 159. In some embodiments, an Int12 recombinase comprises the amino acid sequence SEQ ID NO: 159. In some embodiments, a nucleic acid sequence encoding for an Int 12 recombinase comprises a nucleic acid sequence having at least 80% (e.g., at least 80%, at least 85%, at least 90%, at least 95%, or at least 99%) identity to the nucleic acid sequence of SEQ ID NO: 201. In some embodiments, a nucleic acid sequence encoding for an Int12 recombinase comprises the nucleic acid sequence of SEQ ID NO: 201. In some embodiments, an expression control component comprises a polynucleic acid encoding for an Int 12 recombinase operably linked to a promoter (as described herein).

In some embodiments, an expression control component comprises an Int13 recombinase (or a polynucleic acid molecule encoding the same). In some embodiments, an Int 13 recombinase comprises an amino acid sequence having at least 80% (e.g., at least 80%, at least 85%, at least 90%, at least 95%, or at least 99%) identity to the amino acid sequence of SEQ ID NO: 160. In some embodiments, an Int13 recombinase comprises the amino acid sequence SEQ ID NO: 160. In some embodiments, a nucleic acid sequence encoding for an Int 13 recombinase comprises a nucleic acid sequence having at least 80% (e.g., at least 80%, at least 85%, at least 90%, at least 95%, or at least 99%) identity to the nucleic acid sequence of SEQ ID NO: 202. In some embodiments, a nucleic acid sequence encoding for an Int13 recombinase comprises the nucleic acid sequence of SEQ ID NO: 202. In some embodiments, an expression control component comprises a polynucleic acid encoding for an Int13 recombinase operably linked to a promoter (as described herein).

In some embodiments, an expression control component comprises an Int14 recombinase (or a polynucleic acid molecule encoding the same). In some embodiments, an Int 14 recombinase comprises an amino acid sequence having at least 80% (e.g., at least 80%, at least 85%, at least 90%, at least 95%, or at least 99%) identity to the amino acid sequence of SEQ ID NO: 161. In some embodiments, an Int14 recombinase comprises the amino acid sequence SEQ ID NO: 161. In some embodiments, a nucleic acid sequence encoding for an Int14 recombinase comprises a nucleic acid sequence having at least 80% (e.g., at least 80%, at least 85%, at least 90%, at least 95%, or at least 99%) identity to the nucleic acid sequence of SEQ ID NO: 203. In some embodiments, a nucleic acid sequence encoding for an Int14 recombinase comprises the nucleic acid sequence of SEQ ID NO: 203. In some embodiments, an expression control component comprises a polynucleic acid encoding for an Int 14 recombinase operably linked to a promoter (as described herein).

In some embodiments, an expression control component comprises an Int15 recombinase (or a polynucleic acid molecule encoding the same). In some embodiments, an Int 15 recombinase comprises an amino acid sequence having at least 80% (e.g., at least 80%, at least 85%, at least 90%, at least 95%, or at least 99%) identity to the amino acid sequence of SEQ ID NO: 162. In some embodiments, an Int15 recombinase comprises the amino acid sequence SEQ ID NO: 162. In some embodiments, a nucleic acid sequence encoding for an Int 15 recombinase comprises a nucleic acid sequence having at least 80% (e.g., at least 80%, at least 85%, at least 90%, at least 95%, or at least 99%) identity to the nucleic acid sequence of SEQ ID NO: 204. In some embodiments, a nucleic acid sequence encoding for an Int15 recombinase comprises the nucleic acid sequence of SEQ ID NO: 204. In some embodiments, an expression control component comprises a polynucleic acid encoding for an Int 15 recombinase operably linked to a promoter (as described herein).

In some embodiments, an expression control component comprises an Int16 recombinase (or a polynucleic acid molecule encoding the same). In some embodiments, an Int 16 recombinase comprises an amino acid sequence having at least 80% (e.g., at least 80%, at least 85%, at least 90%, at least 95%, or at least 99%) identity to the amino acid sequence of SEQ ID NO: 163. In some embodiments, an Int16 recombinase comprises the amino acid sequence SEQ ID NO: 163. In some embodiments, a nucleic acid sequence encoding for an Int16 recombinase comprises a nucleic acid sequence having at least 80% (e.g., at least 80%, at least 85%, at least 90%, at least 95%, or at least 99%) identity to the nucleic acid sequence of SEQ ID NO: 205. In some embodiments, a nucleic acid sequence encoding for an Int16 recombinase comprises the nucleic acid sequence of SEQ ID NO: 205. In some embodiments, an expression control component comprises a polynucleic acid encoding for an Int 16 recombinase operably linked to a promoter (as described herein).

In some embodiments, an expression control component comprises an Int17 recombinase (or a polynucleic acid molecule encoding the same). In some embodiments, an Int 17 recombinase comprises an amino acid sequence having at least 80% (e.g., at least 80%, at least 85%, at least 90%, at least 95%, or at least 99%) identity to the amino acid sequence of SEQ ID NO: 164. In some embodiments, an Int 17 recombinase comprises the amino acid sequence SEQ ID NO: 164. In some embodiments, a nucleic acid sequence encoding for an Int 17 recombinase comprises a nucleic acid sequence having at least 80% (e.g., at least 80%, at least 85%, at least 90%, at least 95%, or at least 99%) identity to the nucleic acid sequence of SEQ ID NO: 206. In some embodiments, a nucleic acid sequence encoding for an Int17 recombinase comprises the nucleic acid sequence of SEQ ID NO: 206. In some embodiments, an expression control component comprises a polynucleic acid encoding for an Int17 recombinase operably linked to a promoter (as described herein).

In some embodiments, an expression control component comprises an Int18 recombinase (or a polynucleic acid molecule encoding the same). In some embodiments, an Int 18 recombinase comprises an amino acid sequence having at least 80% (e.g., at least 80%, at least 85%, at least 90%, at least 95%, or at least 99%) identity to the amino acid sequence of SEQ ID NO: 165. In some embodiments, an Int18 recombinase comprises the amino acid sequence SEQ ID NO: 165. In some embodiments, a nucleic acid sequence encoding for an Int 18 recombinase comprises a nucleic acid sequence having at least 80% (e.g., at least 80%, at least 85%, at least 90%, at least 95%, or at least 99%) identity to the nucleic acid sequence of SEQ ID NO: 207. In some embodiments, a nucleic acid sequence encoding for an Int18 recombinase comprises the nucleic acid sequence of SEQ ID NO: 207. In some embodiments, an expression control component comprises a polynucleic acid encoding for an Int 18 recombinase operably linked to a promoter (as described herein).

In some embodiments, an expression control component comprises an Int19 recombinase (or a polynucleic acid molecule encoding the same). In some embodiments, an Int 19 recombinase comprises an amino acid sequence having at least 80% (e.g., at least 80%, at least 85%, at least 90%, at least 95%, or at least 99%) identity to the amino acid sequence of SEQ ID NO: 166. In some embodiments, an Int19 recombinase comprises the amino acid sequence SEQ ID NO: 166. In some embodiments, a nucleic acid sequence encoding for an Int 19 recombinase comprises a nucleic acid sequence having at least 80% (e.g., at least 80%, at least 85%, at least 90%, at least 95%, or at least 99%) identity to the nucleic acid sequence of SEQ ID NO: 208. In some embodiments, a nucleic acid sequence encoding for an Int19 recombinase comprises the nucleic acid sequence of SEQ ID NO: 208. In some embodiments, an expression control component comprises a polynucleic acid encoding for an Int 19 recombinase operably linked to a promoter (as described herein).

In some embodiments, an expression control component comprises an Int20 recombinase (or a polynucleic acid molecule encoding the same). In some embodiments, an Int20 recombinase comprises an amino acid sequence having at least 80% (e.g., at least 80%, at least 85%, at least 90%, at least 95%, or at least 99%) identity to the amino acid sequence of SEQ ID NO: 167. In some embodiments, an Int20 recombinase comprises the amino acid sequence SEQ ID NO: 167. In some embodiments, a nucleic acid sequence encoding for an Int20 recombinase comprises a nucleic acid sequence having at least 80% (e.g., at least 80%, at least 85%, at least 90%, at least 95%, or at least 99%) identity to the nucleic acid sequence of SEQ ID NO: 209. In some embodiments, a nucleic acid sequence encoding for an Int20 recombinase comprises the nucleic acid sequence of SEQ ID NO: 209. In some embodiments, an expression control component comprises a polynucleic acid encoding for an Int20 recombinase operably linked to a promoter (as described herein).

In some embodiments, an expression control component comprises an Int21 recombinase (or a polynucleic acid molecule encoding the same). In some embodiments, an Int21 recombinase comprises an amino acid sequence having at least 80% (e.g., at least 80%, at least 85%, at least 90%, at least 95%, or at least 99%) identity to the amino acid sequence of SEQ ID NO: 168. In some embodiments, an Int21 recombinase comprises the amino acid sequence SEQ ID NO: 168. In some embodiments, a nucleic acid sequence encoding for an Int21 recombinase comprises a nucleic acid sequence having at least 80% (e.g., at least 80%, at least 85%, at least 90%, at least 95%, or at least 99%) identity to the nucleic acid sequence of SEQ ID NO: 210. In some embodiments, a nucleic acid sequence encoding for an Int21 recombinase comprises the nucleic acid sequence of SEQ ID NO: 210. In some embodiments, an expression control component comprises a polynucleic acid encoding for an Int21 recombinase operably linked to a promoter (as described herein).

In some embodiments, an expression control component comprises an Int22 recombinase (or a polynucleic acid molecule encoding the same). In some embodiments, an Int22 recombinase comprises an amino acid sequence having at least 80% (e.g., at least 80%, at least 85%, at least 90%, at least 95%, or at least 99%) identity to the amino acid sequence of SEQ ID NO: 169. In some embodiments, an Int22 recombinase comprises the amino acid sequence SEQ ID NO: 169. In some embodiments, a nucleic acid sequence encoding for an Int22 recombinase comprises a nucleic acid sequence having at least 80% (e.g., at least 80%, at least 85%, at least 90%, at least 95%, or at least 99%) identity to the nucleic acid sequence of SEQ ID NO: 211. In some embodiments, a nucleic acid sequence encoding for an Int22 recombinase comprises the nucleic acid sequence of SEQ ID NO: 211. In some embodiments, an expression control component comprises a polynucleic acid encoding for an Int22 recombinase operably linked to a promoter (as described herein).

In some embodiments, an expression control component comprises an Int23 recombinase (or a polynucleic acid molecule encoding the same). In some embodiments, an Int23 recombinase comprises an amino acid sequence having at least 80% (e.g., at least 80%, at least 85%, at least 90%, at least 95%, or at least 99%) identity to the amino acid sequence of SEQ ID NO: 170. In some embodiments, an Int23 recombinase comprises the amino acid sequence SEQ ID NO: 170. In some embodiments, a nucleic acid sequence encoding for an Int23 recombinase comprises a nucleic acid sequence having at least 80% (e.g., at least 80%, at least 85%, at least 90%, at least 95%, or at least 99%) identity to the nucleic acid sequence of SEQ ID NO: 212. In some embodiments, a nucleic acid sequence encoding for an Int23 recombinase comprises the nucleic acid sequence of SEQ ID NO: 212. In some embodiments, an expression control component comprises a polynucleic acid encoding for an Int23 recombinase operably linked to a promoter (as described herein).

In some embodiments, an expression control component comprises an Int24 recombinase (or a polynucleic acid molecule encoding the same). In some embodiments, an Int24 recombinase comprises an amino acid sequence having at least 80% (e.g., at least 80%, at least 85%, at least 90%, at least 95%, or at least 99%) identity to the amino acid sequence of SEQ ID NO: 171. In some embodiments, an Int24 recombinase comprises the amino acid sequence SEQ ID NO: 171. In some embodiments, a nucleic acid sequence encoding for an Int24 recombinase comprises a nucleic acid sequence having at least 80% (e.g., at least 80%, at least 85%, at least 90%, at least 95%, or at least 99%) identity to the nucleic acid sequence of SEQ ID NO: 213. In some embodiments, a nucleic acid sequence encoding for an Int24 recombinase comprises the nucleic acid sequence of SEQ ID NO: 213. In some embodiments, an expression control component comprises a polynucleic acid encoding for an Int24 recombinase operably linked to a promoter (as described herein).

In some embodiments, an expression control component comprises an Int25 recombinase (or a polynucleic acid molecule encoding the same). In some embodiments, an Int25 recombinase comprises an amino acid sequence having at least 80% (e.g., at least 80%, at least 85%, at least 90%, at least 95%, or at least 99%) identity to the amino acid sequence of SEQ ID NO: 172. In some embodiments, an Int25 recombinase comprises the amino acid sequence SEQ ID NO: 172. In some embodiments, a nucleic acid sequence encoding for an Int25 recombinase comprises a nucleic acid sequence having at least 80% (e.g., at least 80%, at least 85%, at least 90%, at least 95%, or at least 99%) identity to the nucleic acid sequence of SEQ ID NO: 214. In some embodiments, a nucleic acid sequence encoding for an Int25 recombinase comprises the nucleic acid sequence of SEQ ID NO: 214. In some embodiments, an expression control component comprises a polynucleic acid encoding for an Int25 recombinase operably linked to a promoter (as described herein).

In some embodiments, an expression control component comprises an Int26 recombinase (or a polynucleic acid molecule encoding the same). In some embodiments, an Int26 recombinase comprises an amino acid sequence having at least 80% (e.g., at least 80%, at least 85%, at least 90%, at least 95%, or at least 99%) identity to the amino acid sequence of SEQ ID NO: 173. In some embodiments, an Int26 recombinase comprises the amino acid sequence SEQ ID NO: 173. In some embodiments, a nucleic acid sequence encoding for an Int26 recombinase comprises a nucleic acid sequence having at least 80% (e.g., at least 80%, at least 85%, at least 90%, at least 95%, or at least 99%) identity to the nucleic acid sequence of SEQ ID NO: 215. In some embodiments, a nucleic acid sequence encoding for an Int26 recombinase comprises the nucleic acid sequence of SEQ ID NO: 215. In some embodiments, an expression control component comprises a polynucleic acid encoding for an Int26 recombinase operably linked to a promoter (as described herein).

In some embodiments, an expression control component comprises an Int27 recombinase (or a polynucleic acid molecule encoding the same). In some embodiments, an Int27 recombinase comprises an amino acid sequence having at least 80% (e.g., at least 80%, at least 85%, at least 90%, at least 95%, or at least 99%) identity to the amino acid sequence of SEQ ID NO: 174. In some embodiments, an Int27 recombinase comprises the amino acid sequence SEQ ID NO: 174. In some embodiments, a nucleic acid sequence encoding for an Int27 recombinase comprises a nucleic acid sequence having at least 80% (e.g., at least 80%, at least 85%, at least 90%, at least 95%, or at least 99%) identity to the nucleic acid sequence of SEQ ID NO: 216. In some embodiments, a nucleic acid sequence encoding for an Int27 recombinase comprises the nucleic acid sequence of SEQ ID NO: 216. In some embodiments, an expression control component comprises a polynucleic acid encoding for an Int27 recombinase operably linked to a promoter (as described herein).

In some embodiments, an expression control component comprises an Int28 recombinase (or a polynucleic acid molecule encoding the same). In some embodiments, an Int28 recombinase comprises an amino acid sequence having at least 80% (e.g., at least 80%, at least 85%, at least 90%, at least 95%, or at least 99%) identity to the amino acid sequence of SEQ ID NO: 175. In some embodiments, an Int28 recombinase comprises the amino acid sequence SEQ ID NO: 175. In some embodiments, a nucleic acid sequence encoding for an Int28 recombinase comprises a nucleic acid sequence having at least 80% (e.g., at least 80%, at least 85%, at least 90%, at least 95%, or at least 99%) identity to the nucleic acid sequence of SEQ ID NO: 217. In some embodiments, a nucleic acid sequence encoding for an Int28 recombinase comprises the nucleic acid sequence of SEQ ID NO: 217. In some embodiments, an expression control component comprises a polynucleic acid encoding for an Int28 recombinase operably linked to a promoter (as described herein).

In some embodiments, an expression control component comprises an Int29 recombinase (or a polynucleic acid molecule encoding the same). In some embodiments, an Int29 recombinase comprises an amino acid sequence having at least 80% (e.g., at least 80%, at least 85%, at least 90%, at least 95%, or at least 99%) identity to the amino acid sequence of SEQ ID NO: 176. In some embodiments, an Int29 recombinase comprises the amino acid sequence SEQ ID NO: 176. In some embodiments, a nucleic acid sequence encoding for an Int29 recombinase comprises a nucleic acid sequence having at least 80% (e.g., at least 80%, at least 85%, at least 90%, at least 95%, or at least 99%) identity to the nucleic acid sequence of SEQ ID NO: 218. In some embodiments, a nucleic acid sequence encoding for an Int29 recombinase comprises the nucleic acid sequence of SEQ ID NO: 218. In some embodiments, an expression control component comprises a polynucleic acid encoding for an Int29 recombinase operably linked to a promoter (as described herein).

In some embodiments, an expression control component comprises an Int30 recombinase (or a polynucleic acid molecule encoding the same). In some embodiments, an Int30 recombinase comprises an amino acid sequence having at least 80% (e.g., at least 80%, at least 85%, at least 90%, at least 95%, or at least 99%) identity to the amino acid sequence of SEQ ID NO: 177. In some embodiments, an Int30 recombinase comprises the amino acid sequence SEQ ID NO: 177. In some embodiments, a nucleic acid sequence encoding for an Int30 recombinase comprises a nucleic acid sequence having at least 80% (e.g., at least 80%, at least 85%, at least 90%, at least 95%, or at least 99%) identity to the nucleic acid sequence of SEQ ID NO: 219. In some embodiments, a nucleic acid sequence encoding for an Int30 recombinase comprises the nucleic acid sequence of SEQ ID NO: 219. In some embodiments, an expression control component comprises a polynucleic acid encoding for an Int30 recombinase operably linked to a promoter (as described herein).

In some embodiments, an expression control component comprises an Int31 recombinase (or a polynucleic acid molecule encoding the same). In some embodiments, an Int31 recombinase comprises an amino acid sequence having at least 80% (e.g., at least 80%, at least 85%, at least 90%, at least 95%, or at least 99%) identity to the amino acid sequence of SEQ ID NO: 178. In some embodiments, an Int31 recombinase comprises the amino acid sequence SEQ ID NO: 178. In some embodiments, a nucleic acid sequence encoding for an Int31 recombinase comprises a nucleic acid sequence having at least 80% (e.g., at least 80%, at least 85%, at least 90%, at least 95%, or at least 99%) identity to the nucleic acid sequence of SEQ ID NO: 220. In some embodiments, a nucleic acid sequence encoding for an Int31 recombinase comprises the nucleic acid sequence of SEQ ID NO: 220. In some embodiments, an expression control component comprises a polynucleic acid encoding for an Int31 recombinase operably linked to a promoter (as described herein).

In some embodiments, an expression control component comprises an Int32 recombinase (or a polynucleic acid molecule encoding the same). In some embodiments, an Int32 recombinase comprises an amino acid sequence having at least 80% (e.g., at least 80%, at least 85%, at least 90%, at least 95%, or at least 99%) identity to the amino acid sequence of SEQ ID NO: 179. In some embodiments, an Int32 recombinase comprises the amino acid sequence SEQ ID NO: 179. In some embodiments, a nucleic acid sequence encoding for an Int32 recombinase comprises a nucleic acid sequence having at least 80% (e.g., at least 80%, at least 85%, at least 90%, at least 95%, or at least 99%) identity to the nucleic acid sequence of SEQ ID NO: 221. In some embodiments, a nucleic acid sequence encoding for an Int32 recombinase comprises the nucleic acid sequence of SEQ ID NO: 221. In some embodiments, an expression control component comprises a polynucleic acid encoding for an Int32 recombinase operably linked to a promoter (as described herein).

In some embodiments, an expression control component comprises an Int33 recombinase (or a polynucleic acid molecule encoding the same). In some embodiments, an Int33 recombinase comprises an amino acid sequence having at least 80% (e.g., at least 80%, at least 85%, at least 90%, at least 95%, or at least 99%) identity to the amino acid sequence of SEQ ID NO: 180. In some embodiments, an Int33 recombinase comprises the amino acid sequence SEQ ID NO: 180. In some embodiments, a nucleic acid sequence encoding for an Int33 recombinase comprises a nucleic acid sequence having at least 80% (e.g., at least 80%, at least 85%, at least 90%, at least 95%, or at least 99%) identity to the nucleic acid sequence of SEQ ID NO: 222. In some embodiments, a nucleic acid sequence encoding for an Int33 recombinase comprises the nucleic acid sequence of SEQ ID NO: 222. In some embodiments, an expression control component comprises a polynucleic acid encoding for an Int33 recombinase operably linked to a promoter (as described herein).

In some embodiments, an expression control component comprises an Int34 recombinase (or a polynucleic acid molecule encoding the same). In some embodiments, an Int34 recombinase comprises an amino acid sequence having at least 80% (e.g., at least 80%, at least 85%, at least 90%, at least 95%, or at least 99%) identity to the amino acid sequence of SEQ ID NO: 181. In some embodiments, an Int34 recombinase comprises the amino acid sequence SEQ ID NO: 181. In some embodiments, a nucleic acid sequence encoding for an Int34 recombinase comprises a nucleic acid sequence having at least 80% (e.g., at least 80%, at least 85%, at least 90%, at least 95%, or at least 99%) identity to the nucleic acid sequence of SEQ ID NO: 223. In some embodiments, a nucleic acid sequence encoding for an Int34 recombinase comprises the nucleic acid sequence of SEQ ID NO: 223. In some embodiments, an expression control component comprises a polynucleic acid encoding for an Int34 recombinase operably linked to a promoter (as described herein).

c. Transcriptional Activator

In some embodiments, an AAV production system further comprises a transcriptional activator (or a polynucleic acid molecule encoding the same). As used herein, the term “transcriptional activator” refers to a transcription factor that binds to and regulates expression of an inducible promoter of an AAV production system (e.g., an inducible promoter operably linked to a nucleic acid sequence encoding for an AAV gene product, an inducible promoter operably linked to a nucleic acid encoding for a recombinase, etc.). Exemplary transcriptional activators, and their corresponding promoter recognition sites, are known to those having skill in the art and include, but are not limited to, TetOn-3G, TetOn-V16, TetOff-Advanced, VanR-VP16, TtgR-VP16, Ph1F-VP16, and the cumate cTA and rcTA. In some embodiments, the transcriptional activator is operably linked to a promoter (as described herein). In some embodiments, the transcriptional activator binds to its corresponding promoter recognition site when exposed to a small molecule inducer. In some embodiments, the small molecule inducer is selected from the group consisting of doxycycline, vanillate, phloretin, rapamycin, abscisic acid, gibberellic acid acetoxymethyl ester, and cumate.

In some embodiments, an AAV production system comprises two or more recombinases (or polynucleic acid molecules encoding the same).

In some embodiments, an AAV production system comprises a TetOff-Advanced transcriptional activator (or a polynucleic acid molecule encoding the same). In some embodiments, TetOff-Advanced comprises an amino acid sequence having at least 80% (e.g., at least 80%, at least 85%, at least 90%, at least 95%, or at least 99%) identity to the amino acid sequence of SEQ ID NO: 224. In some embodiments, TetOff-Advanced comprises the amino acid sequence SEQ ID NO: 224. In some embodiments, an AAV production system comprises a polynucleic acid encoding for a TetOff-Advanced transcriptional activator operably linked to a promoter (as described herein).

In some embodiments, an AAV production system comprises a VanR-VP16 transcriptional activator (or a polynucleic acid molecule encoding the same). In some embodiments, VanR-VP16 comprises an amino acid sequence having at least 80% (e.g., at least 80%, at least 85%, at least 90%, at least 95%, or at least 99%) identity to the amino acid sequence of SEQ ID NO: 225. In some embodiments, VanR-VP16 comprises the amino acid sequence SEQ ID NO: 225. In some embodiments, an AAV production system comprises a polynucleic acid encoding for a VanR-VP16 transcriptional activator operably linked to a promoter (as described herein).

In some embodiments, an AAV production system comprises a TtgR-VP16 transcriptional activator (or a polynucleic acid molecule encoding the same). In some embodiments, TtgR-VP16 comprises an amino acid sequence having at least 80% (e.g., at least 80%, at least 85%, at least 90%, at least 95%, or at least 99%) identity to the amino acid sequence of SEQ ID NO: 226. In some embodiments, TtgR-VP16 comprises the amino acid sequence SEQ ID NO: 226. In some embodiments, an AAV production system comprises a polynucleic acid encoding for a TtgR-VP16 transcriptional activator operably linked to a promoter (as described herein).

In some embodiments, an AAV production system comprises a Ph1F-VP16 transcriptional activator (or a polynucleic acid molecule encoding the same). In some embodiments, Ph1F-VP16 comprises an amino acid sequence having at least 80% (e.g., at least 80%, at least 85%, at least 90%, at least 95%, or at least 99%) identity to the amino acid sequence of SEQ ID NO: 227. In some embodiments, Ph1F-VP16 comprises the amino acid sequence SEQ ID NO: 227. In some embodiments, an AAV production system comprises a polynucleic acid encoding for a Ph1F-VP16 transcriptional activator operably linked to a promoter (as described herein).

In some embodiments, an AAV production system comprises a cTA transcriptional activator (or a polynucleic acid molecule encoding the same). In some embodiments, cTA comprises an amino acid sequence having at least 80% (e.g., at least 80%, at least 85%, at least 90%, at least 95%, or at least 99%) identity to the amino acid sequence of SEQ ID NO: 228. In some embodiments, cTA comprises the amino acid sequence SEQ ID NO: 228. In some embodiments, an AAV production system comprises a polynucleic acid encoding for a cTA transcriptional activator operably linked to a promoter (as described herein).

d. Transfer Polynucleic Acid Molecule

In some embodiments, an AAV production system further comprises a transfer polynucleic acid molecule. In some embodiments, a transfer polynucleic acid molecule comprises, from 5′ to 3′: (i) a nucleic acid sequence of a 5′ inverted tandem repeat; (ii) a central nucleic acid; and (iii) a nucleic acid sequence of a 3′ inverted tandem repeat. In some embodiments, the nucleic acid sequence is a plasmid or a vector.

In some embodiments, a central nucleic acid of the transfer polynucleic acid molecule comprises a multiple cloning site. Exemplary multiple cloning sites are known to those having ordinary skill in the art. A multiple cloning site can be used for cloning a payload molecule (or gene of interest)—or an expression cassette encoding a payload molecule-into the transfer nucleic acid molecule prior to the generation of viral vectors in a host cell.

In some embodiments, a central nucleic acid of the transfer polynucleic acid molecule comprises a gene product of interest.

e. Selection Marker

A polynucleic acid of the AAV production system may further comprise a nucleic acid sequence encoding for a selection marker. As used herein, the term “selection marker” or refers to a protein that-when introduced into or expressed in a cell-confers a trait that is suitable for selection. As used herein, the term “selection cassette” refers to a nucleic acid sequence encoding a selection marker operably linked to a promoter (as described herein) and a terminator.

A selection marker may be a fluorescent protein. Examples of fluorescent proteins are known in the art (e.g., TagBFP, EBFP2, EGFP, EYFP, mKO2, or Sirius). See e.g., U.S. Pat. No. 5,874,304; Patent No.: EP 0969284 A1; Pub. No.: US 2010/167394 A—the entireties of which are incorporated here by reference.

Alternatively, or in addition, a selection marker may be an antibiotic resistance protein. Examples of antibiotic resistance proteins are known in the art (e.g., facilitating puromycin, hygromycin, neomycin, zeocin, blasticidin, or phleomycin selection). See e.g., Pub. No.: WO 1997/15668 A2; Pub. No.: WO 1997/43900 A1—the entireties of which are incorporated here by reference.

Alternatively, or in addition, a selection marker may be an auxotrophic selection marker (e.g., glutamine synthetase).

f. Percent Identity

As used herein, the term “percent identity” (or “% identity”) refers to a relationship between the sequences of two polypeptides or polynucleotides, as determined by sequence comparison (alignment). In some embodiments, identity is determined across the entire length of a sequence. In some embodiments, identity is determined over a region of a sequence. Identity of related polypeptides or nucleic acid sequences can be readily calculated by those having ordinary skill in the art. For example, the percent identity of two sequences (e.g., nucleic acid or amino acid sequences) may be determined using BLAST®, NBLAST®, XBLAST®, Gapped BLAST®, and Clustal Omega programs, using default parameters of the respective programs. In some embodiments, the identity of two polypeptides is determined by aligning the two amino acid sequences, calculating the number of identical amino acids, and dividing by the length of one of the amino acid sequences. In some embodiments, the identity of two nucleic acids is determined by aligning the two nucleotide sequences and calculating the number of identical nucleotides and dividing by the length of one of the nucleic acids.

### II. Engineered Cells for AAV Production

In aspects, the disclosure relates to engineered cells for AAV production. In some embodiments, the engineered cell may comprise any part (and any combination of parts) of the AAV production systems described herein.

For example, an engineered cell may comprise at least a portion of the AAV production component. For example, and as described above, an AAV production component may comprise multiple nucleic acid molecules. In such embodiments, an engineered cell comprises one or more of said multiple nucleic acid molecules—each of which may be located extra-chromosomally or stably integrated into the genome of the engineered cell. In some embodiments, an engineered cell comprises the entire AAV production component.

Alternatively, or in addition, an engineered cell may comprise the expression control component of the AAV production system. In such embodiments, an engineered cell comprises one or more of said multiple nucleic acid molecules—each of which may be located extra-chromosomally or stably integrated into the genome of the engineered cell. In some embodiments, an engineered cell comprises the entire expression control component.

Alternatively, or in addition, an engineered cell may comprise a polynucleic acid molecule comprising the nucleic acid sequence of a transcriptional activator, which may be located extra-chromosomally or stably integrated into the genome of the engineered cell.

Alternatively, or in addition, an engineered cell may comprise a transfer polynucleic acid molecule, which may be located extra-chromosomally or stably integrated into the genome of the engineered cell.

In some embodiments, an AAV production system comprises: (a) an engineered cell comprising an AAV production component comprising one or more heterologous nucleic acid molecules that collectively encode the genes required for AAV production and (b) an expression control component capable of controlling expression of at least one gene required for AAV production.

As used herein, the term “stably integrated” refers to an exogenous nucleic acid sequence, nucleic acid molecule, construct, gene, or nucleic acid sequence that has been inserted into the genome of and organism (e.g. the engineered cell as described herein) and is passed on to future generations after cell division. It is to be understood that any nucleic acid sequence, nucleic acid molecule, construct, gene or nucleic acid sequence described herein may be stably integrated. In some embodiments, any nucleic acid sequence, nucleic acid molecule, construct gene or nucleic acid sequence may be integrated into the genome using random integration, targeted integration, or transposon-mediated integration. It is to be understood that any of the stably integrated nucleic acid molecules described herein may comprise IR/DR sequences that are capable of binding the Sleeping Beauty transposase. Stable integration using the Sleeping Beauty transposase is described in Mátés, Lajos, et al. Nature genetics 41.6 (2009): 753-761 which is incorporated by reference in its entirety. In some embodiments, a IR/DR sequence comprises a Sleeping Beauty 100X (SB100X) IR/DR.

An engineered cell described herein may further comprise a landing pad. As used herein, the term “landing pad” refers to a heterologous nucleic acid molecule sequence that facilitates the targeted insertion of a “payload” sequence into a specific locus (or multiple loci) of the cell's genome. Accordingly, the landing pad is integrated into the genome of the cell. A fixed integration site is desirable to reduce the variability between experiments that may be caused by positional epigenetic effects or proximal regulatory elements. The ability to control payload copy number is also desirable to modulate expression levels of the payload without changing any genetic components.

In some embodiments, the landing pad is located at a safe harbor site in the genome of the engineered cell. As used herein, the term “safe harbor site” refers to a location in the genome where genes or genetic elements can be introduced without disrupting the expression or regulation of adjacent genes and/or adjacent genomic elements do not disrupt expression or regulation of the introduced genes or genetic elements. Examples of safe harbor sites are known to those having skill in the art and include, but are not limited to, AAVS1, ROSA26, COSMIC, H11, CCR5, and LiPS-A3S. See e.g., Gaidukov et al., Nucleic Acids Res. 2018 May 4; 46(8):4072-4086; U.S. Pat. Nos. 8,980,579 B2; 10,017,786 B2; 9,932,607 B2; Pub. No.: US 2013/280222 A; Pub. No.: WO 2017/180669 A1—the entireties of which are incorporated herein. In some embodiments, the safe harbor site is a known site. In other embodiments, the safe harbor site is a previously undisclosed site. See “Methods of Identifying High-Expressing Genomic Loci and Uses Thereof” herein. In some embodiments, an engineered cell described herein comprises a landing pad that is integrated at a safe harbor locus selected from the group consisting of AAVS1, ROSA26, COSMIC, H11, CCR5, and LiPS-A3S.

In some embodiments, the engineered cell is derived from a HEK293 cell. In some embodiments, the engineered HEK293 cell comprises a landing pad that is integrated at a safe harbor locus selected from the group consisting of AAVS1, ROSA26, COSMIC, H11, CCR5, and LiPS-A3S.

Each of the landing pads described herein comprises at least one recombination site. Recombinase attachment sites for various integrases have been identified previously. For example, a landing pad may comprise recombinase attachment sites corresponding to a Bxb1 integrase, lambda-integrase, Cre recombinase, F1p recombinase, gamma-delta resolvase, Tn3 resolvase, φC31 integrase, or R4 integrase. Exemplary recombination site sequences are known in the art (e.g., attP, attB, attR, attL, Lox, and Frt).

The landing pads described herein may comprise one or more expression cassettes.

### III. Kits

In some aspects, the disclosure relates to kits comprising an AAV production system described herein and/or an engineered cell described herein.

In some embodiments, a kit comprises one or more nucleic acid molecules collectively comprising an AAV production system.

In some embodiments, the kit further comprises a small molecule inducer corresponding to a chemically inducible promoter of the AAV production system. In some embodiments, a small molecule inducer is doxycycline, vanillate, phloretin, rapamycin, abscisic acid, gibberellic acid acetoxymethyl ester, and cumate.

In some embodiments, a kit comprises a nucleic acid molecule comprising a nucleic acid sequence of a transcriptional activator operably linked to a nucleic acid sequence of a promoter, wherein the transcriptional activator, when expressed in the presence of the small molecule inducer, binds to a chemically inducible promoter of the AAV production system, optionally wherein an engineered cell comprises the nucleic acid molecule comprising the nucleic acid sequence of the transcriptional activator. In some embodiments, the transcriptional activator is selected from the group consisting of TetOn-3G, TetOn-V16, TetOff-Advanced, VanR-VP16, TtgR-VP16, Ph1F-VP16, and the cumate cTA and rcTA.

In some embodiments, the kit may further comprise instructions for use of the cells.

### IV. Methods of Using Engineered Cells for AAV Production

In some aspects, the present disclosure provides methods for producing AAV using an AAV production system described herein that comprises: (a) an AAV production component collectively encode gene products required for generation of an AAV in a recombinant host cell; and (b) an expression control component. In some embodiments, the method of AAV production comprises transfecting or stably integrating into an engineered cell any combination of the one or more nucleic acid molecules collectively comprising the AAV production component and the expression control component as described herein. In some embodiments, the method of AAV production further comprises transfecting a nucleic acid molecule comprising a payload for AAV delivery (e.g. a therapeutic DNA sequence) as described above. In some embodiments, the method comprises growing the engineered cell to a confluency that is optimal for AAV production. An optimal confluency may be dependent, for example, on the type of cell the engineered cell is derived from. The skilled person will know or be able to determine the optimal confluency for AAV production. In some embodiments, the method comprises harvesting the AAV produced from the culture of engineered cells using methods that are well known to those of skill in the art.

### EXAMPLES

Viral vectors are a promising gene delivery modality for cell and gene therapy. The production of viral vectors normally entails transient transfection of plasmids into cell culture. However, stable integration of genes necessary to produce therapeutic viral vectors into the genome offers several advantages compared to traditional production via transient transfection. Since cells amplify the viral genes during their own cell division, large quantities of DNA and transfection reagent no longer need to be procured for the transfection process, reducing costs. Also since the DNA is already within the nucleus, viral titers may be higher and more consistent due to minimal numbers of “untransfected” cells and reduced variation associated with transfection steps. The simpler production process also saves scientist time.

However, several genes required for adeno-associated viral (AAV) vector production have been demonstrated by others to be cytostatic or cytotoxic, namely Rep, E2A and E4. The cytotoxic and cytostatic nature of these proteins has hampered the development of stable AAV producer cell lines in the widely used HEK293 cell line, since the native expression of adenovirus E1 genes in HEK293 cells upregulates expression of these toxic genes. Cells stably transfected with these genes fail to survive selection steps or have silenced expression, resulting in an inability to produce relevant quantities of AAV.

The following designs introduce inducible control of cytostatic or cytotoxic genes using recombinases. Each of the described constructs can be integrated into the genome using random integration, targeted integration, or transposon-mediated integration.

### Example 1: AAV Expression Control with a Recombinase

### Description of Approach and Genetic Schematic:

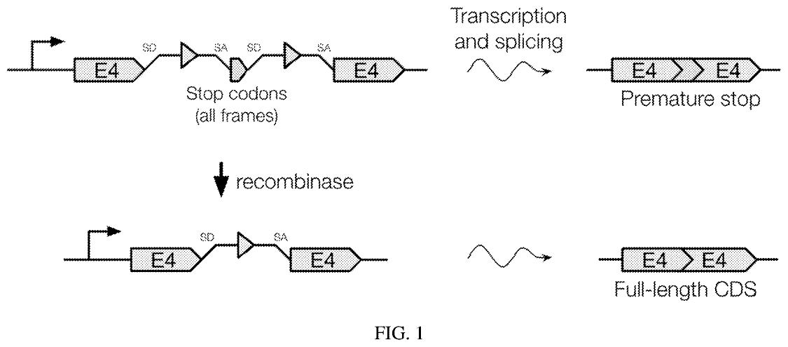

The AAV production systems described in this example utilize a stop codon-containing exon flanked by introns bearing recombinase sites (e.g., lox sites). Stop codons may be inserted into all three reading frames to ensure proper translational termination. This lox-flanked exon is inserted into AAV helper genes or AAV rep/cap genes in order to downregulate expression, minimizing toxicity. In the absence of recombinase, the 5′ intron is spliced out, resulting in a transcript with a premature stop codon. In the presence of recombinase, the exon is recombined out, resulting in a single functional intron that when spliced results in the original coding sequence (FIG. 1). Excisable exons can be placed within one or several AAV helper or AAV rep/cap genes in combination to further reduce background AAV production and toxicity.

### Preliminary Data and Experiment Description:

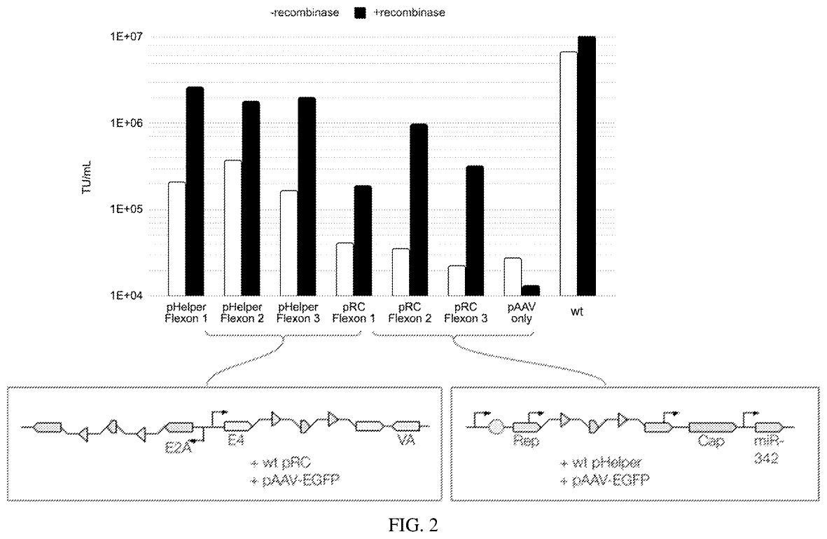

Adherent HEK293T cells were co-transfected with standard control plasmids: EGFP-expressing transfer plasmid, pRepCap, and pHelper. Additionally, variants of pRep, pCap and pHelper containing excisable exons (e.g., pHelper Flexon 1, pHelper Flexon 2, pHelper Flexon 3, pRC Flexon 1, pRC Flexon 2, and pHelper Flexon 3) were used instead of standard control plasmids to test the ability to produce AAV through additional co-transfection of a Cre expression plasmid (FIG. 2). Positive control samples were also prepared containing standard AAV2 pRepCap, pHelper plasmids, and pAAV plasmids, and negative control samples containing only pAAV transfer plasmid. 72 hours after transfection, AAV was harvested by two freeze thaw cycles in a dry ice isopropanol bath. Virus stock was serially diluted 1-, 10- and 100-fold and then 10 μL of the resulting viral stocks were transduced by addition to 5e4 HEK293FT cells plated in a 96-well plate. 72 hours after transduction, transduced cells were harvested and percentage of EGFP positive cells was determined by flow cytometry and used to calculate transducing units per mL (TU/mL) (FIG. 2; pHelper Flexon 1, 2.06E+05 TU/mL without recombinase, 2.60E+06 TU/mL with recombinase; pHelper Flexon 2, 3.74E+05 TU/mL without recombinase, 1.77E+06 TU/mL with recombinase; pHelper Flexon 3, 1.65E+05 TU/mL without recombinase, 1.96E+06 TU/mL with recombinase; pRC Flexon 1, 4.03E+04 1.65E+05 TU/mL without recombinase, 1.86E+05 TU/mL with recombinase; pRC Flexon 2, 3.48E+04 TU/mL without recombinase, 9.71E+05 TU/mL with recombinase; pRC Flexon 3, 2.20E+04 TU/mL without recombinase, 3.15E+05 TU/mL with recombinase; pAAV only 2.73E+04 TU/mL without recombinase, 1.29E+04 TU/mL with recombinase; wt, 6.59E+06 TU/mL without recombinase, 1.02E+07 TU/mL with recombinase).

TABLE 1
Exemplary Intron Splice Donors/Acceptors

| SEQ ID | | |
|---|---|---|
| NO: | Description. | Sequence |
| 1 | Intron Splice | GTAAGTTAAATTAACATCGC |
| | Donor A | |
| 2 | Intron Splice | GTGAGTTGATATAACTCACC |
| | Donor B | |
| 3 | Intron Splice | GGCTGACATGATCTCACATATGAATACCTCCTCCTTCTCCT |
| | Acceptor A | TCTTCCTTGCAG |
| 4 | Intron Splice | ATGGCTGACTATACCTCACGCATCATTGGCATCCCCTCTCT |
| | Acceptor B | CCCTCTCTCTCACAG |

TABLE 2
Exemplary Recombinase Attachment Sites

| SEQ ID | | |
|---|---|---|
| NO: | Description. | Sequence |
| 5 | PhiC31 attB [AA] | GTGCGGGTGCCAGGGCGTGCCCAAGGGCTCCCCGGGCGC |
| | | GTACTCC |
| 6 | PhiC31 attB [AC] | GTGCGGGTGCCAGGGCGTGCCCACGGGCTCCCCGGGCGC |
| | | GTACTCC |
| 7 | PhiC31 attB [AG] | GTGCGGGTGCCAGGGCGTGCCCAGGGGCTCCCCGGGCGC |
| | | GTACTCC |
| 8 | PhiC31 attB [AT] | GTGCGGGTGCCAGGGCGTGCCCATGGGCTCCCCGGGCGCG |
| | | TACTCC |
| 9 | PhiC31 attB [CA] | GTGCGGGTGCCAGGGCGTGCCCCAGGGCTCCCCGGGCGC |
| | | GTACTCC |
| 10 | PhiC31 attB [CC] | GTGCGGGTGCCAGGGCGTGCCCCCGGGCTCCCCGGGCGCG |
| | | TACTCC |
| 11 | PhiC31 attB [CG] | GTGCGGGTGCCAGGGCGTGCCCCGGGGCTCCCCGGGCGC |
| | | GTACTCC |
| 12 | PhiC31 attB [CT] | GTGCGGGTGCCAGGGCGTGCCCCTGGGCTCCCCGGGCGCG |
| | | TACTCC |
| 13 | PhiC31 attB [GA] | GTGCGGGTGCCAGGGCGTGCCCGAGGGCTCCCCGGGCGC |
| | | GTACTCC |
| 14 | PhiC31 attB [GC] | GTGCGGGTGCCAGGGCGTGCCCGCGGGCTCCCCGGGCGC |
| | | GTACTCC |
| 15 | PhiC31 attB [GG] | GTGCGGGTGCCAGGGCGTGCCCGGGGGCTCCCCGGGCGC |
| | | GTACTCC |
| 16 | PhiC31 attB [GT] | GTGCGGGTGCCAGGGCGTGCCCGTGGGCTCCCCGGGCGCG |
| | | TACTCC |
| 17 | PhiC31 attB [TA] | GTGCGGGTGCCAGGGCGTGCCCTAGGGCTCCCCGGGCGCG |
| | | TACTCC |
| 18 | PhiC31 attB [TC] | GTGCGGGTGCCAGGGCGTGCCCTCGGGCTCCCCGGGCGCG |
| | | TACTCC |
| 19 | PhiC31 attB [TG] | GTGCGGGTGCCAGGGCGTGCCCTGGGGCTCCCCGGGCGCG |
| | | TACTCC |
| 20 | PhiC31 attB [TT] | GTGCGGGTGCCAGGGCGTGCCCTTGGGCTCCCCGGGCGCG |
| | | TACTCC |
| 21 | PhiC31 attP [AA] | AGTGCCCCAACTGGGGTAACCTAAGAGTTCTCTCAGTTGG |
| | | GGGCGT |
| 22 | PhiC31 attP [AC] | AGTGCCCCAACTGGGGTAACCTACGAGTTCTCTCAGTTGG |
| | | GGGCGT |
| 23 | PhiC31 attP [AG] | AGTGCCCCAACTGGGGTAACCTAGGAGTTCTCTCAGTTGG |
| | | GGGCGT |
| 24 | PhiC31 attP [AT] | AGTGCCCCAACTGGGGTAACCTATGAGTTCTCTCAGTTGG |
| | | GGGCGT |
| 25 | PhiC31 attP [CA] | AGTGCCCCAACTGGGGTAACCTCAGAGTTCTCTCAGTTGG |
| | | GGGCGT |
| 26 | PhiC31 attP [CC] | AGTGCCCCAACTGGGGTAACCTCCGAGTTCTCTCAGTTGG |
| | | GGGCGT |
| 27 | PhiC31 attP [CG] | AGTGCCCCAACTGGGGTAACCTCGGAGTTCTCTCAGTTGG |
| | | GGGCGT |
| 28 | PhiC31 attP [CT] | AGTGCCCCAACTGGGGTAACCTCTGAGTTCTCTCAGTTGG |
| | | GGGCGT |
| 29 | PhiC31 attP [GA] | AGTGCCCCAACTGGGGTAACCTGAGAGTTCTCTCAGTTGG |
| | | GGGCGT |
| 30 | PhiC31 attP [GC] | AGTGCCCCAACTGGGGTAACCTGCGAGTTCTCTCAGTTGG |
| | | GGGCGT |
| 31 | PhiC31 attP [GG] | AGTGCCCCAACTGGGGTAACCTGGGAGTTCTCTCAGTTGG |
| | | GGGCGT |
| 32 | PhiC31 attP [GT] | AGTGCCCCAACTGGGGTAACCTGTGAGTTCTCTCAGTTGG |
| | | GGGCGT |
| 33 | PhiC31 attP [TA] | AGTGCCCCAACTGGGGTAACCTTAGAGTTCTCTCAGTTGG |
| | | GGGCGT |
| 34 | PhiC31 attP [TC] | AGTGCCCCAACTGGGGTAACCTTCGAGTTCTCTCAGTTGG |
| | | GGGCGT |
| 35 | PhiC31 attP [TG] | AGTGCCCCAACTGGGGTAACCTTGGAGTTCTCTCAGTTGG |
| | | GGGCGT |
| 36 | PhiC31 attP [TT] | AGTGCCCCAACTGGGGTAACCTTTGAGTTCTCTCAGTTGG |
| | | GGGCGT |
| 37 | Bxb1 attB [AA] | GGCTTGTCGACGACGGCGAACTCCGTCGTCAGGATCAT |
| 38 | Bxb1 attB [AC] | GGCTTGTCGACGACGGCGACCTCCGTCGTCAGGATCAT |
| 39 | Bxb1 attB [AG] | GGCTTGTCGACGACGGCGAGCTCCGTCGTCAGGATCAT |
| 40 | Bxb1 attB [AT] | GGCTTGTCGACGACGGCGATCTCCGTCGTCAGGATCAT |
| 41 | Bxb1 attB [CA] | GGCTTGTCGACGACGGCGCACTCCGTCGTCAGGATCAT |
| 42 | Bxb1 attB [CC] | GGCTTGTCGACGACGGCGCCCTCCGTCGTCAGGATCAT |
| 43 | Bxb1 attB [CG] | GGCTTGTCGACGACGGCGCGCTCCGTCGTCAGGATCAT |
| 44 | Bxb1 attB [CT] | GGCTTGTCGACGACGGCGCTCTCCGTCGTCAGGATCAT |
| 45 | Bxb1 attB [GA] | GGCTTGTCGACGACGGCGGACTCCGTCGTCAGGATCAT |
| 46 | Bxb1 attB [GC] | GGCTTGTCGACGACGGCGGCCTCCGTCGTCAGGATCAT |
| 47 | Bxb1 attB [GG] | GGCTTGTCGACGACGGCGGGCTCCGTCGTCAGGATCAT |
| 48 | Bxb1 attB [GT] | GGCTTGTCGACGACGGCGGTCTCCGTCGTCAGGATCAT |
| 49 | Bxb1 attB [TA] | GGCTTGTCGACGACGGCGTACTCCGTCGTCAGGATCAT |
| 50 | Bxb1 attB [TC] | GGCTTGTCGACGACGGCGTCCTCCGTCGTCAGGATCAT |
| 51 | Bxb1 attB [TG] | GGCTTGTCGACGACGGCGTGCTCCGTCGTCAGGATCAT |
| 52 | Bxb1 attB [TT] | GGCTTGTCGACGACGGCGTTCTCCGTCGTCAGGATCAT |
| 53 | Bxb1 attP [AA] | GGTTTGTCTGGTCAACCACCGCGAACTCAGTGGTGTACGG |
| | | TACAAACC |
| 54 | Bxb1 attP [AC] | GGTTTGTCTGGTCAACCACCGCGACCTCAGTGGTGTACGG |
| | | TACAAACC |
| 55 | Bxb1 attP [AG] | GGTTTGTCTGGTCAACCACCGCGAGCTCAGTGGTGTACGG |
| | | TACAAACC |
| 56 | Bxb1 attP [AT] | GGTTTGTCTGGTCAACCACCGCGATCTCAGTGGTGTACGG |
| | | TACAAACC |
| 57 | Bxb1 attP [CA] | GGTTTGTCTGGTCAACCACCGCGCACTCAGTGGTGTACGG |
| | | TACAAACC |
| 58 | Bxb1 attP [CC] | GGTTTGTCTGGTCAACCACCGCGCCCTCAGTGGTGTACGG |
| | | TACAAACC |
| 59 | Bxb1 attP [CG] | GGTTTGTCTGGTCAACCACCGCGCGCTCAGTGGTGTACGG |
| | | TACAAACC |
| 60 | Bxb1 attP [CT] | GGTTTGTCTGGTCAACCACCGCGCTCTCAGTGGTGTACGG |
| | | TACAAACC |
| 61 | Bxb1 attP [GA] | GGTTTGTCTGGTCAACCACCGCGGACTCAGTGGTGTACGG |
| | | TACAAACC |
| 62 | Bxb1 attP [GC] | GGTTTGTCTGGTCAACCACCGCGGCCTCAGTGGTGTACGG |
| | | TACAAACC |
| 63 | Bxb1 attP [GG] | GGTTTGTCTGGTCAACCACCGCGGGCTCAGTGGTGTACGG |
| | | TACAAACC |
| 64 | Bxb1 attP [GT] | GGTTTGTCTGGTCAACCACCGCGGTCTCAGTGGTGTACGG |
| | | TACAAACC |
| 65 | Bxb1 attP [TA] | GGTTTGTCTGGTCAACCACCGCGTACTCAGTGGTGTACGG |
| | | TACAAACC |
| 66 | Bxb1 attP [TC] | GGTTTGTCTGGTCAACCACCGCGTCCTCAGTGGTGTACGG |
| | | TACAAACC |
| 67 | Bxb1 attP [TG] | GGTTTGTCTGGTCAACCACCGCGTGCTCAGTGGTGTACGG |
| | | TACAAACC |
| 68 | Bxb1 attP [TT] | GGTTTGTCTGGTCAACCACCGCGTTCTCAGTGGTGTACGG |
| | | TACAAACC |
| 69 | lox66 | ATAACTTCGTATAGCATACATTATACGAACGGTA |
| 70 | lox71 | TACCGttcgtataGCATACATtatacgaagttat |
| 71 | lox511 | ataacttcgtataatgtatActatacgaagttat |
| 72 | lox2272 | ataacttcgtataGgATACtTtatacgaagttat |
| 73 | lox5171 | ataacttcgtataatgtGtActatacgaagttat |
| 74 | loxKR3 | ataacttcgtataGCATACATtatacCTTgttat |
| 75 | loxM2/71 | TACCGTTCGTATATGGTTTCTTATACGAAGTTAT |
| 76 | loxN | ATAACTTCGTATAgtatacctTATACGAAGTTAT |
| 77 | loxP | ataacttcgtatagcatacattatacgaagttat |
| 78 | VloxP | TCAATTTCTGAGAACTGTCATTCTCGGAAATTGA |
| 79 | Vlox2272 | TCAATTTCTGAGAAGTGTCTTTCTCGGAAATTGA |
| 80 | VloxM1 (v1) | TCAATTTCCGAGAACTGTCATTCTCGGAAATTGA |
| 81 | VloxM1 (v2) | TCAATTTCTGAGAACTGTCATTCTCAGAAATTGA |
| 82 | Vlox43R | TCAATTTCTGAGAACTGTCATTCTCGGAATACCT |
| 83 | Vlox43L | CGTGATTCTGAGAACTGTCATTCTCGGAAATTGA |
| 84 | FRT | gaagttcctattctctagaaagtataggaacttc |
| 85 | FRT F1 | gaagttcctattctctagatagtataggaacttc |
| 86 | FRT F2 | gaagttcctattctctacttagtataggaacttc |
| 87 | FRT F3 | gaagttcctattcttcaaatagtataggaacttc |
| 88 | FRT F4 | gaagttcctattctctagaaggtataggaacttc |
| 89 | FRT F5 | gaagttcctattcttcaaaaggtataggaacttc |
| 90 | FRT F10 | gaagttcctattcactagaatgtataggaacttc |
| 91 | FRT F11 | gaagttcctattctgaactaagtataggaacttc |
| 92 | FRT F12 | gaagttcctattctttctgaagtataggaacttc |
| 93 | FRT F13 | gaagttcctattctcatataagtataggaacttc |
| 94 | FRT F14 | Gaagttcctattctatcagaagtataggaacttc |
| 95 | FRT F15 | gaagttcctattcttataggagtataggaacttc |
| 96 | FRT F16 | gaagttcctattctccgggcagtataggaacttc |

TABLE 3
Exemplary AAV Gene Product Amino Acid Sequences

| SEQ ID | | |
|---|---|---|
| NO: | Description. | Sequence |
| 97 | Rep52 (wt) | MELVGWLVDKGITSEKQWIQEDQASYISFNAASNSRSQIKAA |
| | | LDNAGKIMSLTKTAPDYLVGQQPVEDISSNRIYKILELNGYD |
| | | PQYAASVFLGWATKKFGKRNTIWLFGPATTGKTNIAEAIAHT |
| | | VPFYGCVNWTNENFPFNDCVDKMVIWWEEGKMTAKVVES |
| | | AKAILGGSKVRVDQKCKSSAQIDPTPVIVTSNTNMCAVIDGN |
| | | STTFEHQQPLQDRMFKFELTRRLDHDFGKVTKQEVKDFFRW |
| | | AKDHVVEVEHEFYVKKGGAKKRPAPSDADISEPKRVRESVA |
| | | QPSTSDAEASINYADRYQNKCSRHVGMNLMLFPCRQCERM |
| | | NQNSNICFTHGQKDCLECFPVSESQPVSVVKKAYQKLCYIHH |
| | | IMGKVPDACTACDLVNVDLDDCIFEQ |
| 98 | Rep40 (wt) | MELVGWLVDKGITSEKQWIQEDQASYISFNAASNSRSQIKAA |
| | | LDNAGKIMSLTKTAPDYLVGQQPVEDISSNRIYKILELNGYD |
| | | PQYAASVFLGWATKKFGKRNTIWLFGPATTGKTNIAEAIAHT |
| | | VPFYGCVNWTNENFPFNDCVDKMVIWWEEGKMTAKVVES |
| | | AKAILGGSKVRVDQKCKSSAQIDPTPVIVTSNTNMCAVIDGN |
| | | STTFEHQQPLQDRMFKFELTRRLDHDFGKVTKQEVKDFFRW |
| | | AKDHVVEVEHEFYVKKGGAKKRPAPSDADISEPKRVRESVA |
| | | QPSTSDAEASINYADRLARGHSL |
| 99 | Rep78 (wt) | MPGFYEIVIKVPSDLDEHLPGISDSFVNWVAEKEWELPPDSD |
| | | MDLNLIEQAPLTVAEKLQRDFLTEWRRVSKAPEALFFVQFEK |
| | | GESYFHMHVLVETTGVKSMVLGRFLSQIREKLIQRIYRGIEPT |
| | | LPNWFAVTKTRNGAGGGNKVVDECYIPNYLLPKTQPELQW |
| | | AWTNMEQYLSACLNLTERKRLVAQHLTHVSQTQEQNKENQ |
| | | NPNSDAPVIRSKTSARYMELVGWLVDKGITSEKQWIQEDQA |
| | | SYISFNAASNSRSQIKAALDNAGKIMSLTKTAPDYLVGQQPV |
| | | EDISSNRIYKILELNGYDPQYAASVFLGWATKKFGKRNTIWL |
| | | FGPATTGKTNIAEAIAHTVPFYGCVNWTNENFPFNDCVDKM |
| | | VIWWEEGKMTAKVVESAKAILGGSKVRVDQKCKSSAQIDPT |
| | | PVIVTSNTNMCAVIDGNSTTFEHQQPLQDRMFKFELTRRLDH |
| | | DFGKVTKQEVKDFFRWAKDHVVEVEHEFYVKKGGAKKRP |
| | | APSDADISEPKRVRESVAQPSTSDAEASINYADRYQNKCSRH |
| | | VGMNLMLFPCRQCERMNQNSNICFTHGQKDCLECFPVSESQ |
| | | PVSVVKKAYQKLCYIHHIMGKVPDACTACDLVNVDLDDCIF |
| | | EQ |
| 100 | Rep68 (wt) | MPGFYEIVIKVPSDLDEHLPGISDSFVNWVAEKEWELPPDSD |
| | | MDLNLIEQAPLTVAEKLQRDFLTEWRRVSKAPEALFFVQFEK |
| | | GESYFHMHVLVETTGVKSMVLGRFLSQIREKLIQRIYRGIEPT |
| | | LPNWFAVTKTRNGAGGGNKVVDECYIPNYLLPKTQPELQW |
| | | AWTNMEQYLSACLNLTERKRLVAQHLTHVSQTQEQNKENQ |
| | | NPNSDAPVIRSKTSARYMELVGWLVDKGITSEKQWIQEDQA |
| | | SYISFNAASNSRSQIKAALDNAGKIMSLTKTAPDYLVGQQPV |
| | | EDISSNRIYKILELNGYDPQYAASVFLGWATKKFGKRNTIWL |
| | | FGPATTGKTNIAEAIAHTVPFYGCVNWTNENFPFNDCVDKM |
| | | VIWWEEGKMTAKVVESAKAILGGSKVRVDQKCKSSAQIDPT |
| | | PVIVTSNTNMCAVIDGNSTTFEHQQPLQDRMFKFELTRRLDH |
| | | DFGKVTKQEVKDFFRWAKDHVVEVEHEFYVKKGGAKKRP |
| | | APSDADISEPKRVRESVAQPSTSDAEASINYADRLARGHSL |
| 101 | E2A (wt) | MASREEEQRETTPERGRGAARRPPTMEDVSSPSPSPPPPRAPP |
| | | KKRLRRRLESEDEEDSSQDALVPRTPSPRPSTSTADLAIASKK |
| | | KKKRPSPKPERPPSPEVIVDSEEEREDVALQMVGFSNPPVLIK |
| | | HGKGGKRTVRRLNEDDPVARGMRTQEEKEESSEAESESTVI |
| | | NPLSLPIVSAWEKGMEAARALMDKYHVDNDLKANFKLLPD |
| | | QVEALAAVCKTWLNEEHRGLQLTFTSNKTFVTMMGRFLQA |
| | | YLQSFAEVTYKHHEPTGCALWLHRCAEIEGELKCLHGSIMIN |
| | | KEHVIEMDVTSENGQRALKEQSSKAKIVKNRWGRNVVQISN |
| | | TDARCCVHDAACPANQFSGKSCGMFFSEGAKAQVAFKQIKA |
| | | FMQALYPNAQTGHGHLLMPLRCECNSKPGHAPFLGRQLPKL |
| | | TPFALSNAEDLDADLISDKSVLASVHHPALIVFQCCNPVYRN |
| | | SRAQGGGPNCDFKISAPDLLNALVMVRSLWSENFTELPRMV |
| | | VPEFKWSTKHQYRNVSLPVAHSDARQNPFDF |
| 102 | E4 ORF6 (wt) | MTTSGVPFGMTLRPTRSRLSRRTPYSRDRLPPFETETRATILE |
| | | DHPLLPECNTLTMHNVSYVRGLPCSVGFTLIQEWVVPWDMV |
| | | LTREEL VILRKCMHVCLCCANIDIMTSMMIHGYESWALHCH |
| | | CSSPGSLQCIAGGQVLASWFRMVVDGAMFNQRFIWYREVV |
| | | NYNMPKEVMFMSSVFMRGRHLIYLRLWYDGHVGSVVPAM |
| | | SFGYSALHCGILNNIVVLCCSYCADLSEIRVRCCARRTRRLM |
| | | LRAVRIIAEETTAMLYSCRTERRRQQFIRALLQHHRPILMHD |
| | | YDSTPM |
| 103 | E4 ORF6 (splice | atgactacgtccggcgttccatttggcatgacactacgaccaacacgatctcggttgtctcggcgc |
| | site removed) | actccgtacagtagggatcgcctacctccttttgagacagagacccgcgctaccatactggagga |
| | | tcatccgctgctgcccgaatgtaacactttgacaatgcacaaTgtTTCCtacgtgcgaggtctt |
| | | ccctgcagtgtgggatttacgctgattcaggaatgggttgttccctgggatatggttctgacgcgg |
| | | gaggagcttgtaatcctgaggaagtgtatgcacgtgtgcctgtgttgtgccaacattgatatcatga |
| | | cgagcatgatgatccatggttacgagtcctgggctctccactgtcattgttccagtcccggttccct |
| | | gcagtgcatagccggcgggcaggttttggccagctggtttaggatggtggtggatggcgccatg |
| | | tttaatcagaggtttatatggtaccgggaggtggtgaattacaacatgccaaaagaggtaatgttta |
| | | tgtccagcgtgtttatgaggggtcgccacttaatctacctgcgcttgtggtatgatggccacgtgg |
| | | gttctgtggtccccgccatgagctttggatacagcgccttgcactgtgggattttgaacaatattgt |
| | | ggtgctgtgctgcagttactgtgctgatttaagtgagatcagggtgcgctgctgtgcccggagga |
| | | caaggcgtctcatgctgcgggcggtgcgaatcatcgctgaggagaccactgccatgttgtattcc |
| | | tgcaggacggagcggcggcggcagcagtttattcgcgcgctgctgcagcaccaccgccctatc |
| | | ctgatgcacgattatgactctacccccatgTAGtaa |
| 104 | VARNA | CGACGTAATCCGTAGATGTACCTGGACATCCAGGTGATGC |
| | | CGGCGGCGGTGGTGGAGGCGCGCGGAAAGTCGCGGACGC |
| | | GGTTCCAGATGTTGCGCAGCGGCAAAAAGTGCTCCATGGT |
| | | CGGGACGCTCTGGCCGGTGAGGCGTGCGCAGTCGTTGACG |
| | | CTCTAGACCGTGCAAAAGGAGAGCCTGTAAGCGGGCACT |
| | | CTTCCGTGGTCTGGTGGATAAATTCGCAAGGGTATCATGG |
| | | CGGACGACCGGGGTTCGAACCCCGGATCCGGCCGTCCGCC |
| | | GTGATCCATGCGGTTACCGCCCGCGTGTCGAACCCAGGTG |
| | | TGCGACGTCAGACAACGGGGGAGCGCTCCTTTTGGCTTCC |
| | | TTCCAGGCGCGGCGGCTGCTGCGCTAGCTTTTTTGGCCACT |
| | | GGCCGCGCGCGGCGTAAGCGGTTAGGCTGGAAAGCGAAA |
| | | GCATTAAGTGGCTCGCTCCCTGTAGCCGGAGGGTTATTTT |
| | | CCAAGGGTTGAGTCGCAGGACCCCCGGTTCGAGTCTCGGG |
| | | CCGGCCGGACTGCGGCGAACGGGGGTTTGCCTCCCCGTCA |
| | | TGCAAGACCCCGCTTGCAAATTCCTCCGGAAACAGGGACG |
| | | AGCCCCTTTTTTGCTTTTCCCAGATGCATCCGGTGCTGCGG |
| | | CAGATGCGCCCCCCTCCTCAGCAGCGGCAAGAGCAAGAG |
| | | CAGCGGCAGACATGCAGGGCACCCTCCCCTTCTCCTACCG |
| | | CGTCAGGAGGGGCAACATCC |
| 105 | VP1 (wt) | MAADGYLPDWLEDTLSEGIRQWWKLKPGPPPPKPAERHKD |
| | | DSRGLVLPGYKYLGPFNGLDKGEPVNEADAAALEHDKAYD |
| | | RQLDSGDNPYLKYNHADAEFQERLKEDTSFGGNLGRAVFQA |
| | | KKRVLEPLGLVEEPVKTAPGKKRPVEHSPVEPDSSSGTGKAG |
| | | QQPARKRLNFGQTGDADSVPDPQPLGQPPAAPSGLGTNTMA |
| | | TGSGAPMADNNEGADGVGNSSGNWHCDSTWMGDRVITTST |
| | | RTWALPTYNNHLYKQISSQSGASNDNHYFGYSTPWGYFDFN |
| | | RFHCHFSPRDWQRLINNNWGFRPKRLNFKLFNIQVKEVTQN |
| | | DGTTTIANNLTSTVQVFTDSEYQLPYVLGSAHQGCLPPFPAD |
| | | VFMVPQYGYLTLNNGSQAVGRSSFYCLEYFPSQMLRTGNNF |
| | | TFSYTFEDVPFHSSYAHSQSLDRLMNPLIDQYLYYLSRTNTPS |
| | | GTTTQSRLQFSQAGASDIRDQSRNWLPGPCYRQQRVSKTSA |
| | | DNNNSEYSWTGATKYHLNGRDSLVNPGPAMASHKDDEEKF |
| | | FPQSGVLIFGKQGSEKTNVDIEKVMITDEEEIRTTNPVATEQY |
| | | GSVSTNLQRGNRQAATADVNTQGVLPGMVWQDRDVYLQG |
| | | PIWAKIPHTDGHFHPSPLMGGFGLKHPPPQILIKNTPVPANPS |
| | | TTFSAAKFASFITQYSTGQVSVEIEWELQKENSKRWNPEIQYT |
| | | SNYNKSVNVDFTVDTNGVYSEPRPIGTRYLTRNL |
| 106 | VP2 (wt) | TAPGKKRPVEHSPVEPDSSSGTGKAGQQPARKRLNFGQTGD |
| | | ADSVPDPQPLGQPPAAPSGLGTNTMATGSGAPMADNNEGA |
| | | DGVGNSSGNWHCDSTWMGDRVITTSTRTWALPTYNNHLYK |
| | | QISSQSGASNDNHYFGYSTPWGYFDFNRFHCHFSPRDWQRLI |
| | | NNNWGFRPKRLNFKLFNIQVKEVTQNDGTTTIANNLTSTVQ |
| | | VFTDSEYQLPYVLGSAHQGCLPPFPADVFMVPQYGYLTLNN |
| | | GSQAVGRSSFYCLEYFPSQMLRTGNNFTFSYTFEDVPFHSSY |
| | | AHSQSLDRLMNPLIDQYLYYLSRTNTPSGTTTQSRLQFSQAG |
| | | ASDIRDQSRNWLPGPCYRQQRVSKTSADNNNSEYSWTGATK |
| | | YHLNGRDSLVNPGPAMASHKDDEEKFFPQSGVLIFGKQGSE |
| | | KTNVDIEKVMITDEEEIRTTNPVATEQYGSVSTNLQRGNRQA |
| | | ATADVNTQGVLPGMVWQDRDVYLQGPIWAKIPHTDGHFHP |
| | | SPLMGGFGLKHPPPQILIKNTPVPANPSTTFSAAKFASFITQYS |
| | | TGQVSVEIEWELQKENSKRWNPEIQYTSNYNKSVNVDFTVD |
| | | TNGVYSEPRPIGTRYLTRNL |
| 107 | VP3 (wt) | MATGSGAPMADNNEGADGVGNSSGNWHCDSTWMGDRVIT |
| | | TSTRTWALPTYNNHLYKQISSQSGASNDNHYFGYSTPWGYF |
| | | DFNRFHCHFSPRDWQRLINNNWGFRPKRLNFKLFNIQVKEV |
| | | TQNDGTTTIANNLTSTVQVFTDSEYQLPYVLGSAHQGCLPPF |
| | | PADVFMVPQYGYLTLNNGSQAVGRSSFYCLEYFPSQMLRTG |
| | | NNFTFSYTFEDVPFHSSYAHSQSLDRLMNPLIDQYLYYLSRT |
| | | NTPSGTTTQSRLQFSQAGASDIRDQSRNWLPGPCYRQQRVSK |
| | | TSADNNNSEYSWTGATKYHLNGRDSLVNPGPAMASHKDDE |
| | | EKFFPQSGVLIFGKQGSEKTNVDIEKVMITDEEEIRTTNPVAT |
| | | EQYGSVSTNLQRGNRQAATADVNTQGVLPGMVWQDRDVY |
| | | LQGPIWAKIPHTDGHFHPSPLMGGFGLKHPPPQILIKNTPVPA |
| | | NPSTTFSAAKFASFITQYSTGQVSVEIEWELQKENSKRWNPEI |
| | | QYTSNYNKSVNVDFTVDINGVYSEPRPIGTRYLTRNL |
| 108 | AAP (wt) | LETQTQYLTPSLSDSHQQPPLVWELIRWLQAVAHQWQTITR |
| | | APTEWVIPREIGIAIPHGWATESSPPAPEPGPCPPTTTTSTNKFP |
| | | ANQEPRTTITTLATAPLGGILTSTDSTATFHHVTGKDSSTTTG |
| | | DSDPRDSTSSSLTFKSKRSRRMTVRRRLPITLPARFRCLLTRST |
| | | SSRTSSARRIKDASRRSQQTSSWCHSMDTSP |
| 109 | MAAP (wt) | LAHHHQSPQSGIRTTAGVLCFLGTSTSDPSTDSTRESRSTRQT |
| | | PRPSSTTKPTTGSSTAETTRTSSTTTPTRSFRSALKKIRLLGAT |
| | | SDEQSSRRKRGFLNLWAWLRNLLRRLREKRGR |
| 110 | WT Rep (nucleic | atgccggggttttacgagattgtgattaaggtccccagcgaccttgacgagcatctgcccggcatt |
| | acid sequence) | tctgacagctttgtgaactgggtggccgagaaggaatgggagttgccgccagattctgacatgg |
| | | atctgaatctgattgagcaggcacccctgaccgtggccgagaagctgcagcgcgactttctgac |
| | | ggaatggcgccgtgtgagtaaggccccggaggcccttttctttgtgcaatttgagaagggagag |
| | | agctacttccacatgcacgtgctcgtggaaaccaccggggtgaaatccatggttttgggacgtttc |
| | | ctgagtcagattcgcgaaaaactgattcagagaatttaccgcgggatcgagccgactttgccaaa |
| | | ctggttcgcggtcacaaagaccagaaatggcgccggaggcgggaacaaggtggtggatgagt |
| | | gctacatccccaattacttgctccccaaaacccagcctgagctccagtgggcgtggactaatatg |
| | | gaacagtatttaagcgcctgtttgaatctcacggagcgtaaacggttggtggcgcagcatctgac |
| | | gcacgtgtcgcagacgcaggagcagaacaaagagaatcagaatcccaattctgatgcgccggt |
| | | gatcagatcaaaaacttcagccaggtacatggagctggtcgggtggctcgtggacaaggggatt |
| | | acctcggagaagcagtggatccaggaggaccaggcctcatacatctccttcaatgcggcctcca |
| | | actcgcggtcccaaatcaaggctgccttggacaatgcgggaaagattatgagcctgactaaaac |
| | | cgcccccgactacctggtgggccagcagcccgtggaggacatttccagcaatcggatttataaa |
| | | attttggaactaaacgggtacgatccccaatatgcggcttccgtctttctgggatgggccacgaaa |
| | | aagttcggcaagaggaacaccatctggctgtttgggcctgcaactaccgggaagaccaacatcg |
| | | cggaggccatagcccacactgtgcccttctacgggtgcgtaaactggaccaatgagaactttccc |
| | | ttcaacgactgtgtcgacaagatggtgatctggtgggaggaggggaagatgaccgccaaggtc |
| | | gtggagtcggccaaagccattctcggaggaagcaaggtgcgcgtggaccagaaatgcaagtc |
| | | ctcggcccagatagacccgactcccgtgatcgtcacctccaacaccaacatgtgcgccgtgatt |
| | | gacgggaactcaacgaccttcgaacaccagcagccgttgcaagaccggatgttcaaatttgaac |
| | | tcacccgccgtctggatcatgactttgggaaggtcaccaagcaggaagtcaaagactttttccgg |
| | | tgggcaaaggatcacgtggttgaggtggagcatgaattctacgtcaaaaagggtggagccaag |
| | | aaaagacccgcccccagtgacgcagatataagtgagcccaaacgggtgcgcgagtcagttgc |
| | | gcagccatcgacgtcagacgcggaagcttcgatcaactacgcagacaggtaccaaaacaaatg |
| | | ttctcgtcacgtgggcatgaatctgatgctgtttccctgcagacaatgcgagagaatgaatcagaa |
| | | ttcaaatatctgcttcactcacggacagaaagactgtttagagtgctttcccgtgtcagaatctcaac |
| | | ccgtttctgtcgtcaaaaaggcgtatcagaaactgtgctacattcatcatatcatgggaaaggtgc |
| | | cagacgcttgcactgcctgcgatctggtcaatgtggatttggatgactgcatctttgaacaataaat |
| | | gatttaaatcaggtatggctgccgatggttatcttccagattggctcgaggacactctctctga |
| 111 | WT Rep (amino | MPGFYEIVIKVPSDLDEHLPGISDSFVNWVAEKEWELPPDSD |
| | acid Sequence) | MDLNLIEQAPLTVAEKLQRDFLTEWRRVSKAPEALFFVQFEK |
| | | GESYFHMHVLVETTGVKSMVLGRFLSQIREKLIQRIYRGIEPT |
| | | LPNWFAVTKTRNGAGGGNKVVDECYIPNYLLPKTQPELQW |
| | | AWTNMEQYLSACLNLTERKRLVAQHLTHVSQTQEQNKENQ |
| | | NPNSDAPVIRSKTSARYMELVGWLVDKGITSEKQWIQEDQA |
| | | SYISFNAASNSRSQIKAALDNAGKIMSLTKTAPDYLVGQQPV |
| | | EDISSNRIYKILELNGYDPQYAASVFLGWATKKFGKRNTIWL |
| | | FGPATTGKTNIAEAIAHTVPFYGCVNWTNENFPFNDCVDKM |
| | | VIWWEEGKMTAKVVESAKAILGGSKVRVDQKCKSSAQIDPT |
| | | PVIVTSNTNMCAVIDGNSTTFEHQQPLQDRMFKFELTRRLDH |
| | | DFGKVTKQEVKDFFRWAKDHVVEVEHEFYVKKGGAKKRP |
| | | APSDADISEPKRVRESVAQPSTSDAEASINYADRYQNKCSRH |
| | | VGMNLMLFPCRQCERMNQNSNICFTHGQKDCLECFPVSESQ |
| | | PVSVVKKAYQKLCYIHHIMGKVPDACTACDLVNVDLDDCIF |
| | | EQMIIRYGCRWLSSRLARGHSL |

TABLE 4
Exemplary Portions of AAV Gene Products

| SEQ ID | | |
|---|---|---|
| NO: | Description. | Sequence |
| 112 | pRC Flexon 1 Rep | MPGFYEIVIKVPSDLDEHLPGISDSFVNWVAEKEWELPPDSD |
| | portion 1 | MDLNLIEQAPLTVAEKLQRDFLTEWRRVSKAPEALFFVQFEK |
| | | GESYFHMHVLVETTGVKSMVLGRFLSQIREKLIQRIYRGIEPT |
| | | LPNWFAVTKTRNGAGGGNKVVDECYIPNYLLPKTQPELQW |
| | | AWTNMEQYLSACLNLTERKRLVAQHLTHVSQTQEQNKENQ |
| | | NPNSDAPVIRSKTSARYMELVGWLVDKGITSEKQWIQ |
| 113 | pRC Flexon 1 Rep | EDQASYISFNAASNSRSQIKAALDNAGKIMSLTKTAPDYLVG |
| | portion 2 | QQPVEDISSNRIYKILELNGYDPQYAASVFLGWATKKFGKRN |
| | | TIWLFGPATTGKTNIAEAIAHTVPFYGCVNWTNENFPFNDCV |
| | | DKMVIWWEEGKMTAKVVESAKAILGGSKVRVDQKCKSSAQ |
| | | IDPTPVIVTSNTNMCAVIDGNSTTFEHQQPLQDRMFKFELTRR |
| | | LDHDFGKVTKQEVKDFFRWAKDHVVEVEHEFYVKKGGAK |
| | | KRPAPSDADISEPKRVRESVAQPSTSDAEASINYADRYQNKC |
| | | SRHVGMNLMLFPCRQCERMNQNSNICFTHGQKDCLECFPVS |
| | | ESQPVSVVKKAYQKLCYIHHIMGKVPDACTACDLVNVDLD |
| | | DCIFEQMIIRYGCRWLSSRLARGHSL |
| 114 | pRC Flexon 2 Rep | MPGFYEIVIKVPSDLDEHLPGISDSFVNWVAEKEWELPPDSD |
| | portion 1 | MDLNLIEQAPLTVAEKLQRDFLTEWRRVSKAPEALFFVQFEK |
| | | GESYFHMHVLVETTGVKSMVLGRFLSQIREKLIQRIYRGIEPT |
| | | LPNWFAVTKTRNGAGGGNKVVDECYIPNYLLPKTQPELQW |
| | | AWTNMEQYLSACLNLTERKRLVAQHLTHVSQTQEQNKENQ |
| | | NPNSDAPVIRSKTSARYMELVGWLVDK |
| 115 | pRC Flexon 2 Rep | GITSEKQWIQEDQASYISFNAASNSRSQIKAALDNAGKIMSLT |
| | portion 2 | KTAPDYLVGQQPVEDISSNRIYKILELNGYDPQYAASVFLGW |
| | | ATKKFGKRNTIWLFGPATTGKTNIAEAIAHTVPFYGCVNWT |
| | | NENFPFNDCVDKMVIWWEEGKMTAKVVESAKAILGGSKVR |
| | | VDQKCKSSAQIDPTPVIVTSNTNMCAVIDGNSTTFEHQQPLQ |
| | | DRMFKFELTRRLDHDFGKVTKQEVKDFFRWAKDHVVEVEH |
| | | EFYVKKGGAKKRPAPSDADISEPKRVRESVAQPSTSDAEASI |
| | | NYADRYQNKCSRHVGMNLMLFPCRQCERMNQNSNICFTHG |
| | | QKDCLECFPVSESQPVSVVKKAYQKLCYIHHIMGKVPDACT |
| | | ACDLVNVDLDDCIFEQMIIRYGCRWLSSRLARGHSL |
| 116 | pRC Flexon 3 Rep | MPGFYEIVIKVPSDLDEHLPGISDSFVNWVAEKEWELPPDSD |
| | portion 1 | MDLNLIEQAPLTVAEKLQRDFLTEWRRVSKAPEALFFVQFEK |
| | | GESYFHMHVLVETTGVKSMVLGRFLSQIREKLIQRIYRGIEPT |
| | | LPNWFAVTKTRNGAGGGNKVVDECYIPNYLLPKTQPELQW |
| | | AWTNMEQYLSACLNLTERKRLVAQHLTHVSQTQEQNKENQ |
| | | NPNSDAPVIRSKTSARYMELVGWLVDKGITSEKQWIQEDQ |
| 117 | pRC Flexon 3 Rep | ASYISFNAASNSRSQIKAALDNAGKIMSLTKTAPDYLVGQQP |
| | portion 2 | VEDISSNRIYKILELNGYDPQYAASVFLGWATKKFGKRNTIW |
| | | LFGPATTGKTNIAEAIAHTVPFYGCVNWTNENFPFNDCVDK |
| | | MVIWWEEGKMTAKVVESAKAILGGSKVRVDQKCKSSAQID |
| | | PTPVIVTSNTNMCAVIDGNSTTFEHQQPLQDRMFKFELTRRL |
| | | DHDFGKVTKQEVKDFFRWAKDHVVEVEHEFYVKKGGAKK |
| | | RPAPSDADISEPKRVRESVAQPSTSDAEASINYADRYQNKCS |
| | | RHVGMNLMLFPCRQCERMNQNSNICFTHGQKDCLECFPVSE |
| | | SQPVSVVKKAYQKLCYIHHIMGKVPDACTACDLVNVDLDD |
| | | CIFEQ |
| 118 | pHelper Flexon 1 | MASREEEQRETTPERGRGAARRPPTMEDVSSPSPSPPPPRAPP |
| | E2A portion 1 | KKRL |
| 119 | pHelper Flexon 1 | RRRLESEDEEDSSQDALVPRTPSPRPSTSTADLAIASKKKKKR |
| | E2A portion 2 | PSPKPERPPSPEVIVDSEEEREDVALQMVGFSNPPVLIKHGKG |
| | | GKRTVRRLNEDDPVARGMRTQEEKEESSEAESESTVINPLSL |
| | | PIVSAWEKGMEAARALMDKYHVDNDLKANFKLLPDQVEAL |
| | | AAVCKTWLNEEHRGLQLTFTSNKTFVTMMGRFLQAYLQSF |
| | | AEVTYKHHEPTGCALWLHRCAEIEGELKCLHGSIMINKEHVI |
| | | EMDVTSENGQRALKEQSSKAKIVKNRWGRNVVQISNTDAR |
| | | CCVHDAACPANQFSGKSCGMFFSEGAKAQVAFKQIKAFMQ |
| | | ALYPNAQTGHGHLLMPLRCECNSKPGHAPFLGRQLPKLTPF |
| | | ALSNAEDLDADLISDKSVLASVHHPALIVFQCCNPVYRNSRA |
| | | QGGGPNCDFKISAPDLLNALVMVRSLWSENFTELPRMVVPE |
| | | FKWSTKHQYRNVSLPVAHSDARQNPFDF |
| 120 | pHelper Flexon 2 | MASREEEQRETTPERGRGAARRPPTMEDVSSPSPSPPPPRAPP |
| | E2A portion 1 | KKRLRRRLESE |
| 121 | pHelper Flexon 2 | DEEDSSQDALVPRTPSPRPSTSTADLAIASKKKKKRPSPKPER |
| | E2A portion 2 | PPSPEVIVDSEEEREDVALQMVGFSNPPVLIKHGKGGKRTVR |
| | | RLNEDDPVARGMRTQEEKEESSEAESESTVINPLSLPIVSAWE |
| | | KGMEAARALMDKYHVDNDLKANFKLLPDQVEALAAVCKT |
| | | WLNEEHRGLQLTFTSNKTFVTMMGRFLQAYLQSFAEVTYK |
| | | HHEPTGCALWLHRCAEIEGELKCLHGSIMINKEHVIEMDVTS |
| | | ENGQRALKEQSSKAKIVKNRWGRNVVQISNTDARCCVHDA |
| | | ACPANQFSGKSCGMFFSEGAKAQVAFKQIKAFMQALYPNAQ |
| | | TGHGHLLMPLRCECNSKPGHAPFLGRQLPKLTPFALSNAEDL |
| | | DADLISDKSVLASVHHPALIVFQCCNPVYRNSRAQGGGPNC |
| | | DFKISAPDLLNALVMVRSLWSENFTELPRMVVPEFKWSTKH |
| | | QYRNVSLPVAHSDARQNPFDF |
| 122 | pHelper Flexon 3 | MASREEEQRETTPERGRGAARRPPTMEDVSSPSPSPPPPRAPP |
| | E2A portion 1 | KKRLRRRLE |
| 123 | pHelper Flexon 3 | SEDEEDSSQDALVPRTPSPRPSTSTADLAIASKKKKKRPSPKP |
| | E2A portion 2 | ERPPSPEVIVDSEEEREDVALQMVGFSNPPVLIKHGKGGKRT |
| | | VRRLNEDDPVARGMRTQEEKEESSEAESESTVINPLSLPIVSA |
| | | WEKGMEAARALMDKYHVDNDLKANFKLLPDQVEALAAVC |
| | | KTWLNEEHRGLQLTFTSNKTFVTMMGRFLQAYLQSFAEVTY |
| | | KHHEPTGCALWLHRCAEIEGELKCLHGSIMINKEHVIEMDVT |
| | | SENGQRALKEQSSKAKIVKNRWGRNVVQISNTDARCCVHD |
| | | AACPANQFSGKSCGMFFSEGAKAQVAFKQIKAFMQALYPNA |
| | | QTGHGHLLMPLRCECNSKPGHAPFLGRQLPKLTPFALSNAED |
| | | LDADLISDKSVLASVHHPALIVFQCCNPVYRNSRAQGGGPNC |
| | | DFKISAPDLLNALVMVRSLWSENFTELPRMVVPEFKWSTKH |
| | | QYRNVSLPVAHSDARQNPFDF |
| 124 | pHelper Flexon 1 | MTTSGVPFGMTLRPTRSRLSRRTPYS |
| | E4ORF6 portion 1 | |
| 125 | pHelper Flexon 1 | RDRLPPFETETRATILEDHPLLPECNTLTMHNVSYVRGL |
| | E4ORF6 portion 2 | PCSVGFTLIQEWVVPWDMVLTREELVILRKCMHVCLCC |
| | | ANIDIMTSMMIHGYESWALHCHCSSPGSLQCIAGGQVL |
| | | ASWFRMVVDGAMFNQRFIWYREVVNYNMPKEVMFM |
| | | SSVFMRGRHLIYLRLWYDGHVGSVVPAMSFGYSALHC |
| | | GILNNIVVLCCSYCADLSEIRVRCCARRTRRLMLRAVRII |
| | | AEETTAMLYSCRTERRRQQFIRALLQHHRPILMHDYDS |
| | | TPM |
| 126 | pHelper Flexon 2 | MTTSGVPFGMTLRPTRSRLSRRTPYSRDRLPPFETETRATILE |
| | E4ORF6 portion 1 | |
| 127 | pHelper Flexon 2 | DHPLLPECNTLTMHNVSYVRGLPCSVGFTLIQEWVVPWDMV |
| | E4ORF6 portion 2 | LTREELVILRKCMHVCLCCANIDIMTSMMIHGYESWALHCH |
| | | CSSPGSLQCIAGGQVLASWFRMVVDGAMFNQRFIWYREVV |
| | | NYNMPKEVMFMSSVFMRGRHLIYLRLWYDGHVGSVVPAM |
| | | SFGYSALHCGILNNIVVLCCSYCADLSEIRVRCCARRTRRLM |
| | | LRAVRIIAEETTAMLYSCRTERRRQQFIRALLQHHRPILMHD |
| | | YDSTPM |
| 128 | pHelper Flexon 3 | MTTSGVPFGMTLRPTRSRLSRRTPY |
| | E4ORF6 portion 1 | |
| 129 | pHelper Flexon 3 | SRDRLPPFETETRATILEDHPLLPECNTLTMHNVSYVRGLPCS |
| | E4ORF6 portion 2 | VGFTLIQEWVVPWDMVLTREELVILRKCMHVCLCCANIDIM |
| | | TSMMIHGYESWALHCHCSSPGSLQCIAGGQVLASWFRMVV |
| | | DGAMFNQRFIWYREVVNYNMPKEVMFMSSVFMRGRHLIYL |
| | | RLWYDGHVGSVVPAMSFGYSALHCGILNNIVVLCCSYCADL |
| | | SEIRVRCCARRTRRLMLRAVRIIAEETTAMLYSCRTERRRQQ |
| | | FIRALLQHHRPILMHDYDSTPM |

TABLE 5
Exemplary Exon Sequences for Excisable Elements

| SEQ ID | | |
|---|---|---|
| NO: | Description. | Sequence |
| 130 | Exon 1 | GGTTCTGGCTAAgttaaattaatatatctgggttgatggtactggagaaggactgcgct |
| | | gcaaaacccgcaccctggactgtgagcccaagtgtgtagaagag |
| 131 | Exon 2 | GGCTCAGGTTAAgttaaattaattacctgagtggaattttgatggctctagtacctttcag |
| | | tctgagggctccaacagtgacatctatctcagccctgtagaagag |

TABLE 6
Exemplary Excisable element Sequences

| SEQ ID | | |
|---|---|---|
| NO: | Description. | Sequence |
| 132 | Excisable | gtaagttaacttgaacggcactgataacttcgtatagcatacattatacgaagttatacgaatgacg |
| | element 1 | CTGACatggcctaacctatgaaaaccctgtcttttgcctttctctccacagGGTTCTGG |
| | | CTAAgttaaattaatatatctgggttgatggtactggagaaggactgcgctgcaaaacccgca |
| | | ccctggactgtgagcccaagtgtgtagaagaggtaagtccatgccataacttcgtatagcatacat |
| | | tatacgaagttattactttggccCTCATatgatatatcgatacggtatgcctettGccctcAtcc |
| | | GgTag |
| 133 | Excisable | gtaagctaacctgaaccctggtcataacttcgtataGgATACtTtatacgaagttatctgcaac |
| | element 2 | ttcCTGACatggactaacccacttgaaccttgtcttctgccttttcctctacagGGCTCAG |
| | | GTTAAgttaaattaattacctgagtggaattttgatggctctagtacctttcagtctgagggctc |
| | | caacagtgacatctatctcagccctgtagaagaggtaagtaaaaacaataacttcgtataGgAT |
| | | ACtTtatacgaagttattcaaccaaatCTGACacgatctatcgaacttgcgctAcgcccGa |
| | | cAcctcctAtctgTag |

TABLE 7
Exemplary AAV Production Component Sequences

| SEQ ID | | |
|---|---|---|
| NO: | Description. | Sequence |
| 134 | pAI-4819 | atggatccggtacccaactccatgcttaacagtccccaggtacagcccaccctgcgtcgcaacc |
| | pHelper | aggaacagctctacagcttcctggagcgccactcgccctacttccgcagccacagtgcgcagat |
| | Flexon 1 | taggagcgccacttctttttgtcacttgaaaaacatgtaaaaataatgtactaggagacactttcaat |
| | | aaaggcaaatgtttttatttgtacactctcgggtgattatttaccccccacccttgccgtctgcgccgt |
| | | ttaaaaatcaaaggggttctgccgcgcatcgctatgcgccactggcagggacacgttgcgatact |
| | | ggtgtttagtgctccacttaaactcaggcacaaccatccgcggcagctcggtgaagttttcactcc |
| | | acaggctgcgcaccatcaccaacgcgtttagcaggtcgggcgccgatatcttgaagtcgcagtt |
| | | ggggcctccgccctgcgcgcgcgagttgcgatacacagggttgcagcactggaacactatcag |
| | | cgccgggtggtgcacgctggccagcacgctcttgtcggagatcagatccgcgtccaggtcctc |
| | | cgcgttgctcagggcgaacggagtcaactttggtagctgccttcccaaaaagggtgcatgccca |
| | | ggctttgagttgcactcgcaccgtagtggcatcagaaggtgaccgtgcccggtctgggcgttag |
| | | gatacagcgcctgcatgaaagccttgatctgcttaaaagccacctgagcctttgcgccttcagag |
| | | aagaacatgccgcaagacttgccggaaaactgattggccggacaggccgcgtcatgcacgca |
| | | gcaccttgcgtcggtgttggagatctgcaccacatttcggccccaccggttcttcacgatcttggc |
| | | cttgctagactgctccttcagcgcgcgctgcccgttttcgctcgtcacatccatttcaatcacgtgct |
| | | ccttatttatcataatgctcccgtgtagacacttaagctcgccttcgatctcagcgcagcggtgcag |
| | | ccacaacgcgcagcccgtgggctcgtggtgcttgtaggttacctctgcaaacgactgcaggtac |
| | | gcctgcaggaatcgccccatcatcgtcacaaaggtcttgttgctggtgaaggtcagctgcaaccc |
| | | gcggtgctcctcgtttagccaggtcttgcatacggccgccagagcttccacttggtcaggcagta |
| | | gcttgaagtttgcctttagatcgttatccacgtggtacttgtccatcaacgcgcgcgcagcctccat |
| | | gcccttctcccacgcagacacgatcggcaggctcagcgggtttatcaccgtgctttcactttccgc |
| | | ttcactggactcttccttttcctcttgcgtccgcataccccgcgccactgggtcgtcttcattcagcc |
| | | gccgcaccgtgcgcttacctcccttgccgtgcttgattagcaccggtgggttgctgaaacccacc |
| | | atttgtagcgccacatcttctctttcttcctcgctgtccacgatcacctctggggatggcgggcgct |
| | | cgggcttgggagaggggcgcttctttttctttttggacgcaatggccaaatccgccgtcgaggtc |
| | | gatggccgcgggctgggtgtgcgcggcaccagcgcatcttgtgacgagtcttcttcgtcctcgg |
| | | actcgagacgccgcctAcCggaTgagggCaagaggcataccgtatcgatatatcatATG |
| | | AGggccaaagtaataacttcgtataatgtatgctatacgaagttatggcatggacttacctcttcta |
| | | cacacttgggctcacagtccagggtgcgggttttgcagcgcagtccttctccagtaccatcaacc |
| | | cagatatattaatttaacTTAGCCAGAACCctgtggagagaaaggcaaaagacaggg |
| | | ttttcataggttaggccatGTCAGcgtcattcgtataacttcgtataatgtatgctatacgaagtt |
| | | atcagtgccgttcaagttaacttacctcagccgcttttttgggggcgcgcggggaggcggcggc |
| | | gacggcgacggggacgacacgtcctccatggttggtggacgtcgcgccgcaccgcgtccgcg |
| | | ctcgggggtggtttcgcgctgctcctcttcccgactggccatttccttctcctataggcagaaaaag |
| | | atcatggagtcagtcgagaaggaggacagcctaaccgccccctttgagttcgccaccaccgcct |
| | | ccaccgatgccgccaacgcgcctaccaccttccccgtcgaggcacccccgcttgaggaggag |
| | | gaagtgattatcgagcaggacccaggttttgtaagcgaagacgacgaggatcgctcagtaccaa |
| | | cagaggataaaaagcaagaccaggacgacgcagaggcaaacgaggaacaagtcgggggg |
| | | gggaccaaaggcatggcgactacctagatgtgggagacgacgtgctgttgaagcatctgcagc |
| | | gccagtgcgccattatctgcgacgcgttgcaagagcgcagcgatgtgcccctcgccatagcgg |
| | | atgtcagccttgcctacgaacgccacctgttctcaccgcgcgtaccccccaaacgccaagaaaa |
| | | cggcacatgcgagcccaacccgcgcctcaacttctaccccgtatttgccgtgccagaggtgcttg |
| | | ccacctatcacatctttttccaaaactgcaagatacccctatcctgccgtgccaaccgcagccgag |
| | | cggacaagcagctggccttgcggcagggcgctgtcatacctgatatcgcctcgctcgacgaagt |
| | | gccaaaaatctttgagggtcttggacgcgacgagaaacgcgcggcaaacgctctgcaacaaga |
| | | aaacagcgaaaatgaaagtcactgtggagtgctggtggaacttgagggtgacaacgcgcgcct |
| | | agccgtgctgaaacgcagcatcgaggtcacccactttgcctacccggcacttaacctacccccc |
| | | aaggttatgagcacagtcatgagcgagctgatcgtgcgccgtgcacgacccctggagagggat |
| | | gcaaacttgcaagaacaaaccgaggagggcctacccgcagttggcgatgagcagctggcgcg |
| | | ctggcttgagacgcgcgagcctgccgacttggaggagcgacgcaagctaatgatggccgcag |
| | | tgcttgttaccgtggagcttgagtgcatgcagcggttctttgctgacccggagatgcagcgcaag |
| | | ctagaggaaacgttgcactacacctttcgccagggctacgtgcgccaggcctgcaaaatttccaa |
| | | cgtggagctctgcaacctggtctcctaccttggaattttgcacgaaaaccgcctcgggcaaaacg |
| | | tgcttcattccacgctcaagggcgaggcgcgccgcgactacgtccgcgactgcgtttacttatttc |
| | | tgtgctacacctggcaaacggccatgggcgtgtggcagcaatgcctggaggagcgcaacctaa |
| | | aggagctgcagaagctgctaaagcaaaacttgaaggacctatggacggccttcaacgagcgct |
| | | ccgtggccgcgcacctggcggacattatcttccccgaacgcctgcttaaaaccctgcaacaggg |
| | | tctgccagacttcaccagtcaaagcatgttgcaaaactttaggaactttatcctagagcgttcagga |
| | | attctgcccgccacctgctgtgcgcttcctagcgactttgtgcccattaagtaccgtgaatgccctc |
| | | cgccgctttggggtcactgctaccttctgcagctagccaactaccttgcctaccactccgacatcat |
| | | ggaagacgtgagcggtgacggcctactggagtgtcactgtcgctgcaacctatgcaccccgca |
| | | ccgctccctggtctgcaattcgcaactgcttagcgaaagtcaaattatcggtacctttgagctgca |
| | | gggtccctcgcctgacgaaaagtccgcggctccggggttgaaactcactccggggctgtggac |
| | | gtcggcttaccttcgcaaatttgtacctgaggactaccacgcccacgagattaggttctacgaaga |
| | | ccaatcccgcccgccaaatgcggagcttaccgcctgcgtcattacccagggccacatccttggc |
| | | caattgcaagccatcaacaaagcccgccaagagtttctgctacgaaagggacggggggtttacc |
| | | tggacccccagtccggcgaggagctcaacccaatccccccgccgccgcagccctatcagcag |
| | | ccgcgggcccttgcttcccaggatggcacccaaaaagaagctgcagctgccgccgccgccac |
| | | ccacggacgaggaggaatactgggacagtcaggcagaggaggttttggacgaggaggagga |
| | | gatgatggaagactgggacagcctagacgaagcttccgaggccgaagaggtgtcagacgaaa |
| | | caccgtcaccctcggtcgcattcccctcgccggcgccccagaaattggcaaccgttcccagcat |
| | | cgctacaacctccgctcctcaggcgccgccggcactgcctgttcgccgacccaaccgtagatg |
| | | ggacaccactggaaccagggccggtaagtctaagcagccgccgccgttagcccaagagcaac |
| | | aacagcgccaaggctaccgctcgtggcgcgggcacaagaacgccatagttgcttgcttgcaag |
| | | actgtgggggcaacatctccttcgcccgccgctttcttctctaccatcacggcgtggccttccccc |
| | | gtaacatcctgcattactaccgtcatctctacagcccctactgcaccggcggcagcggcagcgg |
| | | cagcaacagcagcggtcacacagaagcaaaggcgaccggatagcaagactctgacaaagcc |
| | | caagaaatccacagcggcggcagcagcaggaggaggagcgctgcgtctggcgcccaacga |
| | | acccgtatcgacccgcgagcttagaaataggatttttcccactctgtatgctatatttcaacaaagc |
| | | aggggccaagaacaagagctgaaaataaaaaacaggtctctgcgctccctcacccgcagctgc |
| | | ctgtatcacaaaagcgaagatcagcttcggcgcacgctggaagacgcggaggctctcttcagca |
| | | aatactgcgcgctgactcttaaggactagtttcgcgccctttctcaaatttaagcgcgaaaactacg |
| | | tcatctccagcggccacacccggcgccagcacctgtcgtcagcgccattatgagcaaggaaatt |
| | | cccacgccctacatgtggagttaccagccacaaatgggacttgcggctggagctgcccaagact |
| | | actcaacccgaataaactacatgagcgcgggaccccacatgatatcccgggtcaacggaatcc |
| | | gcgcccaccgaaaccgaattctcctcgaacaggcggctattaccaccacacctcgtaataacctt |
| | | aatccccgtagttggcccgctgccctggtgtaccaggaaagtcccgctcccaccactgtggtact |
| | | tcccagagacgcccaggccgaagttcagatgactaactcaggggcgcagcttgcgggcggctt |
| | | tcgtcacagggtgcggtcgcccgggcgttttagggcggagtaacttgcatgtattgggaattgta |
| | | gtttttttaaaatgggaagtgacgtatcgtgggaaaacggaagtgaagatttgaggaagttgtggg |
| | | ttttttggctttcgtttctgggcgtaggttcgcgtgcggttttctgggtgttttttgtggactttaaccgtt |
| | | acgtcattttttagtcctatatatactcgctctgtacttggccctttttacactgtgactgattgagctgg |
| | | tgccgtgtcgagtggtgttttttaataggtttttttactggtaaggctgactgttatggctgccgctgtg |
| | | gaagcgctgtatgttgttctggagcgggagggtgctattttgcctaggcaggagggtttttcaggt |
| | | gtttatgtgtttttctctcctattaattttgttatacctcctatgggggctgtaatgttgtctctacgcctgc |
| | | gggtatgtattcccccgggctatttcggtcgctttttagcactgaccgatgttaaccaacctgatgtg |
| | | tttaccgagtcttacattatgactccggacatgaccgaggaactgtcggtggtgctttttaatcacg |
| | | gtgaccagtttttttacggtcacgccggcatggccgtagtccgtcttatgcttataagggttgtttttc |
| | | ctgttgtaagacaggcttctaatgtttaaatgtttttttttttgttattttattttgtgtttaatgcaggaacc |
| | | cgcagacatgtttgagagaaaaatggtgtctttttctgtggtggttccggaacttacctgcctttatct |
| | | gcatgagcatgactacgatgtgcttgcttttttgcgcgaggctttgcctgattttttgagcagcacctt |
| | | gcattttatatcgccgcccatgcaacaagcttacataggggctacgctggttagcatagctccgag |
| | | tatgcgtgtcataatcagtgtgggttcttttgtcatggttcctggcggggaagtggccgcgctggtc |
| | | cgtgcagacctgcacgattatgttcagctggccctgcgaagggacctacgggatcgcggtatttt |
| | | tgttaatgttccgcttttgaatcttatacaggtctgtgaggaacctgaatttttgcaatcatgattcgct |
| | | gcttgaggctgaaggtggagggcgctctggagcagatttttacaatggccggacttaatattcgg |
| | | gatttgcttagagacatattgataaggtggcgagatgaaaattatttgggcatggttgaaggtgctg |
| | | gaatgtttatagaggagattcaccctgaagggtttagcctttacgtccacttggacgtgagggcag |
| | | tttgccttttggaagccattgtgcaacatcttacaaatgccattatctgttctttggctgtagagtttga |
| | | ccacgccaccggaggggagcgcgttcacttaatagatcttcattttgaggttttggataatcttttgg |
| | | aataaaaaaaaaaaaacatggttcttccagctcttcccgctcctcccgtgtgtgactcgcagaacg |
| | | aatgtgtaggttggctgggtgtggcttattctgcggtggtggatgttatcagggcagcggcgcatg |
| | | aaggagtttacatagaacccgaagccagggggcgcctggatgctttgagagagtggatatacta |
| | | caactactacacagagcgagctaagcgacgagaccggagacgcagatctgtttgtcacgcccg |
| | | cacctggttttgcttcaggaaatatgactacgtccggcgttccatttggcatgacactacgaccaac |
| | | acgatctcggttgtctcggcgcactccgtacagtaggtaagctaacctgaaccctggtcataactt |
| | | cgtataGgATACtTtatacgaagttatctgcaacttcCTGACatggactaacccacttga |
| | | accttgtcttctgccttttcctctacagGGCTCAGGTTAAgttaaattaattacctgagtgg |
| | | aattttgatggctctagtacctttcagtctgagggctccaacagtgacatctatctcagccctgtaga |
| | | agaggtaagtaaaaacaataacttcgtataGgATACtTtatacgaagttattcaaccaaatC |
| | | TGACacgatctatcgaacttgcgctAcgcccGacAcctcctAtctgTagggatcgcctac |
| | | ctccttttgagacagagacccgcgctaccatactggaggatcatccgctgctgcccgaatgtaac |
| | | actttgacaatgcacaacgtgagttacgtgcgaggtcttccctgcagtgtgggatttacgctgattc |
| | | aggaatgggttgttccctgggatatggttctgacgcgggaggagcttgtaatcctgaggaagtgt |
| | | atgcacgtgtgcctgtgttgtgccaacattgatatcatgacgagcatgatgatccatggttacgagt |
| | | cctgggctctccactgtcattgttccagtcccggttccctgcagtgcatagccggcgggcaggttt |
| | | tggccagctggtttaggatggtggtggatggcgccatgtttaatcagaggtttatatggtaccggg |
| | | aggtggtgaattacaacatgccaaaagaggtaatgtttatgtccagcgtgtttatgaggggtcgcc |
| | | acttaatctacctgcgcttgtggtatgatggccacgtgggttctgtggtccccgccatgagctttgg |
| | | atacagcgccttgcactgtgggattttgaacaatattgtggtgctgtgctgcagttactgtgctgattt |
| | | aagtgagatcagggtgcgctgctgtgcccggaggacaaggcgtctcatgctgcgggcggtgc |
| | | gaatcatcgctgaggagaccactgccatgttgtattcctgcaggacggagcggcggcggcagc |
| | | agtttattcgcgcgctgctgcagcaccaccgccctatcctgatgcacgattatgactctacccccat |
| | | gtaggcgtggacttccccttcgccgcccgttgagcaaccgcaagttggacagcagcctgtggct |
| | | cagcagctggacagcgacatgaacttaagcgagctgcccggggagtttattaatatcactgatga |
| | | gcgtttggctcgacaggaaaccgtgtggaatataacacctaagaatatgtctgttacccatgatat |
| | | gatgctttttaaggccagccggggagaaaggactgtgtactctgtgtgttgggagggaggtggc |
| | | aggttgaatactagggttctgtgagtttgattaaggtacggtgatcaatataagctatgtggtggtg |
| | | gggctatactactgaatgaaaaatgacttgaaattttctgcaattgaaaaataaacacgttgaaaca |
| | | taacatgcaacaggttcacgattctttattcctgggcaatgtaggagaaggtgtaagagttggtag |
| | | caaaagtttcagtggtgtattttccactttcccaggaccatgtaaaagacatagagtaagtgcttac |
| | | ctcgctagtttctgtggattcactagaatcgatgtaggatgttgcccctcctgacgcggtaggaga |
| | | aggggagggtgccctgcatgtctgccgctgctcttgctcttgccgctgctgaggaggggggcg |
| | | catctgccgcagcaccggatgcatctgggaaaagcaaaaaaggggctcgtccctgtttccgga |
| | | ggaatttgcaagcggggtcttgcatgacggggaggcaaacccccgttcgccgcagtccggcc |
| | | ggcccgagactcgaaccgggggtcctgcgactcaacccttggaaaataaccctccggctacag |
| | | ggagcgagccacttaatgctttcgctttccagcctaaccgcttacgccgcgcgcggccagtggc |
| | | caaaaaagctagcgcagcagccgccgcgcctggaaggaagccaaaaggagcgctcccccgt |
| | | tgtctgacgtcgcacacctgggttcgacacgcgggcggtaaccgcatggatcacggcggacg |
| | | gccggatccggggttcgaaccccggtcgtccgccatgatacccttgcgaatttatccaccagacc |
| | | acggaagagtgcccgcttacaggctctccttttgcacggtctagagcgtcaacgactgcgcacg |
| | | cctcaccggccagagcgtcccgaccatggagcactttttgccgctgcgcaacatctggaaccgc |
| | | gtccgcgactttccgcgcgcctccaccaccgccgccggcatcacctggatgtccaggtacatct |
| | | acggattacgtcgacgtttaaaccatatgatcagctcactcaaaggggtaat |
| 135 | pAI-4820 | atggatccggtacccaactccatgcttaacagtccccaggtacagcccaccctgcgtcgcaacc |
| | pHelper | aggaacagctctacagcttcctggagcgccactcgccctacttccgcagccacagtgcgcagat |
| | Flexon 2 | taggagcgccacttctttttgtcacttgaaaaacatgtaaaaataatgtactaggagacactttcaat |
| | | aaaggcaaatgtttttatttgtacactctcgggtgattatttaccccccacccttgccgtctgcgccgt |
| | | ttaaaaatcaaaggggttctgccgcgcatcgctatgcgccactggcagggacacgttgcgatact |
| | | ggtgtttagtgctccacttaaactcaggcacaaccatccgcggcagctcggtgaagttttcactcc |
| | | acaggctgcgcaccatcaccaacgcgtttagcaggtcgggcgccgatatcttgaagtcgcagtt |
| | | ggggcctccgccctgcgcgcgcgagttgcgatacacagggttgcagcactggaacactatcag |
| | | cgccgggtggtgcacgctggccagcacgctcttgtcggagatcagatccgcgtccaggtcctc |
| | | cgcgttgctcagggcgaacggagtcaactttggtagctgccttcccaaaaagggtgcatgccca |
| | | ggctttgagttgcactcgcaccgtagtggcatcagaaggtgaccgtgcccggtctgggcgttag |
| | | gatacagcgcctgcatgaaagccttgatctgcttaaaagccacctgagcctttgcgccttcagag |
| | | aagaacatgccgcaagacttgccggaaaactgattggccggacaggccgcgtcatgcacgca |
| | | gcaccttgcgtcggtgttggagatctgcaccacatttcggccccaccggttcttcacgatcttggc |
| | | cttgctagactgctccttcagcgcgcgctgcccgttttcgctcgtcacatccatttcaatcacgtgct |
| | | ccttatttatcataatgctcccgtgtagacacttaagctcgccttcgatctcagcgcagcggtgcag |
| | | ccacaacgcgcagcccgtgggctcgtggtgcttgtaggttacctctgcaaacgactgcaggtac |
| | | gcctgcaggaatcgccccatcatcgtcacaaaggtcttgttgctggtgaaggtcagctgcaaccc |
| | | gcggtgctcctcgtttagccaggtcttgcatacggccgccagagcttccacttggtcaggcagta |
| | | gcttgaagtttgcctttagatcgttatccacgtggtacttgtccatcaacgcgcgcgcagcctccat |
| | | gcccttctcccacgcagacacgatcggcaggctcagcgggtttatcaccgtgctttcactttccgc |
| | | ttcactggactcttccttttcctcttgcgtccgcataccccgcgccactgggtcgtcttcattcagcc |
| | | gccgcaccgtgcgcttacctcccttgccgtgcttgattagcaccggtgggttgctgaaacccacc |
| | | atttgtagcgccacatcttctctttcttcctcgctgtccacgatcacctctggggatggcgggcgct |
| | | cgggcttgggagaggggcgcttctttttctttttggacgcaatggccaaatccgccgtcgaggtc |
| | | gatggccgcgggctgggtgtgcgcggcaccagcgcatcttgtgacgagtcttcttcgtcctAc |
| | | CggaTgagggCaagaggcataccgtatcgatatatcatATGAGggccaaagtaataactt |
| | | cgtataatgtatgctatacgaagttatggcatggacttacctcttctacacacttgggctcacagtcc |
| | | agggtgcgggttttgcagcgcagtccttctccagtaccatcaacccagatatattaatttaacTT |
| | | AGCCAGAACCctgtggagagaaaggcaaaagacagggttttcataggttaggccatG |
| | | TCAGcgtcattcgtataacttcgtataatgtatgctatacgaagttatcagtgccgttcaagttaa |
| | | cttacctcggactcgagacgccgcctcagccgcttttttgggggcgcgcggggaggcggcggc |
| | | gacggcgacggggacgacacgtcctccatggttggtggacgtcgcgccgcaccgcgtccgcg |
| | | ctcgggggtggtttcgcgctgctcctcttcccgactggccatttccttctcctataggcagaaaaag |
| | | atcatggagtcagtcgagaaggaggacagcctaaccgccccctttgagttcgccaccaccgcct |
| | | ccaccgatgccgccaacgcgcctaccaccttccccgtcgaggcacccccgcttgaggaggag |
| | | gaagtgattatcgagcaggacccaggttttgtaagcgaagacgacgaggatcgctcagtaccaa |
| | | cagaggataaaaagcaagaccaggacgacgcagaggcaaacgaggaacaagtcgggcggg |
| | | gggaccaaaggcatggcgactacctagatgtgggagacgacgtgctgttgaagcatctgcagc |
| | | gccagtgcgccattatctgcgacgcgttgcaagagcgcagcgatgtgcccctcgccatagcgg |
| | | atgtcagccttgcctacgaacgccacctgttctcaccgcgcgtaccccccaaacgccaagaaaa |
| | | cggcacatgcgagcccaacccgcgcctcaacttctaccccgtatttgccgtgccagaggtgcttg |
| | | ccacctatcacatctttttccaaaactgcaagatacccctatcctgccgtgccaaccgcagccgag |
| | | cggacaagcagctggccttgcggcagggcgctgtcatacctgatatcgcctcgctcgacgaagt |
| | | gccaaaaatctttgagggtcttggacgcgacgagaaacgcgcggcaaacgctctgcaacaaga |
| | | aaacagcgaaaatgaaagtcactgtggagtgctggtggaacttgagggtgacaacgcgcgcct |
| | | agccgtgctgaaacgcagcatcgaggtcacccactttgcctacccggcacttaacctacccccc |
| | | aaggttatgagcacagtcatgagcgagctgatcgtgcgccgtgcacgacccctggagagggat |
| | | gcaaacttgcaagaacaaaccgaggagggcctacccgcagttggcgatgagcagctggcgcg |
| | | ctggcttgagacgcgcgagcctgccgacttggaggagcgacgcaagctaatgatggccgcag |
| | | tgcttgttaccgtggagcttgagtgcatgcagcggttctttgctgacccggagatgcagcgcaag |
| | | ctagaggaaacgttgcactacacctttcgccagggctacgtgcgccaggcctgcaaaatttccaa |
| | | cgtggagctctgcaacctggtctcctaccttggaattttgcacgaaaaccgcctcgggcaaaacg |
| | | tgcttcattccacgctcaagggcgaggcgcgccgcgactacgtccgcgactgcgtttacttatttc |
| | | tgtgctacacctggcaaacggccatgggcgtgtggcagcaatgcctggaggagcgcaacctaa |
| | | aggagctgcagaagctgctaaagcaaaacttgaaggacctatggacggccttcaacgagcgct |
| | | ccgtggccgcgcacctggcggacattatcttccccgaacgcctgcttaaaaccctgcaacaggg |
| | | tctgccagacttcaccagtcaaagcatgttgcaaaactttaggaactttatcctagagcgttcagga |
| | | attctgcccgccacctgctgtgcgcttcctagcgactttgtgcccattaagtaccgtgaatgccctc |
| | | cgccgctttggggtcactgctaccttctgcagctagccaactaccttgcctaccactccgacatcat |
| | | ggaagacgtgagcggtgacggcctactggagtgtcactgtcgctgcaacctatgcaccccgca |
| | | ccgctccctggtctgcaattcgcaactgcttagcgaaagtcaaattatcggtacctttgagctgca |
| | | gggtccctcgcctgacgaaaagtccgcggctccggggttgaaactcactccggggctgtggac |
| | | gtcggcttaccttcgcaaatttgtacctgaggactaccacgcccacgagattaggttctacgaaga |
| | | ccaatcccgcccgccaaatgcggagcttaccgcctgcgtcattacccagggccacatccttggc |
| | | caattgcaagccatcaacaaagcccgccaagagtttctgctacgaaagggacggggggtttacc |
| | | tggacccccagtccggcgaggagctcaacccaatccccccgccgccgcagccctatcagcag |
| | | ccgcgggcccttgcttcccaggatggcacccaaaaagaagctgcagctgccgccgccgccac |
| | | ccacggacgaggaggaatactgggacagtcaggcagaggaggttttggacgaggaggagga |
| | | gatgatggaagactgggacagcctagacgaagcttccgaggccgaagaggtgtcagacgaaa |
| | | caccgtcaccctcggtcgcattcccctcgccggcgccccagaaattggcaaccgttcccagcat |
| | | cgctacaacctccgctcctcaggcgccgccggcactgcctgttcgccgacccaaccgtagatg |
| | | ggacaccactggaaccagggccggtaagtctaagcagccgccgccgttagcccaagagcaac |
| | | aacagcgccaaggctaccgctcgtggcgcgggcacaagaacgccatagttgcttgcttgcaag |
| | | actgtgggggcaacatctccttcgcccgccgctttcttctctaccatcacggcgtggccttccccc |
| | | gtaacatcctgcattactaccgtcatctctacagcccctactgcaccggcggcagcggcagcgg |
| | | cagcaacagcagcggtcacacagaagcaaaggcgaccggatagcaagactctgacaaagcc |
| | | caagaaatccacagcggcggcagcagcaggaggaggagcgctgcgtctggcgcccaacga |
| | | acccgtatcgacccgcgagcttagaaataggatttttcccactctgtatgctatatttcaacaaagc |
| | | aggggccaagaacaagagctgaaaataaaaaacaggtctctgcgctccctcacccgcagctgc |
| | | ctgtatcacaaaagcgaagatcagcttcggcgcacgctggaagacgcggaggctctcttcagca |
| | | aatactgcgcgctgactcttaaggactagtttcgcgccctttctcaaatttaagcgcgaaaactacg |
| | | tcatctccagcggccacacccggcgccagcacctgtcgtcagcgccattatgagcaaggaaatt |
| | | cccacgccctacatgtggagttaccagccacaaatgggacttgcggctggagctgcccaagact |
| | | actcaacccgaataaactacatgagcgcgggaccccacatgatatcccgggtcaacggaatcc |
| | | gcgcccaccgaaaccgaattctcctcgaacaggcggctattaccaccacacctcgtaataacctt |
| | | aatccccgtagttggcccgctgccctggtgtaccaggaaagtcccgctcccaccactgtggtact |
| | | tcccagagacgcccaggccgaagttcagatgactaactcaggggcgcagcttgcgggcggctt |
| | | tcgtcacagggtgcggtcgcccgggcgttttagggcggagtaacttgcatgtattgggaattgta |
| | | gtttttttaaaatgggaagtgacgtatcgtgggaaaacggaagtgaagatttgaggaagttgtggg |
| | | ttttttggctttcgtttctgggcgtaggttcgcgtgcggttttctgggtgttttttgtggactttaaccgtt |
| | | acgtcattttttagtcctatatatactcgctctgtacttggccctttttacactgtgactgattgagctgg |
| | | tgccgtgtcgagtggtgttttttaataggtttttttactggtaaggctgactgttatggctgccgctgtg |
| | | gaagcgctgtatgttgttctggagcgggagggtgctattttgcctaggcaggagggtttttcaggt |
| | | gtttatgtgtttttctctcctattaattttgttatacctcctatgggggctgtaatgttgtctctacgcctg |
| | | cgggtatgtattcccccgggctatttcggtcgctttttagcactgaccgatgttaaccaacctgatgtg |
| | | tttaccgagtcttacattatgactccggacatgaccgaggaactgtcggtggtgctttttaatcacg |
| | | gtgaccagtttttttacggtcacgccggcatggccgtagtccgtcttatgcttataagggttgtttttc |
| | | ctgttgtaagacaggcttctaatgtttaaatgtttttttttttgttattttattttgtgtttaatgcaggaa |
| | | cccgcagacatgtttgagagaaaaatggtgtctttttctgtggtggttccggaacttacctgcctttatct |
| | | gcatgagcatgactacgatgtgcttgcttttttgcgcgaggctttgcctgattttttgagcagcacctt |
| | | gcattttatatcgccgcccatgcaacaagcttacataggggctacgctggttagcatagctccgag |
| | | tatgcgtgtcataatcagtgtgggttcttttgtcatggttcctggcggggaagtggccgcgctggtc |
| | | cgtgcagacctgcacgattatgttcagctggccctgcgaagggacctacgggatcgcggtatttt |
| | | tgttaatgttccgcttttgaatcttatacaggtctgtgaggaacctgaatttttgcaatcatgattcgct |
| | | gcttgaggctgaaggtggagggcgctctggagcagatttttacaatggccggacttaatattcgg |
| | | gatttgcttagagacatattgataaggggcgagatgaaaattatttgggcatggttgaaggtgctg |
| | | gaatgtttatagaggagattcaccctgaagggtttagcctttacgtccacttggacgtgagggcag |
| | | tttgccttttggaagccattgtgcaacatcttacaaatgccattatctgttctttggctgtagagtttga |
| | | ccacgccaccggaggggagcgcgttcacttaatagatcttcattttgaggttttggataatcttttgg |
| | | aataaaaaaaaaaaaacatggttcttccagctcttcccgctcctcccgtgtgtgactcgcagaacg |
| | | aatgtgtaggttggctgggtgtggcttattctgcggtggtggatgttatcagggcagcggcgcatg |
| | | aaggagtttacatagaacccgaagccagggggcgcctggatgctttgagagagtggatatacta |
| | | caactactacacagagcgagctaagcgacgagaccggagacgcagatctgtttgtcacgcccg |
| | | cacctggttttgcttcaggaaatatgactacgtccggcgttccatttggcatgacactacgaccaac |
| | | acgatctcggttgtctcggcgcactccgtacagtagggatcgcctacctccttttgagacagaga |
| | | cccgcgctaccatactggaggtaagctaacctgaaccctggtcataacttcgtataGgATACt |
| | | TtatacgaagttatctgcaacttcCTGACatggactaacccacttgaaccttgtcttctgcctttt |
| | | cctctacagGGCTCAGGTTAAgttaaattaattacctgagtggaattttgatggctctagt |
| | | acctttcagtctgagggctccaacagtgacatctatctcagccctgtagaagaggtaagtaaaaac |
| | | aataacttcgtataGgATACtTtatacgaagttattcaaccaaatCTGACacgatctatcg |
| | | aacttgcgctAcgcccGacAcctcctAtctgTaggatcatccgctgctgcccgaatgtaaca |
| | | ctttgacaatgcacaacgtgagttacgtgcgaggtcttccctgcagtgtgggatttacgctgattca |
| | | ggaatgggttgttccctgggatatggttctgacgcgggaggagcttgtaatcctgaggaagtgtat |
| | | gcacgtgtgcctgtgttgtgccaacattgatatcatgacgagcatgatgatccatggttacgagtc |
| | | ctgggctctccactgtcattgttccagtcccggttccctgcagtgcatagccgggggcaggtttt |
| | | ggccagctggtttaggatggtggtggatggcgccatgtttaatcagaggtttatatggtaccggga |
| | | ggtggtgaattacaacatgccaaaagaggtaatgtttatgtccagcgtgtttatgaggggtcgcca |
| | | cttaatctacctgcgcttgtggtatgatggccacgtgggttctgtggtccccgccatgagctttgga |
| | | tacagcgccttgcactgtgggattttgaacaatattgtggtgctgtgctgcagttactgtgctgattta |
| | | agtgagatcagggtgcgctgctgtgcccggaggacaaggcgtctcatgctgcgggcggtgcg |
| | | aatcatcgctgaggagaccactgccatgttgtattcctgcaggacggagcggcggcggcagca |
| | | gtttattcgcgcgctgctgcagcaccaccgccctatcctgatgcacgattatgactctacccccat |
| | | gtaggcgtggacttccccttcgccgcccgttgagcaaccgcaagttggacagcagcctgtggct |
| | | cagcagctggacagcgacatgaacttaagcgagctgcccggggagtttattaatatcactgatga |
| | | gcgtttggctcgacaggaaaccgtgtggaatataacacctaagaatatgtctgttacccatgatat |
| | | gatgctttttaaggccagccggggagaaaggactgtgtactctgtgtgttgggagggaggtggc |
| | | aggttgaatactagggttctgtgagtttgattaaggtacggtgatcaatataagctatgtggtggtg |
| | | gggctatactactgaatgaaaaatgacttgaaattttctgcaattgaaaaataaacacgttgaaaca |
| | | taacatgcaacaggttcacgattctttattcctgggcaatgtaggagaaggtgtaagagttggtag |
| | | caaaagtttcagtggtgtattttccactttcccaggaccatgtaaaagacatagagtaagtgcttac |
| | | ctcgctagtttctgtggattcactagaatcgatgtaggatgttgcccctcctgacgcggtaggaga |
| | | aggggagggtgccctgcatgtctgccgctgctcttgctcttgccgctgctgaggaggggggcg |
| | | catctgccgcagcaccggatgcatctgggaaaagcaaaaaaggggctcgtccctgtttccgga |
| | | ggaatttgcaagcggggtcttgcatgacggggaggcaaacccccgttcgccgcagtccggcc |
| | | ggcccgagactcgaaccgggggtcctgcgactcaacccttggaaaataaccctccggctacag |
| | | ggagcgagccacttaatgctttcgctttccagcctaaccgcttacgccgcgcgcggccagtggc |
| | | caaaaaagctagcgcagcagccgccgcgcctggaaggaagccaaaaggagcgctcccccgt |
| | | tgtctgacgtcgcacacctgggttcgacacgcgggcggtaaccgcatggatcacggcggacg |
| | | gccggatccggggttcgaaccccggtcgtccgccatgatacccttgcgaatttatccaccagacc |
| | | acggaagagtgcccgcttacaggctctccttttgcacggtctagagcgtcaacgactgcgcacg |
| | | cctcaccggccagagcgtcccgaccatggagcactttttgccgctgcgcaacatctggaaccgc |
| | | gtccgcgactttccgcgcgcctccaccaccgccgccggcatcacctggatgtccaggtacatct |
| | | acggattacgtcgacgtttaaaccatatgatcagctcactcaaaggcggtaat |
| 136 | pAI-4821 | atggatccggtacccaactccatgcttaacagtccccaggtacagcccaccctgcgtcgcaacc |
| | pHelper | aggaacagctctacagcttcctggagcgccactcgccctacttccgcagccacagtgcgcagat |
| | Flexon 3 | taggagcgccacttctttttgtcacttgaaaaacatgtaaaaataatgtactaggagacactttcaat |
| | | aaaggcaaatgtttttatttgtacactctcgggtgattatttaccccccacccttgccgtctgcgccgt |
| | | ttaaaaatcaaaggggttctgccgcgcatcgctatgcgccactggcagggacacgttgcgatact |
| | | ggtgtttagtgctccacttaaactcaggcacaaccatccgcggcagctcggtgaagttttcactcc |
| | | acaggctgcgcaccatcaccaacgcgtttagcaggtcgggcgccgatatcttgaagtcgcagtt |
| | | ggggcctccgccctgcgcgcgcgagttgcgatacacagggttgcagcactggaacactatcag |
| | | cgccgggtggtgcacgctggccagcacgctcttgtcggagatcagatccgcgtccaggtcctc |
| | | cgcgttgctcagggcgaacggagtcaactttggtagctgccttcccaaaaagggtgcatgccca |
| | | ggctttgagttgcactcgcaccgtagtggcatcagaaggtgaccgtgcccggtctgggcgttag |
| | | gatacagcgcctgcatgaaagccttgatctgcttaaaagccacctgagcctttgcgccttcagag |
| | | aagaacatgccgcaagacttgccggaaaactgattggccggacaggccgcgtcatgcacgca |
| | | gcaccttgcgtcggtgttggagatctgcaccacatttcggccccaccggttcttcacgatcttggc |
| | | cttgctagactgctccttcagcgcgcgctgcccgttttcgctcgtcacatccatttcaatcacgtgct |
| | | ccttatttatcataatgctcccgtgtagacacttaagctcgccttcgatctcagcgcagcggtgcag |
| | | ccacaacgcgcagcccgtgggctcgtggtgcttgtaggttacctctgcaaacgactgcaggtac |
| | | gcctgcaggaatcgccccatcatcgtcacaaaggtcttgttgctggtgaaggtcagctgcaaccc |
| | | gcggtgctcctcgtttagccaggtcttgcatacggccgccagagcttccacttggtcaggcagta |
| | | gcttgaagtttgcctttagatcgttatccacgtggtacttgtccatcaacgcgcgcgcagcctccat |
| | | gcccttctcccacgcagacacgatcggcaggctcagcgggtttatcaccgtgctttcactttccgc |
| | | ttcactggactcttccttttcctcttgcgtccgcataccccgcgccactgggtcgtcttcattcagcc |
| | | gccgcaccgtgcgcttacctcccttgccgtgcttgattagcaccggtgggttgctgaaacccacc |
| | | atttgtagcgccacatcttctctttcttcctcgctgtccacgatcacctctggggatggcgggcgct |
| | | cgggcttgggagaggggcgcttctttttctttttggacgcaatggccaaatccgccgtcgaggtc |
| | | gatggccgcgggctgggtgtgcgcggcaccagcgcatcttgtgacgagtcttcttcgtcctcgg |
| | | actAcCggaTgagggCaagaggcataccgtatcgatatatcatATGAGggccaaagta |
| | | ataacttcgtataatgtatgctatacgaagttatggcatggacttacctcttctacacacttgggctca |
| | | cagtccagggtgcgggttttgcagcgcagtccttctccagtaccatcaacccagatatattaattta |
| | | acTTAGCCAGAACCctgtggagagaaaggcaaaagacagggttttcataggttaggc |
| | | catGTCAGcgtcattcgtataacttcgtataatgtatgctatacgaagttatcagtgccgttcaa |
| | | gttaacttacctcgagacgccgcctcagccgcttttttgggggcgcgcggggaggcggcggcg |
| | | acggcgacggggacgacacgtcctccatggttggtggacgtcgcgccgcaccgcgtccgcgc |
| | | tcgggggtggtttcgcgctgctcctcttcccgactggccatttccttctcctataggcagaaaaaga |
| | | tcatggagtcagtcgagaaggaggacagcctaaccgccccctttgagttcgccaccaccgcctc |
| | | caccgatgccgccaacgcgcctaccaccttccccgtcgaggcacccccgcttgaggaggagg |
| | | aagtgattatcgagcaggacccaggttttgtaagcgaagacgacgaggatcgctcagtaccaac |
| | | agaggataaaaagcaagaccaggacgacgcagaggcaaacgaggaacaagtcgggcgggg |
| | | ggaccaaaggcatggcgactacctagatgtgggagacgacgtgctgttgaagcatctgcagcg |
| | | ccagtgcgccattatctgcgacgcgttgcaagagcgcagcgatgtgcccctcgccatagcggat |
| | | gtcagccttgcctacgaacgccacctgttctcaccgcgcgtaccccccaaacgccaagaaaacg |
| | | gcacatgcgagcccaacccgcgcctcaacttctaccccgtatttgccgtgccagaggtgcttgcc |
| | | acctatcacatctttttccaaaactgcaagatacccctatcctgccgtgccaaccgcagccgagcg |
| | | gacaagcagctggccttgcggcagggcgctgtcatacctgatatcgcctcgctcgacgaagtgc |
| | | caaaaatctttgagggtcttggacgcgacgagaaacgcgcggcaaacgctctgcaacaagaaa |
| | | acagcgaaaatgaaagtcactgtggagtgctggtggaacttgagggtgacaacgcgcgcctag |
| | | ccgtgctgaaacgcagcatcgaggtcacccactttgcctacccggcacttaacctaccccccaa |
| | | ggttatgagcacagtcatgagcgagctgatcgtgcgccgtgcacgacccctggagagggatgc |
| | | aaacttgcaagaacaaaccgaggagggcctacccgcagttggcgatgagcagctggcgcgct |
| | | ggcttgagacgcgcgagcctgccgacttggaggagcgacgcaagctaatgatggccgcagtg |
| | | cttgttaccgtggagcttgagtgcatgcagcggttctttgctgacccggagatgcagcgcaagct |
| | | agaggaaacgttgcactacacctttcgccagggctacgtgcgccaggcctgcaaaatttccaac |
| | | gtggagctctgcaacctggtctcctaccttggaattttgcacgaaaaccgcctcgggcaaaacgt |
| | | gcttcattccacgctcaagggcgaggcgcgccgcgactacgtccgcgactgcgtttacttatttct |
| | | gtgctacacctggcaaacggccatgggcgtgtggcagcaatgcctggaggagcgcaacctaa |
| | | aggagctgcagaagctgctaaagcaaaacttgaaggacctatggacggccttcaacgagcgct |
| | | ccgtggccgcgcacctggcggacattatcttccccgaacgcctgcttaaaaccctgcaacaggg |
| | | tctgccagacttcaccagtcaaagcatgttgcaaaactttaggaactttatcctagagcgttcagga |
| | | attctgcccgccacctgctgtgcgcttcctagcgactttgtgcccattaagtaccgtgaatgccctc |
| | | cgccgctttggggtcactgctaccttctgcagctagccaactaccttgcctaccactccgacatcat |
| | | ggaagacgtgagcggtgacggcctactggagtgtcactgtcgctgcaacctatgcaccccgca |
| | | ccgctccctggtctgcaattcgcaactgcttagcgaaagtcaaattatcggtacctttgagctgca |
| | | gggtccctcgcctgacgaaaagtccgcggctccggggttgaaactcactccggggctgtggac |
| | | gtcggcttaccttcgcaaatttgtacctgaggactaccacgcccacgagattaggttctacgaaga |
| | | ccaatcccgcccgccaaatgcggagcttaccgcctgcgtcattacccagggccacatccttggc |
| | | caattgcaagccatcaacaaagcccgccaagagtttctgctacgaaagggacggggggtttacc |
| | | tggacccccagtccggcgaggagctcaacccaatccccccgccgccgcagccctatcagcag |
| | | ccgcgggcccttgcttcccaggatggcacccaaaaagaagctgcagctgccgccgccgccac |
| | | ccacggacgaggaggaatactgggacagtcaggcagaggaggttttggacgaggaggagga |
| | | gatgatggaagactgggacagcctagacgaagcttccgaggccgaagaggtgtcagacgaaa |
| | | caccgtcaccctcggtcgcattcccctcgccggcgccccagaaattggcaaccgttcccagcat |
| | | cgctacaacctccgctcctcaggcgccgccggcactgcctgttcgccgacccaaccgtagatg |
| | | ggacaccactggaaccagggccggtaagtctaagcagccgccgccgttagcccaagagcaac |
| | | aacagcgccaaggctaccgctcgtggcgcgggcacaagaacgccatagttgcttgcttgcaag |
| | | actgtgggggcaacatctccttcgcccgccgctttcttctctaccatcacggcgtggccttccccc |
| | | gtaacatcctgcattactaccgtcatctctacagcccctactgcaccggcggcagcggcagcgg |
| | | cagcaacagcagcggtcacacagaagcaaaggcgaccggatagcaagactctgacaaagcc |
| | | caagaaatccacagcggcggcagcagcaggaggaggagcgctgcgtctggcgcccaacga |
| | | acccgtatcgacccgcgagcttagaaataggatttttcccactctgtatgctatatttcaacaaagc |
| | | aggggccaagaacaagagctgaaaataaaaaacaggtctctgcgctccctcacccgcagctgc |
| | | ctgtatcacaaaagcgaagatcagcttcggcgcacgctggaagacgcggaggctctcttcagca |
| | | aatactgcgcgctgactcttaaggactagtttcgcgccctttctcaaatttaagcgcgaaaactacg |
| | | tcatctccagcggccacacccggcgccagcacctgtcgtcagcgccattatgagcaaggaaatt |
| | | cccacgccctacatgtggagttaccagccacaaatgggacttgcggctggagctgcccaagact |
| | | actcaacccgaataaactacatgagcgcgggaccccacatgatatcccgggtcaacggaatcc |
| | | gcgcccaccgaaaccgaattctcctcgaacaggcggctattaccaccacacctcgtaataacctt |
| | | aatccccgtagttggcccgctgccctggtgtaccaggaaagtcccgctcccaccactgtggtact |
| | | tcccagagacgcccaggccgaagttcagatgactaactcaggggcgcagcttgcgggcggctt |
| | | tcgtcacagggtgcggtcgcccgggcgttttagggcggagtaacttgcatgtattgggaattgta |
| | | gtttttttaaaatgggaagtgacgtatcgtgggaaaacggaagtgaagatttgaggaagttgtggg |
| | | ttttttggctttcgtttctgggcgtaggttcgcgtgcggttttctgggtgttttttgtggactttaaccgtt |
| | | acgtcattttttagtcctatatatactcgctctgtacttggccctttttacactgtgactgattgagctgg |
| | | tgccgtgtcgagtggtgttttttaataggtttttttactggtaaggctgactgttatggctgccgctgtg |
| | | gaagcgctgtatgttgttctggagcgggagggtgctattttgcctaggcaggagggtttttcaggt |
| | | gtttatgtgtttttctctcctattaattttgttatacctcctatgggggctgtaatgttgtctctacgcctg |
| | | cgggtatgtattcccccgggctatttcggtcgctttttagcactgaccgatgttaaccaacctgatgtg |
| | | tttaccgagtcttacattatgactccggacatgaccgaggaactgtcggtggtgctttttaatcacg |
| | | gtgaccagtttttttacggtcacgccggcatggccgtagtccgtcttatgcttataagggttgtttttc |
| | | ctgttgtaagacaggcttctaatgtttaaatgtttttttttttgttattttattttgtgtttaatgcaggaa |
| | | cccgcagacatgtttgagagaaaaatggtgtctttttctgtggtggttccggaacttacctgcctttatct |
| | | gcatgagcatgactacgatgtgcttgcttttttgcgcgaggctttgcctgattttttgagcagcacctt |
| | | gcattttatatcgccgcccatgcaacaagcttacataggggctacgctggttagcatagctccgag |
| | | tatgcgtgtcataatcagtgtgggttcttttgtcatggttcctggcggggaagtggccgcgctggtc |
| | | cgtgcagacctgcacgattatgttcagctggccctgcgaagggacctacgggatcgcggtatttt |
| | | tgttaatgttccgcttttgaatcttatacaggtctgtgaggaacctgaatttttgcaatcatgattcgct |
| | | gcttgaggctgaaggtggagggcgctctggagcagatttttacaatggccggacttaatattcgg |
| | | gatttgcttagagacatattgataaggtggcgagatgaaaattatttgggcatggttgaaggtgctg |
| | | gaatgtttatagaggagattcaccctgaagggtttagcctttacgtccacttggacgtgagggcag |
| | | tttgccttttggaagccattgtgcaacatcttacaaatgccattatctgttctttggctgtagagtttga |
| | | ccacgccaccggaggggagcgcgttcacttaatagatcttcattttgaggttttggataatcttttgg |
| | | aataaaaaaaaaaaaacatggttcttccagctcttcccgctcctcccgtgtgtgactcgcagaacg |
| | | aatgtgtaggttggctgggtgtggcttattctgcggtggtggatgttatcagggcagcggcgcatg |
| | | aaggagtttacatagaacccgaagccagggggcgcctggatgctttgagagagtggatatacta |
| | | caactactacacagagcgagctaagcgacgagaccggagacgcagatctgtttgtcacgcccg |
| | | cacctggttttgcttcaggaaatatgactacgtccggcgttccatttggcatgacactacgaccaac |
| | | acgatctcggttgtctcggcgcactccgtacaggtaagctaacctgaaccctggtcataacttcgt |
| | | ataGgATACtTtatacgaagttatctgcaacttcCTGACatggactaacccacttgaacct |
| | | tgtcttctgccttttcctctacagGGCTCAGGTTAAgttaaattaattacctgagtggaatt |
| | | ttgatggctctagtacctttcagtctgagggctccaacagtgacatctatctcagccctgtagaaga |
| | | ggtaagtaaaaacaataacttcgtataGgATACtTtatacgaagttattcaaccaaatCTG |
| | | ACacgatctatcgaacttgcgctAcgcccGacAcctcctAtctgTagtagggatcgcctac |
| | | ctccttttgagacagagacccgcgctaccatactggaggatcatccgctgctgcccgaatgtaac |
| | | actttgacaatgcacaacgtgagttacgtgcgaggtcttccctgcagtgtgggatttacgctgattc |
| | | aggaatgggttgttccctgggatatggttctgacgcgggaggagcttgtaatcctgaggaagtgt |
| | | atgcacgtgtgcctgtgttgtgccaacattgatatcatgacgagcatgatgatccatggttacgagt |
| | | cctgggctctccactgtcattgttccagtcccggttccctgcagtgcatagccggcgggcaggttt |
| | | tggccagctggtttaggatggtggtggatggcgccatgtttaatcagaggtttatatggtaccggg |
| | | aggtggtgaattacaacatgccaaaagaggtaatgtttatgtccagcgtgtttatgaggggtcgcc |
| | | acttaatctacctgcgcttgtggtatgatggccacgtgggttctgtggtccccgccatgagctttgg |
| | | atacagcgccttgcactgtgggattttgaacaatattgtggtgctgtgctgcagttactgtgctgattt |
| | | aagtgagatcagggtgcgctgctgtgcccggaggacaaggcgtctcatgctgcgggcggtgc |
| | | gaatcatcgctgaggagaccactgccatgttgtattcctgcaggacggagcggcggcggcagc |
| | | agtttattcgcgcgctgctgcagcaccaccgccctatcctgatgcacgattatgactctacccccat |
| | | gtaggcgtggacttccccttcgccgcccgttgagcaaccgcaagttggacagcagcctgtggct |
| | | cagcagctggacagcgacatgaacttaagcgagctgcccggggagtttattaatatcactgatga |
| | | gcgtttggctcgacaggaaaccgtgtggaatataacacctaagaatatgtctgttacccatgatat |
| | | gatgctttttaaggccagccggggagaaaggactgtgtactctgtgtgttgggagggaggtggc |
| | | aggttgaatactagggttctgtgagtttgattaaggtacggtgatcaatataagctatgtggtggtg |
| | | gggctatactactgaatgaaaaatgacttgaaattttctgcaattgaaaaataaacacgttgaaaca |
| | | taacatgcaacaggttcacgattctttattcctgggcaatgtaggagaaggtgtaagagttggtag |
| | | caaaagtttcagtggtgtattttccactttcccaggaccatgtaaaagacatagagtaagtgcttac |
| | | ctcgctagtttctgtggattcactagaatcgatgtaggatgttgcccctcctgacgcggtaggaga |
| | | aggggagggtgccctgcatgtctgccgctgctcttgctcttgccgctgctgaggaggggggcg |
| | | catctgccgcagcaccggatgcatctgggaaaagcaaaaaaggggctcgtccctgtttccgga |
| | | ggaatttgcaagcggggtcttgcatgacggggaggcaaacccccgttcgccgcagtccggcc |
| | | ggcccgagactcgaaccgggggtcctgcgactcaacccttggaaaataaccctccggctacag |
| | | ggagcgagccacttaatgctttcgctttccagcctaaccgcttacgccgcgcgcggccagtggc |
| | | caaaaaagctagcgcagcagccgccgcgcctggaaggaagccaaaaggagcgctcccccgt |
| | | tgtctgacgtcgcacacctgggttcgacacgcgggcggtaaccgcatggatcacggcggacg |
| | | gccggatccggggttcgaaccccggtcgtccgccatgatacccttgcgaatttatccaccagacc |
| | | acggaagagtgcccgcttacaggctctccttttgcacggtctagagcgtcaacgactgcgcacg |
| | | cctcaccggccagagcgtcccgaccatggagcactttttgccgctgcgcaacatctggaaccgc |
| | | gtccgcgactttccgcgcgcctccaccaccgccgccggcatcacctggatgtccaggtacatct |
| | | acggattacgtcgacgtttaaaccatatgatcagctcactcaaaggcggtaat |
| 137 | pAI-4822 pRC | gccgccatgccggggttttacgagattgtgattaaggtccccagcgaccttgacgagcatctgcc |
| | Flexon 1 | cggcatttctgacagctttgtgaactgggtggccgagaaggaatgggagttgccgccagattctg |
| | | acatggatctgaatctgattgagcaggcacccctgaccgtggccgagaagctgcagcgcgactt |
| | | tctgacggaatggcgccgtgtgagtaaggccccggaggcccttttctttgtgcaatttgagaagg |
| | | gagagagctacttccacatgcacgtgctcgtggaaaccaccggggtgaaatccatggttttggg |
| | | acgtttcctgagtcagattcgcgaaaaactgattcagagaatttaccgcgggatcgagccgacttt |
| | | gccaaactggttcgcggtcacaaagaccagaaatggcgccggaggcgggaacaaggtggtg |
| | | gatgagtgctacatccccaattacttgctccccaaaacccagcctgagctccagtgggcgtggac |
| | | taatatggaacagtatttaagcgcctgtttgaatctcacggagcgtaaacggttggtggcgcagca |
| | | tctgacgcacgtgtcgcagacgcaggagcagaacaaagagaatcagaatcccaattctgatgc |
| | | gccggtgatcagatcaaaaacttcagccaggtacatggagctggtcgggggctcgtggacaa |
| | | ggggattacctcggagaagcagtggatccaggtaagctaacctgaaccctggtcataacttcgta |
| | | taGgATACtTtatacgaagttatctgcaacttcCTGACatggactaacccacttgaacctt |
| | | gtcttctgccttttcctctacagGGCTCAGGTTAAgttaaattaattacctgagtggaattt |
| | | tgatggctctagtacctttcagtctgagggctccaacagtgacatctatctcagccctgtagaaga |
| | | ggtaagtaaaaacaataacttcgtataGgATACtTtatacgaagttattcaaccaaatCTG |
| | | ACacgatctatcgaacttgcgctAcgcccGacAcctcctAtctgTaggaggaccaggcct |
| | | catacatctccttcaatgcggcctccaactcgcggtcccaaatcaaggctgccttggacaatgcg |
| | | ggaaagattatgagcctgactaaaaccgcccccgactacctggtgggccagcagcccgtggag |
| | | gacatttccagcaatcggatttataaaattttggaactaaacgggtacgatccccaatatgcggctt |
| | | ccgtctttctgggatgggccacgaaaaagttcggcaagaggaacaccatctggctgtttgggcct |
| | | gcaactaccgggaagaccaacatcgcggaggccatagcccacactgtgcccttctacgggtgc |
| | | gtaaactggaccaatgagaactttcccttcaacgactgtgtcgacaagatggtgatctggtggga |
| | | ggaggggaagatgaccgccaaggtcgtggagtcggccaaagccattctcggaggaagcaag |
| | | gtgcgcgtggaccagaaatgcaagtcctcggcccagatagacccgactcccgtgatcgtcacct |
| | | ccaacaccaacatgtgcgccgtgattgacgggaactcaacgaccttcgaacaccagcagccgtt |
| | | gcaagaccggatgttcaaatttgaactcacccgccgtctggatcatgactttgggaaggtcacca |
| | | agcaggaagtcaaagactttttccggtgggcaaaggatcacgtggttgaggtggagcatgaatt |
| | | ctacgtcaaaaagggtggagccaagaaaagacccgcccccagtgacgcagatataagtgagc |
| | | ccaaacgggtgcgcgagtcagttgcgcagccatcgacgtcagacgcggaagcttcgatcaact |
| | | acgcagacaggtaccaaaacaaatgttctcgtcacgtgggcatgaatctgatgctgtttccctgca |
| | | gacaatgcgagagaatgaatcagaattcaaatatctgcttcactcacggacagaaagactgtttag |
| | | agtgctttcccgtgtcagaatctcaacccgtttctgtcgtcaaaaaggcgtatcagaaactgtgcta |
| | | cattcatcatatcatgggaaaggtgccagacgcttgcactgcctgcgatctggtcaatgtggattt |
| | | ggatgactgcatctttgaacaataaatgatttaaatcaggtatggctgccgatggttatcttccagat |
| | | tggctcgaggacactctctctgaaggaataagacagtggtggaagctcaaacctggcccaccac |
| | | caccaaagcccgcagagcggcataaggacgacagcaggggtcttgtgcttcctgggtacaagt |
| | | acctcggacccttcaacggactcgacaagggagagccggtcaacgaggcagacgccgcggc |
| | | cctcgagcacgacaaagcctacgaccggcagctcgacagcggagacaacccgtacctcaagt |
| | | acaaccacgccgacgcggagtttcaggagcgccttaaagaagatacgtcttttgggggcaacct |
| | | cggacgagcagtcttccaggcgaaaaagagggttcttgaacctctgggcctggttgaggaacct |
| | | gttaagacggctccgggaaaaaagaggccggtagagcactctcctgtggagccagactcctcc |
| | | tcgggaaccggaaaggcgggccagcagcctgcaagaaaaagattgaattttggtcagactgga |
| | | gacgcagactcagtacctgacccccagcctctcggacagccaccagcagccccctctggtctg |
| | | ggaactaatacgatggctacaggcagtggcgcaccaatggcagacaataacgagggcgccga |
| | | cggagtgggtaattcctcgggaaattggcattgcgattccacatggatgggcgacagagtcatca |
| | | ccaccagcacccgaacctgggccctgcccacctacaacaaccacctctacaaacaaatttccag |
| | | ccaatcaggagcctcgaacgacaatcactactttggctacagcaccccttgggggtattttgactt |
| | | caacagattccactgccacttttcaccacgtgactggcaaagactcatcaacaacaactggggatt |
| | | ccgacccaagagactcaacttcaagctctttaacattcaagtcaaagaggtcacgcagaatgacg |
| | | gtacgacgacgattgccaataaccttaccagcacggttcaggtgtttactgactcggagtaccag |
| | | ctcccgtacgtcctcggctcggcgcatcaaggatgcctcccgccgttcccagcagacgtcttcat |
| | | ggtgccacagtatggatacctcaccctgaacaacgggagtcaggcagtaggacgctcttcatttt |
| | | actgcctggagtactttccttctcagatgctgcgtaccggaaacaactttaccttcagctacactttt |
| | | gaggacgttcctttccacagcagctacgctcacagccagagtctggaccgtctcatgaatcctctc |
| | | atcgaccagtacctgtattacttgagcagaacaaacactccaagtggaaccaccacgcagtcaa |
| | | ggcttcagttttctcaggccggagcgagtgacattcgggaccagtctaggaactggcttcctgga |
| | | ccctgttaccgccagcagcgagtatcaaagacatctgcggataacaacaacagtgaatactcgt |
| | | ggactggagctaccaagtaccacctcaatggcagagactctctggtgaatccgggcccggccat |
| | | ggcaagccacaaggacgatgaagaaaagttttttcctcagagcggggttctcatctttgggaagc |
| | | aaggctcagagaaaacaaatgtggacattgaaaaggtcatgattacagacgaagaggaaatca |
| | | ggacaaccaatcccgtggctacggagcagtatggttctgtatctaccaacctccagagaggcaa |
| | | cagacaagcagctaccgcagatgtcaacacacaaggcgttcttccaggcatggtctggcagga |
| | | cagagatgtgtaccttcaggggcccatctgggcaaagattccacacacggacggacattttcacc |
| | | cctctcccctcatgggtggattcggacttaaacaccctcctccacagattctcatcaagaacaccc |
| | | cggtacctgcgaatccttcgaccaccttcagtgcggcaaagtttgcttccttcatcacacagtactc |
| | | cacgggacaggtcagcgtggagatcgagtgggagctgcagaaggaaaacagcaaacgctgg |
| | | aatcccgaaattcagtacacttccaactacaacaagtctgttaatgtggactttactgtggacactaa |
| | | tggcgtgtattcagagcctcgccccattggcaccagatacctgactcgtaatctgtaattgcttgtta |
| | | atcaataaaccgtttaattcgtttcagttgaactttggtctctgcgtatttctttcttatctagtttccatg |
| | | gctac |
| 138 | pAI-4823 pRC | gccgccatgccggggttttacgagattgtgattaaggtccccagcgaccttgacgagcatctgcc |
| | Flexon 2 | cggcatttctgacagctttgtgaactgggtggccgagaaggaatgggagttgccgccagattctg |
| | | acatggatctgaatctgattgagcaggcacccctgaccgtggccgagaagctgcagcgcgactt |
| | | tctgacggaatggcgccgtgtgagtaaggccccggaggcccttttctttgtgcaatttgagaagg |
| | | gagagagctacttccacatgcacgtgctcgtggaaaccaccggggtgaaatccatggttttggg |
| | | acgtttcctgagtcagattcgcgaaaaactgattcagagaatttaccgcgggatcgagccgacttt |
| | | gccaaactggttcgcggtcacaaagaccagaaatggcgccggaggcgggaacaaggtggtg |
| | | gatgagtgctacatccccaattacttgctccccaaaacccagcctgagctccagtgggcgtggac |
| | | taatatggaacagtatttaagcgcctgtttgaatctcacggagcgtaaacggttggtggcgcagca |
| | | tctgacgcacgtgtcgcagacgcaggagcagaacaaagagaatcagaatcccaattctgatgc |
| | | gccggtgatcagatcaaaaacttcagccaggtacatggagctggtcgggtggctcgtggacaa |
| | | ggtaagctaacctgaaccctggtcataacttcgtataGgATACtTtatacgaagttatctgca |
| | | acttcCTGACatggactaacccacttgaaccttgtcttctgccttttcctctacagGGCTCA |
| | | GGTTAAgttaaattaattacctgagtggaattttgatggctctagtacctttcagtctgagggct |
| | | ccaacagtgacatctatctcagccctgtagaagaggtaagtaaaaacaataacttcgtataGgA |
| | | TACtTtatacgaagttattcaaccaaatCTGACacgatctatcgaacttgcgctAcgccc |
| | | GacAcctcctAtctgTaggggattacctcggagaagcagtggatccaggaggaccaggcct |
| | | catacatctccttcaatgcggcctccaactcgcggtcccaaatcaaggctgccttggacaatgcg |
| | | ggaaagattatgagcctgactaaaaccgcccccgactacctggtgggccagcagcccgtggag |
| | | gacatttccagcaatcggatttataaaattttggaactaaacgggtacgatccccaatatgcggctt |
| | | ccgtctttctgggatgggccacgaaaaagttcggcaagaggaacaccatctggctgtttgggcct |
| | | gcaactaccgggaagaccaacatcgcggaggccatagcccacactgtgcccttctacgggtgc |
| | | gtaaactggaccaatgagaactttcccttcaacgactgtgtcgacaagatggtgatctggtggga |
| | | ggaggggaagatgaccgccaaggtcgtggagtcggccaaagccattctcggaggaagcaag |
| | | gtgcgcgtggaccagaaatgcaagtcctcggcccagatagacccgactcccgtgatcgtcacct |
| | | ccaacaccaacatgtgcgccgtgattgacgggaactcaacgaccttcgaacaccagcagccgtt |
| | | gcaagaccggatgttcaaatttgaactcacccgccgtctggatcatgactttgggaaggtcacca |
| | | agcaggaagtcaaagactttttccggtgggcaaaggatcacgtggttgaggtggagcatgaatt |
| | | ctacgtcaaaaagggtggagccaagaaaagacccgcccccagtgacgcagatataagtgagc |
| | | ccaaacgggtgcgcgagtcagttgcgcagccatcgacgtcagacgcggaagcttcgatcaact |
| | | acgcagacaggtaccaaaacaaatgttctcgtcacgtgggcatgaatctgatgctgtttccctgca |
| | | gacaatgcgagagaatgaatcagaattcaaatatctgcttcactcacggacagaaagactgtttag |
| | | agtgctttcccgtgtcagaatctcaacccgtttctgtcgtcaaaaaggcgtatcagaaactgtgcta |
| | | cattcatcatatcatgggaaaggtgccagacgcttgcactgcctgcgatctggtcaatgtggattt |
| | | ggatgactgcatctttgaacaataaatgatttaaatcaggtatggctgccgatggttatcttccagat |
| | | tggctcgaggacactctctctgaaggaataagacagtggtggaagctcaaacctggcccaccac |
| | | caccaaagcccgcagagcggcataaggacgacagcaggggtcttgtgcttcctgggtacaagt |
| | | acctcggacccttcaacggactcgacaagggagagccggtcaacgaggcagacgccgcggc |
| | | cctcgagcacgacaaagcctacgaccggcagctcgacagcggagacaacccgtacctcaagt |
| | | acaaccacgccgacgcggagtttcaggagcgccttaaagaagatacgtcttttgggggcaacct |
| | | cggacgagcagtcttccaggcgaaaaagagggttcttgaacctctgggcctggttgaggaacct |
| | | gttaagacggctccgggaaaaaagaggccggtagagcactctcctgtggagccagactcctcc |
| | | tcgggaaccggaaaggcgggccagcagcctgcaagaaaaagattgaattttggtcagactgga |
| | | gacgcagactcagtacctgacccccagcctctcggacagccaccagcagccccctctggtctg |
| | | ggaactaatacgatggctacaggcagtggcgcaccaatggcagacaataacgagggcgccga |
| | | cggagtgggtaattcctcgggaaattggcattgcgattccacatggatgggcgacagagtcatca |
| | | ccaccagcacccgaacctgggccctgcccacctacaacaaccacctctacaaacaaatttccag |
| | | ccaatcaggagcctcgaacgacaatcactactttggctacagcaccccttgggggtattttgactt |
| | | caacagattccactgccacttttcaccacgtgactggcaaagactcatcaacaacaactggggatt |
| | | ccgacccaagagactcaacttcaagctctttaacattcaagtcaaagaggtcacgcagaatgacg |
| | | gtacgacgacgattgccaataaccttaccagcacggttcaggtgtttactgactcggagtaccag |
| | | ctcccgtacgtcctcggctcggcgcatcaaggatgcctcccgccgttcccagcagacgtcttcat |
| | | ggtgccacagtatggatacctcaccctgaacaacgggagtcaggcagtaggacgctcttcatttt |
| | | actgcctggagtactttccttctcagatgctgcgtaccggaaacaactttaccttcagctacactttt |
| | | gaggacgttcctttccacagcagctacgctcacagccagagtctggaccgtctcatgaatcctctc |
| | | atcgaccagtacctgtattacttgagcagaacaaacactccaagtggaaccaccacgcagtcaa |
| | | ggcttcagttttctcaggccggagcgagtgacattcgggaccagtctaggaactggcttcctgga |
| | | ccctgttaccgccagcagcgagtatcaaagacatctgcggataacaacaacagtgaatactcgt |
| | | ggactggagctaccaagtaccacctcaatggcagagactctctggtgaatccgggcccggccat |
| | | ggcaagccacaaggacgatgaagaaaagttttttcctcagagcggggttctcatctttgggaagc |
| | | aaggctcagagaaaacaaatgtggacattgaaaaggtcatgattacagacgaagaggaaatca |
| | | ggacaaccaatcccgtggctacggagcagtatggttctgtatctaccaacctccagagaggcaa |
| | | cagacaagcagctaccgcagatgtcaacacacaaggcgttcttccaggcatggtctggcagga |
| | | cagagatgtgtaccttcaggggcccatctgggcaaagattccacacacggacggacattttcacc |
| | | cctctcccctcatgggtggattcggacttaaacaccctcctccacagattctcatcaagaacaccc |
| | | cggtacctgcgaatccttcgaccaccttcagtgcggcaaagtttgcttccttcatcacacagtactc |
| | | cacgggacaggtcagcgtggagatcgagtgggagctgcagaaggaaaacagcaaacgctgg |
| | | aatcccgaaattcagtacacttccaactacaacaagtctgttaatgtggactttactgtggacactaa |
| | | tggcgtgtattcagagcctcgccccattggcaccagatacctgactcgtaatctgtaattgcttgtta |
| | | atcaataaaccgtttaattcgtttcagttgaactttggtctctgcgtatttctttcttatctagtttccatg |
| | | gctac |
| 139 | pAI-4824 pRC | gccgccatgccggggttttacgagattgtgattaaggtccccagcgaccttgacgagcatctgcc |
| | Flexon 3 | cggcatttctgacagctttgtgaactgggtggccgagaaggaatgggagttgccgccagattctg |
| | | acatggatctgaatctgattgagcaggcacccctgaccgtggccgagaagctgcagcgcgactt |
| | | tctgacggaatggcgccgtgtgagtaaggccccggaggcccttttctttgtgcaatttgagaagg |
| | | gagagagctacttccacatgcacgtgctcgtggaaaccaccggggtgaaatccatggttttggg |
| | | acgtttcctgagtcagattcgcgaaaaactgattcagagaatttaccgcgggatcgagccgacttt |
| | | gccaaactggttcgcggtcacaaagaccagaaatggcgccggaggcgggaacaaggtggtg |
| | | gatgagtgctacatccccaattacttgctccccaaaacccagcctgagctccagtgggcgtggac |
| | | taatatggaacagtatttaagcgcctgtttgaatctcacggagcgtaaacggttggtggcgcagca |
| | | tctgacgcacgtgtcgcagacgcaggagcagaacaaagagaatcagaatcccaattctgatgc |
| | | gccggtgatcagatcaaaaacttcagccaggtacatggagctggtcgggtggctcgtggacaa |
| | | ggggattacctcggagaagcagtggatccaggaggaccaggtaagctaacctgaaccctggtc |
| | | ataacttcgtataGgATACtTtatacgaagttatctgcaacttcCTGACatggactaaccc |
| | | acttgaaccttgtcttctgccttttcctctacagGGCTCAGGTTAAgttaaattaattacct |
| | | gagtggaattttgatggctctagtacctttcagtctgagggctccaacagtgacatctatctcagcc |
| | | ctgtagaagaggtaagtaaaaacaataacttcgtataGgATACtTtatacgaagttattcaac |
| | | caaatCTGACacgatctatcgaacttgcgctAcgcccGacAcctcctAtctgTaggcctc |
| | | atacatctccttcaatgcggcctccaactcgcggtcccaaatcaaggctgccttggacaatgcgg |
| | | gaaagattatgagcctgactaaaaccgcccccgactacctggtgggccagcagcccgtggagg |
| | | acatttccagcaatcggatttataaaattttggaactaaacgggtacgatccccaatatgcggcttc |
| | | cgtctttctgggatgggccacgaaaaagttcggcaagaggaacaccatctggctgtttgggcctg |
| | | caactaccgggaagaccaacatcgcggaggccatagcccacactgtgcccttctacgggtgcg |
| | | taaactggaccaatgagaactttcccttcaacgactgtgtcgacaagatggtgatctggtgggag |
| | | gaggggaagatgaccgccaaggtcgtggagtcggccaaagccattctcggaggaagcaaggt |
| | | gcgcgtggaccagaaatgcaagtcctcggcccagatagacccgactcccgtgatcgtcacctc |
| | | caacaccaacatgtgcgccgtgattgacgggaactcaacgaccttcgaacaccagcagccgttg |
| | | caagaccggatgttcaaatttgaactcacccgccgtctggatcatgactttgggaaggtcaccaa |
| | | gcaggaagtcaaagactttttccggtgggcaaaggatcacgtggttgaggtggagcatgaattct |
| | | acgtcaaaaagggtggagccaagaaaagacccgcccccagtgacgcagatataagtgagccc |
| | | aaacgggtgcgcgagtcagttgcgcagccatcgacgtcagacgcggaagcttcgatcaactac |
| | | gcagacaggtaccaaaacaaatgttctcgtcacgtgggcatgaatctgatgctgtttccctgcaga |
| | | caatgcgagagaatgaatcagaattcaaatatctgcttcactcacggacagaaagactgtttagag |
| | | tgctttcccgtgtcagaatctcaacccgtttctgtcgtcaaaaaggcgtatcagaaactgtgctaca |
| | | ttcatcatatcatgggaaaggtgccagacgcttgcactgcctgcgatctggtcaatgtggatttgg |
| | | atgactgcatctttgaacaataaatgatttaaatcaggtatggctgccgatggttatcttccagattg |
| | | gctcgaggacactctctctgaaggaataagacagtggtggaagctcaaacctggcccaccacc |
| | | accaaagcccgcagagcggcataaggacgacagcaggggtcttgtgcttcctgggtacaagta |
| | | cctcggacccttcaacggactcgacaagggagagccggtcaacgaggcagacgccgcggcc |
| | | ctcgagcacgacaaagcctacgaccggcagctcgacagcggagacaacccgtacctcaagta |
| | | caaccacgccgacgcggagtttcaggagcgccttaaagaagatacgtcttttgggggcaacctc |
| | | ggacgagcagtcttccaggcgaaaaagagggttcttgaacctctgggcctggttgaggaacctg |
| | | ttaagacggctccgggaaaaaagaggccggtagagcactctcctgtggagccagactcctcctc |
| | | gggaaccggaaaggcgggccagcagcctgcaagaaaaagattgaattttggtcagactggag |
| | | acgcagactcagtacctgacccccagcctctcggacagccaccagcagccccctctggtctgg |
| | | gaactaatacgatggctacaggcagtggcgcaccaatggcagacaataacgagggcgccgac |
| | | ggagtgggtaattcctcgggaaattggcattgcgattccacatggatgggcgacagagtcatcac |
| | | caccagcacccgaacctgggccctgcccacctacaacaaccacctctacaaacaaatttccagc |
| | | caatcaggagcctcgaacgacaatcactactttggctacagcaccccttgggggtattttgacttc |
| | | aacagattccactgccacttttcaccacgtgactggcaaagactcatcaacaacaactggggattc |
| | | cgacccaagagactcaacttcaagctctttaacattcaagtcaaagaggtcacgcagaatgacgg |
| | | tacgacgacgattgccaataaccttaccagcacggttcaggtgtttactgactcggagtaccagct |
| | | cccgtacgtcctcggctcggcgcatcaaggatgcctcccgccgttcccagcagacgtcttcatg |
| | | gtgccacagtatggatacctcaccctgaacaacgggagtcaggcagtaggacgctcttcatttta |
| | | ctgcctggagtactttccttctcagatgctgcgtaccggaaacaactttaccttcagctacacttttg |
| | | aggacgttcctttccacagcagctacgctcacagccagagtctggaccgtctcatgaatcctctca |
| | | tcgaccagtacctgtattacttgagcagaacaaacactccaagtggaaccaccacgcagtcaag |
| | | gcttcagttttctcaggccggagcgagtgacattcgggaccagtctaggaactggcttcctggac |
| | | cctgttaccgccagcagcgagtatcaaagacatctgcggataacaacaacagtgaatactcgtg |
| | | gactggagctaccaagtaccacctcaatggcagagactctctggtgaatccgggcccggccatg |
| | | gcaagccacaaggacgatgaagaaaagttttttcctcagagcggggttctcatctttgggaagca |
| | | aggctcagagaaaacaaatgtggacattgaaaaggtcatgattacagacgaagaggaaatcag |
| | | gacaaccaatcccgtggctacggagcagtatggttctgtatctaccaacctccagagaggcaac |
| | | agacaagcagctaccgcagatgtcaacacacaaggcgttcttccaggcatggtctggcaggac |
| | | agagatgtgtaccttcaggggcccatctgggcaaagattccacacacggacggacattttcaccc |
| | | ctctcccctcatgggtggattcggacttaaacaccctcctccacagattctcatcaagaacacccc |
| | | ggtacctgcgaatccttcgaccaccttcagtgcggcaaagtttgcttccttcatcacacagtactcc |
| | | acgggacaggtcagcgtggagatcgagtgggagctgcagaaggaaaacagcaaacgctgga |
| | | atcccgaaattcagtacacttccaactacaacaagtctgttaatgtggactttactgtggacactaat |
| | | ggcgtgtattcagagcctcgccccattggcaccagatacctgactcgtaatctgtaattgcttgtta |
| | | atcaataaaccgtttaattcgtttcagttgaactttggtctctgcgtatttctttcttatctagtttccatgg |
| | | ctac |

TABLE 8
Exemplary Recombinase Amino Acid Sequences

| SEQ ID | | |
|---|---|---|
| NO: | Description. | Sequence |
| 140 | PhiC31 (contains | MDTYAGAYDRQSRERENSSAASPATQRSANEDKAADLQRE |
| | a C-terminal | VERDGGRFRFVGHFSEAPGTSAFGTAERPEFERILNECRAGR |
| | NLS) | LNMIIVYDVSRFSRLKVMDAIPIVSELLALGVTIVSTQEGVFR |
| | | QGNVMDLIHLIMRLDASHKESSLKSAKILDTKNLQRELGGY |
| | | VGGKAPYGFELVSETKEITRNGRMVNVVINKLAHSTTPLTGP |
| | | FEFEPDVIRWWWREIKTHKHLPFKPGSQAAIHPGSITGLCKR |
| | | MDADAVPTRGETIGKKTASSAWDPATVMRILRDPRIAGFAA |
| | | EVIYKKKPDGTPTTKIEGYRIQRDPITLRPVELDCGPIIEPAEW |
| | | YELQAWLDGRGRGKGLSRGQAILSAMDKLYCECGAVMTSK |
| | | RGEESIKDSYRCRRRKVVDPSAPGQHEGTCNVSMAALDKFV |
| | | AERIFNKIRHAEGDEETLALLWEAARRFGKLTEAPEKSGERA |
| | | NLVAERADALNALEELYEDRAAGAYDGPVGRKHFRKQQAA |
| | | LTLRQQGAEERLAELEAAEAPKLPLDQWFPEDADADPTGPK |
| | | SWWGRASVDDKRVFVGLFVDKIVVTKSTTGRGQGTPIEKRA |
| | | SITWAKPPTDDDEDDAQDGTEDVAAPKKKRKV* |
| 141 | Cre | MSNLLTVHQNLPALPVDATSDEVRKNLMDMFRDRQAFSEH |
| | | TWKMLLSVCRSWAAWCKLNNRKWFPAEPEDVRDYLLYLQ |
| | | ARGLAVKTIQQHLGQLNMLHRRSGLPRPSDSNAVSLVMRRI |
| | | RKENVDAGERAKQALAFERTDFDQVRSLMENSDRCQDIRNL |
| | | AFLGIAYNTLLRIAEIARIRVKDISRTDGGRMLIHIGRTKTLVS |
| | | TAGVEKALSLGVTKLVERWISVSGVADDPNNYLFCRVRKNG |
| | | VAAPSATSQLSTRALEGIFEATHRLIYGAKDDSGQRYLAWSG |
| | | HSARVGAARDMARAGVSIPEIMQAGGWTNVNIVMNYIRNL |
| | | DSETGAMVRLLEDGD* |
| 142 | VCre (contains a | MIENQLSLLGDFSGVRPDDVKTAIQAAQKKGINVAENEQFK |
| | C-terminal NLS) | AAFEHLLNEFKKREERYSPNTLRRLESAWTCFVDWCLANHR |
| | | HSLPATPDTVEAFFIERAEELHRNTLSVYRWAISRVHRVAGC |
| | | PDPCLDIYVEDRLKAIARKKVREGEAVKQASPFNEQHLLKLT |
| | | SLWYRSDKLLLRRNLALLAVAYESMLRASELANIRVSDMEL |
| | | AGDGTAILTIPITKTNHSGEPDTCILSQDVVSLLMDYTEAGKL |
| | | DMSSDGFLFVGVSKHNTCIKPKKDKQTGEVLHKPITTKTVEG |
| | | VFYSAWETLDLGRQGVKPFTAHSARVGAAQDLLKKGYNTL |
| | | QIQQSGRWSSGAMVARYGRAILARDGAMAHSRVKTRSAPM |
| | | QWGKDEKDPKKKRKV* |
| 143 | Flp (contains a C- | MSQFDILCKTPPKVLVRQFVERFERPSGEKIASCAAELTYLC |
| | terminal NLS) | WMITHNGTAIKRATFMSYNTIISNSLSFDIVNKSLQFKYKTQK |
| | | ATILEASLKKLIPAWEFTIIPYNGQKHQSDITDIVSSLQLQFESS |
| | | EEADKGNSHSKKMLKALLSEGESIWEITEKILNSFEYTSRFTK |
| | | TKTLYQFLFLATFINCGRFSDIKNVDPKSFKLVQNKYLGVIIQ |
| | | CLVTETKTSVSRHIYFFSARGRIDPLVYLDEFLRNSEPVLKRV |
| | | NRTGNSSSNKQEYQLLKDNLVRSYNKALKKNAPYPIFAIKN |
| | | GPKSHIGRHLMTSFLSMKGLTELTNVVGNWSDKRASAVART |
| | | TYTHQITAIPDHYFALVSRYYAYDPISKEMIALKDETNPIEEW |
| | | QHIEQLKGSAEGSIRYPAWNGIISQEVLDYLSSYINRRIPKKK |
| | | RKV* |
| 144 | Bxb1 (contains a | MRALVVIRLSRVTDATTSPERQLESCQQLCAQRGWDVVGVA |
| | C-terminal NLS) | EDLDVSGAVDPFDRKRRPNLARWLAFEEQPFDVIVAYRVDR |
| | | LTRSIRHLQQLVHWAEDHKKLVVSATEAHFDTTTPFAAVVI |
| | | ALMGTVAQMELEAIKERNRSAAHFNIRAGKYRGSLPPWGYL |
| | | PTRVDGEWRLVPDPVQRERILEVYHRVVDNHEPLHLVAHDL |
| | | NRRGVLSPKDYFAQLQGREPQGREWSATALKRSMISEAMLG |
| | | YATLNGKTVRDDDGAPLVRAEPILTREQLEALRAELVKTSR |
| | | AKPAVSTPSLLLRVLFCAVCGEPAYKFAGGGRKHPRYRCRS |
| | | MGFPKHCGNGTVAMAEWDAFCEEQVLDLLGDAERLEKVW |
| | | VAGSDSAVELAEVNAELVDLTSLIGSPAYRAGSPQREALDAR |
| | | IAALAARQEELEGLEARPSGWEWRETGQRFGDWWREQDTA |
| | | AKNTWLRSMNVRLTFDVRGGLTRTIDFGDLQEYEQHLRLGS |
| | | VVERLHTGMSPKKKRKV* |
| 145 | TP901 | MTKKVAIYTRVSTTNQAEEGFSIDEQIDRLTKYAEAMGWQV |
| | | SDTYTDAGFSGAKLERPAMQRLINDIENKAFDTVLVYKLDR |
| | | LSRSVRDTLYLVKDVFTKNKIDFISLNESIDTSSAMGSLFLTIL |
| | | SAINEFERENIKERMTMGKLGRAKSGKSMMWTKTAFGYYH |
| | | NRKTGILEIVPLQATIVEQIFTDYLSGISLTKLRDKLNESGHIG |
| | | KDIPWSYRTLRQTLDNPVYCGYIKFKDSLFEGMHKPIIPYETY |
| | | LKVQKELEERQQQTYERNNNPRPFQAKYMLSGMARCGYCG |
| | | APLKIVLGHKRKDGSRTMKYHCANRFPRKTKGITVYNDNKK |
| | | CDSGTYDLSNLENTVIDNLIGFQENNDSLLKIINGNNQPILDTS |
| | | SFKKQISQIDKKIQKNSDLYLNDFITMDELKDRTDSLQAEKK |
| | | LLKAKISENKENDSTDVFELVKTQLGSIPINELSYDNKKKIVN |
| | | NLVSKVDVTADNVDIIFKFQLA* |
| 146 | R4 | MNRGGPTVRADIYVRISLDRTGEELGVERQEESCRELCKSLG |
| | | MEVGQVWVDNDLSATKKNVVRPDFEAMIASNPQAIVCWHT |
| | | DRLIRVTRDLERVIDLGVNVHAVMAGHLDLSTPAGRAVART |
| | | VTAWATYEGEQKAERQKLANIQNARAGKPYTPGIRPFGYGD |
| | | DHMTIVTAEADAIRDGAKMILDGWSLSAVARYWEELKLQSP |
| | | RSMAAGGKGWSLRGVKKVLTSPRYVGRSSYLGEVVGDAQ |
| | | WPPILDPDVYYGVVAILNNPDRFSGGPRTGRTPGTLLAGIAL |
| | | CGECGKTVSGRGYRGVLVYGCKDTHTRTPRSIADGRASSST |
| | | LARLMFPDFLPGLLASGQAEDGQSAASKHSEAQTLRERLDG |
| | | LATAYAEGAISLSQMTAGSEALRKKLEVIEADLVGSAGIPPF |
| | | DPVAGVAGLISGWPTTPLPTRRAWVDFCLVVTLNTQKGRHA |
| | | SSMTVDDHVTIEWRDVAE* |
| 147 | Dre (contains an | MPKKKRKVGSSELIISGSSGGFLRNIGKEYQEAAENFMRFMN |
| | N-terminal NLS) | DQGAYAPNTLRDLRLVFHSWARWCHARQLAWFPISPEMAR |
| | | EYFLQLHDADLASTTIDKHYAMLNMLLSHCGLPPLSDDKSV |
| | | SLAMRRIRREAATEKGERTGQAIPLRWDDLKLLDVLLSRSER |
| | | LVDLRNRAFLFVAYNTLMRMSEISRIRVGDLDQTGDTVTLHI |
| | | SHTKTITTAAGLDKVLSRRTTAVLNDWLDVSGLREHPDAVL |
| | | FPPIHRSNKARITTTPLTAPAMEKIFSDAWVLLNKRDATPNK |
| | | GRYRTWTGHSARVGAAIDMAEKQVSMVEIMQEGTWKKPET |
| | | LMRYLRRGGVSVGANSRLMDS* |
| 148 | Int1 | MTNPASRPKAYSYIRMSSAIQIKGDSFRRQAEASAKYAAEHD |
| | | LDLIDDYKLADLGVSAFKSDNLTTGALGRFVAECEAGEIEAG |
| | | SFLLIESLDRLSRDKILDAFSLFARILKTGVKIVTLSDGQVYDG |
| | | SSDQVGSIYYAISVMIRSNDESKIKSTRGLANWSQKRKLAAE |
| | | HGVKMSSQCPAWLKLSVDRKSYLIDKERAKIVQRIFEASASG |
| | | KGANLITKELNRDKVPTFGRGALWAEAFVSKTLRNRAVLGE |
| | | FQPGQYVSGKRQPAGDPIPGYFPPVIEEELFDIVQASLRGRLL |
| | | AGGRRGEGQSNIFTHVAFCGYCGSKMRHRSKGSRVKGNPPH |
| | | RYLTCFNRFNGPGCDCKPLPYAAFERSFLTFVRDVDLRGLLE |
| | | GAKRKSEAKTIADRITVNEEKVRKADERIRDYLIKIEGAPDLA |
| | | EIFMERIRELKAEKDDLVRSIEESNDALSKIKSDNVTDEELAS |
| | | LISTFQNPCGENRIRLADRIKSIIERIDVYPNGEIRKDDPAIDLV |
| | | RASGDPDAEKIIAAMNAGSRLKDDPYFIVTFRNGAVQTVVPN |
| | | PSNPDDIRVSVYAGEKTRRVEGSAYEYESD |
| 149 | Int2 | MPIAPEFLSLAYPGQEFPAYLYGRASRDPKRKGRSVQSQLDE |
| | | GRATCLDAGWPIAGEFKDVDRSASAYARRTRDEFEEMIAGI |
| | | QAGECRILVAFEASRYYRDLEAYVRLRRVCREAGVLLCYNG |
| | | QVYDLSKSADRKATAQDAVNAEGEADDIRERNLRTTRLNA |
| | | KRGGAHGPVPDGYKRRYDPDSGDLVDQIPHPDRAGLITEIFR |
| | | RAAAAEPLAAICRDLNERGETTHRGKAWQRHHLHAILRNPA |
| | | YIGHRRHLGVDTGKGMWAPICDDEDFAETFQAVQEILSLPG |
| | | RQLSPGPEAQHLQTGIALCGEHPDEPPLRSVTVRGRTNYNCS |
| | | TRYDVAMREDRMDAFVEESVITWLASDEAVAAFEDNTDDE |
| | | RTRKARIRLKVLEEQLEAAQKQARTLRPDGMGMLLSIDSLA |
| | | GLEAELTPQIDKARQESRSLHVPALLRDLLGKPRADVDRAW |
| | | NEALTLPQRRMILRMVVTIRLFKAGSRGVRAIEPGRITLSYVG |
| | | EPGFKPVGGNRAKQ |
| 150 | Int3 | MRKVAIYSRVSTINQAEEGYSIQGQIEALTKYCEAMEWKIYK |
| | | NYSDAGFSGGKLERPAITELIEDGKNNKFDTIL VYKLDRLSR |
| | | NVKDTLYLVKDVFTANNIHFVSLKENIDTSSAMGNLFLTLLS |
| | | AIAEFEREQIKERMQFGVMNRAKSGKTTAWKTPPYGYRYNK |
| | | DEKTLSVNELEAANVRQMFDMIISGCSIMSITNYARDNFVGN |
| | | TWTHVKVKRILENETYKGLVKYREQTFSGDHQAIIDEKTYN |
| | | KAQIALAHRTDTKTNTRPFQGKYMLSHIAKCGYCGAPLKVC |
| | | TGRAKNDGTRRQTYVCVNKTESLARRSVNNYNNQKICNTG |
| | | RYEKKHIEKYVIDVLYKLQHDKEYLKKIKKDDNIIDITPLKKE |
| | | IEIIDKKINRLNDLYINDLIDLPKLKKDIEELNHLKDDYNKAIK |
| | | LNYLDKKNEDSLGMLMDNLDIRKSSYDVQSRIVKQLIDRVE |
| | | VTMDNIDIIFKF |
| 151 | Int4 | MITTRKVAIYVRVSTTNQAEEGYSIQGQIDSLIKYCEAMGWII |
| | | YEEYTDAGFSGGKIDRPAMSKLITDAKHKRFDTIL VYKLDRL |
| | | SRSVRDTLYLVKDVFNQNNIHFVSLQENIDTSSAMGNLFLTL |
| | | LSAIAEFEREQITERMTMGKIGRAKSGKTMAWTYTPFGYDY |
| | | NKEKGELILDPAKAPIVKMIYTDYLKGMSIQKIVDKLNKMD |
| | | YNGKDCTWFPHGVKHLLDNPVYYGMTRYNNKLFPGNHQPII |
| | | TKELFDKTQRERQRRRLGIEENHYTIPFQAKYMLSKFLRCRQ |
| | | CGSRMGLELGRPRKKEGKRSKKYYCLNSRPKRTASCDTPLY |
| | | DAETLEDYVLHEIAKIQKDPSIASRQKHIEDHELKYKRERIEA |
| | | NINKTVNQLSKLNNLYLNDLITLEDLKTQTNTLIAKKRLLEN |
| | | ELDKTCDNDDELDRQETIADFLALPDVWTMDYEGQKYAVE |
| | | LLVQRVKVDRDNIDIHWTF |
| 152 | Int5 | MPGMTTETGPDPAGLIDLFCRKSKAVKSRANGAGQRRKQEI |
| | | SIAAQETLGRKVAALLGMQVRHVWKEVGSASRFRKGKARD |
| | | DQSKALKALESGEVGALWCYRLDRWDRGGAGAILKIIEPED |
| | | GMPRRLLFGWDEDTGRPVLDSTNKRDRGELIRRAEEAREEA |
| | | EKLSERVRDTKAHQRENGEWVNARAPYGLRVVLVTVSDEE |
| | | GDEYDERKLAADDEDAGGPDGLTKAEAARLVFTLPVTDRLS |
| | | YAGTAHAMNTREIPSPTGGPWIAVTVRDMIQNPAYAGWQTT |
| | | GRQDGKQRRLTFYNGEGKRVSVMHGPPLVTDEEQEAAKAA |
| | | VKGEDGVGVPLDGSDHDTRRKHLLSGRMRCPGCGGSCSYS |
| | | GNGYRCWRSSVKGGCPAPTYVARKSVEEYVAFRWAAKLAA |
| | | SEPDDPFVIAVADRWAALTHPQASEDEKYAKAAVREAEKNL |
| | | GRLLRDRQNGVYDGPAEQFFAPAYQEALSTLQAAKDAVSES |
| | | SASAAVDVSWIVDSSDYEELWLRATPTMRNAIIDTCIDEIWV |
| | | AKGQRGRPFDGDERVKIKWAART |
| 153 | Int6 | MQLDATLTLRDEGLSAFHQRHIKQGALGVFLRAIEDGRIQPG |
| | | SVLIVEGLDRLSRAEPIQAQAQLAQIINAGITVVTASDGREYN |
| | | RERLKAQPMDLVYSLLVMIRAHEESDTKSKRVKAAIRRQCE |
| | | GWVAGTWRGIIRNGKDPHWVRLGEHGKFEHVPERVLAVRT |
| | | MIDLFLEGHGAIEITRRLTEQNLYVSNAGNYSVHMYRIVRNQ |
| | | ALIGEKRISVDGEEFRLDGYYPPILTREEFAELQQTMSERGRR |
| | | KGKGEIPNIITGLSITVCGYCGRAMTTQNSKARAPKGKSVVR |
| | | RLSCPMNSFNEGCPIGGSCESEIVERALMRYCSDQFNLSRLLE |
| | | GDDGTARRTAQLAVARQRASDIEAQIQRVTDALLSDDGKAP |
| | | AAFTRRARELETQLEEQRREIEALEHQIAASSAHGIPAAAEA |
| | | WAQLVDGVLALDYDARMKARQLVADTFRKIVVYQRGFAPI |
| | | DDAAADRWKRSGTIGLMLVTKRGGMRLLNVDRRTGCWQA |
| | | EDDLDPSLIPSDGLPMLPLDA |
| 154 | Int7 | MKVAIYVRVSTDEQAKEGFSIPAQRERLRAFCASQGWEIVQE |
| | | YIEEGWSAKDLDRPQMQRLLKDIKKGNIDIVLVYRLDRLTRS |
| | | VLDLYLLLQTFEKYNVAFRSATEVYDTSTAMGRLFITLVAAL |
| | | AQWERENLAERVKFGIEQMIDEGKKPGGHSPYGYKFDKDFN |
| | | CTIIEEEADVVRMIYRMYCDGYGYRSIADRLNELMVKPRIAK |
| | | EWNHNSVRDILTNDIYIGTYRWGDKVVPNNHPPIISETLFKK |
| | | AQKEKEKRGVDRKRVGKFLFTGLLQCGNCGGHKMQGHFD |
| | | KREQKTYYRCTKCHRITNEKNILEPLLDEIQLLITSKEYFMSK |
| | | FSDRYDQQEVVDVSALTKELEKIKRQKEKWYDLYMDDRNPI |
| | | PKEELFAKINELNKKEEEIYSKLSEVEEDKEPVEEKYNRLSK |
| | | MIDFKQQFEQANDFTKKELLFSIFEKIVIYREKGKLKKITLDY |
| | | TLK |
| 155 | Int8 | MKVAVYCRVSTLEQKEHGHSIEEQERKLKSFCDINDWTVYD |
| | | TYIDAGYSGAKRDRPELQRLMNDINKFDLVLVYKLDRLTRN |
| | | VRDLLDLLEIFEKNDVSFRSATEVYDTTTAMGRLFVTLVGA |
| | | MAEWERETIRERTQMGKLAALRKGIMLTTPPFYYDRVDNKF |
| | | VPNKYKDVILWAYDEAMKGQSAKAIARKLNNSDIPPPNNTQ |
| | | WQGRTITHALRNPFTRGHFDWGGVHIENNHEPIITDEMYEKV |
| | | KDRLNERVNTKKVRHTSIFRGKLVCPVCNARLTLNSHKKKS |
| | | NSGYIFVKQYYCNNCKVTPNLKPVYIKEKEVIKVFYNYLKRF |
| | | DLEKYEVTQKQNEPEITIDINKVMEQRKRYHKLYASGLMQE |
| | | DELFDLIKETDQTIAEYEKQNENREVKQYDIEDIKQYKDLLL |
| | | EMWDISSDEDKEDFIKMAIKNIYFEYIIGTGNTSRKRNSLKITS |
| | | IEFY |
| 156 | Int9 | MKVAIYTRVSTLEQKEKGHSIEEQERKLRAYSDINDWKIHKV |
| | | YTDAGYSGAKKDRPALQEMLNEIDNFDLVLVYKLDRLTRSV |
| | | KDLLEILELFENKNVLFRSATEVYDTTSAMGRLFVTLVGAM |
| | | AEWERTTIQERTAMGRRASARKGLAKTVPPFYYDRVNDKFV |
| | | PNEYKKVLRFAVEEAKKGTSLREITIKLNNSKYKAPLGKNW |
| | | HRSVIGNALTSPVARGHLVFGDIFVENTHEAIISEEEYEEIKLR |
| | | ISEKTNSTIVKHNAIFRSKLLCPNCNQKLTLNTVKHTPKNKEV |
| | | WYSKLYFCSNCKNTKNKNACNIDEGEVLKQFYNYLKQFDLT |
| | | SYKIENQPKEIEDVGIDIEKLRKERARCQTLFIEGMMDKDEAF |
| | | PIISRIDKEIHEYEKRKDNDKGKTFNYEKIKNFKYSLLNGWEL |
| | | MEDELKTEFIKMAIKNIHFEYVKGIKGKRQNSLKITGIEFY |
| 157 | Int10 | MITTNKVAIYVRVSTTNQVEEGYSIDEQKDKLSSYCDIKDWN |
| | | VYKVYTDGGFSGSNTDRPALESLIKDAKKRKFDTVLVYKLD |
| | | RLSRSQKDTLHLIEDVFIKNGIEFLSLQENFDTSTPFGKAMIGL |
| | | LSVFAQLEREQIKERMQLGKLGRAKSGKSMMWAKTSYGYD |
| | | YHKETGTVTINPAQALTIKFIFESYLRGRSITKLRDDLNEKYP |
| | | KHVPWSYRAVRTILDNPVYCGFNQYKGEIYPGNHEPIISKEE |
| | | YDKTQSELKIRQRTAAENVNPRPFQAKYILSGIAQCGYCGAP |
| | | LKIMLGVKRKDGSRLKKYECHQRHPRTLRGVTTYNDNKKC |
| | | DSGFYYKDKLEAYVLKEISKLQDDADYLDKIFSGDNAETIDR |
| | | ESYKKQIEELSKKLSRLNDLYIDDRITLEELQSKSAEFISMRGT |
| | | LETELENDPALRKNKRKADMRKLLNAEKVFSMDYESQKVL |
| | | VRRLINKVKVTAEDIVINWKI |
| 158 | Int11 | MLRCAIYIRVSTEEQAMHGLSMDAQKADLTDYAKKHNYEII |
| | | DYYVDSGKTARKRLSKRKDLQRMIEDVKLNKIDIIIFTKLDR |
| | | WFRNVRDYYKIQEVLEDHNVDWKTIFENYDTSTANGRLHIN |
| | | IMLSVAQDEADRTSERIKRVFENKLKNNEPTSGSLPIGYKIKE |
| | | KSIIIDEEKAPIAKDVFDFYYYHQSQTKVFKEILNKYNLSLCE |
| | | KTIRRMLENKLYIGIYREHENFCPPLIDKNKFDEVQLILKRRNI |
| | | KYIPTKRIFLFTSLLICKECRHKMIGNAQIRNTKAGKIEYILYR |
| | | CNQSYARHTCNHRKVIYENKIETYLLNNIESELKKFIYDYELE |
| | | DIPKVKNKVNKTNIKRKLEKLKELYINDLIDIDMYKEDYKKY |
| | | TEILNTKEEKIEQRNLQPLKDFLNSDFKSLYSSISREEKRLLW |
| | | RGIISEIQIDCNNDITIIPHP |
| 159 | Int12 | MKVAIYTRVSSAEQANEGYSIHEQKKKLISYCEIHDWNEYK |
| | | VFTDAGISGGSMKRPALQKLMKHLSSFDLVLVYKLDRLTRN |
| | | VRDLLDMLEEFEQYNVSFKSATEVFDTTSAIGKLFITMVGAM |
| | | AEWERETIRERSLFGSRAAVREGNYIREAPFCYDNIEGKLHP |
| | | NEYAKVIDLIVSMFKKGISANEIARRLNSSKVHVPNKKSWNR |
| | | NSLIRLMRSPVLRGHTKYGDMLIENTHEPVLSEHDYNAINNA |
| | | ISSKTHKSKVKHHAIFRGALVCPQCNRRLHLYAGTVKDRKG |
| | | YKYDVRRYKCETCSKNKDVKNVSFNESEVENKFVNLLKSYE |
| | | LNKFHIRKVEPVKKIEYDIDKINKQKINYTRSWSLGYIEDDEY |
| | | FELMEEINATKKMIEEQTTENKQSVSKEQIQSINNFILKGWEE |
| | | LTIKDKEELILSTVDKIEFNFIPKDKKHKTNTLDINNIHFKF |
| 160 | Int13 | MAVGIYIRVSTQEQASEGHSIESQKKKLASYCEIQGWDDYRF |
| | | YIEEGISGKNTNRPKLKLLMEHIEKGKINILLVYRLDRLTRSVI |
| | | DLHKLLNFLQEHGCAFKSATETYDTTTANGRMSMGIVSLLA |
| | | QWETENMSERIKLNLEHKVLVEGERVGAIPYGFDLSDDEKL |
| | | VKNEKSAILLDMVERVENGWSVNRIVNYLNLTNNDRNWSP |
| | | NGVLRLLRNPALYGATRWNDKIAENTHEGIISKERFNRLQQI |
| | | LADRSIHHRRDVKGTYIFQGVLRCPVCDQTLSVNRFIKKRKD |
| | | GTEYCGVLYRCQPCIKQNKYNLAIGEARFLKALNEYMSTVE |
| | | FQTVEDEVIPKKSEREMLESQLQQIARKREKYQKAWASDLM |
| | | SDDEFEKLMVETRETYDECKQKLESCEDPIKIDETYLKEIVY |
| | | MFHQTFNDLESEKQKEFISKFIRTIRYTVKEQQPIRPDKSKTG |
| | | KGKQKVIITEVEFYQ |
| 161 | Int14 | MTVGIYIRVSTEEQVKEGFSISAQKEKLKAYCTAQGWEDFKF |
| | | YVDEGKSAKDMHRPLLQEMISHIKKGLIDTVLVYKLDRLTRS |
| | | VVDLHNLLSIFDEFNCAFKSATEVYDTSSAMGRFFITIISSVAQ |
| | | FERENTSERVSFGMAEKVRQGEYIPLAPFGYTKGTDGKLIVN |
| | | KIEKEIFLQVVEMVSTGYSLRQTCEYLTNIGLKTRRSNDVWK |
| | | VSTLIWMLKNPAVYGAIKWNNEIYENTHEPLIDKATFNKVA |
| | | KILSIRSKSTTSRRGHVHHIFKNRLICPACGKRLSGLRTKYINK |
| | | NKETFYNNNYRCATCKEHRRPAVQISEQKIEKAFIDYISNYTL |
| | | NKANISSKKLDNNLRKQEMIQKEIISLQRKREKFQKAWAADL |
| | | MNDDEFSKLMIDTKMEIDAAEDRKKEYDVSLFVSPEDIAKR |
| | | NNILRELKINWTSLSPTEKTDFISMFIEGIEYVKDDENKAVITK |
| | | ISFL |
| 162 | Int15 | MKAAIYIRVSTQEQIENYSIQAQTEKLTALCRSKDWDVYDIFI |
| | | DGGYSGSNMNRPALNEMLSKLHEIDAVVVYRLDRLSRSQRD |
| | | TITLIEEYFLKNNVEFVSLSETLDTSSPFGRAMIGILSVFAQLE |
| | | RETIRDRMVMGKIKRIEAGLPLTTAKGRTFGYDVIDTKLYIN |
| | | EEEAKQLQMIYDIFEEEKSITTLQKRLKKLGFKVKSYSSYNN |
| | | WLTNDLYCGYVSYADKVHTKGVHEPIISEEQFYRVQEIFSRM |
| | | GKNPNMNRDSASLLNNLVVCGKCGLGFVHRRKDTISRGKK |
| | | YHYRYYSCKTYKHTHELEKCGNKIWRADKLEELIIDRVNNY |
| | | SFASRNVDKEDELDNLNEKLKTEHKKKKRLFDLYISGSYEVS |
| | | ELDAMMADIDAQINYYEAQIEANEELKKNKKIQENLADLAT |
| | | VDFDSLEFREKQLYLKSLINKIYIDGEQVTIEWL |
| 163 | Int16 | MKGESELDKKAAIYIRVSTQEQATEGYSIQAQTDRLIKYVEA |
| | | KDFILYKKYIDAGYSASKLERPAMQDLIQDVQSKKVDVVIV |
| | | YKLDRLSRSQKDTMYLIEDIFRPNDVELISMQESFDTSTAFGS |
| | | ATVGMLSVFAQLERKSISERMITGRVERAKKGFYHTGGQDR |
| | | PPAGYQFNSDNQLIINEYEAAAIKDLFRLYNDGLGKSSISEYL |
| | | KKNYPGKNKWLPSSIDRMLKNSLYIGKVKFSGAEYDGIHEPII |
| | | DEVTFYKTQKEIARRKQTNTKRYNYVALLGGLCECGICGAK |
| | | MANRRAVGRKGKVYRYYRCYSKKGSPKHMMKTDGCSSKA |
| | | QQQFIIDEAVINNLKNIDVEAELKRRSAPQTNTSLISSQIESIDK |
| | | QINKLIDLFQVDSMPLDVISEKIDKLNKEKQSMEKLLERKNK |
| | | LDKTELQHRFDVLKSFDWDNSSIESKRVVIEMLVQKVIIHDN |
| | | SIEIILVE |
| 164 | Int17 | MRTNEHNFHNIEEEIKHVAVYLRLSRGEDESELDNHKTRLLN |
| | | RCELNNWSYELYKEIGSGSTIDDRPVMQKLLTDVEKNLYDA |
| | | VLVVDLDRLSRGNGTDNDRILYSMKVSETLIVVESPYQVLD |
| | | ANNESDEEIILFKGFFARFEFKQINKRMREGKKLAQSRGQWV |
| | | NSVTPYGYIVNKTTKKLTPSEEEAKVVIMIKDFFFEGKSTSDI |
| | | AWELNKRKIKPRRATEWRSSSIANILQNEVYVGNIVYNKSVG |
| | | NKKPSKSKTRVTTPYRRLPEEEWRRVYNAHQPLYSKEEFDRI |
| | | KQYFECNVKSHKGSEVRTYALTGLCKTPDGKTMRVTQGKK |
| | | GTDDDLYLFPKKNKHGDSSIYKGISYNVVYETLKEVILQVKD |
| | | YLDSVLDQNENKDLVEELKEELMKKEDELETIQKAKNRIVQ |
| | | GFLIGLYDEQDSIELKVEKEKEIDEKEKEIEAIKMKIDNAKTV |
| | | NNSIKKTKIERLLSDVQSAESEKEINRFYKTLIKEIIVDRTDEN |
| | | EAKIKVNFL |
| 165 | Int18 | MITTNKVAIYVRVSTTNQVEEGYSIDEQKDKLEAYCKIKDW |
| | | KIYDVYVDGGFSGANTQRPELERLISDVKRKKVDIVLVYKLD |
| | | RLSRSQKDTLFLIEDVFAKNDVAFISLQENFDTSTPFGKASIG |
| | | MLSVFAQLEREQIKERMMLGKEGRAKNGKSMSWTTIAFGY |
| | | DYSKETGVLSVNPTQALIVNRIFTEYLNGKPVVKIIRDLNAEG |
| | | HVGRKRPWGETITKYLLKNETYLGKVKYKDKVYEGQHEPII |
| | | TQELFDLVQLEVERRQISAYEKYNNPRPFRAKYMLSGLMKC |
| | | GYCGASLGLRYTRKDKNGISHHKYQCRNRHSKDLEKRCESG |
| | | WYSKEELERGVIKELERIKFDPKYKNETLAKKEETIKVEEIKK |
| | | QLERINNQVSKLTELYLDEIITRKELDEKNDKIKTERQFLEEQ |
| | | LENQKSNVLSIRKRKLTRLLKDFDVEKLSYEDASKIVKNIIKE |
| | | IIVTKDGMSITLDF |
| 166 | Int19 | MGKSITVIPAKKVQTSVLHQDRKKIKVAAYCRVSTDQEEQLS |
| | | SYENQVNYYREFISKHEDYELVDIYADEGISATNTKKRDAFN |
| | | RLIQDCRAGKVDRILVKSISRFARNTLDCIKYVRELKELGVG |
| | | VTFEKENIDSLDSKGEVLLTILSSLAQDESRSISENATWGIRKK |
| | | FERGEVRVNTTKFMGYDKDENGRLIINPQQAETVKFIYEKFL |
| | | EGYSPESIAKYLNDNEIPGWTGKANWYPSAIQKMLQNEKYK |
| | | GDALLQKTFTVDFLTKKRVQNDGQVNQYYVENSHEAIIDEE |
| | | TWETVQLEMARRKTYRDEHQLKSYIMQSEDNPFTTKVFCGA |
| | | CGSAFGRKNWATSRGKRKVWQCNNRYRIKGVEGCYSSHLD |
| | | EATLEQIFLKALELLSENIDLLDGKWEKILAENRLLDKHYSM |
| | | ALSDLLRQEQIDFNPSDMCRVLDHIRIGLDGEITVCLLEGTEV |
| | | DL |
| 167 | Int20 | MRTVRRIQPIKSPCKPRFKVAAYARVSDSRLHHSLSTQISYYN |
| | | RLIQAHPDWELVGIYYDEGISGKEQSNRQGFLNLIKDCEDGK |
| | | IDRIITKSIARFGRNTVELLTTVRQLRLKNIGVTFEKENIDSLSS |
| | | EGELMLTLLASVAQEESQNLSENIRWRIQKKFEKGIPHTPQD |
| | | MYGYRWDGEQYQIEPNEAKVIRKVFKWYLDGDSVQQIVDK |
| | | LNQEQVLTRLGNPFTVASIREFFKQEAYFGRLVLQKTYREAF |
| | | SRNPKRNKGQRNKYIIENAHEPIVTKEYFDLVLHEKERRNQL |
| | | MHQESHLNKGIFRDKISCSECGCLMIVKVDSKQVNKTVRYY |
| | | CRTRNRFGASSCSCRTLGEKRLLASFKSKLGIVPDKEWVENN |
| | | IKHIEYDFGYRILRVTPVKGRKYLIEIREGRY |
| 168 | Int21 | MRNKVAIYVRVSTASQADEGYSIDEQKSKLEAYCEIKDWKI |
| | | YDTYIDGGFSGANTQRPELERLISDAKRKKIDIVLVYKLDRLS |
| | | RSQKDTLFLIEDVFAKNDVAFISLQENFDTSTPFGKASIGMLS |
| | | VFAQLEREQIKERMMLGKEGRAKNGKSMSWTTIPFGYDYSK |
| | | ETGILSVNPTQALIVKRIFTEYLNGKSVVKIIRDLNAEGHVGR |
| | | KRPWGETITKYLLKNETYLGKSKYKGKVFEGQHDAIISQELF |
| | | DLVQLEVEKRQISAFEKYNNPRPFRAKYMLSGLMKCGYCGA |
| | | SLGLYVAPKNKNGVSKYKYQCRHRYHKDKAIRCNSGWYSK |
| | | DELEKRVIKELERLKFDPKYKKETLAKKDETIKVEDIKKQLE |
| | | RINKQVSKLTELYLDEVITRKDLDEKNAKIKTERQYLEEQLE |
| | | NQKSNVMSIRKRKLSRLLKDFDIEKLSYEEASKIVKSVIKEIV |
| | | VTKDDMTITLDF |
| 169 | Int22 | MKVATYVRVSTDEQAKEGFSIPAQRERLRAFCESQGWEIVEE |
| | | YIEEGWSAKDLDRPQMQRLLKDIKKGNIDIVLVYRLDRLTRS |
| | | VLDLYLLLQTFEKYNVAFRSATEVYDTSTAMGRLFITLVAAL |
| | | AQWERENLAERVKFGIEQMIDEGKKPGGHSPYGYKFDKDFN |
| | | CTIIEDEANTVRMIYRMYCDGYGYHSIAKRLNELGIKPRIAKE |
| | | WNHNSVRDILTNDIYIGTYRWGNKVVLNNHPPIISETLFRKV |
| | | QKEKEKRRVDRTRVGKFLLTGLLYCGNCNGHKMQGTFDKR |
| | | EQKTYYRCLKCNRITNEKNILEPLLDEIQLLITSKEYFMSKFS |
| | | DQYDQKEEVDVSALKKELEKIKRQKEKWYDLYMDDRNPIP |
| | | KEDLFAKINELNKKEEEIYNKLNEVEPEDKEPVEEKYNRLSK |
| | | MIDFKQQFEQANDFTKKELLFSIFEKIVIYREKGKLKKITLDY |
| | | TLK |
| 170 | Int23 | MLRVALYIRVSTEEQALNGDSIRTQIEALEQYSKENDFNIVG |
| | | KYIDEGCSATNLKRPNLQRLLRDVEKDKVDLVLMTKIDRLS |
| | | RGVKNYYKIMETLEKHKCDWKTILENYDSSTAAGRLHINIM |
| | | LSVAENEAAQTSERIKFVFQDKLRRKEVISGTIPIGYKIENKHL |
| | | VIDKEKKYIVKAIFDEYEKSGSVRTLIETINNLHGELYSYNKI |
| | | KNILRNELYIGIYNKRGFYVEDYCEPIISKKQFKQIQRILEKNK |
| | | KTTPNKNIHYHIFSGLLKCKECGYTLKGNSSNVGEKLYLSYR |
| | | CSTFYLNKNCVHNVTHNEKHIENYLLTNLKPQLHKHMVKLE |
| | | AQNEKIRRNKKSNKKDEKKKIMKKLDKIKDLYLEDLIDKET |
| | | YRKDYEKLQSQLDNITEEQESQIIDTSHIKKFLDIDINEMYSDL |
| | | SRVERRRFWLSIIDYIEIDNNKNITINFI |
| 171 | Int24 | MKITLLYYIKKFNIYCNRYLSQQINISVDIIGFYQFKNVTNSVT |
| | | DVLKRGDNLDRICIYLRKSRADEELEKTIGVGETLSKHRKAL |
| | | LKFAKEKKLNIMEIKEEIVSADSIFFRPKMIELLKEVENNQYT |
| | | GVLVMDIQRLGRGDTEDQGIIARIFKESHTKIITPMKTYDLDD |
| | | DLDEDYFEFESFMGRKEYKMIKKRMQGGRVRSVEDGNYIAT |
| | | NPPFGYDIHWINKSRTLKFNSKESEIVKLIFKLYTEGNGAGTIS |
| | | NYLNSLGYKTKFGNNFSNSSIIFILKNPVYIGKITWKKKDIRKS |
| | | KDPHKVKDTRTRDKSEWIIADGKHEPIIDEKIWNKAQEILNN |
| | | KYHIPYKIANGPANPLAGVVICSKCNSKMVMRKYGKKLPHL |
| | | ICNNKECNNKSARFDYIEKAVLEGLDEYLKNYKVNVKANNK |
| | | TSDIEPYEQQSNALNKELILLNEQKLKLFDFLEREIYTEEIFLE |
| | | RSKNLDERINTTTLAINKIKKILDNEKKKNNKNDIVKFEKILE |
| | | GYKKTNDIQKKNELMKSLVFKIEYKKEQHQRNDGLLYIYFL |
| | | SFCVRCISYLTQFISFFVYPYRILEIYLTFSFFIISYEH |
| 172 | Int25 | MRICMYLRKSRADEELEKTLGEGETLSKHRKALLKFAKEKN |
| | | LNIVEIKEEIVSGESLFFRPKMLELLKEIENKQYSGVLVMDMQ |
| | | RLGRGNMQDQGIILETFKKSNTKIITPMKTYDLSNDFDEEYSE |
| | | FEAFMSRKELKMINRRMQGGRVRSVEDGNYIATNAPYGYDI |
| | | HWINKARTLKPNQKESEIVKLIFKLYIEGNGAGTIAKHLNSLG |
| | | YKTKFGNSFNNSSIIFILKNPVYIGKITWKKKDIRKSKDPNKV |
| | | KDTRTRDKSEWIIVDGKHDPIIDQITWKQAQEILNNRYHVPY |
| | | KLVNGPANPLAGLIICTTCKSKMVMRKLRGTDRILCKNNKC |
| | | NNISNRFDAVEKSVVESLENYLKAYKVNLPELNKTSNLKLYE |
| | | QQISTLKKELKILNEQKLKLFDFLERGIYDEDTFLKRSKNLDE |
| | | RIEITNESLSNLNQIIAKENKAIKKEDIIKFEKVLDSYKSTADIR |
| | | LKNELMKTLIFKIEYTKNKKGNDFKIKVFPKLKPLNI |
| 173 | Int26 | MIAAIYSRKSKFTGKGESVENQIEMCKEYLKRNFNNIDDIEIY |
| | | EDEGFSGKDTNRPKFKKMIKAAKNKKFNILICYRLDRISRNV |
| | | ADFSNTIEELQKYNIDFISIKEQFDTSTPMGRAMMNIAAVFAQ |
| | | LERETIAERIKDNMVELAKTGRWLGGTSPLGYKSEPIEYSNE |
| | | DGKSKKMYKLTEVENEMNIVKLIYKLYLEKRGFSSVATYLC |
| | | KNKYKGKNGGEFSRETARQIVINPVYCISDKTIFKWFKSKGA |
| | | TTYGTPDGIHGLMVYNKREGGKKDKPINEWIIAVGKHRGVIS |
| | | SDIWLKCQNLIQQNNAKSSPRSGTGEKFLLSGMVVCKECGS |
| | | GMSSWSHFNKKTNFMERYYRCNLRNRASNRCSTKMLNAYK |
| | | AEEYVANYLKELDINAIKKMYHSNKKNIIDYDAKYEVNKLN |
| | | KSIEENKKIIQGIIKKIALFDDLDILGMLKNELERLKKENDEM |
| | | KIKLKELKSILELEDEEEIFLSTMEENISNFKKFYDFVNITQKRI |
| | | LIKGLVESIVWDTGGEEKILEINLIGSNTKLPSGKVKRRE |
| 174 | Int27 | MSKKVAIYTRVSTTNQAEEGYSIDEQIDKLKMYCEAMDWK |
| | | VSEIYTDAGFTGSKLTRPAMEKMITDIGLKKFDTVIVYKLDR |
| | | LSRSVRDTLYLVKDVFTKNEIDFISLSESIDTSSAMGSLFLTILS |
| | | AINEFERENIKERMTMGKIGRAKSGKSMMWAKTAFGYSHN |
| | | QETGILEINPLEASIVEQIFNEYLKGTSITKLRDKLNEDGHIAK |
| | | ELPWSYRTIRQTLDNPVYCGYIKYKNNTFEGLHKPIISHETYL |
| | | SVQKELEARQQQTYEKNNNPRPFQAKYLLSGIARCGYCGAP |
| | | LRIVLGHRRKDGSRTMKYQCVNRFPRKTKGVTTYNDNKKC |
| | | DSGAYDMQWIEDIVLKTLNGFQKSDKKLRKILNIKEESKVDT |
| | | SGFQKQLKSINNKIQKNSDLYLNDFITMDDLKKRTEMLQGE |
| | | KKLIQARINEVDKPSTSEIFDLVKSELGETTISKISYEDKKKIV |
| | | NNLISKVDVTADNIDIIFKFQLA |
| 175 | Int28 | MNEQKDKLKKYCEIKDWTIVKEYVDPGRSGSNINRPSMQQL |
| | | IKDADTGLYDAVLVYKLDRLSRSQKDTLYLIEDVFQKNNIHF |
| | | ISLSENFDTSTAFGKAMIGILSVFAQLEREQIKERMSMGRVGR |
| | | AKSGKIMEFNNPAFGYEVDGDNYKVDPLRAEIVKRIYKMYL |
| | | SGTSINKIKETLNLEGHIGNKKNWSDTRIRYILSNPTYLGKIRY |
| | | DGKTYDGKFSPIIDEETFNKTQNELKERQTATYKRFNMKLRP |
| | | FQSKYMLSGLLRCGYCGATLFVNSYVYNGKRKLRYNCPSTY |
| | | KSKQKTRTYKIMDPNCPFKLVYAKDLEPAVINEIKNLALNPQ |
| | | SIQKPVKKKPDIDVEAIQKELAKVRKQQQRLIDLYVISDDVNI |
| | | DNISKKSADLKLQEETLKKQLAPLEEPNDDDKIVAFNEILAQI |
| | | KDIDSLDYDKQKFIVKKLIKKIDVWNDNKIKIHWNI |
| 176 | Int29 | MKTAIYLRKSRADLEAEARGEGETLAKHRSTLLKIAKEMNL |
| | | NVLSVREEIVSGESLVKRPEMLALLEEIEDNKYDAVLCMDM |
| | | DRLGRGGMKEQGIILETFKRSNTKIMTPRKTYDLNDEWDEE |
| | | YSEFEAFMARKELKIITRRMQRGRIASVEAGNYLGTHAPFGY |
| | | DIHRLNKRERTLTINSEEASVVRMIFDWYANEDMGASAIRNK |
| | | LNDLGYKSKLGNDWNPYSILDILKNNIYIGKVTWQKRKEVK |
| | | RPDAVKRSCARQDKSDWIIADGKHEPIIPESLFEQAQEKLNSR |
| | | YHVPYNTNGIKNPLAGIIKCSKCGYSMVQRYPKNRKETMDC |
| | | KHRGCENKSSYTELIEKRLLEALKEWYINYKADFEAHKQGD |
| | | KLKETQVIQMNEAALRKLEKELVDVQKQKNNLHDLLERGV |
| | | YTVDMFLERSQVISDRINEITSTMENLKKEIKTEIKKEKVKKD |
| | | TIPQVEHVLDLYFKTDDPKKKNSLLKSVLEKAVYKKEKWQR |
| | | LDDFELVLYPKLPQDGDI |
| 177 | Int30 | MYRPESLDVCIYLRKSRKDVEEERRAIEEGSSYNALERHRKR |
| | | LFAIAKAENHNIIDIFEEVASGESIQERPQMQQLLRKLEGNEID |
| | | GVLVIDLDRLGRGDMLDAGMIDRAFRYSSTKIITPTDVYDPD |
| | | DESWELVFGIKSLISRQELKSITKRLQNGRIDSVKEGKHIGKK |
| | | PPYGYLKDENLRLYPDPEKAWIVKKIFELMCDGKGRQMIAA |
| | | ELDRLGIDPPVTKRGAWDSSTITSIIKNEVYTGVIVWGKFKHK |
| | | KRNGKYTRHKNPQEKWIMYENAHEPIISKELFDAANEAHSS |
| | | RHKPAVITSKKLTNPLAGILKCKLCGYTMLIQTRKDRPHNYL |
| | | RCNNPACKGKQKQSVFNLVEEKLLYSLQQIVDEYQAQKVEE |
| | | VEIDDSKLISFKEKAIISKEKELKELQAQKGNLHDLLEQGIYT |
| | | VEIFLERQKNLVERITSIENDIEVLQKEIETEQIKEHNKTEFIPA |
| | | LKTVIESYHKTTNIELKNQLLKTILSTVTYYRHPDWKTNEFEI |
| | | QVYFKI |
| 178 | Int31 | MKYLALHENSRIAVYSRKSREDRDSEDTLAKHRNELEYLIKR |
| | | ENFKNVQWFEKVVSGETIDERPMFSLLLPRIENGEFDAVCAV |
| | | AMDRLSRGSQIDSGRILEAFKQSGTLFITPKKTYDLSIEGDEM |
| | | LSEFESIIARSEYRAIKRRTINGKKNATREGRLHSGSVPYGYK |
| | | WDKNLKAAVVVEEKKKIYRMMIKWFLEEEYSCTVIAEMLN |
| | | ELKVPSPSGRSIWYGEVVSEILSNDFHRGYVWFGKYKKSKSN |
| | | NSIVQNKNLDEVLIAKGHHETMKTDEEHALILNRIEKLRTYK |
| | | VAGRRLNMNTHRLSGIVRCPYCHKAQAIEQPKGRRKHVRKC |
| | | LRKSAERTKECEETKGIHEEVLFQSIMKEIKKYNESLFSPTEQ |
| | | DVNDDSYTAQLIGLREKAVKKAKGRIERIKEMYLDGDISKTE |
| | | YKEKLKISQETLQKAENELAELIASTEFQNALSAETKKEKWS |
| | | HHKVQEMIESTDGMSNSEINLILKMLISHVTYTVEDLGDGTK |
| | | NLNIKVYYN |
| 179 | Int32 | MDPQHKPTRALIVIRLSRLTDETTSPERQLEACERFCAARGW |
| | | EVVGVAEDLDVSAGTTSPFERPSLSQWIGDGKDNPGRIGEFD |
| | | TVVFYRVDRLVRRVRHLHDVIAWSERFDVNMVSATESHFDL |
| | | STTIGALIAQLVASFAEMELEGISQRATSAHRHNVQLGKFVG |
| | | GSPPFGYMPEETPDGWRLVHDPDVVPIILEVVDRVLEGEPLR |
| | | RITDDLNARGATTARDLVKQRKGKETEGHKWHSNVLKRRL |
| | | MSPAMLGYALRREPLTDSKGKPKLSAKGAKLYGPEEIVRGP |
| | | DGLPVQRAEPILPKPLFDRVVAELEARELQKEPTKRINSMLLR |
| | | VLYCGVCGQPVYRAKGQGGRSDRYRCRSIQDGANCGNPSV |
| | | LTYELDDLVEESILVLMGDSERLAHVWNPGEDNASELAEVE |
| | | ARLADRTGLIGVGAYKAGTPQRATLDTLIEADAKLYERLKA |
| | | ATPRPAGWTWEPTGETFAEWWAALDTGARNVYLRNMGVR |
| | | VTYDKRPVPEQVSAGEKPRVHLELGEVRKMAEQVAVIGTIG |
| | | TLTRNYTRLGEIGITHVDIDAGSGKAVFVTKSGERFELPLNIP |
| | | EE |
| 180 | Int33 | MKAIAIYARKSLFTGKGDSIGAQVDTCKRFIDYKFANEDYEI |
| | | RTFKDEGWSGKTTDRPDFTNMVNLIKSKKIDYVITYKLDRIG |
| | | RTARDLHNFLYELDNLGIVYLSATEPYDTTTSAGRFMISILAA |
| | | MAQMERERLAERVKSGMIQIAKKGRWLGGQCPLGFDSKREI |
| | | YIDDMGKERQMMRLTPNKEEIKIVKLIYDKYLEMGSMSQVR |
| | | KYCLENSIRGKNGGDFSTNTLKQLLTSPIYVKSSDNIFKYLES |
| | | QNINVFGTPNGNGMLTENKTKEIRIERDKSEWIAAVGKHKGII |
| | | DDNKWLQIQQQLQQQSEKQIKSSGRQGTTSTGLLSGIIKCSK |
| | | CGNNLLIKTGHKSKKNPGTTYSYYVCGKKDNSYGHKCDNK |
| | | NVRTDEADSAVITQLKLYNKELLIKNLKEALIQNEKTDTDNI |
| | | EILESKLKEKEKAVSNLVKKLSLIDDESISNIILNEVTNINKEIN |
| | | DIKLQLSNETLKINEVTKATLDTEIYIKILENFNKKIDDITDPIE |
| | | KMNLLKSALESVEWNGDSGEFKINLIGSKKK |
| 181 | Int34 | MKVAIYTRVSTLEQREKGHSIDEQERKLRSFCDINDWTVKD |
| | | VYVDAGFSGAKRDRPELTRLLDDISEFDLVLVYKLDRLTRSV |
| | | RDLLDLLEVFENNNVAFRSATEVYDTTTAIGRLFVTLVGAM |
| | | AEWERETIRERSLMGKRAAIKKGMILTAPPFYYDRVNNTYIP |
| | | NQYKDVVLDVYNKVKKGYSIAHIARLYNNSDVKPPNGNEE |
| | | WTTRMLMHALRNPVTRGHYQWGEIYIEDSHEPIITDEMYNTI |
| | | IDRLDKHTNTKVVAHTSVFRGKLICPNCGYALTLNSQKRKR |
| | | KNDTIVYKTYYCNNCKITKGMKPHHITETETLRVFKDHLSKI |
| | | DLKQYETQEKEKQSHVTIDLSKVMEQRKRYHKLYASGMMQ |
| | | ENELFELIKETDEMIEEYEKQRKQVDVKEFDICKIKEIKDVLL |
| | | KSWDIFTLEDKADFIQMSIKAINIEYTKLKRGKSSNSMKIKDI |
| | | EFY |

TABLE 9
Exemplary Recombinase Nucleic Acid Sequences

| SEQ ID | | |
|---|---|---|
| NO: | Description. | Sequence |
| 182 | PhiC31 (contains | ATGGATACCTACGCCGGAGCCTACGACAGACAGAGCCGG |
| | a C-terminal | GAGAGAGAGAACAGCAGCGCCGCCAGCCCCGCCACCCAG |
| | NLS) | AGAAGCGCCAACGAGGATAAGGCCGCCGATCTGCAGAGA |
| | | GAGGTGGAGAGGGACGGCGGCAGATTCAGATTTGTGGGC |
| | | CACTTCAGCGAGGCCCCTGGCACCAGCGCCTTCGGCACCG |
| | | CCGAGAGGCCCGAGTTCGAGAGAATCCTGAACGAGTGTA |
| | | GGGCCGGCAGGCTGAACATGATCATCGTGTACGACGTGTC |
| | | CCGGTTCAGCAGGCTGAAGGTGATGGACGCCATCCCTATC |
| | | GTGTCCGAGCTGCTGGCCCTGGGCGTGACCATCGTGTCCA |
| | | CCCAGGAAGGCGTCTTTAGACAGGGCAACGTGATGGACCT |
| | | GATCCACCTGATCATGAGGCTGGACGCCAGCCACAAGGA |
| | | GAGCAGCCTGAAAAGCGCCAAGATCCTGGACACCAAGAA |
| | | CCTGCAGAGGGAGCTGGGCGGCTATGTGGGCGGCAAGGC |
| | | CCCCTACGGCTTCGAGCTGGTGTCCGAAACCAAGGAGATC |
| | | ACCCGGAACGGCAGGATGGTGAACGTGGTGATCAACAAG |
| | | CTGGCCCACAGCACCACCCCCCTGACCGGCCCCTTCGAGT |
| | | TTGAGCCCGACGTGATCAGGTGGTGGTGGCGGGAGATCA |
| | | AGACCCACAAGCACCTGCCTTTCAAGCCCGGCAGCCAGGC |
| | | CGCCATCCACCCCGGCAGCATCACCGGCCTGTGTAAGAGA |
| | | ATGGACGCCGACGCCGTGCCCACCAGAGGCGAAACCATC |
| | | GGCAAGAAAACCGCCAGCAGCGCCTGGGACCCCGCCACC |
| | | GTGATGAGAATCCTGAGGGACCCTAGGATCGCCGGCTTCG |
| | | CCGCCGAGGTGATCTACAAGAAGAAGCCCGACGGCACCC |
| | | CCACCACCAAGATCGAGGGCTACAGAATCCAGAGGGACC |
| | | CCATCACCCTGAGGCCTGTGGAGCTGGACTGTGGCCCTAT |
| | | CATCGAGCCTGCCGAGTGGTACGAGCTGCAGGCCTGGCTG |
| | | GACGGCAGAGGCAGAGGCAAGGGCCTGAGCAGAGGCCAG |
| | | GCCATCCTGAGCGCCATGGACAAGCTGTACTGTGAGTGTG |
| | | GCGCCGTGATGACCAGCAAGAGAGGCGAGGAGAGCATCA |
| | | AGGACAGCTACCGGTGCCGGAGAAGAAAGGTGGTGGACC |
| | | CCAGCGCCCCTGGCCAGCACGAGGGCACCTGTAATGTGAG |
| | | CATGGCCGCCCTGGACAAGTTCGTGGCCGAGCGGATCTTC |
| | | AACAAGATCCGGCACGCCGAGGGCGACGAGGAAACCCTG |
| | | GCCCTGCTGTGGGAGGCCGCCAGAAGATTCGGCAAGCTG |
| | | ACCGAGGCCCCCGAAAAGAGCGGCGAGAGGGCCAACCTG |
| | | GTGGCCGAGAGAGCCGACGCCCTGAACGCCCTGGAGGAG |
| | | CTGTACGAGGACAGAGCCGCCGGAGCCTATGACGGCCCT |
| | | GTGGGCAGGAAGCACTTCAGAAAGCAGCAGGCCGCCCTG |
| | | ACCCTGAGACAGCAGGGCGCCGAGGAAAGACTGGCCGAG |
| | | CTGGAGGCCGCCGAGGCCCCTAAGCTGCCCCTGGATCAGT |
| | | GGTTCCCCGAGGATGCCGACGCCGACCCCACCGGCCCCAA |
| | | GTCCTGGTGGGGCAGAGCCAGCGTGGACGACAAGAGGGT |
| | | GTTCGTGGGCCTGTTCGTGGATAAGATCGTGGTGACCAAG |
| | | AGCACCACCGGCAGGGGCCAGGGCACCCCCATCGAGAAG |
| | | AGAGCCAGCATCACCTGGGCCAAGCCTCCCACCGACGAC |
| | | GACGAGGATGACGCCCAGGACGGCACCGAGGACGTGGCC |
| | | GCCCCCAAGAAAAAGCGGAAGGTGTAA |
| 183 | Cre | ATGTCCAATCTGCTGACCGTGCACCAGAACCTGCCTGCTC |
| | | TGCCCGTGGACGCCACCAGCGACGAGGTGCGCAAGAACC |
| | | TGATGGACATGTTCCGCGACCGCCAGGCCTTCAGCGAGCA |
| | | CACCTGGAAGATGCTGCTGAGCGTGTGCCGCAGCTGGGCC |
| | | GCCTGGTGCAAGCTGAACAACCGCAAGTGGTTCCCCGCCG |
| | | AGCCCGAGGACGTGCGCGACTACCTGCTGTACCTGCAGGC |
| | | CCGCGGCCTGGCCGTGAAAACCATCCAGCAGCACCTGGGC |
| | | CAGCTGAACATGCTGCACCGCCGCAGCGGCCTGcctAGGCC |
| | | ATCTGACTCTAATGCCGTGTCTCTGGTCATGCGGCGGATC |
| | | CGGAAAGAAAACGTGGACGCCGGCGAGAGAGCTAAGCAG |
| | | GCTCTGGCTTTCGAGAGAACCGACTTCGACCAAGTGCGGT |
| | | CCCTGATGGAAAACTCCGACCGGTGCCAGGATATCCGGAA |
| | | CCTGGCTTTTCTGGGAATCGCCTACAACACCCTGCTGCGG |
| | | ATCGCTGAGATCGCCCGGATCAGAGTGAAGGACATCTCTA |
| | | GAACCGACGGCGGCAGAATGCTGATCCACATCGGCAGAA |
| | | CAAAGACCCTGGTGTCCACAGCTGGCGTGGAAAAGGCTCT |
| | | GTCTCTGGGCGTGACCAAGCTGGTGGAACGGTGGATTTCT |
| | | GTGTCCGGCGTGGCCGACGATCCCAACAACTACCTGTTCT |
| | | GCAGAGTCCGGAAGAACGGCGTGGCAGCCCCTTCTGCTAC |
| | | ATCCCAGCTGTCTACAAGAGCCCTGGAAGGCATCTTCGAG |
| | | GCTACCCACAGACTGATCTACGGCGCCAAGGACGATAGC |
| | | GGCCAGAGATATTTGGCTTGGAGCGGCCACTCCGCTAGAG |
| | | TGGGAGCTGCTAGAGATATGGCTAGAGCCGGCGTGTCCAT |
| | | TCCTGAGATCATGCAAGCTGGCGGCTGGACCAACGTGAAC |
| | | ATCGTGATGAACTACATCCGCAACCTGGACTCCGAGACAG |
| | | GCGCTATGGTTCGACTGCTGGAAGATGGCGACTGA |
| 184 | VCre (contains a | ATGATCGAGAACCAGCTGAGCCTGCTGGGCGACTTTTCTG |
| | C-terminal NLS) | GCGTGCGGCCCGACGATGTGAAAACCGCCATTCAGGCCGC |
| | | CCAGAAAAAGGGCATCAACGTGGCCGAGAACGAGCAGTT |
| | | CAAGGCCGCCTTCGAGCATCTGCTGAACGAGTTCAAGAAG |
| | | CGGGAAGAGAGATACAGCCCCAACACCCTGCGGCGGCTG |
| | | GAAAGCGCCTGGACCTGCTTCGTGGATTGGTGCCTGGCCA |
| | | ACCACAGACACAGCCTGCCTGCCACCCCCGATACCGTGGA |
| | | AGCCTTCTTCATCGAGCGGGCCGAGGAACTGCACCGGAAC |
| | | ACCCTGAGCGTGTACAGATGGGCCATCAGCCGGGTGCACA |
| | | GAGTGGCCGGATGCCCTGATCCCTGCCTGGACATCTACGT |
| | | GGAAGATCGGCTGAAGGCCATTGCCCGGAAGAAAGTGCG |
| | | GGAAGGCGAGGCCGTGAAGCAGGCCAGCCCTTTCAACGA |
| | | GCAGCATCTGCTGAAGCTGACCAGCCTGTGGTACAGAAGC |
| | | GACAAGCTGCTGCTGCGGCGGAACCTGGCTCTGCTGGCTG |
| | | TGGCCTACGAGAGCATGCTGAGAGCCAGCGAGCTGGCCA |
| | | ACATCCGGGTGTCCGATATGGAACTGGCCGGCGACGGAA |
| | | CCGCCATCCTGACCATCCCTATCACCAAGACCAACCACTC |
| | | CGGCGAGCCCGATACCTGCATCCTGTCCCAGGATGTGGTG |
| | | TCCCTGCTGATGGACTACACCGAGGCCGGCAAGCTGGATA |
| | | TGAGCAGCGACGGCTTCCTGTTCGTGGGCGTGTCCAAGCA |
| | | CAACACCTGTATCAAGCCCAAGAAGGACAAGCAGACCGG |
| | | CGAGGTGCTGCACAAGCCCATCACCACCAAGACAGTGGA |
| | | AGGCGTGTTCTACAGCGCCTGGGAGACACTGGACCTGGGC |
| | | AGACAGGGCGTGAAGCCTTTCACAGCCCACAGCGCCAGA |
| | | GTGGGAGCCGCTCAGGACCTGCTGAAGAAGGGCTACAAT |
| | | ACCCTGCAGATCCAGCAGTCCGGCCGGTGGTCTAGCGGAG |
| | | CCATGGTGGCCAGATACGGCAGAGCCATCCTGGCTAGGG |
| | | ATGGCGCTATGGCCCACAGCAGAGTGAAAACCAGATCCG |
| | | CCCCCATGCAGTGGGGCAAGGACGAGAAGGACCCCAAGA |
| | | AAAAGCGGAAGGTGTGA |
| 185 | Flp (contains a C- | ATGAGCCAGTTCGACATCCTGTGCAAGACCCCCCCCAAGG |
| | terminal NLS) | TGCTGGTGCGGCAGTTCGTGGAGAGATTCGAGAGGCCCAG |
| | | CGGCGAGAAGATCGCCAGCTGTGCCGCCGAGCTGACCTAC |
| | | CTGTGCTGGATGATCACCCACAACGGCACCGCCATCAAGA |
| | | GGGCCACCTTCATGAGCTACAACACCATCATCAGCAACAG |
| | | CCTGAGCTTCGACATCGTGAACAAGAGCCTGCAGTTCAAG |
| | | TACAAGACCCAGAAGGCCACCATCCTGGAGGCCAGCCTG |
| | | AAGAAGCTGATCCCCGCCTGGGAGTTCACCATCATCCCTT |
| | | ACAACGGCCAGAAGCACCAGAGCGACATCACCGACATCG |
| | | TGTCCAGCCTGCAGCTGCAGTTCGAGAGCAGCGAGGAGG |
| | | CCGACAAGGGCAACAGCCACAGCAAGAAGATGCTGAAGG |
| | | CCCTGCTGTCCGAGGGCGAGAGCATCTGGGAGATCACCGA |
| | | GAAGATCCTGAACAGCTTCGAGTACACCAGCAGGTTCACC |
| | | AAGACCAAGACCCTGTACCAGTTCCTGTTCCTGGCCACAT |
| | | TCATCAACTGCGGCAGGTTCAGCGACATCAAGAACGTGGA |
| | | CCCCAAGAGCTTCAAGCTGGTGCAGAACAAGTACCTGGGC |
| | | GTGATCATTCAGTGCCTGGTGACCGAAACCAAGACAAGCG |
| | | TGTCCAGGCACATCTACTTTTTCAGCGCCAGAGGCAGGAT |
| | | CGACCCCCTGGTGTACCTGGACGAGTTCCTGAGGAACAGC |
| | | GAGCCCGTGCTGAAGAGAGTGAACAGGACCGGCAACAGC |
| | | AGCAGCAACAAGCAGGAGTACCAGCTGCTGAAGGACAAC |
| | | CTGGTGCGCAGCTACAACAAGGCCCTGAAGAAGAACGCC |
| | | CCCTACCCCATCTTCGCTATCAAGAACGGCCCTAAGAGCC |
| | | ACATCGGCAGGCACCTGATGACCAGCTTTCTGAGCATGAA |
| | | GGGCCTGACCGAGCTGACAAACGTGGTGGGCAACTGGAG |
| | | CGACAAGAGGGCCTCCGCCGTGGCCAGGACCACCTACAC |
| | | CCACCAGATCACCGCCATCCCCGACCACTACTTCGCCCTG |
| | | GTGTCCAGGTACTACGCCTACGACCCCATCAGCAAGGAGA |
| | | TGATCGCCCTGAAGGACGAAACCAACCCCATCGAGGAGT |
| | | GGCAGCACATCGAGCAGCTGAAGGGCAGCGCCGAGGGCA |
| | | GCATCAGATACCCCGCCTGGAACGGCATCATCAGCCAGGA |
| | | GGTGCTGGACTACCTGAGCAGCTACATCAACAGGCGGATC |
| | | CCCAAGAAAAAGCGGAAGGTGTGA |
| 186 | Bxb1 (contains a | ATGCGAGCCCTGGTGGTCATTCGCCTGAGCAGAGTCACAG |
| | C-terminal NLS) | ACGCTACTACAAGCCCTGAGCGGCAGCTGGAGTCCTGTCA |
| | | GCAGCTGTGCGCACAGCGAGGATGGGATGTGGTCGGAGT |
| | | GGCAGAGGATCTGGACGTGAGCGGGGCTGTCGATCCATTC |
| | | GACCGAAAGCGGAGGCCCAACCTGGCACGATGGCTGGCT |
| | | TTCGAGGAACAGCCCTTTGATGTGATCGTCGCCTACAGAG |
| | | TGGACAGGCTGACACGCTCAATTCGACATCTGCAGCAGCT |
| | | GGTGCATTGGGCCGAGGATCACAAGAAACTGGTGGTCAG |
| | | CGCAACTGAAGCCCACTTCGACACCACAACTCCTTTTGCC |
| | | GCTGTGGTCATCGCACTGATGGGCACCGTGGCCCAGATGG |
| | | AGCTGGAAGCTATCAAGGAGCGAAACCGGAGCGCAGCCC |
| | | ATTTCAATATTCGGGCCGGGAAATACAGAGGCAGCCTGCC |
| | | CCCTTGGGGCTATCTGCCTACCCGGGTGGATGGGGAGTGG |
| | | AGACTGGTGCCAGACCCCGTCCAGAGAGAGAGGATTCTG |
| | | GAAGTGTACCACAGAGTGGTGGACAACCACGAACCACTG |
| | | CATCTGGTGGCCCACGATCTGAATAGGCGCGGAGTCCTGT |
| | | CTCCAAAGGACTATTTTGCTCAGCTGCAGGGAAGGGAGCC |
| | | ACAGGGACGAGAATGGAGTGCTACCGCACTGAAGCGGTC |
| | | TATGATCAGTGAGGCTATGCTGGGCTATGCAACTCTGAAT |
| | | GGGAAAACCGTGAGAGATGATGACGGAGCACCACTGGTG |
| | | CGGGCTGAGCCTATTCTGACAAGAGAGCAGCTGGAAGCTC |
| | | TGAGGGCAGAACTGGTGAAAACCAGTAGGGCCAAGCCTG |
| | | CTGTGTCAACACCAAGCCTGCTGCTGCGAGTGCTGTTCTG |
| | | CGCAGTCTGTGGCGAGCCAGCATACAAATTTGCCGGCGGG |
| | | GGAAGGAAGCATCCCCGCTATCGATGCCGGAGCATGGGG |
| | | TTCCCTAAGCACTGTGGAAACGGCACTGTGGCTATGGCCG |
| | | AATGGGACGCCTTTTGTGAGGAACAGGTGCTGGATCTGCT |
| | | GGGGGACGCAGAGCGCCTGGAAAAAGTGTGGGTCGCTGG |
| | | AAGCGATTCCGCTGTGGAGCTGGCAGAAGTCAATGCCGA |
| | | GCTGGTGGACCTGACCTCCCTGATCGGATCTCCTGCATAC |
| | | AGGGCAGGCTCCCCACAGCGAGAAGCTCTGGATGCACGA |
| | | ATTGCTGCACTGGCAGCTCGACAGGAGGAACTGGAGGGG |
| | | CTGGAAGCCAGACCCTCTGGATGGGAGTGGCGAGAAACA |
| | | GGCCAGCGGTTTGGGGATTGGTGGAGGGAGCAGGACACA |
| | | GCAGCCAAGAACACTTGGCTGAGATCCATGAATGTCAGGC |
| | | TGACTTTCGACGTGCGAGGAGGACTGACCCGAACAATCGA |
| | | TTTTGGCGACCTGCAGGAGTATGAACAGCATCTGCGCCTG |
| | | GGAAGTGTGGTCGAGCGACTGCACACCGGCATGTCACCCA |
| | | AGAAAAAGCGGAAGGTGTGA |
| 187 | TP901 | ATGACCAAGAAGGTGGCCATCTACACCAGAGTGTCCACCA |
| | | CCAACCAGGCCGAGGAAGGCTTCAGCATCGACGAGCAGA |
| | | TCGACCGGCTGACCAAATACGCCGAGGCCATGGGATGGC |
| | | AGGTGTCCGATACCTACACCGACGCCGGCTTTAGCGGCGC |
| | | CAAGCTGGAAAGACCCGCCATGCAGCGGCTGATCAACGA |
| | | CATCGAGAACAAGGCCTTCGACACCGTGCTGGTGTACAAG |
| | | CTGGACAGGCTGAGCAGAAGCGTGCGGGACACCCTGTAC |
| | | CTCGTGAAGGACGTGTTCACCAAGAACAAGATCGACTTCA |
| | | TCAGCCTGAACGAGAGCATCGACACCAGCAGCGCTATGG |
| | | GCAGCCTGTTCCTGACCATCCTGAGCGCCATCAACGAGTT |
| | | CGAGCGCGAGAACATCAAAGAACGGATGACCATGGGCAA |
| | | GCTGGGCAGAGCCAAGAGCGGCAAGAGCATGATGTGGAC |
| | | CAAGACCGCCTTCGGCTACTACCACAACAGAAAGACCGG |
| | | CATCCTGGAAATAGTGCCACTGCAGGCCACCATCGTGGAA |
| | | CAGATCTTCACCGACTACCTGAGCGGCATCTCCCTGACCA |
| | | AGCTGAGAGACAAGCTGAACGAGTCCGGCCACATCGGCA |
| | | AGGACATCCCTTGGAGCTACCGGACCCTGCGGCAGACCCT |
| | | GGACAACCCTGTGTACTGCGGCTACATCAAGTTCAAGGAC |
| | | TCCCTGTTCGAGGGCATGCACAAGCCCATCATCCCTTACG |
| | | AGACATACCTGAAGGTGCAGAAAGAGCTGGAAGAGAGAC |
| | | AGCAGCAGACCTACGAGCGGAACAACAACCCCAGACCCT |
| | | TCCAGGCCAAGTACATGCTGTCCGGCATGGCCAGATGCGG |
| | | CTACTGTGGCGCCCCTCTGAAGATCGTGCTGGGCCACAAG |
| | | AGAAAGGACGGCAGCCGGACCATGAAGTACCACTGCGCC |
| | | AACCGGTTCCCTAGAAAGACCAAGGGCATCACCGTGTACA |
| | | ACGACAACAAGAAGTGCGACAGCGGCACCTACGACCTGA |
| | | GCAACCTGGAAAACACCGTGATCGACAACCTGATCGGCTT |
| | | CCAGGAAAACAACGACAGCCTGCTGAAGATCATCAACGG |
| | | CAACAACCAGCCCATCCTGGACACCTCCAGCTTCAAGAAG |
| | | CAGATCAGCCAGATCGACAAGAAGATCCAGAAGAACAGC |
| | | GACCTGTACCTGAACGATTTCATCACCATGGACGAGCTGA |
| | | AGGACCGGACCGACTCTCTGCAGGCCGAGAAGAAGCTGC |
| | | TGAAGGCCAAGATCTCTGAGAACAAGTTCAACGATAGCA |
| | | CCGACGTGTTCGAGCTCGTGAAAACACAGCTGGGCTCCAT |
| | | CCCCATCAATGAGCTGAGCTACGATAACAAGAAAAAGAT |
| | | TGTGAACAACCTGGTGTCTAAGGTGGACGTGACCGCCGAC |
| | | AACGTGGACATCATCTTCAAGTTCCAGCTGGCCTGA |
| 188 | R4 | ATGAACAGAGGAGGCCCTACCGTGCGGGCCGACATCTAC |
| | | GTGCGGATCAGCCTGGATAGAACCGGCGAGGAACTCGGC |
| | | GTGGAAAGACAGGAGGAATCTTGTAGAGAACTGTGCAAG |
| | | AGCCTGGGCATGGAAGTCGGACAAGTGTGGGTCGACAAC |
| | | GACCTGTCCGCTACTAAGAAGAACGTGGTGCGCCCCGATT |
| | | TTGAGGCCATGATCGCCAGCAACCCCCAGGCCATCGTGTG |
| | | CTGGCACACAGACCGGCTGATCAGGGTGACCCGGGACCTC |
| | | GAGCGGGTGATCGACCTGGGCGTGAACGTGCACGCCGTG |
| | | ATGGCTGGCCACCTGGACCTGAGCACCCCTGCCGGCAGAG |
| | | CCGTCGCCCGGACCGTGACAGCCTGGGCCACCTACGAGGG |
| | | AGAGCAGAAGGCCGAGAGACAGAAGCTGGCCAACATCCA |
| | | GAATGCCAGAGCGGGAAAACCTTATACACCTGGCATCCG |
| | | GCCCTTCGGCTATGGCGATGACCACATGACCATCGTGACA |
| | | GCCGAAGCCGATGCCATTAGAGATGGCGCCAAGATGATC |
| | | CTGGACGGCTGGAGCCTGAGCGCCGTGGCCCGGTACTGGG |
| | | AGGAACTGAAGCTGCAGAGCCCTAGAAGCATGGCCGCTG |
| | | GCGGCAAGGGCTGGTCCCTGAGAGGAGTGAAGAAAGTGC |
| | | TGACCAGCCCACGGTACGTGGGCAGAAGCAGCTACCTGG |
| | | GCGAAGTGGTGGGCGACGCCCAGTGGCCTCCAATCCTGGA |
| | | TCCTGACGTGTACTACGGCGTGGTCGCCATCCTGAACAAC |
| | | CCTGACAGATTCAGCGGCGGCCCCAGAACCGGCAGGACC |
| | | CCTGGTACACTGCTGGCTGGGATCGCCCTGTGTGGCGAGT |
| | | GCGGCAAAACCGTGTCCGGCAGAGGCTACAGAGGCGTTC |
| | | TGGTGTACGGCTGCAAGGACACCCACACCAGAACACCAA |
| | | GAAGCATCGCCGATGGCAGAGCCTCTAGCTCAACACTGGC |
| | | AAGACTGATGTTCCCCGACTTCCTGCCTGGCCTGCTGGCC |
| | | AGCGGACAGGCCGAGGACGGACAATCTGCTGCTTCTAAG |
| | | CACAGCGAGGCTCAGACCCTGAGAGAGAGACTGGACGGC |
| | | CTGGCCACCGCCTACGCCGAGGGCGCCATCTCCCTGTCTC |
| | | AGATGACCGCCGGCAGCGAGGCTCTGCGGAAGAAGCTGG |
| | | AAGTGATCGAGGCTGACCTGGTGGGAAGCGCCGGAATTC |
| | | CTCCTTTCGACCCTGTGGCCGGCGTTGCCGGCCTGATCTCT |
| | | GGATGGCCCACCACCCCTCTGCCCACACGTAGAGCTTGGG |
| | | TGGATTTCTGCCTGGTCGTGACCCTTAATACCCAGAAAGG |
| | | CCGGCACGCCAGCAGCATGACAGTGGACGATCATGTGAC |
| | | GATCGAGTGGCGGGACGTGGCTGAATGA |
| 189 | Dre (contains an | ATGCCTAAGAAGAAGAGAAAGGTGGGCAGCAGCGAGCTG |
| | N-terminal NLS) | ATCATCTCTGGCTCTAGCGGAGGATTTCTGAGAAACATCG |
| | | GCAAGGAATACCAGGAGGCCGCTGAAAACTTCATGAGAT |
| | | TCATGAACGACCAGGGCGCTTACGCCCCTAATACCCTGAG |
| | | AGATCTGCGGCTGGTGTTCCACAGCTGGGCCAGATGGTGC |
| | | CACGCCAGACAGCTGGCCTGGTTCCCCATCAGCCCCGAGA |
| | | TGGCCAGAGAGTACTTCCTGCAGCTGCACGACGCTGATCT |
| | | GGCATCTACCACCATCGACAAGCACTACGCCATGCTGAAC |
| | | ATGCTGCTGAGTCACTGTGGACTGCCTCCTCTGTCTGATGA |
| | | CAAGAGCGTGTCTCTGGCCATGCGGCGCATTAGAAGGGA |
| | | AGCCGCCACAGAAAAGGGCGAGAGAACCGGCCAGGCTAT |
| | | CCCCCTGAGATGGGACGACCTGAAGCTGCTGGACGTGCTG |
| | | CTCTCCCGGAGCGAGCGGCTGGTCGACCTGAGAAATAGG |
| | | GCCTTCCTGTTCGTGGCCTACAACACCCTGATGAGAATGA |
| | | GCGAGATCAGCAGAATCCGGGTGGGCGACCTGGATCAAA |
| | | CAGGCGATACCGTGACCCTCCATATCAGCCACACCAAGAC |
| | | CATCACCACAGCCGCCGGCCTGGACAAAGTGCTGAGCCG |
| | | GCGGACCACAGCCGTGCTGAATGACTGGCTGGATGTGTCC |
| | | GGACTGCGGGAACACCCCGATGCTGTTCTGTTTCCTCCAA |
| | | TCCACAGAAGCAACAAGGCCAGAATCACAACAACACCTC |
| | | TGACCGCTCCTGCCATGGAAAAGATCTTCAGCGACGCCTG |
| | | GGTCCTGCTGAACAAGCGGGACGCCACCCCTAACAAAGG |
| | | CAGATATAGAACCTGGACCGGCCACAGCGCCAGAGTGGG |
| | | AGCCGCTATCGACATGGCCGAGAAACAGGTGTCCATGGTG |
| | | GAAATCATGCAGGAGGGCACCTGGAAGAAACCAGAGACA |
| | | CTCATGCGGTACCTGCGGAGAGGCGGCGTGTCCGTGGGCG |
| | | CCAACAGCAGACTGATGGACAGCTGA |
| 190 | Int | ATGACAAACCCCGCCAGCAGGCCTAAGGCCTACTCCTACA |
| | | TCAGAATGTCCTCCGCCATCCAGATCAAGGGCGACTCCTT |
| | | CCGGCGGCAGGCCGAGGCTTCCGCCAAGTACGCTGCCGA |
| | | GCACGACCTGGATCTGATCGACGATTACAAACTGGCCGAT |
| | | CTGGGGGTGTCCGCCTTCAAGTCCGACAACCTGACCACCG |
| | | GCGCTCTGGGGCGGTTCGTGGCCGAGTGCGAGGCGGGAG |
| | | AAATCGAGGCTGGATCCTTTCTGCTGATCGAATCCCTGGA |
| | | CAGGCTGTCGAGAGACAAGATCCTGGACGCCTTCAGCCTG |
| | | TTTGCCAGAATTCTGAAAACCGGTGTTAAGATCGTCACCC |
| | | TGTCTGACGGCCAAGTGTACGACGGCTCCAGCGACCAGGT |
| | | GGGCTCTATCTACTACGCTATCAGCGTGATGATCCGGAGC |
| | | AACGACGAGTCTAAAATCAAGTCCACCAGAGGACTGGCC |
| | | AACTGGTCCCAGAAGAGAAAGCTGGCTGCAGAACACGGC |
| | | GTGAAGATGTCCTCCCAGTGTCCCGCCTGGCTGAAGCTGT |
| | | CTGTGGATAGAAAGTCCTACCTGATCGACAAGGAAAGGG |
| | | CTAAGATCGTGCAGAGAATCTTCGAGGCCTCTGCCTCTGG |
| | | CAAAGGCGCCAATCTGATCACCAAGGAACTGAACCGGGA |
| | | CAAGGTGCCTACCTTCGGCAGAGGCGCCCTGTGGGCCGAA |
| | | GCCTTTGTGTCCAAGACCCTGCGGAACCGGGCCGTGTTAG |
| | | GAGAGTTCCAGCCTGGCCAGTACGTGTCTGGTAAGAGACA |
| | | GCCCGCTGGCGACCCAATCCCTGGCTACTTCCCTCCTGTG |
| | | ATCGAAGAGGAGCTGTTCGATATCGTGCAAGCCTCCCTGA |
| | | GAGGCCGCCTCCTCGCTGGCGGCAGAAGAGGCGAGGGCC |
| | | AGTCCAACATCTTCACCCATGTAGCCTTCTGCGGCTACTGC |
| | | GGCTCCAAGATGAGACACAGAAGCAAGGGCAGCAGAGTG |
| | | AAGGGCAACCCCCCTCACAGATACCTGACCTGTTTCAACA |
| | | GATTCAACGGCCCAGGCTGCGACTGCAAGCCCCTGCCTTA |
| | | CGCCGCTTTCGAGCGCTCTTTCCTGACTTTCGTGCGGGATG |
| | | TGGACCTGAGAGGCCTGCTGGAAGGCGCCAAGAGAAAGT |
| | | CCGAGGCCAAGACCATCGCTGACAGAATCACCGTGAACG |
| | | AGGAAAAAGTCAGAAAAGCTGATGAGAGAATCCGCGACT |
| | | ACCTGATCAAGATCGAAGGAGCTCCTGACCTGGCCGAGAT |
| | | CTTCATGGAACGGATCAGAGAGCTGAAGGCTGAGAAGGA |
| | | CGACCTGGTCAGATCTATCGAAGAGTCCAACGACGCTCTG |
| | | TCCAAGATCAAATCTGACAACGTGACAGACGAGGAGCTG |
| | | GCTAGCTTGATCTCTACCTTTCAGAACCCTTGCGGAGAGA |
| | | ATCGGATCAGACTGGCCGACCGGATAAAGTCCATCATCGA |
| | | GAGAATCGACGTGTATCCCAACGGCGAAATCCGGAAGGA |
| | | CGACCCTGCCATCGATCTGGTCCGGGCTTCTGGCGATCCT |
| | | GACGCTGAGAAGATCATCGCCGCCATGAACGCCGGCTCTA |
| | | GACTGAAGGACGACCCTTACTTCATCGTGACCTTCCGGAA |
| | | TGGCGCTGTGCAGACCGTGGTGCCTAACCCTTCCAACCCT |
| | | GATGATATTCGGGTTTCTGTGTACGCAGGCGAAAAGACCC |
| | | GACGGGTGGAAGGCTCTGCCTATGAGTACGAGTCCGAT |
| 191 | Int2 | ATGCCTATCGCCCCTGAGTTCCTGTCTCTGGCCTACCCCGG |
| | | ACAAGAGTTCCCTGCCTACCTGTACGGCAGAGCCTCTAGA |
| | | GATCCTAAGCGGAAGGGCAGATCTGTGCAGAGCCAGCTG |
| | | GACGAAGGCAGAGCCACATGCCTGGATGCCGGCTGGCCT |
| | | ATTGCCGGCGAATTTAAGGACGTGGATCGGTCCGCTTCTG |
| | | CTTACGCCAGACGGACACGGGACGAATTCGAGGAGATGA |
| | | TCGCTGGCATCCAGGCCGGAGAGTGCAGGATTCTGGTCGC |
| | | CTTCGAGGCAAGCAGATACTACCGGGACCTGGAGGCTTAT |
| | | GTTCGGCTGCGGAGAGTGTGCAGAGAGGCCGGCGTCCTCC |
| | | TGTGCTACAACGGCCAGGTGTACGACCTGTCCAAGTCCGC |
| | | CGACAGAAAGGCCACCGCTCAGGACGCTGTGAACGCCGA |
| | | GGGAGAAGCTGACGACATCAGAGAACGGAACCTGAGAAC |
| | | CACCAGACTGAATGCTAAGAGAGGCGGCGCCCACGGCCC |
| | | TGTGCCTGATGGCTACAAGAGAAGATACGACCCCGACTCT |
| | | GGCGACCTGGTGGACCAGATCCCTCATCCTGATAGAGCGG |
| | | GCCTGATCACCGAGATCTTCCGGCGCGCTGCCGCTGCTGA |
| | | GCCCCTGGCTGCTATCTGTCGGGATCTGAACGAGAGAGGC |
| | | GAGACAACCCACAGGGGAAAAGCTTGGCAGAGACACCAC |
| | | CTGCACGCCATCCTGAGAAATCCCGCCTACATCGGCCACC |
| | | GGAGGCATCTGGGCGTGGACACCGGCAAAGGTATGTGGG |
| | | CTCCTATCTGCGACGACGAGGACTTCGCCGAAACCTTCCA |
| | | GGCCGTGCAGGAGATCTTATCTTTGCCAGGCAGACAGCTG |
| | | TCTCCTGGCCCAGAAGCTCAGCACCTGCAGACCGGAATCG |
| | | CCCTGTGTGGCGAGCACCCTGACGAGCCTCCTCTGAGATC |
| | | CGTGACCGTGCGCGGCCGGACCAACTACAACTGCTCCACC |
| | | AGATATGATGTGGCCATGAGAGAAGATCGGATGGACGCC |
| | | TTCGTGGAAGAGTCCGTGATCACCTGGCTGGCCTCCGACG |
| | | AAGCCGTGGCTGCCTTTGAGGACAACACCGACGATGAGC |
| | | GGACACGGAAGGCCCGGATCCGGCTGAAGGTGCTGGAGG |
| | | AACAGCTGGAAGCCGCCCAGAAGCAGGCTAGAACCCTGC |
| | | GGCCTGACGGCATGGGCATGCTGCTGTCCATCGACTCCCT |
| | | GGCTGGCCTGGAAGCCGAGCTGACCCCTCAAATCGACAA |
| | | GGCCAGACAAGAATCCCGGAGCCTGCACGTGCCCGCTCTG |
| | | CTGAGAGATCTGCTGGGCAAGCCTAGAGCCGACGTCGACC |
| | | GGGCCTGGAACGAGGCTCTAACCCTGCCCCAGCGGCGGAT |
| | | GATCCTAAGAATGGTGGTGACCATCAGACTGTTCAAAGCT |
| | | GGCTCTAGAGGCGTGCGGGCCATCGAGCCTGGCCGGATCA |
| | | CCCTGTCCTACGTGGGCGAGCCAGGCTTCAAGCCCGTGGG |
| | | CGGCAACCGGGCCAAGCAG |
| 192 | Int3 | ATGAGAAAGGTGGCCATCTACAGCCGGGTGTCCACCATCA |
| | | ACCAGGCCGAAGAGGGCTATTCTATCCAGGGCCAAATCG |
| | | AGGCCCTGACCAAGTACTGCGAGGCTATGGAATGGAAGA |
| | | TCTACAAAAACTACTCCGACGCCGGCTTCTCCGGAGGCAA |
| | | GCTCGAAAGACCCGCTATAACCGAGCTGATTGAGGACGG |
| | | CAAGAACAACAAGTTTGACACCATCCTGGTGTACAAGCTG |
| | | GATCGGCTGTCCCGGAACGTGAAGGACACACTCTACCTGG |
| | | TTAAAGATGTGTTCACCGCTAACAACATCCACTTCGTGTCT |
| | | CTTAAGGAGAACATCGATACTTCCTCTGCCATGGGAAACC |
| | | TGTTCCTGACCCTGCTGTCTGCTATCGCCGAGTTCGAGAG |
| | | AGAACAGATCAAGGAGCGGATGCAGTTCGGTGTGATGAA |
| | | CCGGGCTAAGTCCGGCAAAACAACAGCTTGGAAAACCCC |
| | | TCCTTACGGCTACAGATACAACAAGGACGAAAAGACCCT |
| | | GTCTGTCAACGAGCTGGAAGCCGCCAACGTCAGACAGAT |
| | | GTTCGACATGATCATCTCCGGCTGTAGCATCATGTCCATC |
| | | ACCAACTACGCCCGGGACAACTTTGTGGGCAACACCTGGA |
| | | CCCACGTGAAGGTGAAGCGGATCCTGGAAAACGAAACCT |
| | | ACAAGGGCCTGGTCAAGTACAGAGAGCAGACATTTTCTGG |
| | | CGACCACCAGGCAATCATCGATGAGAAAACCTACAATAA |
| | | GGCCCAGATCGCTCTGGCTCATAGAACCGACACCAAGACA |
| | | AACACCAGACCATTCCAGGGCAAGTACATGCTGTCTCATA |
| | | TCGCCAAGTGCGGCTACTGTGGCGCTCCTCTGAAAGTGTG |
| | | CACCGGCAGAGCCAAGAACGATGGCACCAGACGGCAAAC |
| | | CTACGTGTGCGTGAACAAGACCGAGTCCCTGGCCAGAAG |
| | | GAGCGTGAATAATTATAACAACCAGAAGATCTGCAACAC |
| | | CGGCCGCTACGAGAAGAAGCACATCGAGAAGTATGTGAT |
| | | CGACGTGCTGTACAAGCTGCAGCACGACAAAGAGTACCT |
| | | GAAAAAGATCAAAAAGGACGATAATATCATCGACATCAC |
| | | CCCTCTGAAGAAAGAAATCGAGATCATCGACAAGAAGAT |
| | | CAACAGACTGAACGACCTGTACATCAACGATCTGATCGAT |
| | | CTGCCCAAGCTGAAAAAGGATATCGAGGAACTGAACCAC |
| | | CTGAAGGACGACTACAACAAGGCCATCAAGCTGAACTAC |
| | | CTGGACAAGAAGAATGAGGATTCTCTGGGCATGCTGATGG |
| | | ACAACCTGGACATCCGGAAAAGCTCCTACGACGTGCAGTC |
| | | CAGAATCGTGAAGCAGCTGATCGACAGAGTGGAAGTGAC |
| | | CATGGACAATATCGACATTATCTTCAAGTTC |
| 193 | Int4 | ATGATCACAACCAGAAAGGTTGCCATCTATGTGAGAGTGT |
| | | CCACCACCAACCAGGCTGAAGAAGGCTACTCCATCCAGG |
| | | GCCAGATCGACTCCCTGATTAAGTACTGCGAGGCTATGGG |
| | | CTGGATCATCTACGAGGAGTACACCGACGCTGGCTTCTCC |
| | | GGCGGAAAAATCGATCGGCCTGCCATGAGTAAGCTGATC |
| | | ACCGATGCCAAGCACAAGAGATTCGATACAATCCTGGTGT |
| | | ACAAGCTGGACAGACTGAGCAGATCCGTGCGGGACACAC |
| | | TGTACCTGGTCAAGGATGTGTTCAACCAGAACAACATCCA |
| | | CTTCGTGTCCCTGCAGGAGAATATCGACACCTCCAGCGCC |
| | | ATGGGAAACCTGTTCCTGACCCTGCTCTCTGCTATCGCCG |
| | | AGTTCGAGAGAGAGCAGATCACCGAGCGGATGACCATGG |
| | | GCAAGATCGGCAGAGCCAAGTCTGGCAAGACCATGGCCT |
| | | GGACCTACACCCCTTTTGGCTACGACTATAACAAAGAGAA |
| | | GGGCGAGCTGATCCTGGATCCTGCTAAGGCCCCCATCGTG |
| | | AAGATGATCTACACCGACTACCTGAAGGGTATGAGCATCC |
| | | AAAAGATCGTGGACAAACTAAACAAGATGGACTACAACG |
| | | GCAAGGACTGCACCTGGTTCCCACACGGCGTGAAACATCT |
| | | GCTGGACAATCCTGTGTACTACGGCATGACTAGATATAAC |
| | | AACAAGCTGTTTCCTGGCAACCACCAGCCAATCATCACCA |
| | | AGGAACTGTTTGACAAGACCCAGCGCGAGAGACAGAGAA |
| | | GAAGGCTGGGCATCGAAGAGAATCACTACACCATACCTTT |
| | | CCAGGCCAAATACATGCTGTCTAAGTTCCTGAGATGCAGA |
| | | CAGTGCGGCTCTAGAATGGGCCTGGAGCTGGGCAGACCTC |
| | | GGAAGAAAGAGGGAAAGCGGTCCAAGAAGTACTACTGTC |
| | | TGAACTCCAGGCCCAAGAGAACCGCCTCCTGCGACACCCC |
| | | TCTGTACGATGCTGAAACCCTGGAAGATTACGTGCTGCAC |
| | | GAGATCGCCAAAATCCAGAAGGACCCTTCTATCGCTTCTC |
| | | GGCAAAAACACATCGAAGATCATGAATTGAAATACAAGC |
| | | GGGAACGGATCGAGGCCAACATCAACAAGACCGTGAACC |
| | | AGCTGTCCAAGCTGAACAACCTGTACCTGAATGACCTGAT |
| | | CACCCTCGAGGACCTGAAAACCCAGACCAACACCCTGATT |
| | | GCTAAGAAGCGACTGCTGGAAAACGAGCTGGACAAGACC |
| | | TGTGACAACGACGACGAGCTCGACAGACAAGAGACAATC |
| | | GCCGACTTCCTGGCTCTGCCTGACGTGTGGACAATGGATT |
| | | ACGAGGGCCAGAAGTACGCCGTGGAACTGCTGGTGCAGA |
| | | GAGTGAAGGTGGACCGGGACAACATCGACATCCACTGGA |
| | | CCTTC |
| 194 | Int5 | ATGCCTGGCATGACCACCGAAACCGGCCCCGATCCTGCCG |
| | | GCCTGATCGACCTGTTCTGCAGAAAAAGCAAAGCTGTCAA |
| | | GTCCAGAGCCAATGGCGCTGGACAGCGGAGAAAGCAAGA |
| | | AATCTCCATCGCCGCCCAGGAAACCCTGGGCCGAAAGGTG |
| | | GCTGCCCTGCTCGGCATGCAGGTGCGGCATGTGTGGAAGG |
| | | AAGTGGGATCTGCTTCTCGGTTTAGAAAGGGCAAGGCTCG |
| | | GGACGACCAGTCCAAGGCCCTGAAGGCCCTGGAATCTGG |
| | | CGAGGTGGGCGCTCTGTGGTGCTACCGGCTGGATAGATGG |
| | | GACAGAGGCGGCGCTGGAGCCATCCTGAAGATCATCGAG |
| | | CCTGAGGACGGCATGCCCCGGCGGCTGCTGTTTGGCTGGG |
| | | ATGAGGACACCGGCAGACCTGTCCTGGACTCCACCAACAA |
| | | GCGGGATCGGGGCGAGCTGATTAGACGGGCCGAGGAGGC |
| | | CAGAGAAGAAGCCGAAAAGCTGTCCGAGAGAGTCAGAGA |
| | | TACAAAAGCCCACCAGAGAGAGAACGGCGAGTGGGTGAA |
| | | CGCCAGAGCCCCTTACGGCCTGAGAGTGGTGCTGGTGACC |
| | | GTGTCCGACGAGGAAGGCGACGAGTACGACGAGCGGAAG |
| | | CTGGCTGCCGACGATGAGGACGCTGGCGGCCCTGACGGTC |
| | | TGACCAAGGCTGAAGCCGCTAGACTGGTGTTCACCCTGCC |
| | | TGTGACCGACAGACTCTCTTACGCCGGCACCGCTCACGCC |
| | | ATGAACACCAGAGAGATCCCATCTCCCACCGGCGGACCCT |
| | | GGATCGCCGTTACCGTGCGGGACATGATCCAGAACCCCGC |
| | | CTACGCTGGCTGGCAGACCACAGGCAGACAGGACGGCAA |
| | | GCAGCGGAGACTGACCTTCTATAACGGCGAAGGCAAACG |
| | | CGTGTCCGTGATGCACGGCCCTCCTCTGGTCACAGACGAG |
| | | GAGCAGGAAGCCGCCAAGGCAGCCGTGAAGGGAGAGGAT |
| | | GGCGTGGGCGTGCCACTGGACGGCTCTGACCACGACACCC |
| | | GGCGGAAGCACCTGCTGTCTGGCCGGATGCGGTGTCCTGG |
| | | CTGTGGCGGCAGCTGCTCCTACTCCGGCAACGGCTACAGA |
| | | TGCTGGCGGTCCTCCGTGAAGGGCGGCTGCCCTGCTCCAA |
| | | CCTACGTGGCTCGCAAGTCTGTGGAAGAGTATGTGGCCTT |
| | | CCGGTGGGCTGCCAAGCTGGCCGCCTCCGAGCCTGACGAT |
| | | CCTTTCGTGATCGCCGTGGCCGATCGGTGGGCCGCTCTGA |
| | | CCCACCCTCAGGCTTCCGAAGATGAGAAGTACGCCAAGGC |
| | | CGCAGTGAGGGAGGCCGAGAAGAACCTGGGCAGACTGCT |
| | | AAGAGACAGACAGAATGGCGTGTACGATGGACCTGCCGA |
| | | ACAGTTCTTCGCCCCTGCTTACCAGGAGGCTCTGTCTACAC |
| | | TGCAGGCCGCTAAGGACGCCGTGTCTGAGTCCTCCGCCTC |
| | | TGCCGCTGTGGACGTGAGCTGGATCGTGGACAGCAGCGAC |
| | | TACGAGGAACTGTGGCTGAGAGCTACCCCTACCATGAGAA |
| | | ACGCTATCATCGACACATGCATCGACGAGATCTGGGTCGC |
| | | GAAAGGCCAGAGAGGCAGACCTTTCGACGGGGACGAGAG |
| | | AGTGAAGATCAAGTGGGCCGCTAGGACT |
| 195 | Int6 | ATGCAGCTGGACGCCACCCTGACACTGCGGGACGAGGGC |
| | | CTGAGCGCTTTCCACCAGAGACACATCAAGCAGGGTGCTC |
| | | TGGGAGTGTTCCTGAGAGCTATCGAGGACGGCCGGATCCA |
| | | GCCTGGCTCCGTGCTGATCGTGGAAGGCCTGGACAGACTC |
| | | TCTAGAGCCGAGCCCATCCAAGCTCAGGCCCAGCTGGCCC |
| | | AGATCATCAACGCCGGCATCACCGTGGTGACCGCCTCTGA |
| | | TGGCCGAGAGTACAACCGGGAAAGACTGAAAGCCCAACC |
| | | TATGGACCTTGTGTACTCCCTGCTGGTGATGATCAGAGCT |
| | | CACGAGGAATCCGACACCAAGTCCAAGCGGGTGAAGGCC |
| | | GCCATCAGGCGGCAGTGCGAGGGCTGGGTCGCTGGCACA |
| | | TGGCGGGGCATCATCCGGAACGGCAAGGACCCTCACTGG |
| | | GTCAGACTGGGCGAGCACGGCAAGTTCGAGCATGTGCCTG |
| | | AGCGGGTGCTGGCTGTGCGGACAATGATCGACCTGTTCCT |
| | | GGAAGGCCACGGCGCCATCGAGATCACCAGGCGGCTGAC |
| | | CGAGCAGAACCTGTACGTGTCCAACGCCGGCAACTACTCT |
| | | GTGCACATGTACAGAATCGTGAGAAACCAGGCTCTGATCG |
| | | GCGAGAAGAGAATCTCCGTGGATGGAGAAGAGTTCCGGC |
| | | TGGACGGCTACTACCCTCCAATCCTGACCAGAGAAGAATT |
| | | TGCCGAACTGCAGCAGACCATGTCCGAGAGAGGCAGACG |
| | | GAAGGGCAAAGGCGAGATCCCTAACATCATCACAGGACT |
| | | GTCCATCACAGTGTGCGGCTATTGTGGCAGAGCCATGACC |
| | | ACCCAGAACTCTAAGGCTCGCGCCCCTAAGGGAAAAAGC |
| | | GTGGTCAGACGGCTGTCCTGCCCCATGAATTCCTTCAACG |
| | | AGGGATGTCCTATCGGCGGCTCTTGCGAGTCTGAGATCGT |
| | | CGAGAGAGCCCTCATGAGATACTGCTCCGACCAGTTCAAT |
| | | CTGTCTCGGTTGCTGGAGGGCGACGACGGCACCGCCCGGC |
| | | GGACCGCTCAACTGGCTGTGGCTAGACAAAGAGCATCTGA |
| | | CATCGAAGCCCAGATCCAGCGCGTGACCGACGCCCTCCTG |
| | | AGCGACGACGGCAAGGCTCCTGCCGCCTTTACCCGCAGAG |
| | | CTCGCGAGCTGGAAACCCAGCTGGAGGAACAGAGAAGAG |
| | | AGATCGAGGCTCTGGAACACCAGATCGCCGCTAGCTCTGC |
| | | TCATGGCATCCCCGCCGCCGCTGAGGCCTGGGCTCAGCTG |
| | | GTTGACGGCGTGCTGGCCCTGGACTACGATGCTCGGATGA |
| | | AGGCCAGACAGCTGGTGGCCGATACCTTCAGAAAGATCGT |
| | | GGTGTACCAGAGGGGCTTCGCCCCAATCGACGATGCTGCT |
| | | GCCGACAGATGGAAGAGATCCGGCACCATCGGCCTGATG |
| | | CTGGTCACCAAGAGAGGAGGCATGCGGCTGCTGAACGTG |
| | | GACCGGAGAACCGGCTGCTGGCAGGCCGAGGATGACCTG |
| | | GATCCTTCTCTGATTCCTTCCGATGGCCTGCCCATGCTGCC |
| | | TCTGGATGCC |
| 196 | Int7 | ATGAAAGTGGCCATCTACGTGCGGGTTTCCACCGACGAGC |
| | | AGGCCAAAGAAGGTTTCAGCATCCCTGCTCAAAGAGAGC |
| | | GGCTGAGAGCCTTCTGCGCCTCTCAAGGCTGGGAGATCGT |
| | | GCAGGAGTACATCGAGGAGGGCTGGTCCGCTAAGGATCT |
| | | GGACAGACCTCAGATGCAGCGGCTGCTGAAGGACATCAA |
| | | GAAGGGCAATATCGATATCGTGCTGGTGTACAGACTGGAT |
| | | AGGCTGACCAGATCTGTGCTGGATCTGTACCTGCTGCTCC |
| | | AGACCTTCGAGAAGTACAACGTGGCCTTTCGGTCTGCCAC |
| | | CGAGGTGTACGATACAAGCACCGCCATGGGCAGACTGTTT |
| | | ATCACTCTGGTCGCTGCTCTGGCTCAGTGGGAAAGAGAGA |
| | | ACCTGGCCGAGAGAGTGAAGTTCGGCATCGAACAGATGA |
| | | TCGACGAGGGCAAGAAGCCAGGCGGCCATTCTCCTTACGG |
| | | CTACAAGTTTGACAAGGATTTCAACTGTACCATCATCGAG |
| | | GAAGAAGCTGATGTGGTGCGGATGATTTACAGAATGTACT |
| | | GCGACGGCTATGGCTATAGATCCATCGCCGACAGACTGAA |
| | | CGAGCTGATGGTTAAGCCTAGAATCGCCAAGGAGTGGAA |
| | | CCACAACTCCGTCAGAGATATTCTGACCAACGACATCTAC |
| | | ATCGGCACCTACAGATGGGGCGACAAGGTGGTGCCTAAC |
| | | AACCACCCCCCCATCATCTCCGAGACACTGTTTAAGAAGG |
| | | CCCAGAAAGAGAAGGAGAAGCGGGGAGTGGACCGGAAG |
| | | AGAGTGGGCAAGTTCCTGTTCACCGGCCTGCTGCAGTGTG |
| | | GCAACTGCGGCGGACACAAGATGCAGGGCCACTTCGACA |
| | | AGCGCGAGCAGAAAACCTACTACCGGTGCACCAAGTGCC |
| | | ACCGGATCACCAACGAGAAGAACATCTTGGAACCTCTGCT |
| | | GGATGAGATCCAGCTGCTGATCACCTCTAAGGAGTACTTC |
| | | ATGTCCAAGTTCAGCGACAGATACGACCAGCAAGAAGTG |
| | | GTCGACGTGTCCGCTCTCACAAAAGAGCTCGAGAAGATCA |
| | | AGCGGCAGAAGGAAAAGTGGTACGACCTGTACATGGACG |
| | | ACCGGAATCCTATCCCCAAAGAGGAGCTGTTCGCCAAGAT |
| | | CAACGAGCTGAACAAGAAAGAAGAGGAAATCTACTCCAA |
| | | GCTGTCTGAAGTGGAAGAGGACAAAGAGCCTGTGGAAGA |
| | | AAAGTACAACAGACTGTCCAAGATGATCGACTTCAAGCA |
| | | GCAGTTCGAGCAGGCTAATGACTTCACCAAAAAGGAACT |
| | | GCTGTTCTCTATCTTCGAGAAGATCGTGATCTATCGGGAG |
| | | AAGGGAAAGCTGAAAAAGATTACACTGGACTACACCCTG |
| | | AAG |
| 197 | Int8 | ATGAAAGTGGCCGTGTACTGCAGAGTGTCCACCCTCGAGC |
| | | AGAAGGAGCACGGCCATTCTATTGAGGAACAAGAGCGGA |
| | | AGCTGAAGTCCTTCTGCGACATCAACGACTGGACAGTGTA |
| | | CGACACCTACATCGACGCTGGATACTCTGGCGCCAAGCGG |
| | | GACAGACCTGAGCTGCAGCGGCTGATGAACGATATCAAC |
| | | AAGTTCGACCTGGTGCTGGTCTACAAGCTGGACCGGCTGA |
| | | CCAGAAACGTGCGGGATCTGCTGGACCTGCTGGAAATCTT |
| | | CGAGAAGAACGACGTCAGCTTCAGATCCGCCACCGAGGT |
| | | GTACGACACCACCACCGCTATGGGCCGGCTGTTCGTGACC |
| | | CTGGTGGGCGCTATGGCCGAGTGGGAGAGAGAGACAATC |
| | | AGAGAACGGACCCAGATGGGCAAGCTGGCCGCTCTGAGA |
| | | AAGGGCATCATGCTGACCACACCACCTTTTTACTACGACA |
| | | GAGTGGACAACAAGTTCGTGCCTAACAAGTACAAGGACG |
| | | TGATCCTGTGGGCCTACGACGAGGCCATGAAGGGCCAGTC |
| | | CGCTAAGGCCATCGCCAGGAAGCTGAACAACTCCGACATC |
| | | CCTCCCCCTAACAATACCCAGTGGCAGGGCAGAACCATTA |
| | | CCCACGCCCTGCGCAACCCTTTCACCAGAGGCCACTTCGA |
| | | TTGGGGCGGCGTGCACATCGAAAATAACCATGAGCCTATC |
| | | ATCACCGATGAGATGTACGAGAAAGTCAAGGATAGACTG |
| | | AATGAGAGAGTGAACACCAAGAAGGTCCGACACACCTCC |
| | | ATCTTCAGAGGAAAGCTCGTGTGTCCTGTGTGCAACGCCA |
| | | GACTGACACTGAATTCTCACAAGAAGAAGTCCAACTCCGG |
| | | CTACATCTTTGTGAAGCAGTACTACTGTAACAACTGCAAG |
| | | GTGACCCCTAACCTGAAACCTGTGTACATCAAAGAGAAAG |
| | | AAGTGATCAAAGTGTTCTACAACTACCTGAAAAGATTCGA |
| | | CCTGGAAAAGTACGAAGTGACACAGAAACAGAACGAACC |
| | | TGAGATCACCATCGATATCAATAAGGTGATGGAACAGCG |
| | | GAAGAGATACCACAAGCTGTACGCCTCTGGACTGATGCAA |
| | | GAAGATGAACTGTTTGATCTGATCAAGGAAACCGACCAG |
| | | ACCATCGCTGAGTACGAGAAGCAGAACGAGAACCGGGAG |
| | | GTGAAACAGTATGACATCGAAGATATCAAGCAGTATAAG |
| | | GACCTGCTGCTGGAAATGTGGGACATCTCCTCTGACGAGG |
| | | ACAAGGAGGACTTCATCAAGATGGCTATCAAGAACATCTA |
| | | CTTCGAGTATATCATCGGCACCGGCAACACCTCTCGGAAG |
| | | CGGAACAGCCTAAAGATCACTAGCATCGAGTTCTAC |
| 198 | Int9 | ATGAAAGTGGCTATCTACACCAGAGTGTCCACACTGGAAC |
| | | AGAAAGAGAAGGGCCACTCCATCGAGGAGCAGGAAAGAA |
| | | AGCTGAGAGCCTACTCCGACATCAACGACTGGAAGATCCA |
| | | CAAGGTGTACACAGATGCTGGCTACTCTGGCGCTAAGAAA |
| | | GATAGACCTGCCCTGCAAGAGATGCTGAACGAGATCGAC |
| | | AACTTCGACCTGGTGCTGGTTTATAAGCTGGACCGGCTGA |
| | | CAAGATCCGTGAAAGATCTGCTGGAAATCCTGGAACTGTT |
| | | CGAGAACAAGAACGTGTTGTTCAGATCCGCCACCGAGGTG |
| | | TACGACACCACCAGCGCTATGGGCAGACTGTTTGTGACCC |
| | | TGGTCGGCGCCATGGCTGAGTGGGAACGGACCACCATCCA |
| | | GGAGAGAACCGCCATGGGCAGACGGGCCTCTGCTAGAAA |
| | | AGGCCTGGCCAAGACCGTGCCTCCATTCTACTACGACCGG |
| | | GTGAACGATAAGTTCGTGCCCAACGAGTACAAGAAGGTG |
| | | CTGCGGTTCGCCGTGGAAGAGGCCAAGAAGGGCACCTCTC |
| | | TGAGAGAGATCACCATCAAACTTAACAACTCTAAGTACAA |
| | | GGCCCCTCTGGGTAAGAACTGGCACCGGTCTGTGATCGGC |
| | | AACGCTCTGACCTCCCCTGTGGCCAGGGGCCATCTGGTGT |
| | | TCGGCGACATCTTCGTGGAAAACACCCACGAGGCTATCAT |
| | | CTCTGAGGAAGAATATGAAGAGATCAAACTGCGCATCTCC |
| | | GAAAAGACCAACAGCACCATCGTGAAGCACAACGCCATC |
| | | TTCCGGTCCAAGCTCCTGTGCCCCAATTGTAACCAGAAGC |
| | | TCACACTGAACACCGTGAAGCACACCCCTAAAAACAAGG |
| | | AAGTGTGGTACAGCAAGCTGTACTTTTGCTCCAACTGCAA |
| | | GAATACCAAGAACAAGAATGCCTGCAATATCGATGAGGG |
| | | CGAGGTCCTGAAACAGTTCTACAACTACCTGAAGCAGTTT |
| | | GATCTGACCTCCTACAAGATCGAGAACCAGCCTAAGGAG |
| | | ATCGAGGACGTGGGAATCGACATTGAAAAGCTGCGGAAA |
| | | GAGCGGGCCAGATGTCAGACTCTGTTCATCGAAGGAATGA |
| | | TGGACAAGGACGAGGCCTTCCCTATCATCAGCCGGATCGA |
| | | CAAGGAAATCCATGAGTACGAGAAGCGGAAGGATAATGA |
| | | CAAGGGAAAGACATTCAACTACGAGAAGATCAAGAACTT |
| | | CAAATACTCTCTGCTGAACGGCTGGGAGCTGATGGAGGAC |
| | | GAGCTGAAAACCGAATTTATCAAGATGGCCATCAAGAAC |
| | | ATCCACTTCGAGTACGTCAAGGGCATCAAGGGCAAGAGA |
| | | CAGAACTCCCTGAAGATCACCGGCATCGAGTTCTAT |
| 199 | Int10 | ATGATCACAACCAACAAGGTGGCTATCTACGTCAGAGTGT |
| | | CCACCACAAATCAAGTGGAAGAAGGCTACTCCATCGACG |
| | | AGCAGAAGGACAAGCTCTCCTCCTACTGTGACATCAAGGA |
| | | TTGGAACGTGTACAAGGTGTACACCGACGGCGGCTTTTCC |
| | | GGAAGCAACACCGATAGACCTGCCCTGGAATCTCTGATCA |
| | | AGGATGCAAAGAAGCGGAAGTTCGACACCGTGCTGGTGT |
| | | ACAAGCTGGACAGACTGTCCAGATCCCAGAAGGACACCC |
| | | TGCACCTGATCGAGGACGTGTTCATCAAGAACGGCATCGA |
| | | GTTTCTGTCCCTGCAAGAGAACTTCGATACATCTACCCCAT |
| | | TCGGCAAGGCCATGATCGGTCTGCTGTCTGTGTTCGCCCA |
| | | GCTGGAGAGAGAACAGATCAAAGAGCGGATGCAGCTCGG |
| | | CAAGCTGGGCAGAGCTAAGTCTGGAAAGTCCATGATGTG |
| | | GGCCAAAACCAGCTACGGCTACGACTACCACAAGGAAAC |
| | | CGGCACCGTGACGATCAACCCCGCTCAGGCTCTGACAATC |
| | | AAGTTTATCTTCGAGTCTTACCTGAGAGGCAGATCCATCA |
| | | CCAAGCTGAGAGATGACCTGAACGAGAAGTACCCTAAGC |
| | | ACGTGCCTTGGTCCTACAGAGCCGTGAGAACCATCCTGGA |
| | | CAATCCTGTGTACTGTGGCTTCAACCAGTACAAGGGCGAG |
| | | ATCTACCCCGGCAACCACGAGCCTATCATCTCCAAAGAGG |
| | | AGTACGACAAGACCCAGTCCGAGCTGAAGATCCGGCAGC |
| | | GGACCGCTGCTGAGAACGTGAACCCTCGCCCCTTCCAGGC |
| | | CAAGTACATCCTGTCTGGCATTGCCCAGTGCGGATATTGC |
| | | GGCGCTCCTCTGAAAATCATGCTGGGCGTCAAGAGAAAG |
| | | GACGGATCTCGGCTGAAGAAATACGAGTGCCACCAGAGA |
| | | CATCCTAGAACCCTGAGAGGCGTGACCACCTACAACGACA |
| | | ATAAGAAGTGCGACTCGGGCTTCTACTACAAGGACAAGCT |
| | | CGAGGCCTATGTGCTGAAGGAAATCTCTAAGCTGCAGGAC |
| | | GACGCCGATTACCTGGATAAGATCTTCAGCGGCGACAACG |
| | | CCGAGACAATCGACCGCGAGAGCTATAAGAAGCAGATCG |
| | | AAGAACTGTCCAAAAAACTGAGCAGACTGAACGACCTGT |
| | | ACATCGACGACCGGATCACCCTGGAGGAACTGCAGTCTAA |
| | | GTCTGCCGAATTCATCTCCATGCGGGGCACCCTGGAAACC |
| | | GAGTTGGAAAACGATCCTGCTCTGCGGAAGAACAAGCGG |
| | | AAAGCCGACATGAGAAAGCTGCTGAACGCTGAAAAGGTG |
| | | TTCTCTATGGACTACGAGTCCCAGAAAGTTCTGGTGCGGA |
| | | GACTGATCAACAAAGTGAAGGTCACCGCCGAGGATATCG |
| | | TGATCAACTGGAAGATC |
| 200 | Int11 | ATGCTGAGATGCGCCATCTACATCAGAGTGTCCACCGAGG |
| | | AGCAGGCCATGCACGGCCTGTCCATGGACGCTCAGAAAG |
| | | CCGATCTGACCGACTACGCTAAGAAGCACAACTACGAGAT |
| | | CATCGACTACTACGTGGACTCCGGCAAGACCGCCAGAAA |
| | | GAGACTGTCCAAGCGCAAGGACCTGCAGCGGATGATCGA |
| | | GGACGTCAAGCTGAACAAGATCGACATCATCATCTTTACC |
| | | AAGCTGGACAGGTGGTTCCGGAACGTGCGGGACTACTAC |
| | | AAGATCCAAGAGGTGCTGGAGGACCACAACGTCGACTGG |
| | | AAAACCATCTTCGAGAATTACGATACCTCTACCGCTAACG |
| | | GCAGACTGCACATCAACATCATGCTGTCCGTGGCTCAGGA |
| | | CGAGGCCGACAGAACCTCCGAAAGAATCAAACGGGTGTT |
| | | CGAGAACAAGCTGAAGAACAACGAGCCTACATCTGGCTC |
| | | TCTGCCTATCGGCTACAAGATCAAAGAGAAGTCCATCATT |
| | | ATCGATGAGGAAAAGGCCCCTATCGCCAAGGATGTGTTCG |
| | | ATTTCTACTACTACCACCAGTCCCAGACCAAGGTGTTCAA |
| | | AGAAATCCTCAACAAATACAACCTGTCTCTGTGCGAAAAG |
| | | ACCATCCGGAGAATGCTGGAGAATAAGCTGTACATCGGC |
| | | ATCTACAGAGAGCACGAGAACTTCTGTCCTCCTCTGATCG |
| | | ACAAGAACAAGTTCGACGAAGTGCAGCTGATTCTGAAGA |
| | | GGCGGAACATCAAGTATATCCCTACTAAGCGGATCTTTCT |
| | | GTTCACCAGCCTGCTGATCTGCAAGGAGTGTAGACATAAG |
| | | ATGATCGGCAACGCCCAGATCAGAAACACAAAGGCTGGA |
| | | AAGATCGAGTACATCTTGTACCGGTGCAACCAATCTTACG |
| | | CTCGGCACACCTGCAACCACAGAAAGGTGATCTATGAAA |
| | | ACAAGATCGAAACCTATCTGCTGAACAACATCGAGTCCGA |
| | | GCTGAAAAAGTTTATCTACGACTACGAGCTGGAAGATATC |
| | | CCCAAGGTGAAGAACAAAGTGAACAAAACAAATATCAAG |
| | | CGGAAGCTGGAAAAGCTGAAAGAACTGTACATCAACGAC |
| | | CTCATCGACATCGACATGTACAAAGAGGATTACAAGAAGT |
| | | ACACCGAGATCCTGAATACCAAAGAAGAAAAGATCGAAC |
| | | AGAGAAACCTGCAGCCTCTGAAGGACTTCCTGAACTCCGA |
| | | CTTCAAGTCTCTGTACTCCTCCATCTCTAGAGAAGAGAAG |
| | | CGGCTGCTGTGGAGAGGCATAATCAGCGAGATCCAGATC |
| | | GACTGCAATAACGATATCACCATCATCCCCCATCCA |
| 201 | Int12 | ATGAAGGTGGCCATCTACACTAGAGTGTCCTCGGCTGAGC |
| | | AGGCCAACGAGGGATACTCCATCCACGAGCAAAAGAAGA |
| | | AGCTCATCTCCTACTGCGAAATCCACGACTGGAACGAGTA |
| | | CAAAGTGTTCACCGACGCCGGCATCTCTGGCGGCTCTATG |
| | | AAGCGGCCTGCTCTGCAGAAACTGATGAAACATCTGTCTA |
| | | GCTTCGACCTGGTGCTGGTGTACAAGCTGGACAGACTGAC |
| | | CAGAAACGTGCGCGACCTGCTGGATATGCTCGAAGAATTC |
| | | GAGCAGTACAACGTATCTTTCAAGTCCGCCACCGAAGTGT |
| | | TCGACACCACCTCTGCTATCGGCAAGCTGTTCATCACCAT |
| | | GGTGGGCGCTATGGCCGAGTGGGAAAGAGAAACCATCAG |
| | | AGAGCGGAGCCTGTTTGGATCTCGGGCCGCTGTGCGGGAA |
| | | GGCAACTACATCAGAGAGGCTCCTTTCTGCTACGACAACA |
| | | TCGAGGGCAAGCTGCATCCAAACGAATACGCCAAGGTGA |
| | | TCGATCTGATCGTGTCCATGTTCAAGAAGGGCATCTCCGC |
| | | CAATGAGATCGCCAGACGGCTGAACTCCTCCAAGGTGCAC |
| | | GTGCCTAACAAAAAGTCCTGGAACCGGAACAGCCTGATCC |
| | | GGCTCATGAGATCTCCCGTTCTGCGGGGCCACACCAAGTA |
| | | CGGCGACATGCTGATCGAGAACACCCATGAGCCTGTGCTG |
| | | TCCGAACACGACTACAATGCTATCAATAATGCCATCTCCA |
| | | GCAAGACCCACAAGTCCAAGGTCAAGCACCACGCCATCTT |
| | | CAGAGGAGCCCTGGTGTGTCCTCAGTGCAACAGAAGGCTG |
| | | CACCTGTACGCTGGCACAGTGAAGGACCGGAAGGGCTAC |
| | | AAGTACGATGTCAGAAGATACAAGTGCGAGACATGTTCTA |
| | | AGAACAAGGACGTGAAGAACGTGTCCTTCAACGAGTCTG |
| | | AGGTGGAAAACAAGTTCGTGAACCTGCTGAAGTCTTACGA |
| | | GCTGAACAAGTTCCACATCCGGAAAGTGGAACCCGTGAA |
| | | AAAGATCGAGTATGATATCGACAAGATCAACAAGCAGAA |
| | | GATCAACTACACCAGATCTTGGTCCCTGGGCTATATCGAG |
| | | GACGACGAGTACTTCGAGCTGATGGAGGAGATCAACGCC |
| | | ACAAAGAAGATGATCGAGGAACAGACAACCGAGAACAAG |
| | | CAGTCTGTCAGCAAAGAGCAGATCCAGTCCATCAACAACT |
| | | TTATCCTGAAAGGCTGGGAGGAACTGACCATCAAGGATA |
| | | AAGAGGAGCTGATCCTGTCCACCGTGGACAAGATAGAGTT |
| | | CAATTTCATTCCTAAGGATAAGAAGCACAAAACCAACACC |
| | | CTGGACATCAACAACATCCACTTTAAGTTT |
| 202 | Int13 | ATGGCCGTGGGCATCTACATCAGAGTGTCCACCCAGGAGC |
| | | AGGCCTCTGAAGGCCATTCCATCGAGTCCCAGAAAAAGA |
| | | AACTGGCTTCCTACTGCGAGATCCAGGGCTGGGACGACTA |
| | | CCGGTTCTACATCGAGGAAGGCATCTCCGGCAAGAACACA |
| | | AATCGGCCTAAGCTGAAGCTGCTGATGGAACACATCGAG |
| | | AAGGGAAAGATCAACATCCTGCTGGTGTACAGACTGGAT |
| | | AGACTGACAAGATCTGTGATCGACCTGCACAAGCTGCTGA |
| | | ACTTCCTGCAAGAGCACGGCTGTGCCTTCAAGTCCGCCAC |
| | | CGAGACATACGACACCACCACTGCCAACGGCAGAATGTC |
| | | CATGGGCATCGTGTCCCTGCTGGCTCAGTGGGAAACCGAG |
| | | AACATGTCCGAGCGGATCAAGTTGAATCTGGAACATAAG |
| | | GTGCTGGTCGAGGGCGAAAGAGTGGGAGCCATCCCTTAC |
| | | GGCTTCGACCTGTCTGATGATGAAAAGCTGGTGAAGAACG |
| | | AGAAGTCTGCTATCCTGCTGGACATGGTCGAACGGGTGGA |
| | | AAATGGATGGTCCGTGAACAGAATCGTGAACTATCTGAAC |
| | | CTGACCAACAACGACCGCAACTGGAGCCCTAACGGCGTG |
| | | CTGAGGCTGCTGCGGAATCCTGCTCTGTACGGCGCTACCA |
| | | GATGGAACGATAAGATCGCCGAGAACACCCACGAGGGAA |
| | | TCATCAGCAAAGAGAGATTCAACCGGCTGCAGCAGATCCT |
| | | CGCCGACAGATCCATCCACCACCGGCGGGACGTGAAGGG |
| | | CACCTATATCTTCCAAGGCGTGCTGAGATGTCCTGTGTGC |
| | | GACCAGACCCTGTCCGTGAACCGGTTTATTAAGAAGAGAA |
| | | AGGACGGCACCGAGTATTGTGGTGTGCTGTACCGGTGCCA |
| | | GCCTTGCATCAAGCAGAACAAGTACAACCTGGCCATCGGC |
| | | GAGGCCAGATTTCTGAAGGCCCTGAACGAGTACATGTCTA |
| | | CCGTGGAATTCCAGACGGTTGAAGATGAGGTGATACCCAA |
| | | GAAGTCTGAGAGAGAGATGCTGGAGTCTCAGCTGCAGCA |
| | | GATCGCTCGGAAGCGGGAAAAGTACCAGAAGGCTTGGGC |
| | | CAGTGATCTGATGAGCGATGACGAGTTCGAGAAGCTGATG |
| | | GTGGAAACCAGAGAAACCTACGACGAGTGCAAGCAGAAG |
| | | CTCGAGTCCTGCGAGGACCCAATCAAAATCGACGAAACCT |
| | | ACCTGAAAGAAATCGTGTACATGTTCCACCAGACATTCAA |
| | | CGACCTGGAATCCGAGAAGCAGAAAGAGTTCATCAGCAA |
| | | GTTCATCAGAACCATCAGATACACCGTGAAGGAGCAGCA |
| | | GCCCATCAGACCTGACAAGTCTAAGACCGGCAAGGGCAA |
| | | ACAAAAAGTGATCATCACCGAAGTGGAATTTTACCAG |
| 203 | Int14 | ATGACAGTGGGCATCTATATCAGAGTGTCCACCGAGGAAC |
| | | AGGTCAAGGAGGGCTTCTCCATTAGCGCTCAGAAAGAAA |
| | | AGCTGAAGGCCTACTGCACCGCTCAAGGCTGGGAGGACTT |
| | | CAAGTTCTACGTGGACGAAGGCAAGTCTGCCAAGGACAT |
| | | GCACCGGCCCCTGCTCCAAGAGATGATCTCTCATATCAAG |
| | | AAGGGACTGATCGATACCGTGCTGGTGTACAAGCTGGACA |
| | | GACTGACAAGATCCGTGGTGGATCTGCACAACCTGCTGTC |
| | | CATCTTCGACGAATTCAACTGCGCCTTCAAGTCCGCCACA |
| | | GAAGTGTACGACACCTCCAGCGCCATGGGCAGATTCTTCA |
| | | TCACAATCATCTCCTCCGTGGCCCAGTTCGAGCGCGAAAA |
| | | CACCTCCGAAAGAGTGAGCTTTGGCATGGCCGAGAAGGTC |
| | | AGACAGGGCGAGTACATCCCTCTGGCTCCTTTCGGCTATA |
| | | CCAAGGGCACCGACGGAAAGCTGATCGTCAACAAGATCG |
| | | AGAAAGAAATCTTCCTGCAGGTGGTTGAGATGGTGTCTAC |
| | | CGGCTACTCTCTGCGGCAGACCTGCGAGTACCTGACCAAC |
| | | ATCGGCCTGAAAACCCGGAGATCTAATGATGTGTGGAAG |
| | | GTGAGCACCCTGATCTGGATGCTGAAGAACCCCGCCGTGT |
| | | ACGGCGCCATCAAGTGGAATAACGAGATCTACGAGAACA |
| | | CCCACGAGCCTCTGATCGACAAGGCTACCTTCAACAAAGT |
| | | GGCTAAGATCCTGTCTATCAGATCCAAGTCCACCACCTCT |
| | | AGAAGAGGCCACGTGCACCATATCTTTAAGAACCGGCTTA |
| | | TCTGCCCAGCATGTGGAAAGCGGCTGTCTGGCCTGCGGAC |
| | | CAAGTACATCAACAAGAATAAGGAAACTTTCTACAACAA |
| | | CAACTACAGATGTGCTACCTGCAAGGAGCACAGACGGCCT |
| | | GCTGTGCAGATCTCCGAGCAGAAGATCGAGAAGGCCTTTA |
| | | TCGACTACATCTCCAACTACACCCTGAACAAGGCCAACAT |
| | | CAGCTCTAAGAAGCTGGACAACAACTTAAGGAAGCAGGA |
| | | AATGATCCAGAAAGAGATCATCAGCCTGCAGCGGAAGAG |
| | | AGAGAAGTTCCAGAAAGCCTGGGCCGCCGACCTGATGAA |
| | | CGACGATGAGTTCTCCAAACTGATGATCGATACAAAGATG |
| | | GAAATCGACGCTGCTGAGGACCGGAAGAAAGAATACGAC |
| | | GTGTCCCTCTTCGTGTCTCCTGAAGATATCGCCAAGCGGA |
| | | ACAACATCCTGCGGGAGCTGAAGATCAACTGGACCTCTCT |
| | | GTCCCCTACCGAGAAAACCGATTTTATTTCCATGTTCATCG |
| | | AAGGCATCGAGTACGTGAAGGACGACGAGAATAAGGCTG |
| | | TGATCACCAAGATCTCTTTCCTG |
| 204 | Int15 | ATGAAGGCCGCCATCTATATCAGAGTGTCCACCCAGGAAC |
| | | AGATCGAGAATTACAGTATCCAGGCTCAGACCGAGAAAC |
| | | TGACCGCTCTGTGCAGATCCAAGGACTGGGACGTGTACGA |
| | | TATCTTCATCGACGGAGGCTACTCTGGCTCCAACATGAAC |
| | | AGACCCGCCCTGAATGAGATGCTGTCTAAGCTGCACGAAA |
| | | TCGACGCCGTGGTGGTGTACAGGCTGGACAGACTGTCCAG |
| | | ATCCCAGAGAGATACCATCACACTGATCGAAGAGTACTTC |
| | | CTGAAGAACAACGTGGAATTCGTGTCCCTCAGCGAAACCC |
| | | TGGACACTAGCTCTCCATTTGGCAGAGCCATGATCGGCAT |
| | | CCTGTCTGTGTTCGCCCAGCTGGAAAGAGAGACAATCCGG |
| | | GACAGAATGGTCATGGGCAAGATCAAGCGGATCGAGGCT |
| | | GGCCTGCCTCTGACAACCGCCAAGGGCAGAACATTCGGCT |
| | | ATGATGTGATCGACACCAAGCTGTACATCAACGAGGAAG |
| | | AAGCTAAGCAGCTGCAGATGATCTACGACATTTTCGAGGA |
| | | AGAGAAGTCCATCACCACCCTGCAGAAGAGACTCAAAAA |
| | | ACTGGGCTTCAAGGTGAAGTCCTACTCCTCCTACAACAAC |
| | | TGGCTGACCAACGACCTGTACTGCGGCTACGTGTCCTACG |
| | | CCGACAAAGTCCATACCAAGGGCGTGCACGAGCCTATCAT |
| | | CTCTGAAGAACAGTTCTACAGAGTGCAGGAGATCTTCAGC |
| | | CGGATGGGCAAAAATCCTAACATGAACCGGGATTCTGCTA |
| | | GCCTGCTCAACAATCTGGTCGTTTGTGGCAAGTGTGGACT |
| | | GGGATTTGTGCACAGAAGAAAGGACACCATCTCCAGAGG |
| | | TAAGAAGTACCACTACCGGTACTACAGCTGCAAGACCTAC |
| | | AAGCACACCCATGAGCTGGAGAAGTGCGGCAACAAGATC |
| | | TGGCGGGCTGACAAGCTGGAAGAATTGATCATCGATCGCG |
| | | TGAACAACTATTCCTTCGCTTCTCGGAACGTGGACAAAGA |
| | | GGACGAGCTGGACAACCTGAACGAGAAGCTGAAAACCGA |
| | | GCACAAGAAAAAGAAGCGGCTGTTCGACCTGTACATCTCC |
| | | GGCTCTTACGAGGTGTCTGAGCTGGATGCTATGATGGCCG |
| | | ACATCGATGCCCAAATCAACTACTACGAGGCCCAGATCGA |
| | | AGCCAACGAGGAACTGAAGAAGAACAAGAAAATTCAAGA |
| | | GAATCTGGCTGATCTGGCCACCGTGGACTTTGACTCCCTA |
| | | GAGTTCCGGGAAAAGCAGCTGTACCTGAAGTCTCTGATCA |
| | | ACAAGATCTACATCGACGGCGAGCAGGTGACCATCGAGT |
| | | GGCTG |
| 205 | Int16 | ATGAAGGGCGAGTCTGAGCTGGACAAGAAGGCCGCCATC |
| | | TACATCAGAGTTTCTACACAAGAGCAGGCTACAGAGGGCT |
| | | ATTCGATCCAGGCACAAACCGACAGACTGATCAAGTACGT |
| | | GGAAGCCAAGGACTTTATCCTGTATAAGAAGTATATCGAC |
| | | GCCGGCTACAGCGCTTCTAAGCTCGAAAGACCCGCTATGC |
| | | AGGATCTCATCCAGGACGTCCAAAGCAAGAAAGTGGACG |
| | | TGGTCATCGTGTACAAGCTGGATAGACTGTCTAGATCTCA |
| | | GAAGGATACCATGTACCTGATCGAGGACATCTTCCGGCCT |
| | | AACGACGTGGAACTGATCTCTATGCAGGAAAGCTTTGACA |
| | | CCTCCACCGCCTTCGGCTCTGCCACCGTGGGCATGCTGTCC |
| | | GTGTTCGCCCAACTGGAGAGGAAGTCCATCTCCGAAAGAA |
| | | TGATCACAGGCAGAGTGGAGCGGGCTAAGAAAGGCTTCT |
| | | ACCACACCGGCGGCCAGGACAGACCTCCAGCTGGCTACC |
| | | AGTTCAACTCCGACAACCAGCTGATCATCAACGAGTACGA |
| | | GGCCGCTGCTATCAAGGACCTGTTTCGGCTGTACAACGAC |
| | | GGCCTGGGAAAGTCTAGCATCTCCGAGTACCTGAAGAAG |
| | | AACTACCCCGGAAAAAACAAGTGGCTGCCTTCTTCTATCG |
| | | ATCGGATGCTGAAGAACTCCCTGTACATCGGCAAGGTGAA |
| | | GTTCTCCGGCGCCGAGTACGACGGCATCCATGAGCCTATC |
| | | ATAGACGAAGTGACCTTCTACAAGACCCAGAAGGAGATC |
| | | GCCAGACGGAAGCAGACCAACACCAAGAGATACAACTAC |
| | | GTGGCCCTGCTGGGCGGCCTGTGCGAGTGCGGCATCTGTG |
| | | GCGCTAAGATGGCCAACAGACGGGCCGTGGGACGCAAGG |
| | | GTAAGGTGTACCGGTACTACAGATGCTACTCCAAGAAAGG |
| | | ATCTCCTAAGCACATGATGAAAACCGATGGCTGCTCCTCC |
| | | AAGGCCCAGCAGCAGTTCATCATCGACGAGGCTGTGATTA |
| | | ACAACCTGAAGAACATCGACGTCGAAGCCGAACTGAAAC |
| | | GCAGATCTGCTCCTCAGACCAATACCTCTCTGATCTCCAG |
| | | CCAGATCGAGAGCATCGATAAGCAGATTAACAAGCTGAT |
| | | CGACCTGTTCCAGGTGGACTCCATGCCTCTGGATGTGATC |
| | | AGCGAGAAGATCGATAAGCTGAACAAAGAGAAGCAGTCC |
| | | ATGGAAAAACTGCTGGAACGGAAGAATAAGCTGGACAAA |
| | | ACCGAGCTGCAGCACAGATTCGATGTGCTGAAGTCCTTCG |
| | | ACTGGGACAATTCCAGTATCGAGTCCAAGCGGGTGGTGAT |
| | | CGAGATGCTGGTGCAGAAAGTGATCATTCACGACAACTCC |
| | | ATCGAAATCATCCTGGTGGAA |
| 206 | Int17 | ATGCGGACCAACGAGCACAACTTCCACAACATCGAGGAG |
| | | GAGATTAAGCACGTGGCCGTGTACCTGAGACTGTCCCGGG |
| | | GTGAGGATGAGAGCGAGCTGGATAACCACAAGACTCGGC |
| | | TGCTGAACAGATGTGAACTCAACAACTGGTCCTACGAGCT |
| | | GTATAAGGAAATCGGATCTGGCTCTACCATCGATGATAGA |
| | | CCTGTGATGCAGAAACTGCTGACCGATGTGGAAAAGAAC |
| | | CTGTACGACGCCGTGCTGGTGGTGGACCTGGATAGGCTGT |
| | | CGAGAGGCAACGGCACCGACAACGACAGAATCCTGTATT |
| | | CCATGAAAGTGTCCGAAACCCTGATCGTGGTGGAATCCCC |
| | | CTACCAGGTGCTGGACGCTAACAACGAGTCCGACGAAGA |
| | | GATCATCCTGTTTAAGGGCTTCTTCGCCCGGTTCGAGTTCA |
| | | AGCAGATCAATAAGCGGATGAGAGAGGGCAAGAAGCTGG |
| | | CTCAGAGCAGAGGCCAGTGGGTCAACTCCGTGACACCCTA |
| | | CGGCTACATCGTTAACAAGACCACCAAGAAACTGACCCCT |
| | | TCTGAAGAGGAAGCCAAAGTGGTGATCATGATCAAGGAC |
| | | TTCTTCTTTGAAGGCAAGAGCACCTCCGACATCGCTTGGG |
| | | AGCTGAACAAGAGAAAGATCAAGCCTAGACGGGCTACAG |
| | | AATGGCGGTCCTCCTCTATCGCCAATATCCTGCAGAATGA |
| | | AGTGTACGTGGGCAACATCGTGTACAACAAGTCTGTCGGA |
| | | AACAAGAAGCCCTCTAAGTCCAAGACCAGAGTGACCACC |
| | | CCATACAGACGGCTGCCTGAGGAGGAGTGGCGGCGCGTG |
| | | TACAACGCCCACCAGCCTCTGTACTCTAAGGAAGAGTTCG |
| | | ACCGGATCAAGCAGTACTTCGAGTGCAACGTCAAGAGCC |
| | | ATAAGGGATCCGAGGTGCGCACCTACGCCCTGACCGGCCT |
| | | GTGCAAGACCCCTGACGGCAAGACCATGAGAGTGACCCA |
| | | GGGCAAGAAGGGCACCGACGACGACCTGTATCTGTTCCCT |
| | | AAGAAGAACAAGCACGGCGACAGCAGTATCTACAAGGGC |
| | | ATTTCCTACAACGTCGTGTACGAGACACTCAAAGAGGTGA |
| | | TCTTGCAAGTGAAAGACTACCTGGACTCTGTGCTGGACCA |
| | | GAACGAAAATAAGGACCTGGTGGAAGAACTGAAAGAGGA |
| | | ACTGATGAAGAAGGAGGATGAACTGGAAACAATCCAGAA |
| | | GGCCAAGAATCGGATCGTGCAAGGCTTTCTGATCGGCCTG |
| | | TACGACGAGCAGGACTCCATCGAGTTGAAGGTGGAGAAG |
| | | GAGAAAGAGATCGACGAAAAGGAAAAGGAGATCGAGGC |
| | | TATCAAGATGAAGATCGACAATGCAAAAACCGTGAACAA |
| | | CTCCATCAAAAAAACCAAGATCGAGAGACTGCTGTCTGAC |
| | | GTGCAGTCTGCCGAGTCTGAGAAAGAAATCAACCGGTTCT |
| | | ACAAGACCCTGATCAAGGAGATCATCGTGGATAGAACCG |
| | | ATGAAAACGAGGCTAAGATCAAGGTCAACTTCCTG |
| 207 | Int18 | ATGATCACAACAAACAAGGTGGCCATCTACGTGCGGGTGT |
| | | CTACCACCAACCAAGTGGAGGAAGGCTACTCCATCGACG |
| | | AGCAGAAGGACAAGCTGGAGGCTTACTGCAAGATCAAAG |
| | | ACTGGAAGATCTACGATGTGTACGTGGATGGCGGCTTCAG |
| | | CGGCGCCAACACCCAGCGGCCTGAGCTGGAACGGCTGAT |
| | | CTCCGACGTGAAGCGGAAGAAGGTGGACATCGTGCTGGT |
| | | GTATAAGCTGGACAGACTGTCTAGATCCCAGAAGGACAC |
| | | ACTGTTTCTGATCGAGGATGTGTTCGCCAAGAACGACGTG |
| | | GCTTTCATCAGCCTGCAGGAGAACTTCGACACCTCCACCC |
| | | CTTTCGGAAAGGCCTCTATAGGCATGCTGTCTGTGTTTGCT |
| | | CAGCTGGAGCGGGAGCAGATCAAGGAAAGAATGATGCTG |
| | | GGCAAAGAAGGCAGAGCCAAGAATGGCAAGTCCATGTCT |
| | | TGGACCACCATCGCCTTCGGCTACGACTACTCTAAGGAAA |
| | | CCGGCGTGCTGTCCGTGAACCCTACCCAGGCTCTGATCGT |
| | | CAACCGGATCTTCACCGAGTACCTGAACGGCAAGCCTGTG |
| | | GTGAAAATCATCCGGGACCTGAACGCCGAGGGCCATGTG |
| | | GGCAGAAAGCGGCCTTGGGGCGAGACAATCACCAAGTAC |
| | | CTGCTGAAGAACGAGACATACCTGGGCAAGGTTAAGTAT |
| | | AAAGACAAGGTGTACGAGGGCCAGCACGAGCCCATCATC |
| | | ACCCAAGAGCTGTTCGATCTGGTGCAGCTGGAAGTGGAGC |
| | | GGAGACAGATCTCCGCCTACGAAAAGTACAACAACCCCA |
| | | GACCATTCAGAGCTAAGTACATGCTGAGCGGCCTGATGAA |
| | | GTGCGGATACTGTGGCGCTTCTCTGGGCCTGAGATACACC |
| | | AGAAAGGACAAGAACGGCATCTCTCACCACAAGTACCAG |
| | | TGCCGGAATCGGCACTCCAAGGACCTGGAAAAAAGATGC |
| | | GAGTCTGGCTGGTACTCCAAAGAGGAACTCGAGCGCGGA |
| | | GTGATCAAGGAACTGGAACGTATCAAGTTCGATCCTAAGT |
| | | ATAAGAATGAAACCCTGGCCAAGAAAGAGGAAACCATCA |
| | | AAGTGGAAGAGATCAAGAAGCAGCTGGAGCGGATCAACA |
| | | ACCAGGTGTCCAAACTGACCGAGCTGTACCTCGATGAGAT |
| | | CATCACCAGGAAGGAGCTTGATGAAAAGAACGACAAGAT |
| | | CAAGACCGAAAGACAATTCCTGGAGGAGCAGCTGGAGAA |
| | | CCAGAAGTCCAACGTGCTCTCCATCAGAAAGCGGAAACTG |
| | | ACCAGACTGCTGAAGGATTTTGACGTCGAGAAGCTGTCCT |
| | | ACGAGGACGCCTCTAAGATTGTCAAGAACATCATCAAAG |
| | | AAATCATCGTGACTAAGGACGGCATGTCCATCACCCTGGA |
| | | CTTC |
| 208 | Int19 | ATGGGCAAGTCTATCACCGTGATCCCAGCTAAAAAAGTGC |
| | | AGACCTCTGTGCTGCATCAAGACCGGAAGAAGATCAAGG |
| | | TGGCCGCCTACTGTCGGGTGTCCACCGACCAGGAGGAGCA |
| | | GCTGTCCTCCTATGAAAACCAGGTGAACTACTACAGAGAG |
| | | TTCATCTCCAAGCACGAGGACTACGAGCTGGTGGACATCT |
| | | ACGCCGACGAGGGCATCTCCGCAACCAACACCAAGAAGC |
| | | GGGACGCCTTCAACCGGCTGATCCAAGACTGTAGGGCCGG |
| | | AAAGGTCGACAGAATACTGGTGAAGTCCATCTCGAGATTC |
| | | GCCAGAAACACACTGGATTGCATCAAGTACGTGCGGGAG |
| | | CTGAAGGAACTGGGCGTGGGCGTGACCTTCGAGAAAGAG |
| | | AACATCGACAGCCTGGATAGTAAGGGCGAGGTTCTGCTGA |
| | | CCATTCTGAGCTCTCTGGCTCAGGACGAGTCTCGATCTATC |
| | | TCTGAGAACGCCACCTGGGGCATCAGAAAGAAGTTCGAG |
| | | AGAGGCGAAGTGCGCGTCAATACAACAAAGTTCATGGGC |
| | | TACGACAAGGACGAGAACGGCAGACTGATCATCAACCCT |
| | | CAACAGGCTGAAACCGTCAAGTTTATCTACGAGAAATTTC |
| | | TGGAGGGCTACTCCCCCGAGTCCATCGCCAAGTACCTGAA |
| | | CGACAATGAGATCCCTGGCTGGACCGGCAAGGCCAACTG |
| | | GTACCCTTCTGCCATCCAGAAGATGCTGCAGAACGAGAAG |
| | | TACAAGGGCGACGCTCTGCTGCAGAAAACCTTTACCGTGG |
| | | ACTTCCTGACCAAGAAGAGAGTGCAGAACGATGGACAGG |
| | | TGAACCAGTACTACGTGGAAAATTCTCACGAGGCCATCAT |
| | | CGACGAAGAGACATGGGAAACAGTGCAGCTCGAGATGGC |
| | | CAGAAGAAAGACCTACAGAGATGAGCACCAGCTGAAATC |
| | | CTACATCATGCAGTCCGAGGATAACCCCTTCACCACCAAG |
| | | GTGTTCTGCGGCGCTTGTGGCTCCGCTTTCGGCCGGAAGA |
| | | ACTGGGCTACCTCCAGAGGAAAGCGGAAAGTGTGGCAGT |
| | | GCAACAACAGATACCGGATCAAGGGAGTCGAAGGCTGCT |
| | | ACAGCTCCCACCTGGACGAGGCTACCCTCGAACAGATCTT |
| | | CCTGAAAGCCCTGGAACTGCTGTCCGAAAACATCGACCTG |
| | | CTGGATGGCAAGTGGGAGAAGATCCTGGCCGAGAACAGA |
| | | CTGCTTGATAAGCACTATAGCATGGCTTTATCTGATCTGCT |
| | | GCGGCAGGAACAGATCGACTTCAATCCTTCCGACATGTGC |
| | | AGAGTGCTGGACCACATCCGGATCGGCCTGGATGGCGAA |
| | | ATCACCGTGTGCCTGCTGGAAGGTACCGAGGTGGACCTG |
| 209 | Int20 | ATGAGAACAGTCAGACGCATCCAGCCTATCAAGTCTCCTT |
| | | GCAAGCCTAGATTCAAAGTGGCCGCCTATGCTAGAGTGTC |
| | | CGACTCACGCCTGCACCACTCTCTGTCCACCCAGATCTCCT |
| | | ACTACAACAGACTGATCCAGGCCCATCCTGATTGGGAGTT |
| | | GGTCGGAATCTACTACGACGAGGGAATTTCCGGCAAAGA |
| | | GCAGTCCAACAGACAGGGCTTCCTGAATCTGATCAAGGAC |
| | | TGCGAGGACGGCAAGATCGATAGAATCATCACCAAGTCC |
| | | ATCGCCAGATTTGGACGGAACACCGTGGAACTGCTGACCA |
| | | CCGTGCGGCAGCTGAGACTGAAGAACATCGGCGTGACCTT |
| | | CGAGAAGGAAAACATCGACAGCCTGTCCTCTGAAGGCGA |
| | | GCTGATGCTGACACTGCTGGCTTCTGTGGCCCAGGAAGAG |
| | | TCCCAGAACCTGTCTGAGAATATCAGATGGCGGATCCAGA |
| | | AGAAGTTCGAAAAGGGAATCCCTCACACCCCTCAGGACAT |
| | | GTACGGCTATCGGTGGGATGGCGAACAGTACCAGATCGA |
| | | ACCCAACGAGGCCAAGGTGATCCGGAAGGTGTTCAAGTG |
| | | GTACCTGGACGGCGACTCCGTGCAGCAGATCGTGGACAA |
| | | GCTGAACCAGGAGCAGGTGCTGACCCGGCTCGGCAACCC |
| | | CTTCACCGTGGCTAGCATCAGAGAGTTCTTCAAGCAGGAA |
| | | GCTTACTTTGGTAGACTCGTGCTGCAGAAAACCTACAGAG |
| | | AAGCCTTCTCCAGAAATCCAAAGAGGAACAAAGGCCAGA |
| | | GAAACAAGTACATCATCGAGAACGCTCACGAGCCCATCGT |
| | | TACAAAGGAATACTTCGACCTGGTGCTGCATGAGAAAGA |
| | | GCGAAGAAACCAACTGATGCACCAAGAGTCTCACCTGAA |
| | | CAAGGGCATCTTCCGGGATAAGATCTCTTGCTCCGAGTGC |
| | | GGCTGTCTGATGATCGTGAAAGTCGATTCCAAGCAAGTGA |
| | | ACAAGACCGTGCGGTACTACTGCAGAACCAGAAACCGGT |
| | | TCGGCGCTTCTTCCTGCAGCTGTCGGACCCTGGGCGAGAA |
| | | GCGGCTGCTGGCCAGCTTTAAATCCAAGCTGGGCATCGTG |
| | | CCTGACAAGGAGTGGGTGGAAAACAACATCAAGCACATC |
| | | GAGTACGACTTCGGCTACCGGATCCTGCGGGTGACACCTG |
| | | TGAAGGGCAGAAAGTACCTGATCGAGATCAGAGAGGGCA |
| | | GATAC |
| 210 | Int21 | ATGCGGAACAAGGTTGCCATCTACGTCCGGGTGTCCACAG |
| | | CTAGCCAGGCCGACGAGGGCTACTCCATCGACGAACAGA |
| | | AAAGCAAGCTGGAGGCCTACTGCGAGATCAAGGACTGGA |
| | | AGATCTACGACACCTACATCGATGGCGGCTTCTCCGGGGC |
| | | CAACACCCAGAGGCCCGAACTGGAACGGCTGATTTCTGAT |
| | | GCCAAGCGGAAGAAGATTGATATCGTGCTGGTGTACAAG |
| | | CTGGACAGACTGTCCAGATCTCAAAAGGACACACTGTTCC |
| | | TGATCGAGGATGTGTTCGCTAAGAACGACGTGGCTTTCAT |
| | | CAGCCTGCAGGAGAACTTCGACACCTCTACCCCTTTCGGC |
| | | AAGGCCTCCATCGGCATGCTGTCCGTGTTCGCCCAGCTGG |
| | | AGCGCGAACAGATCAAAGAGCGGATGATGCTGGGCAAAG |
| | | AGGGCAGAGCCAAGAATGGCAAGTCCATGTCTTGGACCA |
| | | CCATCCCTTTTGGCTACGACTACTCCAAAGAGACAGGCAT |
| | | CCTGAGCGTGAACCCCACCCAAGCTCTGATCGTGAAGAGA |
| | | ATCTTCACCGAGTACCTGAACGGCAAATCTGTGGTGAAGA |
| | | TCATCCGGGACCTGAATGCCGAGGGCCATGTGGGCCGGA |
| | | AGCGGCCTTGGGGCGAAACCATCACCAAGTATCTGCTGAA |
| | | AAACGAAACCTACCTCGGAAAGTCTAAGTATAAGGGCAA |
| | | GGTATTCGAAGGCCAGCACGACGCCATCATCTCTCAGGAA |
| | | CTGTTTGATCTGGTGCAGCTGGAAGTGGAGAAGAGACAG |
| | | ATCTCCGCCTTCGAGAAGTACAACAACCCTAGACCTTTCC |
| | | GGGCTAAGTACATGCTGTCTGGCCTAATGAAGTGCGGCTA |
| | | CTGCGGCGCTTCTCTGGGACTCTACGTGGCCCCTAAGAAC |
| | | AAGAACGGCGTGAGCAAGTACAAGTACCAGTGTAGACAC |
| | | CGGTACCACAAGGACAAAGCCATCAGATGCAACTCCGGA |
| | | TGGTACTCCAAGGACGAGCTGGAGAAAAGAGTGATCAAA |
| | | GAGCTCGAGCGGCTGAAGTTCGATCCTAAGTACAAGAAA |
| | | GAAACCCTGGCCAAGAAAGATGAGACAATTAAGGTGGAG |
| | | GACATCAAGAAGCAGCTGGAAAGAATCAATAAGCAGGTG |
| | | TCCAAGCTGACCGAGCTGTACCTGGACGAGGTGATCACCA |
| | | GAAAGGACCTGGACGAAAAGAACGCCAAGATCAAGACCG |
| | | AAAGACAGTACCTGGAGGAGCAGCTGGAGAACCAGAAGT |
| | | CCAACGTGATGTCCATCCGAAAGCGGAAGCTGTCTAGACT |
| | | GCTGAAGGACTTCGACATCGAGAAGCTGTCCTACGAGGA |
| | | AGCTTCTAAGATCGTGAAGTCCGTCATCAAGGAAATCGTC |
| | | GTGACCAAGGACGACATGACCATCACTCTGGATTTT |
| 211 | Int22 | ATGAAGGTGGCCACTTACGTGCGCGTGTCCACCGACGAGC |
| | | AGGCTAAGGAGGGCTTCTCCATCCCCGCCCAAAGAGAGC |
| | | GGCTGAGAGCCTTCTGCGAGTCTCAGGGATGGGAAATCGT |
| | | GGAAGAGTACATCGAAGAGGGCTGGTCCGCCAAAGACCT |
| | | GGACAGACCTCAGATGCAGCGGCTGCTCAAGGATATCAA |
| | | GAAGGGCAATATCGACATCGTGCTGGTGTACAGGCTGGAT |
| | | AGACTGACCCGGTCTGTGCTGGATCTGTACCTGCTGCTGC |
| | | AGACCTTTGAGAAGTACAACGTGGCTTTCAGATCCGCTAC |
| | | CGAGGTGTACGACACCTCTACCGCCATGGGCAGACTGTTC |
| | | ATTACCCTTGTGGCCGCCCTGGCTCAGTGGGAGCGGGAGA |
| | | ACCTGGCCGAGAGAGTGAAGTTCGGCATCGAGCAGATGA |
| | | TCGACGAGGGAAAGAAGCCTGGCGGCCACTCTCCATACG |
| | | GATACAAGTTTGACAAGGACTTCAACTGCACCATCATCGA |
| | | GGATGAGGCCAACACCGTGCGGATGATTTACAGAATGTAC |
| | | TGCGACGGCTACGGCTACCACTCCATCGCTAAGCGCCTGA |
| | | ATGAGCTGGGCATCAAGCCTAGAATCGCCAAAGAGTGGA |
| | | ACCACAACAGCGTCCGGGACATCCTGACCAACGACATCTA |
| | | CATCGGCACCTATAGATGGGGCAACAAGGTTGTGCTGAAC |
| | | AACCATCCTCCTATCATCTCCGAGACACTGTTCAGAAAGG |
| | | TGCAGAAAGAAAAAGAAAAGCGGCGGGTGGACCGGACCA |
| | | GAGTGGGCAAGTTTCTGCTGACAGGCCTGCTGTACTGTGG |
| | | CAATTGCAACGGCCACAAGATGCAGGGCACCTTTGACAA |
| | | AAGAGAACAGAAAACCTACTACCGGTGTCTGAAGTGCAA |
| | | CCGGATCACCAACGAGAAGAACATCCTGGAACCTCTGCTG |
| | | GATGAGATCCAGCTGCTGATCACATCTAAAGAGTACTTCA |
| | | TGTCCAAGTTCTCCGACCAGTACGATCAAAAGGAGGAAGT |
| | | GGACGTGTCTGCTCTGAAGAAGGAGCTCGAAAAGATCAA |
| | | GAGACAGAAGGAAAAGTGGTACGACCTGTACATGGACGA |
| | | CAGAAACCCCATCCCTAAGGAAGATCTGTTCGCCAAGATC |
| | | AACGAGCTGAACAAGAAGGAAGAAGAGATCTATAACAAG |
| | | CTGAACGAGGTCGAACCCGAGGACAAGGAGCCTGTCGAA |
| | | GAAAAGTACAACAGACTGAGCAAGATGATCGACTTCAAG |
| | | CAGCAGTTCGAGCAAGCTAATGATTTCACCAAGAAAGAA |
| | | CTGCTGTTCAGCATCTTCGAAAAAATCGTGATCTATCGGG |
| | | AGAAGGGCAAGCTGAAAAAGATCACCCTGGACTACACCC |
| | | TGAAG |
| 212 | Int23 | ATGCTGCGCGTGGCTCTGTATATCAGAGTGTCTACCGAGG |
| | | AGCAGGCCCTGAACGGCGACAGCATCCGGACCCAGATCG |
| | | AGGCCCTGGAACAGTACTCCAAGGAGAACGACTTCAACA |
| | | TCGTGGGCAAGTACATCGACGAGGGCTGTTCTGCCACCAA |
| | | CCTGAAGCGGCCTAATCTGCAAAGACTGCTGCGGGACGTG |
| | | GAAAAAGACAAAGTGGACCTGGTGCTGATGACTAAGATC |
| | | GATCGGCTGTCTAGAGGAGTCAAGAACTACTACAAGATCA |
| | | TGGAAACACTGGAGAAGCACAAGTGCGACTGGAAAACCA |
| | | TCCTGGAAAACTACGACTCCTCCACCGCCGCTGGCAGACT |
| | | CCACATCAACATCATGCTGTCCGTGGCCGAGAACGAGGCT |
| | | GCTCAGACCTCCGAGAGAATCAAGTTCGTGTTCCAGGACA |
| | | AGTTGAGAAGAAAGGAAGTGATCTCTGGTACAATCCCCAT |
| | | CGGCTACAAAATCGAGAATAAGCATCTGGTGATCGATAA |
| | | AGAGAAGAAGTACATTGTGAAGGCCATCTTCGACGAGTA |
| | | CGAGAAGTCTGGCTCCGTTAGGACCCTGATCGAAACCATC |
| | | AACAACCTGCACGGCGAACTGTACTCCTATAACAAGATCA |
| | | AGAACATCCTGAGAAACGAGCTGTACATCGGCATTTACAA |
| | | TAAGAGAGGCTTCTACGTGGAGGACTACTGCGAGCCTATC |
| | | ATCAGCAAGAAGCAGTTCAAGCAGATCCAGCGGATCCTG |
| | | GAAAAGAATAAGAAAACCACACCAAACAAGAACATCCAC |
| | | TACCACATCTTCAGCGGCCTGCTCAAGTGCAAGGAGTGTG |
| | | GCTACACCCTGAAGGGCAACTCCTCCAACGTGGGAGAGA |
| | | AGCTGTACCTGTCTTACAGATGCTCCACCTTTTACCTGAAC |
| | | AAGAACTGCGTGCACAACGTGACCCACAACGAGAAGCAT |
| | | ATTGAGAACTATCTGCTGACCAACCTGAAGCCTCAGCTGC |
| | | ACAAGCACATGGTGAAGCTGGAAGCCCAGAACGAAAAGA |
| | | TCAGACGGAACAAAAAGTCCAACAAGAAGGATGAGAAAA |
| | | AGAAAATCATGAAGAAACTGGATAAGATCAAGGACCTGT |
| | | ACCTGGAGGACCTGATCGATAAAGAAACCTACCGGAAGG |
| | | ACTACGAGAAGCTGCAGTCCCAGCTGGACAACATCACCG |
| | | AGGAACAAGAGTCTCAGATCATCGACACCTCTCACATCAA |
| | | GAAGTTTCTGGACATCGACATCAATGAGATGTACTCTGAT |
| | | CTGAGCAGAGTCGAGCGGCGGAGATTCTGGCTGTCCATCA |
| | | TAGACTACATCGAGATCGATAACAACAAAAACATCACCAT |
| | | CAACTTCATC |
| 213 | Int24 | ATGAAGATCACCCTGCTGTACTACATCAAGAAGTTCAACA |
| | | TCTACTGCAACAGATACCTGAGCCAGCAGATCAACATCTC |
| | | CGTGGACATCATCGGCTTCTACCAGTTCAAGAACGTCACC |
| | | AACTCTGTGACCGACGTGCTGAAGAGAGGTGATAATCTGG |
| | | ACAGAATCTGTATCTACCTGCGGAAGTCCAGAGCCGATGA |
| | | AGAACTGGAAAAGACCATCGGAGTGGGCGAAACCCTGAG |
| | | CAAGCACAGAAAGGCTCTGCTGAAGTTCGCCAAGGAAAA |
| | | GAAGCTGAATATCATGGAAATCAAAGAGGAAATAGTGTC |
| | | CGCTGACTCCATCTTCTTCAGACCTAAGATGATCGAACTG |
| | | CTGAAGGAGGTGGAGAACAACCAGTACACCGGCGTGCTG |
| | | GTTATGGACATCCAGAGACTGGGCAGAGGCGACACCGAG |
| | | GACCAGGGCATCATTGCTAGAATCTTCAAGGAGTCTCACA |
| | | CCAAGATCATCACCCCTATGAAAACCTACGACCTGGACGA |
| | | CGATTTGGACGAGGACTACTTTGAGTTCGAGAGTTTCATG |
| | | GGCCGCAAAGAGTACAAGATGATCAAGAAGCGGATGCAG |
| | | GGCGGCAGAGTGCGGTCCGTGGAAGATGGCAACTACATC |
| | | GCCACCAATCCTCCATTTGGCTACGACATCCACTGGATCA |
| | | ACAAGTCCAGGACACTGAAGTTCAACTCCAAGGAATCTGA |
| | | GATCGTGAAACTGATCTTTAAACTGTATACCGAGGGAAAT |
| | | GGCGCTGGCACCATCTCCAACTACCTGAACTCCCTGGGCT |
| | | ATAAGACCAAGTTCGGCAACAACTTCAGCAACTCTTCTAT |
| | | CATCTTCATCCTGAAGAACCCTGTGTACATCGGAAAGATC |
| | | ACCTGGAAGAAGAAGGACATCAGAAAGTCCAAGGATCCT |
| | | CACAAGGTCAAAGATACCCGGACCAGAGACAAGTCCGAG |
| | | TGGATCATCGCCGACGGCAAGCACGAGCCTATCATCGACG |
| | | AAAAGATCTGGAACAAGGCTCAAGAGATCCTGAACAACA |
| | | AGTACCACATCCCTTACAAGATCGCCAACGGCCCCGCTAA |
| | | CCCTCTGGCCGGAGTGGTGATCTGCTCCAAGTGCAACTCC |
| | | AAAATGGTGATGCGGAAGTACGGCAAGAAGCTGCCTCAT |
| | | CTGATCTGCAATAACAAGGAGTGTAACAATAAGTCCGCCA |
| | | GATTCGACTACATCGAGAAGGCCGTGCTGGAAGGCCTGG |
| | | ACGAGTATCTGAAGAACTACAAAGTGAACGTGAAGGCCA |
| | | ACAACAAAACCAGCGATATCGAGCCCTACGAGCAGCAGT |
| | | CTAACGCCCTGAACAAAGAGCTGATCCTCCTGAACGAGCA |
| | | GAAACTGAAGCTATTCGACTTTTTGGAAAGAGAGATCTAC |
| | | ACAGAAGAGATCTTTCTTGAGAGATCTAAGAACCTGGATG |
| | | AGCGGATCAACACCACCACACTGGCTATAAACAAGATCA |
| | | AGAAAATTCTGGACAACGAGAAAAAGAAGAACAACAAGA |
| | | ACGACATCGTCAAGTTCGAGAAAATCCTGGAAGGCTACA |
| | | AGAAAACCAACGATATCCAGAAGAAAAATGAACTGATGA |
| | | AATCTCTGGTGTTCAAGATCGAGTATAAGAAAGAACAGCA |
| | | CCAGCGGAACGACGGCCTGCTGTACATCTACTTCCTGAGC |
| | | TTCTGCGTGCGGTGCATCTCCTACCTGACACAATTCATTTC |
| | | CTTCTTCGTGTACCCCTACCGGATCCTGGAGATCTACCTGA |
| | | CCTTCTCTTTTTTCATCATCTCTTACGAGCAT |
| 214 | Int25 | ATGCGGATCTGCATGTACCTGCGGAAGTCCAGAGCTGATG |
| | | AGGAACTGGAAAAGACCCTGGGCGAAGGCGAGACTCTGA |
| | | GCAAGCACAGAAAGGCTCTGCTGAAGTTCGCCAAGGAGA |
| | | AAAATCTGAATATCGTGGAGATCAAAGAGGAAATCGTGT |
| | | CTGGTGAGTCCCTGTTCTTCAGACCTAAGATGCTGGAACT |
| | | GCTGAAAGAAATCGAGAACAAACAGTACTCCGGCGTGCT |
| | | CGTGATGGACATGCAGAGACTGGGAAGGGGAAACATGCA |
| | | GGACCAGGGCATCATCCTCGAGACATTTAAGAAATCTAAC |
| | | ACCAAGATCATCACCCCTATGAAAACCTACGACCTGTCTA |
| | | ACGACTTCGACGAAGAGTACTCTGAGTTCGAGGCCTTCAT |
| | | GTCCCGGAAGGAACTTAAGATGATCAATCGGCGGATGCA |
| | | AGGCGGCAGAGTGCGGAGCGTCGAGGACGGCAACTACAT |
| | | CGCTACCAACGCCCCCTACGGCTACGACATCCACTGGATC |
| | | AACAAGGCCAGAACCCTGAAGCCCAACCAGAAGGAATCT |
| | | GAAATCGTCAAGCTGATCTTCAAGCTCTACATCGAGGGCA |
| | | ACGGCGCTGGCACCATCGCTAAGCATCTGAACAGCCTGGG |
| | | CTATAAGACCAAGTTCGGCAACTCCTTCAACAACTCCTCC |
| | | ATCATCTTCATTCTGAAAAACCCTGTGTATATCGGCAAGA |
| | | TCACCTGGAAGAAAAAGGACATTCGGAAGTCCAAGGATC |
| | | CTAACAAAGTGAAGGACACCCGGACCAGAGACAAGTCTG |
| | | AGTGGATCATCGTGGACGGCAAGCACGACCCTATCATCGA |
| | | CCAGATCACCTGGAAGCAGGCTCAAGAGATCCTGAATAA |
| | | CCGGTACCACGTGCCTTACAAGCTGGTCAACGGCCCTGCC |
| | | AACCCCCTGGCCGGCCTGATCATCTGTACCACCTGCAAGT |
| | | CCAAGATGGTGATGAGAAAGCTGAGAGGCACCGACAGAA |
| | | TCCTGTGCAAGAACAACAAGTGCAACAACATCTCCAACAG |
| | | ATTCGATGCCGTGGAAAAGTCCGTGGTGGAATCTCTGGAA |
| | | AACTACCTGAAGGCCTACAAGGTGAACCTGCCTGAGCTGA |
| | | ACAAGACCTCCAACCTGAAACTGTACGAGCAGCAGATCA |
| | | GCACACTGAAGAAAGAACTGAAAATTTTGAACGAACAGA |
| | | AACTGAAGCTGTTCGATTTTCTGGAGCGCGGAATCTACGA |
| | | CGAGGATACCTTCCTGAAGAGATCTAAGAACCTGGACGA |
| | | GAGAATCGAGATCACCAACGAGTCTCTGTCTAATCTGAAT |
| | | CAGATCATCGCCAAGGAGAACAAGGCCATCAAGAAAGAA |
| | | GATATCATCAAGTTTGAGAAGGTGCTGGATAGCTACAAGT |
| | | CCACCGCTGACATCCGGCTGAAAAACGAGCTGATGAAAA |
| | | CCTTAATCTTCAAGATCGAGTACACCAAGAACAAGAAGG |
| | | GCAATGACTTCAAGATCAAGGTGTTCCCTAAGCTGAAGCC |
| | | ACTGAACATC |
| 215 | Int26 | ATGATCGCCGCTATCTACTCTAGAAAGTCTAAATTCACCG |
| | | GCAAGGGCGAGTCCGTGGAAAACCAGATCGAAATGTGCA |
| | | AGGAATACCTGAAGAGAAACTTCAATAACATCGATGACA |
| | | TCGAAATCTACGAGGACGAGGGCTTCTCTGGCAAGGACAC |
| | | CAACCGGCCCAAGTTTAAGAAGATGATCAAGGCCGCTAA |
| | | AAACAAGAAGTTCAACATCCTCATCTGCTACCGGCTGGAC |
| | | AGAATCTCTCGCAACGTGGCTGATTTCAGCAATACCATCG |
| | | AGGAGCTGCAGAAATACAACATCGACTTTATATCCATCAA |
| | | GGAGCAGTTCGATACCAGCACCCCAATGGGCAGAGCCAT |
| | | GATGAACATCGCTGCTGTGTTCGCCCAGCTGGAGCGGGAA |
| | | ACCATCGCCGAGCGGATCAAGGACAACATGGTGGAACTG |
| | | GCCAAGACCGGACGGTGGCTGGGCGGCACCTCTCCTCTGG |
| | | GCTACAAGTCCGAACCCATCGAGTACTCCAATGAGGACGG |
| | | CAAGTCCAAGAAGATGTACAAGCTGACCGAGGTTGAGAA |
| | | CGAGATGAACATCGTGAAGCTGATCTACAAGCTGTACCTG |
| | | GAGAAGAGAGGCTTTAGCTCTGTCGCCACCTACCTGTGCA |
| | | AGAACAAGTACAAAGGCAAGAACGGCGGCGAGTTCTCCA |
| | | GAGAGACAGCTAGGCAAATCGTGATCAATCCTGTGTACTG |
| | | TATCTCCGACAAGACAATCTTCAAGTGGTTCAAATCCAAG |
| | | GGCGCTACCACCTACGGCACACCTGACGGAATTCACGGCC |
| | | TGATGGTGTACAACAAGCGGGAAGGCGGAAAGAAGGACA |
| | | AGCCTATCAACGAGTGGATCATCGCCGTGGGCAAGCATAG |
| | | AGGAGTCATCTCCTCTGATATCTGGCTGAAGTGCCAAAAT |
| | | CTGATCCAGCAGAACAACGCTAAGTCCTCCCCTAGATCCG |
| | | GTACTGGAGAGAAGTTTCTGCTGTCCGGCATGGTGGTGTG |
| | | TAAGGAGTGCGGCTCCGGCATGAGCTCCTGGAGCCACTTC |
| | | AACAAAAAAACCAACTTCATGGAAAGATACTACAGATGC |
| | | AACCTGCGGAATAGAGCCTCCAACCGGTGTTCCACCAAGA |
| | | TGCTGAATGCCTACAAGGCCGAGGAATACGTGGCCAACTA |
| | | CCTCAAGGAACTAGATATCAACGCCATTAAAAAGATGTAC |
| | | CACTCTAACAAGAAGAACATCATCGACTATGACGCCAAGT |
| | | ATGAGGTGAACAAGCTGAACAAGAGCATCGAGGAGAACA |
| | | AGAAGATCATCCAGGGCATCATCAAGAAGATCGCTCTGTT |
| | | CGACGACCTGGATATCCTGGGCATGCTGAAGAACGAACTG |
| | | GAGAGACTGAAAAAAGAAAACGACGAGATGAAGATCAA |
| | | ACTGAAAGAACTGAAGTCCATCCTGGAATTGGAGGATGA |
| | | AGAGGAGATCTTCCTGTCTACCATGGAGGAGAACATCTCT |
| | | AACTTCAAAAAGTTCTACGACTTCGTGAACATCACCCAGA |
| | | AGCGGATTCTGATCAAGGGCCTGGTGGAAAGTATCGTGTG |
| | | GGACACAGGCGGTGAGGAAAAGATCCTGGAGATCAACCT |
| | | GATCGGCTCTAACACCAAGCTGCCTTCCGGCAAGGTGAAG |
| | | CGAAGAGAG |
| 216 | Int27 | ATGTCCAAAAAGGTGGCCATCTATACAAGAGTGTCCACCA |
| | | CCAACCAGGCCGAGGAAGGCTACTCCATCGACGAGCAGA |
| | | TCGACAAGCTGAAAATGTACTGCGAGGCCATGGACTGGA |
| | | AGGTGTCTGAGATCTACACCGACGCCGGCTTCACTGGCTC |
| | | CAAGCTGACCAGACCTGCCATGGAAAAGATGATCACCGA |
| | | CATCGGCCTGAAGAAGTTCGATACCGTGATCGTGTACAAG |
| | | CTGGACAGACTGTCCAGGTCCGTGCGGGATACCCTGTACC |
| | | TGGTCAAGGATGTGTTCACCAAGAATGAGATCGACTTTAT |
| | | CAGCCTGTCTGAGTCTATTGACACCTCCTCCGCTATGGGTT |
| | | CTCTGTTCCTGACAATCCTGAGCGCTATCAACGAGTTCGA |
| | | GAGGGAGAACATAAAAGAACGGATGACCATGGGCAAGAT |
| | | CGGCAGAGCCAAGTCTGGAAAGTCCATGATGTGGGCTAA |
| | | GACCGCCTTCGGCTACTCTCACAACCAAGAGACAGGCATC |
| | | CTGGAAATCAACCCTCTGGAAGCTTCCATCGTGGAACAGA |
| | | TCTTCAACGAGTACCTGAAGGGCACCTCTATCACAAAGCT |
| | | GCGGGACAAGCTGAACGAGGATGGCCACATCGCCAAGGA |
| | | GCTGCCTTGGTCCTACAGAACCATCAGACAGACCCTGGAC |
| | | AACCCCGTGTACTGTGGATACATCAAGTACAAAAACAACA |
| | | CCTTTGAGGGCCTGCACAAGCCCATCATCTCCCACGAAAC |
| | | CTACCTCAGCGTGCAGAAAGAACTGGAAGCCAGACAACA |
| | | GCAGACCTATGAGAAGAACAATAATCCTAGACCATTTCAA |
| | | GCCAAGTATCTGCTGTCTGGCATCGCTAGATGCGGATACT |
| | | GTGGCGCTCCTCTCCGGATCGTGCTGGGCCATCGCCGGAA |
| | | GGACGGCAGTAGAACCATGAAGTACCAGTGCGTGAACAG |
| | | ATTCCCTCGCAAAACCAAGGGCGTGACCACATACAACGAT |
| | | AACAAGAAGTGCGACTCCGGCGCTTACGACATGCAGTGG |
| | | ATCGAGGACATCGTGCTGAAAACCCTGAACGGCTTCCAGA |
| | | AGTCCGACAAAAAGCTGCGGAAGATCCTGAATATCAAGG |
| | | AAGAGTCCAAGGTGGACACCAGCGGATTTCAGAAGCAGC |
| | | TGAAGTCCATCAACAATAAGATCCAGAAGAACTCCGATCT |
| | | GTACCTCAACGACTTCATCACCATGGACGACCTGAAAAAG |
| | | CGGACCGAGATGCTGCAGGGCGAGAAGAAACTGATCCAG |
| | | GCCAGAATCAACGAAGTGGATAAGCCTTCCACATCTGAGA |
| | | TCTTCGACCTGGTCAAGTCTGAGCTGGGCGAAACCACCAT |
| | | CTCTAAGATCTCCTACGAAGATAAGAAGAAGATCGTCAAC |
| | | AACCTGATCTCTAAAGTTGACGTGACCGCCGACAACATCG |
| | | ATATCATCTTCAAGTTCCAGCTGGCT |
| 217 | Int28 | ATGAACGAGCAAAAGGACAAGCTGAAGAAATACTGCGAG |
| | | ATTAAGGACTGGACCATCGTCAAAGAGTACGTCGATCCTG |
| | | GCCGGAGCGGCTCCAACATCAACAGACCATCCATGCAGC |
| | | AGCTCATTAAGGACGCCGATACCGGCCTGTACGACGCTGT |
| | | GCTGGTGTACAAGCTGGACCGGCTGTCTAGATCTCAGAAG |
| | | GACACCCTATATCTGATCGAGGACGTGTTCCAGAAGAACA |
| | | ACATCCACTTCATCTCTCTGTCCGAGAACTTCGACACCTCC |
| | | ACCGCCTTTGGAAAGGCCATGATCGGCATCCTCTCCGTGT |
| | | TCGCCCAGCTGGAAAGAGAGCAGATCAAAGAGCGGATGT |
| | | CTATGGGCAGAGTGGGCAGAGCCAAATCCGGCAAAATCA |
| | | TGGAATTCAACAACCCCGCCTTTGGTTACGAGGTGGATGG |
| | | CGACAACTACAAAGTGGACCCACTGCGGGCCGAGATCGT |
| | | GAAGAGAATCTACAAGATGTACCTGAGCGGCACCTCTATC |
| | | AACAAGATCAAGGAAACCCTGAACCTGGAAGGCCACATC |
| | | GGCAACAAGAAGAACTGGTCCGACACCAGAATCAGATAT |
| | | ATCCTGTCCAATCCCACCTACCTGGGAAAGATCCGGTACG |
| | | ACGGCAAAACCTACGACGGCAAGTTCTCCCCTATCATCGA |
| | | CGAGGAAACCTTCAACAAGACCCAGAACGAACTGAAAGA |
| | | GAGACAGACCGCTACATACAAGAGATTCAACATGAAGCT |
| | | ACGCCCCTTTCAGTCTAAGTACATGCTGTCTGGCCTGCTGA |
| | | GGTGCGGCTACTGCGGCGCTACCCTGTTCGTGAACTCCTA |
| | | TGTGTACAACGGCAAGCGGAAGCTGCGATACAACTGTCCT |
| | | TCTACCTACAAGTCCAAGCAAAAAACACGGACATACAAG |
| | | ATCATGGACCCCAACTGCCCTTTCAAGCTGGTGTACGCCA |
| | | AGGATCTGGAACCTGCTGTGATCAACGAGATCAAGAATCT |
| | | GGCTCTGAACCCTCAGTCCATCCAGAAGCCTGTGAAGAAG |
| | | AAACCTGATATCGATGTGGAAGCCATCCAGAAAGAGCTG |
| | | GCCAAGGTGCGGAAGCAGCAGCAGAGACTGATCGATCTG |
| | | TACGTGATCAGCGACGACGTGAATATCGACAATATCAGCA |
| | | AGAAGTCTGCCGACCTGAAGCTGCAAGAGGAGACACTGA |
| | | AGAAGCAGCTGGCTCCTCTGGAGGAGCCTAACGACGACG |
| | | ATAAGATCGTGGCCTTCAATGAGATTCTGGCTCAGATCAA |
| | | GGATATCGACTCCCTGGACTACGATAAGCAGAAGTTCATC |
| | | GTCAAGAAGCTGATCAAGAAAATCGACGTGTGGAACGAC |
| | | AACAAGATCAAGATCCATTGGAACATC |
| 218 | Int29 | ATGAAAACCGCCATCTACCTGAGAAAGTCTAGAGCCGATC |
| | | TGGAGGCCGAGGCTAGAGGCGAGGGCGAGACACTGGCCA |
| | | AGCACCGGTCGACACTCCTGAAGATCGCCAAGGAGATGA |
| | | ACCTGAACGTGCTGTCTGTGAGAGAAGAAATCGTGTCCGG |
| | | CGAGTCTCTGGTCAAGCGGCCCGAGATGCTGGCTCTGCTG |
| | | GAAGAGATCGAGGACAACAAGTACGACGCCGTGCTGTGC |
| | | ATGGATATGGACAGACTGGGAAGGGGCGGCATGAAGGAA |
| | | CAGGGAATCATCCTGGAAACCTTCAAGCGGTCCAACACCA |
| | | AGATCATGACCCCTAGAAAGACCTACGACCTGAACGACG |
| | | AGTGGGACGAGGAGTACTCTGAGTTTGAGGCCTTCATGGC |
| | | CAGAAAAGAACTTAAGATCATCACCAGAAGAATGCAGAG |
| | | AGGCCGGATCGCCAGCGTGGAAGCTGGCAACTATCTCGGC |
| | | ACCCACGCTCCATTCGGCTATGATATCCACCGGCTGAACA |
| | | AAAGAGAGAGAACCCTGACAATCAACTCCGAGGAGGCCT |
| | | CCGTGGTGCGGATGATCTTCGACTGGTACGCCAACGAGGA |
| | | CATGGGCGCCAGTGCTATCCGGAACAAGCTGAACGACTTG |
| | | GGCTACAAGTCCAAGCTGGGCAATGACTGGAACCCCTACT |
| | | CCATCCTGGATATCCTGAAGAACAACATCTACATCGGCAA |
| | | AGTCACCTGGCAGAAACGTAAGGAAGTGAAGCGGCCTGA |
| | | TGCTGTCAAGAGATCCTGTGCCAGACAGGACAAGTCCGAT |
| | | TGGATCATCGCTGACGGCAAGCACGAGCCTATCATCCCTG |
| | | AGTCCCTGTTCGAGCAGGCCCAAGAGAAGCTGAATTCTCG |
| | | GTACCACGTGCCATACAATACCAACGGCATTAAGAACCCT |
| | | CTGGCTGGAATCATTAAGTGTAGCAAGTGCGGCTACTCCA |
| | | TGGTGCAGAGATACCCTAAGAATCGGAAGGAAACCATGG |
| | | ACTGCAAGCATAGAGGCTGCGAGAACAAGTCTAGCTACA |
| | | CCGAGCTGATCGAGAAGCGCCTCCTGGAAGCTCTGAAGG |
| | | AATGGTACATCAACTACAAGGCTGACTTTGAAGCTCACAA |
| | | GCAGGGCGACAAGCTGAAGGAGACACAAGTGATCCAGAT |
| | | GAACGAGGCTGCCCTGCGGAAGCTGGAAAAAGAACTGGT |
| | | GGACGTGCAGAAGCAGAAGAACAACCTGCACGACCTGCT |
| | | GGAGCGGGGCGTGTACACCGTGGACATGTTCCTGGAAAG |
| | | ATCTCAGGTGATCTCCGACCGGATCAACGAGATCACCTCT |
| | | ACCATGGAAAACCTGAAAAAGGAGATCAAGACCGAAATC |
| | | AAGAAGGAGAAAGTGAAGAAGGACACCATCCCCCAGGTG |
| | | GAGCATGTGCTGGACCTGTACTTCAAGACTGACGATCCTA |
| | | AGAAAAAGAATTCTCTGCTGAAGTCCGTGCTGGAAAAGG |
| | | CCGTGTACAAGAAAGAAAAATGGCAGAGACTGGACGACT |
| | | TCGAGCTGGTTCTGTACCCTAAGCTGCCTCAGGATGGAGA |
| | | CATC |
| 219 | Int30 | ATGTACCGGCCAGAGAGCCTGGACGTGTGCATCTATCTGC |
| | | GCAAGTCTCGGAAAGATGTGGAAGAAGAACGGCGGGCTA |
| | | TTGAAGAGGGCTCCTCCTACAACGCCCTGGAAAGACACAG |
| | | AAAGAGACTGTTCGCCATCGCTAAGGCCGAGAACCACAA |
| | | CATCATCGACATCTTCGAGGAAGTGGCCTCTGGGGAGTCT |
| | | ATCCAAGAGCGGCCTCAGATGCAGCAGCTGCTGCGGAAG |
| | | TTGGAAGGCAACGAGATTGACGGAGTGCTGGTCATCGATC |
| | | TGGATAGGCTGGGCAGAGGCGATATGCTGGACGCTGGCA |
| | | TGATTGACAGAGCCTTCAGATACTCCTCTACCAAGATCAT |
| | | CACCCCTACCGACGTGTACGACCCCGACGACGAGTCCTGG |
| | | GAGCTCGTGTTCGGCATCAAGAGCCTGATCTCCAGACAAG |
| | | AACTGAAGTCCATCACCAAGAGGCTGCAGAACGGCCGGA |
| | | TCGATTCCGTGAAAGAAGGCAAGCACATCGGTAAGAAAC |
| | | CACCTTACGGCTACCTGAAGGATGAGAACCTGAGACTGTA |
| | | CCCTGATCCTGAGAAAGCTTGGATCGTGAAGAAGATCTTC |
| | | GAGCTGATGTGCGACGGCAAAGGCAGACAGATGATCGCC |
| | | GCTGAGCTGGACAGACTGGGCATCGACCCTCCTGTGACCA |
| | | AGCGGGGCGCCTGGGACTCTTCTACAATTACCTCTATCAT |
| | | CAAGAACGAGGTGTACACCGGCGTGATCGTGTGGGGAAA |
| | | GTTCAAGCACAAGAAGCGGAACGGCAAGTACACCAGACA |
| | | TAAGAATCCTCAAGAGAAGTGGATCATGTACGAGAACGC |
| | | TCACGAGCCTATCATCTCTAAGGAACTGTTCGACGCCGCC |
| | | AACGAGGCCCATTCTTCGAGACACAAGCCCGCCGTGATCA |
| | | CTTCCAAGAAACTGACCAACCCCCTGGCCGGCATCCTGAA |
| | | GTGCAAGCTGTGTGGCTACACCATGCTGATCCAGACCCGG |
| | | AAGGACCGGCCTCACAACTACCTGAGATGCAACAACCCC |
| | | GCCTGTAAAGGCAAGCAGAAGCAGTCTGTGTTCAACCTGG |
| | | TTGAGGAAAAGCTCCTGTATAGCCTGCAGCAGATCGTGGA |
| | | CGAGTACCAGGCTCAGAAGGTGGAAGAAGTGGAGATCGA |
| | | CGATTCCAAGCTGATCTCCTTCAAGGAGAAGGCTATCATC |
| | | TCCAAGGAGAAGGAACTCAAAGAACTGCAGGCCCAGAAG |
| | | GGCAACCTGCACGACCTGCTGGAACAGGGCATCTACACA |
| | | GTCGAGATCTTTCTGGAAAGACAGAAGAATCTGGTCGAAA |
| | | GAATCACCTCCATCGAGAACGACATCGAGGTGCTGCAGA |
| | | AGGAGATCGAGACAGAGCAGATCAAGGAGCACAACAAGA |
| | | CCGAGTTTATCCCTGCTCTGAAAACAGTGATCGAGAGCTA |
| | | CCATAAGACCACCAATATCGAGCTGAAGAATCAGCTGCTG |
| | | AAAACCATCCTGTCCACCGTGACCTACTACAGACACCCTG |
| | | ACTGGAAAACCAACGAGTTCGAAATCCAGGTGTACTTTAA |
| | | AATC |
| 220 | Int31 | ATGAAGTACCTGGCTCTGCATGAGAACTCCCGGATCGCCG |
| | | TGTACAGCCGGAAGTCCAGAGAGGACAGAGACTCCGAGG |
| | | ATACCCTGGCCAAGCACCGGAACGAGCTGGAATACCTGAT |
| | | CAAGAGAGAAAACTTCAAAAACGTGCAGTGGTTCGAGAA |
| | | GGTGGTGTCCGGCGAAACCATCGACGAGCGGCCTATGTTC |
| | | TCCCTGCTGCTGCCTAGAATTGAAAACGGCGAGTTCGACG |
| | | CTGTGTGTGCCGTGGCCATGGACCGGCTGTCTAGAGGCTC |
| | | CCAGATCGATTCTGGAAGAATCCTGGAGGCCTTTAAGCAG |
| | | TCCGGCACCCTGTTCATCACCCCTAAGAAAACCTACGACC |
| | | TGTCCATCGAGGGCGACGAGATGCTGTCCGAGTTCGAATC |
| | | CATCATCGCCAGATCTGAGTACAGAGCTATCAAGCGGAGA |
| | | ACCATCAACGGCAAGAAGAATGCCACCCGCGAAGGCCGG |
| | | CTGCACAGCGGATCCGTGCCTTATGGTTACAAGTGGGACA |
| | | AGAACCTGAAAGCTGCTGTCGTGGTGGAAGAGAAGAAGA |
| | | AGATCTATCGGATGATGATTAAGTGGTTTCTGGAAGAAGA |
| | | GTACTCCTGCACCGTGATCGCTGAGATGCTGAATGAACTC |
| | | AAGGTGCCCTCCCCCTCAGGCAGATCTATCTGGTACGGCG |
| | | AGGTGGTGTCTGAGATCCTGTCCAACGACTTCCACAGAGG |
| | | ATACGTGTGGTTCGGCAAGTATAAGAAGTCCAAGAGCAA |
| | | CAACAGCATCGTGCAGAACAAGAACCTGGATGAGGTTCTC |
| | | ATCGCCAAGGGCCACCATGAAACCATGAAAACCGATGAG |
| | | GAGCACGCCCTGATCCTGAACCGGATCGAGAAGCTGCGG |
| | | ACCTACAAGGTGGCTGGCAGACGGCTGAACATGAACACC |
| | | CATAGACTGTCCGGCATCGTGCGGTGTCCTTACTGCCACA |
| | | AGGCTCAGGCCATCGAGCAACCAAAGGGCAGACGGAAGC |
| | | ACGTGAGAAAGTGCCTGAGAAAGTCCGCTGAGAGGACCA |
| | | AAGAGTGCGAGGAAACAAAAGGCATCCACGAGGAAGTGC |
| | | TGTTTCAGTCTATCATGAAAGAGATCAAGAAATACAATGA |
| | | GTCTCTGTTCTCTCCTACCGAGCAGGACGTGAACGACGAC |
| | | TCCTACACTGCCCAGCTGATCGGCCTGAGGGAGAAGGCCG |
| | | TGAAGAAGGCTAAGGGCCGCATCGAGCGGATCAAAGAGA |
| | | TGTACCTGGACGGAGACATCTCCAAAACCGAGTACAAGG |
| | | AAAAGCTGAAGATCAGCCAAGAGACACTGCAGAAGGCTG |
| | | AGAACGAACTTGCCGAACTGATAGCCTCTACAGAGTTCCA |
| | | GAACGCCCTGTCTGCCGAGACAAAGAAAGAGAAGTGGTC |
| | | CCACCACAAGGTGCAGGAAATGATCGAGAGCACCGACGG |
| | | CATGTCCAACTCTGAAATCAACTTGATCCTGAAGATGCTG |
| | | ATCTCTCACGTGACCTACACCGTCGAAGATCTGGGCGATG |
| | | GCACCAAGAATCTGAACATCAAGGTGTACTACAAC |
| 221 | Int32 | ATGGACCCTCAGCACAAGCCTACCCGGGCTCTGATCGTGA |
| | | TCCGGCTGTCCCGGCTGACAGACGAAACCACCTCTCCTGA |
| | | GCGGCAGCTGGAGGCCTGCGAGAGATTCTGCGCCGCAAG |
| | | AGGCTGGGAGGTCGTGGGCGTGGCTGAAGATCTGGACGT |
| | | GTCTGCTGGAACCACCAGCCCCTTCGAGCGGCCTTCTCTG |
| | | AGCCAGTGGATCGGCGATGGTAAGGACAACCCAGGAAGA |
| | | ATCGGCGAGTTCGACACCGTGGTTTTCTACAGAGTGGATC |
| | | GGCTCGTGCGGAGAGTGCGGCACCTGCACGACGTGATCGC |
| | | CTGGAGCGAGCGCTTCGATGTGAACATGGTGTCCGCCACC |
| | | GAGTCTCACTTCGACCTGTCCACAACCATTGGCGCTCTGA |
| | | TCGCTCAGCTGGTGGCCTCCTTCGCCGAGATGGAACTGGA |
| | | GGGCATCTCTCAGAGAGCTACCTCTGCTCACAGACACAAC |
| | | GTGCAGCTGGGCAAGTTCGTGGGCGGCTCCCCTCCTTTCG |
| | | GCTACATGCCTGAAGAAACCCCTGATGGCTGGCGGCTGGT |
| | | GCACGATCCCGACGTCGTGCCCATCATCCTCGAGGTGGTG |
| | | GACAGAGTCCTGGAAGGCGAACCCCTGAGAAGAATCACC |
| | | GACGATCTGAACGCCCGGGGCGCTACAACCGCCCGGGAC |
| | | CTGGTGAAGCAGAGAAAGGGCAAAGAAACCGAGGGCCAC |
| | | AAGTGGCACTCCAACGTGCTGAAGCGGCGGCTGATGTCCC |
| | | CTGCCATGCTGGGCTACGCCCTGAGAAGAGAACCTCTGAC |
| | | CGACTCCAAGGGCAAGCCCAAACTGTCTGCCAAGGGCGC |
| | | CAAGCTGTACGGCCCTGAGGAAATCGTGAGAGGACCTGA |
| | | CGGCCTGCCTGTGCAACGCGCTGAGCCTATCCTGCCTAAG |
| | | CCTCTGTTCGACCGGGTGGTGGCTGAGCTAGAAGCTAGAG |
| | | AGCTACAGAAAGAGCCTACCAAGCGGATCAACTCCATGCT |
| | | GTTGAGAGTGCTGTACTGCGGCGTGTGTGGCCAGCCTGTC |
| | | TACCGGGCAAAAGGACAGGGCGGTAGATCCGACAGATAC |
| | | CGGTGCAGATCCATCCAGGATGGCGCCAACTGTGGCAACC |
| | | CCTCCGTGCTGACCTATGAGCTGGACGACCTGGTGGAAGA |
| | | GTCTATCCTGGTGCTGATGGGCGACTCTGAGAGACTGGCC |
| | | CATGTGTGGAACCCTGGCGAGGACAATGCTAGCGAGCTG |
| | | GCTGAAGTGGAAGCCCGGCTGGCCGACAGAACCGGCCTG |
| | | ATCGGAGTGGGAGCCTACAAGGCTGGCACCCCCCAGAGA |
| | | GCCACCCTGGATACCCTGATCGAGGCTGATGCCAAGCTGT |
| | | ACGAGAGGCTGAAGGCCGCCACCCCTAGACCTGCTGGCTG |
| | | GACCTGGGAACCAACAGGCGAAACCTTCGCCGAGTGGTG |
| | | GGCTGCTCTGGACACCGGCGCCAGAAATGTGTACCTGCGG |
| | | AACATGGGGGTCAGAGTCACCTACGACAAGCGGCCTGTG |
| | | CCAGAGCAGGTGTCCGCCGGCGAGAAGCCTAGAGTGCAT |
| | | CTGGAACTGGGCGAAGTGCGGAAGATGGCCGAACAAGTG |
| | | GCTGTGACCGGCACCATCGGAACACTGACCAGAAACTAC |
| | | ACAAGACTGGGAGAGATCGGCATCACCCACGTGGACATC |
| | | GACGCCGGATCTGGCAAGGCCGTGTTTGTGACAAAGTCCG |
| | | GCGAGCGGTTCGAGCTCCCTCTGAACATCCCTGAGGAA |
| 222 | Int33 | ATGAAGGCTATCGCCATCTACGCCAGAAAGTCTCTGTTCA |
| | | CCGGCAAGGGCGACTCCATTGGCGCCCAGGTGGACACCTG |
| | | CAAGCGGTTCATCGACTACAAGTTCGCCAATGAGGACTAT |
| | | GAGATCCGGACATTTAAGGACGAAGGCTGGTCCGGCAAG |
| | | ACCACTGACAGACCAGATTTTACCAACATGGTGAACCTGA |
| | | TCAAGTCTAAGAAGATCGACTATGTCATCACCTACAAGCT |
| | | GGACCGGATCGGCCGGACAGCTCGGGACCTGCACAACTTC |
| | | CTGTACGAGCTGGATAATCTGGGAATCGTGTACCTGAGCG |
| | | CCACCGAGCCTTACGACACAACCACATCTGCCGGAAGATT |
| | | CATGATCAGCATTCTGGCTGCTATGGCTCAGATGGAACGC |
| | | GAAAGACTGGCCGAGAGAGTGAAGTCCGGCATGATCCAG |
| | | ATCGCCAAGAAGGGAAGATGGCTGGGCGGCCAGTGTCCT |
| | | CTGGGCTTCGACTCTAAGAGAGAGATCTACATCGATGACA |
| | | TGGGGAAAGAGCGGCAGATGATGCGGCTGACCCCTAACA |
| | | AGGAGGAAATCAAGATCGTGAAGCTGATCTACGACAAGT |
| | | ACCTGGAGATGGGATCCATGTCCCAAGTGCGGAAGTACTG |
| | | CCTGGAAAACTCCATCAGAGGCAAGAACGGCGGCGACTT |
| | | CTCCACAAACACCCTGAAGCAGCTGCTGACCTCTCCTATC |
| | | TACGTCAAGTCCTCCGACAACATCTTCAAGTACCTGGAGT |
| | | CTCAGAATATCAATGTGTTCGGCACCCCCAACGGCAACGG |
| | | CATGCTGACCTTCAACAAGACCAAAGAGATCAGGATCGA |
| | | GCGGGACAAGTCCGAGTGGATTGCTGCTGTGGGCAAGCA |
| | | CAAGGGCATCATCGACGATAACAAGTGGCTGCAGATCCA |
| | | GCAGCAGCTGCAGCAGCAGTCTGAAAAGCAGATCAAGAG |
| | | CTCTGGCAGACAGGGCACGACCTCCACCGGCCTGCTGTCC |
| | | GGCATCATCAAGTGCTCCAAGTGCGGCAACAACCTGCTGA |
| | | TCAAGACCGGACACAAGTCCAAGAAAAACCCTGGCACCA |
| | | CCTACTCCTACTACGTGTGTGGCAAGAAGGATAACTCTTA |
| | | CGGCCATAAGTGCGACAACAAAAACGTGAGAACCGACGA |
| | | GGCCGACTCCGCCGTGATCACCCAGCTGAAACTGTACAAC |
| | | AAAGAACTGCTCATCAAAAATCTCAAGGAAGCCCTGATCC |
| | | AAAACGAAAAGACCGATACCGACAACATCGAGATCCTGG |
| | | AGTCCAAATTAAAAGAAAAAGAGAAGGCCGTGTCCAACC |
| | | TGGTGAAAAAGCTGTCTCTGATCGACGACGAGTCCATCAG |
| | | CAATATCATCCTGAACGAGGTTACCAATATCAACAAGGAA |
| | | ATCAACGACATCAAGCTGCAATTGTCTAACGAGACACTGA |
| | | AGATCAACGAAGTGACCAAGGCCACACTGGATACCGAGA |
| | | TCTACATCAAGATCCTGGAGAACTTTAACAAGAAGATCGA |
| | | CGATATCACCGACCCCATCGAAAAGATGAACTTGCTGAAG |
| | | TCTGCTCTGGAATCCGTGGAATGGAACGGCGATTCTGGCG |
| | | AGTTCAAGATCAACCTGATCGGCAGCAAAAAGAAA |
| 223 | Int34 | ATGAAGGTTGCTATCTACACCAGAGTGTCCACCCTGGAGC |
| | | AGCGGGAAAAGGGACACTCTATCGACGAGCAAGAGCGGA |
| | | AACTGAGATCTTTCTGCGACATTAACGACTGGACCGTGAA |
| | | AGATGTGTACGTGGATGCTGGCTTCTCCGGAGCCAAGCGG |
| | | GACAGACCTGAGCTGACCAGACTCCTGGACGACATCTCCG |
| | | AGTTCGACCTGGTGCTGGTCTACAAGCTGGACCGGCTGAC |
| | | AAGAAGCGTCAGAGATCTGCTGGACCTGCTGGAAGTGTTC |
| | | GAGAACAATAACGTGGCCTTCAGATCTGCTACCGAGGTGT |
| | | ACGACACCACCACCGCCATCGGCAGACTGTTCGTGACACT |
| | | CGTGGGCGCCATGGCCGAGTGGGAGAGAGAGACAATCCG |
| | | GGAAAGAAGCCTGATGGGCAAGAGAGCCGCTATTAAGAA |
| | | GGGCATGATCCTGACCGCTCCACCCTTCTACTACGACAGG |
| | | GTGAACAACACCTACATCCCTAACCAGTATAAGGATGTGG |
| | | TCCTCGATGTGTACAACAAGGTCAAGAAAGGCTACTCCAT |
| | | CGCTCATATCGCCAGACTGTACAACAACTCCGACGTGAAG |
| | | CCTCCTAACGGCAACGAGGAATGGACCACCCGGATGCTG |
| | | ATGCACGCCCTGAGAAACCCTGTGACCCGGGGCCACTACC |
| | | AGTGGGGCGAGATCTACATCGAGGACTCTCATGAGCCTAT |
| | | CATCACAGACGAGATGTACAATACAATCATCGACCGCCTG |
| | | GACAAGCACACCAACACCAAGGTGGTGGCCCACACCTCC |
| | | GTGTTTCGGGGCAAGCTGATCTGCCCCAACTGTGGCTACG |
| | | CTCTGACCCTGAACAGCCAGAAGAGAAAGCGGAAGAACG |
| | | ACACCATCGTGTACAAGACCTATTACTGCAATAACTGCAA |
| | | GATCACCAAGGGCATGAAGCCTCACCACATCACCGAGAC |
| | | AGAAACCCTGCGGGTGTTCAAGGACCACCTGTCCAAGATC |
| | | GACCTGAAACAGTACGAAACCCAAGAGAAAGAGAAGCAG |
| | | TCTCACGTGACCATCGATCTGTCTAAAGTGATGGAACAGA |
| | | GAAAGAGATACCACAAGCTGTACGCCTCTGGCATGATGCA |
| | | GGAAAACGAGCTGTTTGAACTGATCAAGGAAACCGACGA |
| | | AATGATCGAAGAGTACGAGAAGCAGCGCAAGCAGGTGGA |
| | | CGTGAAAGAGTTCGACATCTGTAAGATCAAAGAAATCAA |
| | | GGATGTGCTGCTGAAGTCCTGGGACATCTTCACCCTGGAA |
| | | GATAAGGCCGACTTCATCCAGATGTCCATCAAGGCTATCA |
| | | ACATCGAGTATACCAAGCTGAAGCGGGGAAAGAGCTCTA |
| | | ATTCCATGAAGATCAAGGATATCGAGTTTTAC |

TABLE 10
Exemplary Transcriptional Activator Amino
Acid Sequences

| SEQ ID | | |
|---|---|---|
| NO: | Description. | Sequence |
| 224 | TetOff- | MSRLDKSKVINSALELLNEVGIEGLTTRKL |
| | Advanced | AQKLGVEQPTLYWHVKNKRALLDALAIEML |
| | | DRHHTHFCPLEGESWQDFLRNNAKSFRCAL |
| | | LSHRDGAKVHLGTRPTEKQYETLENQLAFL |
| | | CQQGFSLENALYALSAVGHFTLGCVLEDQE |
| | | HQVAKEERETPTTDSMPPLLRQAIELFDHQ |
| | | GAEPAFLFGLELIICGLEKQLKCESGGPAD |
| | | ALDDFDLDMLPADALDDFDLDMLPADALDD |
| | | FDLDMLPG |
| 225 | VanR-VP16 | MDMPRIKPGQRVMMALRKMIASGEIKSGER |
| | | IAEIPTAAALGVSRMPVRIALRSLEQEGLV |
| | | VRLGARGYAARGVSSDQIRDAIEVRGVLEG |
| | | FAARRLAERGMTAETHARFVVLIAEGEALF |
| | | AAGRLNGEDLDRYAAYNQAFHDTLVSAAGN |
| | | GAVESALARNGFEPFAAAGALALDLMDLSA |
| | | EYEHLLAAHRQHQAVLDAVSCGDAEGAERI |
| | | MRDHALAAIRNAKVFEAAASAGAPLGAAWS |
| | | IRADSGGGGPTDALDDFDLDMLPADALDDF |
| | | DLDMLPADALDDFDLDMLPGPPKKKRKV |
| 226 | TtgR-VP16 | MVRRTKEEAQETRAQIIEAAERAFYKRGVA |
| | | RTTLADIAELAGVTRGAIYWHENNKAELVQ |
| | | ALLDSLHETHDHLARASESEDEVDPLGCMR |
| | | KLLLQVFNELVLDARTRRINEILHHKCEFT |
| | | DDMCEIRQQHQSAVLDCHKGITLTLANVVR |
| | | RGQLPGELDAERAAVAMFAYVDGLIRRWLL |
| | | LPDSVDLLGDVEKWVDTGLDMLRLSPALRK |
| | | SGGGGPTDALDDFDLDMLPADALDDFDLDM |
| | | LPADALDDFDLDMLPGPPKKKRKV |
| 227 | Ph1F-VP16 | MARTPSRSSIGSLRSPHTHKAILTSTIEIL |
| | | KECGYSGLSIESVARRAGAGKPTIYRWWTN |
| | | KAALIAEVYENEIEQVRKFPDLGSFKADLD |
| | | FLLHNLWKVWRETICGEAFRCVIAEAQLDP |
| | | VTLTQLKDQFMERRREIPKKLVEDAISNGE |
| | | LPKDINRELLLDMIFGFCWYRLLTEQLTVE |
| | | QDIEEFTFLLINGVCPGTQCSGGGGPTDAL |
| | | DDFDLDMLPADALDDFDLDMLPADALDDFD |
| | | LDMLPGPPKKKRKV |
| 228 | cTA | MSPKRRTQAERAMETQGKLIAAALGVLREK |
| | | GYAGFRIADVPGAAGVSRGAQSHHFPTKLE |
| | | LLLATFEWLYEQITERSRARLAKLKPEDDV |
| | | IQQMLDDAAEFFLDDDFSIGLDLIVAADRD |
| | | PALREGIQRTVERNRFVVEDMWLGVLVSRG |
| | | LSRDDAEDILWLIFNSVRGLVVRSLWQKDK |
| | | ERFERVRNSTLEIARERYAKFKRSGGGGPT |
| | | DALDDFDLDMLPADALDDFDLDMLPADALD |
| | | DFDLDMLPGPPKKKRKV |

What is claimed is:

1. An Adeno-Associated Virus (AAV) production system comprising an expression control component and an AAV production component, wherein:
(a) the expression control component comprises a polynucleic acid molecule encoding for a recombinase; and
(b) the AAV production component comprises one or more polynucleic acid molecules collectively encoding for: Rep52 or Rep40; Rep78 or Rep68; E2A; E4Orf6; VARNA; VP1; VP2; VP3; and AAP; at least one of which comprises a nucleic acid sequence of an excisable element, wherein the excisable element comprises, from 5′ to 3′: (i) a first intron splice donor; (ii) a first recombinase site; (iii) a first intron splice acceptor; (iv) an exon comprising a stop codon; (v) a second intron splice donor; (vi) a second recombinase site; (vii) a second intron splice acceptor; wherein the first recombinase site and the second recombinase site correspond to the recombinase of (a).

2. The AAV production system of claim 1, wherein the first recombinase attachment site comprises a nucleic acid sequence having at least 80% identity with the nucleic acid sequence of any one of SEQ ID NOs: 5-96.

3. The AAV production system of claim 1 or claim 2, wherein the second recombinase attachment site comprises a nucleic acid sequence having at least 80% identity with the nucleic acid sequence of any one of SEQ ID NOs: 5-96.

4. The AAV production system of any one of claims 1-3, wherein the first intron splice donor comprises a nucleic acid sequence having at least 80% identity with the nucleic acid sequence of any one of SEQ ID NOs: 1-2.

5. The AAV production system of any one of claims 1-4, wherein the second intron splice donor comprises a nucleic acid sequence having at least 80% identity with the nucleic acid sequence of any one of SEQ ID NOs: 1-2.

6. The AAV production system of any one of claims 1-5, wherein the first intron splice acceptor comprises a nucleic acid sequence having at least 80% identity with the nucleic acid sequence of any one of SEQ ID NOs: 3-4.

7. The AAV production system of any one of claims 1-6, wherein the second intron splice acceptor comprises a nucleic acid sequence having at least 80% identity with the nucleic acid sequence of any one of SEQ ID NOs: 3-4.

8. The AAV production system of any one of claims 1-7, wherein the first intron splice acceptor of the excisable element is stronger than the second intron splice acceptor of the excisable element.

9. The AAV production system of any one of claims 1-8, wherein the exon of the excisable element comprises a stop codon in all coding frames.

10. The AAV production system of claim 9, wherein the exon of the excisable element comprises a nucleic acid sequence having at least 80% identity with the nucleic acid sequence of any one of SEQ ID NOs: 130-131.

11. The AAV production system of any one of claims 1-10, wherein the nucleic acid sequence encoding for Rep52 or Rep40 and/or the nucleic acid sequence encoding for Rep78 or Rep68 comprises the nucleic acid sequence of the excisable element.

12. The AAV production system of claim 11, wherein the AAV production component comprises the nucleic acid sequence having at least 80% identity with the nucleic acid sequence of any one of SEQ ID NOs: 137-139.

13. The AAV production system of any one of claims 1-12, wherein the nucleic acid sequence encoding for VP1, VP2, and/or VP3 comprises the nucleic acid sequence of the excisable element.

14. The AAV production system of any one of claims 1-13, wherein the nucleic acid sequence encoding for E2A and/or the nucleic acid sequence encoding for E4Orf6 comprises the nucleic acid sequence of the excisable element.

15. The AAV production system of claim 14, wherein the AAV production component comprises the nucleic acid sequence having at least 80% identity with the nucleic acid sequence of any one of SEQ ID NOs: 134-136.

16. The AAV production system of any one of claims 1-15, further comprising a helper component, wherein the helper component comprises one or more polynucleic acid molecules collectively encoding for: UL5, UL8, UL29, UL30, UL42, UL52, UL12, ICP10, ICP4, and ICP22.

17. The AAV production system of any one of claims 1-16, wherein the recombinase is a PhiC31 recombinase, a Cre recombinase, a VCre recombinase, a Flp recombinase, a Bxb1 recombinase, or a TP901 recombinase.

18. The AAV production system of any one of claims 1-17, wherein the recombinase comprises an amino acid sequence having at least 80% identity with the amino acid sequence of any one of SEQ ID NOs: 140-181.

19. An engineered cell comprising an AAV production system according to any one of claims 1-18.

20. The engineered cell of claim 19, wherein one or more polynucleic acid molecules of the AAV production component are stably integrated into the genome of the engineered cell.

21. An engineered cell comprising an Adeno-Associated Virus (AAV) production system having an AAV production component comprising one or more polynucleic acid molecules collectively encoding for: Rep52 or Rep40; Rep78 or Rep68; E2A; E4Orf6; VARNA; VP1; VP2; VP3; and AAP; at least one of which comprises a nucleic acid sequence of an excisable element, wherein the excisable element comprises, from 5′ to 3′: (i) a first intron splice donor; (ii) a first recombinase site; (iii) a first intron splice acceptor; (iv) an exon comprising a stop codon; (v) a second intron splice donor; (vi) a second recombinase site; (vii) a second intron splice acceptor.

22. The engineered cell of claim 21, wherein one or more of the polynucleic acid molecules of the AAV production component are stably integrated into the genome of the engineered cell.

23. The engineered cell of claim 21 or claim 22, wherein the first recombinase attachment site comprises a nucleic acid sequence having at least 80% identity with the nucleic acid sequence of any one of SEQ ID NOs: 5-96.

24. The engineered cell of any one of claims 21-23, wherein the second recombinase attachment site comprises a nucleic acid sequence having at least 80% identity with the nucleic acid sequence of any one of SEQ ID NOs: 5-96.

25. The engineered cell of any one of claims 21-24, wherein the first intron splice donor comprises a nucleic acid sequence having at least 80% identity with the nucleic acid sequence of any one of SEQ ID NOs: 1-2.

26. The engineered cell of any one of claims 21-25, wherein the second intron splice donor comprises a nucleic acid sequence having at least 80% identity with the nucleic acid sequence of any one of SEQ ID NOs: 1-2.

27. The engineered cell of any one of claims 21-26, wherein the first intron splice acceptor comprises a nucleic acid sequence having at least 80% identity with the nucleic acid sequence of any one of SEQ ID NOs: 3-4.

28. The engineered cell of any one of claims 21-27, wherein the second intron splice acceptor comprises a nucleic acid sequence having at least 80% identity with the nucleic acid sequence of any one of SEQ ID NOs: 3-4.

29. The engineered cell of any one of claims 21-28, wherein the first intron splice acceptor of the excisable element is stronger than the second intron splice acceptor of the excisable element.

30. The engineered cell of any one of claims 21-29, wherein the exon of the excisable element comprises a stop codon in all coding frames.

31. The engineered cell of claim 30, wherein the exon of the excisable element comprises a nucleic acid sequence of any one of SEQ ID NOs: 130-131.

32. The engineered cell of any one of claims 21-31, wherein the nucleic acid sequence encoding for Rep52 or Rep40 and/or the nucleic acid sequence encoding for Rep78 or Rep68 comprises the nucleic acid sequence of the excisable element.

33. The engineered cell system of claim 32, wherein the AAV production component comprises a nucleic acid sequence having at least 80% identity with the nucleic acid sequence of any one of SEQ ID NOs: 137-139.

34. The engineered cell of any one of claims 21-33, wherein the nucleic acid sequence encoding for VP1, VP2, and/or VP3 comprises the nucleic acid sequence of the excisable element.

35. The engineered cell of any one of claims 21-34, wherein the nucleic acid sequence encoding for E2A and/or the nucleic acid sequence encoding for E4Orf6 comprises the nucleic acid sequence of the excisable element.

36. The engineered cell of claim 35, wherein the AAV production component comprises the nucleic acid sequence having at least 80% identity with the nucleic acid sequence of any one of SEQ ID NOs: 134-136.

37. The engineered cell of any one of claims 21-36, wherein the AAV production system further comprises a helper component further comprises a helper component, wherein the helper component comprises one or more polynucleic acid molecules collectively encoding for: UL5, UL8, UL29, UL30, UL42, UL52, UL12, ICP10, ICP4, and ICP22.

38. The engineered cell of any one of claims 21-37, wherein the AAV production system further comprises an expression control component, wherein the expression control component comprises a polynucleic acid molecule encoding for a recombinase, wherein the first recombinase site of the excisable element and the second recombinase site of the excisable element correspond to the recombinase.

39. The engineered cell of claim 38, wherein the polynucleic acid molecule of the expression control component is stably integrated into the genome of the engineered cell.

40. The engineered cell of claim 38 or claim 39, wherein the recombinase is a PhiC31 recombinase, a Cre recombinase, a VCre recombinase, a Flp recombinase, a Bxb1 recombinase, or a TP901 recombinase.

41. The engineered cell of any one of claims 38-40, wherein the recombinase comprises an amino acid sequence having at least 80% identity with the amino acid sequence of any one of SEQ ID NOs: 140-181.

42. The engineered cell of any one of claims 19-41, further comprising a transfer polynucleic acid molecule having a sequence encoding, from 5′ to 3′: (i) a nucleic acid sequence of a 5′ inverted tandem repeat; (ii) a multiple cloning site; and (iii) a nucleic acid sequence of a 3′ inverted tandem repeat.

43. The engineered cell of claim 42, wherein the transfer polynucleic acid molecule is stably integrated into the genome of the engineered cell.

44. A kit comprising an Adeno-Associated Virus production system of any one of claims 1-18.

45. A kit comprising an engineered cell of any one of claims 19-41.

46. The kit of claim 44 or claim 45 further comprising a transfer polynucleic acid molecule encoding, from 5′ to 3′: (i) a nucleic acid sequence of a first inverted tandem repeat; (ii) a multiple cloning site; and (iii) a nucleic acid sequence of a second inverted tandem repeat.

47. The kit of claim 46, wherein the transfer polynucleic acid molecule is a plasmid or a vector.

48. A kit comprising an engineered cell of claim 42 or claim 43.

49. A method of producing an AAV vector, comprising expressing, in an engineered cell comprising an AAV production system according to any one of claims 1-18, the recombinase of the expression control component and Rep52 or Rep40, Rep78 or Rep68, E2A, E4Orf6, VARNA, VP1, VP2, VP3, and AAP of the AAV production component.

50. The method of claim 49, further comprising introducing a transfer polynucleic acid molecule into the engineered cell.

51. The method of claim 50, wherein the transfer polynucleic acid molecule comprises a sequence encoding, from 5′ to 3′: (i) a nucleic acid sequence of a first inverted tandem repeat; (ii) a protein and/or an RNA of interest; and (iii) a nucleic acid sequence of a second inverted tandem repeat.

52. The method of claim 51, wherein the transfer polynucleic acid molecule is a plasmid or a vector.

53. A polynucleic acid molecule comprising a nucleic acid sequence having at least 80% identity with the nucleic acid sequence of any one of SEQ ID NOs: 134-139.

54. An engineered cell comprising the polynucleic acid of claim 53.


SEQUENCE LISTING (228 sequences)

SEQ ID NO: 1 - DNA, 20 bp, synthetic construct
  source 1..20: mol_type = other DNA
gtaagttaaattaacatcgc

SEQ ID NO: 2 - DNA, 20 bp, synthetic construct
  source 1..20: mol_type = other DNA
gtgagttgatataactcacc

SEQ ID NO: 3 - DNA, 53 bp, synthetic construct
  source 1..53: mol_type = other DNA
ggctgacatgatctcacatatgaatacctcctccttctccttcttccttgcag

SEQ ID NO: 4 - DNA, 56 bp, synthetic construct
  source 1..56: mol_type = other DNA
atggctgactatacctcacgcatcattggcatcccctctctccctctctctcacag

SEQ ID NO: 5 - DNA, 46 bp, synthetic construct
  source 1..46: mol_type = other DNA
gtgcgggtgccagggcgtgcccaagggctccccgggcgcgtactcc

SEQ ID NO: 6 - DNA, 46 bp, synthetic construct
  source 1..46: mol_type = other DNA
gtgcgggtgccagggcgtgcccacgggctccccgggcgcgtactcc

SEQ ID NO: 7 - DNA, 46 bp, synthetic construct
  source 1..46: mol_type = other DNA
gtgcgggtgccagggcgtgcccaggggctccccgggcgcgtactcc

SEQ ID NO: 8 - DNA, 46 bp, synthetic construct
  source 1..46: mol_type = other DNA
gtgcgggtgccagggcgtgcccatgggctccccgggcgcgtactcc

SEQ ID NO: 9 - DNA, 46 bp, synthetic construct
  source 1..46: mol_type = other DNA
gtgcgggtgccagggcgtgccccagggctccccgggcgcgtactcc

SEQ ID NO: 10 - DNA, 46 bp, synthetic construct
  source 1..46: mol_type = other DNA
gtgcgggtgccagggcgtgcccccgggctccccgggcgcgtactcc

SEQ ID NO: 11 - DNA, 46 bp, synthetic construct
  source 1..46: mol_type = other DNA
gtgcgggtgccagggcgtgccccggggctccccgggcgcgtactcc

SEQ ID NO: 12 - DNA, 46 bp, synthetic construct
  source 1..46: mol_type = other DNA
gtgcgggtgccagggcgtgcccctgggctccccgggcgcgtactcc

SEQ ID NO: 13 - DNA, 46 bp, synthetic construct
  source 1..46: mol_type = other DNA
gtgcgggtgccagggcgtgcccgagggctccccgggcgcgtactcc

SEQ ID NO: 14 - DNA, 46 bp, synthetic construct
  source 1..46: mol_type = other DNA
gtgcgggtgccagggcgtgcccgcgggctccccgggcgcgtactcc

SEQ ID NO: 15 - DNA, 46 bp, synthetic construct
  source 1..46: mol_type = other DNA
gtgcgggtgccagggcgtgcccgggggctccccgggcgcgtactcc

SEQ ID NO: 16 - DNA, 46 bp, synthetic construct
  source 1..46: mol_type = other DNA
gtgcgggtgccagggcgtgcccgtgggctccccgggcgcgtactcc

SEQ ID NO: 17 - DNA, 46 bp, synthetic construct
  source 1..46: mol_type = other DNA
gtgcgggtgccagggcgtgccctagggctccccgggcgcgtactcc

SEQ ID NO: 18 - DNA, 46 bp, synthetic construct
  source 1..46: mol_type = other DNA
gtgcgggtgccagggcgtgccctcgggctccccgggcgcgtactcc

SEQ ID NO: 19 - DNA, 46 bp, synthetic construct
  source 1..46: mol_type = other DNA
gtgcgggtgccagggcgtgccctggggctccccgggcgcgtactcc

SEQ ID NO: 20 - DNA, 46 bp, synthetic construct
  source 1..46: mol_type = other DNA
gtgcgggtgccagggcgtgcccttgggctccccgggcgcgtactcc

SEQ ID NO: 21 - DNA, 46 bp, synthetic construct
  source 1..46: mol_type = other DNA
agtgccccaactggggtaacctaagagttctctcagttgggggcgt

SEQ ID NO: 22 - DNA, 46 bp, synthetic construct
  source 1..46: mol_type = other DNA
agtgccccaactggggtaacctacgagttctctcagttgggggcgt

SEQ ID NO: 23 - DNA, 46 bp, synthetic construct
  source 1..46: mol_type = other DNA
agtgccccaactggggtaacctaggagttctctcagttgggggcgt

SEQ ID NO: 24 - DNA, 46 bp, synthetic construct
  source 1..46: mol_type = other DNA
agtgccccaactggggtaacctatgagttctctcagttgggggcgt

SEQ ID NO: 25 - DNA, 46 bp, synthetic construct
  source 1..46: mol_type = other DNA
agtgccccaactggggtaacctcagagttctctcagttgggggcgt

SEQ ID NO: 26 - DNA, 46 bp, synthetic construct
  source 1..46: mol_type = other DNA
agtgccccaactggggtaacctccgagttctctcagttgggggcgt

SEQ ID NO: 27 - DNA, 46 bp, synthetic construct
  source 1..46: mol_type = other DNA
agtgccccaactggggtaacctcggagttctctcagttgggggcgt

SEQ ID NO: 28 - DNA, 46 bp, synthetic construct
  source 1..46: mol_type = other DNA
agtgccccaactggggtaacctctgagttctctcagttgggggcgt

SEQ ID NO: 29 - DNA, 46 bp, synthetic construct
  source 1..46: mol_type = other DNA
agtgccccaactggggtaacctgagagttctctcagttgggggcgt

SEQ ID NO: 30 - DNA, 46 bp, synthetic construct
  source 1..46: mol_type = other DNA
agtgccccaactggggtaacctgcgagttctctcagttgggggcgt

SEQ ID NO: 31 - DNA, 46 bp, synthetic construct
  source 1..46: mol_type = other DNA
agtgccccaactggggtaacctgggagttctctcagttgggggcgt

SEQ ID NO: 32 - DNA, 46 bp, synthetic construct
  source 1..46: mol_type = other DNA
agtgccccaactggggtaacctgtgagttctctcagttgggggcgt

SEQ ID NO: 33 - DNA, 46 bp, synthetic construct
  source 1..46: mol_type = other DNA
agtgccccaactggggtaaccttagagttctctcagttgggggcgt

SEQ ID NO: 34 - DNA, 46 bp, synthetic construct
  source 1..46: mol_type = other DNA
agtgccccaactggggtaaccttcgagttctctcagttgggggcgt

SEQ ID NO: 35 - DNA, 46 bp, synthetic construct
  source 1..46: mol_type = other DNA
agtgccccaactggggtaaccttggagttctctcagttgggggcgt

SEQ ID NO: 36 - DNA, 46 bp, synthetic construct
  source 1..46: mol_type = other DNA
agtgccccaactggggtaacctttgagttctctcagttgggggcgt

SEQ ID NO: 37 - DNA, 38 bp, synthetic construct
  source 1..38: mol_type = other DNA
ggcttgtcgacgacggcgaactccgtcgtcaggatcat

SEQ ID NO: 38 - DNA, 38 bp, synthetic construct
  source 1..38: mol_type = other DNA
ggcttgtcgacgacggcgacctccgtcgtcaggatcat

SEQ ID NO: 39 - DNA, 38 bp, synthetic construct
  source 1..38: mol_type = other DNA
ggcttgtcgacgacggcgagctccgtcgtcaggatcat

SEQ ID NO: 40 - DNA, 38 bp, synthetic construct
  source 1..38: mol_type = other DNA
ggcttgtcgacgacggcgatctccgtcgtcaggatcat

SEQ ID NO: 41 - DNA, 38 bp, synthetic construct
  source 1..38: mol_type = other DNA
ggcttgtcgacgacggcgcactccgtcgtcaggatcat

SEQ ID NO: 42 - DNA, 38 bp, synthetic construct
  source 1..38: mol_type = other DNA
ggcttgtcgacgacggcgccctccgtcgtcaggatcat

SEQ ID NO: 43 - DNA, 38 bp, synthetic construct
  source 1..38: mol_type = other DNA
ggcttgtcgacgacggcgcgctccgtcgtcaggatcat

SEQ ID NO: 44 - DNA, 38 bp, synthetic construct
  source 1..38: mol_type = other DNA
ggcttgtcgacgacggcgctctccgtcgtcaggatcat

SEQ ID NO: 45 - DNA, 38 bp, synthetic construct
  source 1..38: mol_type = other DNA
ggcttgtcgacgacggcggactccgtcgtcaggatcat

SEQ ID NO: 46 - DNA, 38 bp, synthetic construct
  source 1..38: mol_type = other DNA
ggcttgtcgacgacggcggcctccgtcgtcaggatcat

SEQ ID NO: 47 - DNA, 38 bp, synthetic construct
  source 1..38: mol_type = other DNA
ggcttgtcgacgacggcgggctccgtcgtcaggatcat

SEQ ID NO: 48 - DNA, 38 bp, synthetic construct
  source 1..38: mol_type = other DNA
ggcttgtcgacgacggcggtctccgtcgtcaggatcat

SEQ ID NO: 49 - DNA, 38 bp, synthetic construct
  source 1..38: mol_type = other DNA
ggcttgtcgacgacggcgtactccgtcgtcaggatcat

SEQ ID NO: 50 - DNA, 38 bp, synthetic construct
  source 1..38: mol_type = other DNA
ggcttgtcgacgacggcgtcctccgtcgtcaggatcat

SEQ ID NO: 51 - DNA, 38 bp, synthetic construct
  source 1..38: mol_type = other DNA
ggcttgtcgacgacggcgtgctccgtcgtcaggatcat

SEQ ID NO: 52 - DNA, 38 bp, synthetic construct
  source 1..38: mol_type = other DNA
ggcttgtcgacgacggcgttctccgtcgtcaggatcat

SEQ ID NO: 53 - DNA, 48 bp, synthetic construct
  source 1..48: mol_type = other DNA
ggtttgtctggtcaaccaccgcgaactcagtggtgtacggtacaaacc

SEQ ID NO: 54 - DNA, 48 bp, synthetic construct
  source 1..48: mol_type = other DNA
ggtttgtctggtcaaccaccgcgacctcagtggtgtacggtacaaacc

SEQ ID NO: 55 - DNA, 48 bp, synthetic construct
  source 1..48: mol_type = other DNA
ggtttgtctggtcaaccaccgcgagctcagtggtgtacggtacaaacc

SEQ ID NO: 56 - DNA, 48 bp, synthetic construct
  source 1..48: mol_type = other DNA
ggtttgtctggtcaaccaccgcgatctcagtggtgtacggtacaaacc

SEQ ID NO: 57 - DNA, 48 bp, synthetic construct
  source 1..48: mol_type = other DNA
ggtttgtctggtcaaccaccgcgcactcagtggtgtacggtacaaacc

SEQ ID NO: 58 - DNA, 48 bp, synthetic construct
  source 1..48: mol_type = other DNA
ggtttgtctggtcaaccaccgcgccctcagtggtgtacggtacaaacc

SEQ ID NO: 59 - DNA, 48 bp, synthetic construct
  source 1..48: mol_type = other DNA
ggtttgtctggtcaaccaccgcgcgctcagtggtgtacggtacaaacc

SEQ ID NO: 60 - DNA, 48 bp, synthetic construct
  source 1..48: mol_type = other DNA
ggtttgtctggtcaaccaccgcgctctcagtggtgtacggtacaaacc

SEQ ID NO: 61 - DNA, 48 bp, synthetic construct
  source 1..48: mol_type = other DNA
ggtttgtctggtcaaccaccgcggactcagtggtgtacggtacaaacc

SEQ ID NO: 62 - DNA, 48 bp, synthetic construct
  source 1..48: mol_type = other DNA
ggtttgtctggtcaaccaccgcggcctcagtggtgtacggtacaaacc

SEQ ID NO: 63 - DNA, 48 bp, synthetic construct
  source 1..48: mol_type = other DNA
ggtttgtctggtcaaccaccgcgggctcagtggtgtacggtacaaacc

SEQ ID NO: 64 - DNA, 48 bp, synthetic construct
  source 1..48: mol_type = other DNA
ggtttgtctggtcaaccaccgcggtctcagtggtgtacggtacaaacc

SEQ ID NO: 65 - DNA, 48 bp, synthetic construct
  source 1..48: mol_type = other DNA
ggtttgtctggtcaaccaccgcgtactcagtggtgtacggtacaaacc

SEQ ID NO: 66 - DNA, 48 bp, synthetic construct
  source 1..48: mol_type = other DNA
ggtttgtctggtcaaccaccgcgtcctcagtggtgtacggtacaaacc

SEQ ID NO: 67 - DNA, 48 bp, synthetic construct
  source 1..48: mol_type = other DNA
ggtttgtctggtcaaccaccgcgtgctcagtggtgtacggtacaaacc

SEQ ID NO: 68 - DNA, 48 bp, synthetic construct
  source 1..48: mol_type = other DNA
ggtttgtctggtcaaccaccgcgttctcagtggtgtacggtacaaacc

SEQ ID NO: 69 - DNA, 34 bp, synthetic construct
  source 1..34: mol_type = other DNA
ataacttcgtatagcatacattatacgaacggta

SEQ ID NO: 70 - DNA, 34 bp, synthetic construct
  source 1..34: mol_type = other DNA
taccgttcgtatagcatacattatacgaagttat

SEQ ID NO: 71 - DNA, 34 bp, synthetic construct
  source 1..34: mol_type = other DNA
ataacttcgtataatgtatactatacgaagttat

SEQ ID NO: 72 - DNA, 34 bp, synthetic construct
  source 1..34: mol_type = other DNA
ataacttcgtataggatactttatacgaagttat

SEQ ID NO: 73 - DNA, 34 bp, synthetic construct
  source 1..34: mol_type = other DNA
ataacttcgtataatgtgtactatacgaagttat

SEQ ID NO: 74 - DNA, 34 bp, synthetic construct
  source 1..34: mol_type = other DNA
ataacttcgtatagcatacattataccttgttat

SEQ ID NO: 75 - DNA, 34 bp, synthetic construct
  source 1..34: mol_type = other DNA
taccgttcgtatatggtttcttatacgaagttat

SEQ ID NO: 76 - DNA, 34 bp, synthetic construct
  source 1..34: mol_type = other DNA
ataacttcgtatagtataccttatacgaagttat

SEQ ID NO: 77 - DNA, 34 bp, synthetic construct
  source 1..34: mol_type = other DNA
ataacttcgtatagcatacattatacgaagttat

SEQ ID NO: 78 - DNA, 34 bp, synthetic construct
  source 1..34: mol_type = other DNA
tcaatttctgagaactgtcattctcggaaattga

SEQ ID NO: 79 - DNA, 34 bp, synthetic construct
  source 1..34: mol_type = other DNA
tcaatttctgagaagtgtctttctcggaaattga

SEQ ID NO: 80 - DNA, 34 bp, synthetic construct
  source 1..34: mol_type = other DNA
tcaatttccgagaactgtcattctcggaaattga

SEQ ID NO: 81 - DNA, 34 bp, synthetic construct
  source 1..34: mol_type = other DNA
tcaatttctgagaactgtcattctcagaaattga

SEQ ID NO: 82 - DNA, 34 bp, synthetic construct
  source 1..34: mol_type = other DNA
tcaatttctgagaactgtcattctcggaatacct

SEQ ID NO: 83 - DNA, 34 bp, synthetic construct
  source 1..34: mol_type = other DNA
cgtgattctgagaactgtcattctcggaaattga

SEQ ID NO: 84 - DNA, 34 bp, synthetic construct
  source 1..34: mol_type = other DNA
gaagttcctattctctagaaagtataggaacttc

SEQ ID NO: 85 - DNA, 34 bp, synthetic construct
  source 1..34: mol_type = other DNA
gaagttcctattctctagatagtataggaacttc

SEQ ID NO: 86 - DNA, 34 bp, synthetic construct
  source 1..34: mol_type = other DNA
gaagttcctattctctacttagtataggaacttc

SEQ ID NO: 87 - DNA, 34 bp, synthetic construct
  source 1..34: mol_type = other DNA
gaagttcctattcttcaaatagtataggaacttc

SEQ ID NO: 88 - DNA, 34 bp, synthetic construct
  source 1..34: mol_type = other DNA
gaagttcctattctctagaaggtataggaacttc

SEQ ID NO: 89 - DNA, 34 bp, synthetic construct
  source 1..34: mol_type = other DNA
gaagttcctattcttcaaaaggtataggaacttc

SEQ ID NO: 90 - DNA, 34 bp, synthetic construct
  source 1..34: mol_type = other DNA
gaagttcctattcactagaatgtataggaacttc

SEQ ID NO: 91 - DNA, 34 bp, synthetic construct
  source 1..34: mol_type = other DNA
gaagttcctattctgaactaagtataggaacttc

SEQ ID NO: 92 - DNA, 34 bp, synthetic construct
  source 1..34: mol_type = other DNA
gaagttcctattctttctgaagtataggaacttc

SEQ ID NO: 93 - DNA, 34 bp, synthetic construct
  source 1..34: mol_type = other DNA
gaagttcctattctcatataagtataggaacttc

SEQ ID NO: 94 - DNA, 34 bp, synthetic construct
  source 1..34: mol_type = other DNA
gaagttcctattctatcagaagtataggaacttc

SEQ ID NO: 95 - DNA, 34 bp, synthetic construct
  source 1..34: mol_type = other DNA
gaagttcctattcttataggagtataggaacttc

SEQ ID NO: 96 - DNA, 34 bp, synthetic construct
  source 1..34: mol_type = other DNA
gaagttcctattctccgggcagtataggaacttc

SEQ ID NO: 97 - protein, 397 aa, unidentified
  source 1..397: note = Adeno-Associated Virus
MELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 98 - protein, 312 aa, unidentified
  source 1..312: note = Adeno-Associated Virus
MELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRLARGHSL

SEQ ID NO: 99 - protein, 621 aa, unidentified
  source 1..621: note = Adeno-Associated Virus
MPGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 100 - protein, 536 aa, unidentified
  source 1..536: note = Adeno-Associated Virus
MPGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRLARGHSL

SEQ ID NO: 101 - protein, 529 aa, unidentified
  source 1..529: note = Adeno-Associated Virus
MASREEEQRETTPERGRGAARRPPTMEDVSSPSPSPPPPRAPPKKRLRRRLESEDEEDSSQDALVPRTPSPRPSTSTADLAIASKKKKKRPSPKPERPPSPEVIVDSEEEREDVALQMVGFSNPPVLIKHGKGGKRTVRRLNEDDPVARGMRTQEEKEESSEAESESTVINPLSLPIVSAWEKGMEAARALMDKYHVDNDLKANFKLLPDQVEALAAVCKTWLNEEHRGLQLTFTSNKTFVTMMGRFLQAYLQSFAEVTYKHHEPTGCALWLHRCAEIEGELKCLHGSIMINKEHVIEMDVTSENGQRALKEQSSKAKIVKNRWGRNVVQISNTDARCCVHDAACPANQFSGKSCGMFFSEGAKAQVAFKQIKAFMQALYPNAQTGHGHLLMPLRCECNSKPGHAPFLGRQLPKLTPFALSNAEDLDADLISDKSVLASVHHPALIVFQCCNPVYRNSRAQGGGPNCDFKISAPDLLNALVMVRSLWSENFTELPRMVVPEFKWSTKHQYRNVSLPVAHSDARQNPFDF

SEQ ID NO: 102 - protein, 294 aa, unidentified
  source 1..294: note = Adeno-Associated Virus
MTTSGVPFGMTLRPTRSRLSRRTPYSRDRLPPFETETRATILEDHPLLPECNTLTMHNVSYVRGLPCSVGFTLIQEWVVPWDMVLTREELVILRKCMHVCLCCANIDIMTSMMIHGYESWALHCHCSSPGSLQCIAGGQVLASWFRMVVDGAMFNQRFIWYREVVNYNMPKEVMFMSSVFMRGRHLIYLRLWYDGHVGSVVPAMSFGYSALHCGILNNIVVLCCSYCADLSEIRVRCCARRTRRLMLRAVRIIAEETTAMLYSCRTERRRQQFIRALLQHHRPILMHDYDSTPM

SEQ ID NO: 103 - DNA, 888 bp, synthetic construct
  source 1..888: mol_type = other DNA
atgactacgtccggcgttccatttggcatgacactacgaccaacacgatctcggttgtctcggcgcactccgtacagtagggatcgcctacctccttttgagacagagacccgcgctaccatactggaggatcatccgctgctgcccgaatgtaacactttgacaatgcacaatgtttcctacgtgcgaggtcttccctgcagtgtgggatttacgctgattcaggaatgggttgttccctgggatatggttctgacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgcatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgtctcatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtagtaa

SEQ ID NO: 104 - DNA, 738 bp, unidentified
  source 1..738: mol_type = other DNA; note = Adeno-Associated Virus
cgacgtaatccgtagatgtacctggacatccaggtgatgccggcggcggtggtggaggcgcgcggaaagtcgcggacgcggttccagatgttgcgcagcggcaaaaagtgctccatggtcgggacgctctggccggtgaggcgtgcgcagtcgttgacgctctagaccgtgcaaaaggagagcctgtaagcgggcactcttccgtggtctggtggataaattcgcaagggtatcatggcggacgaccggggttcgaaccccggatccggccgtccgccgtgatccatgcggttaccgcccgcgtgtcgaacccaggtgtgcgacgtcagacaacgggggagcgctccttttggcttccttccaggcgcggcggctgctgcgctagcttttttggccactggccgcgcgcggcgtaagcggttaggctggaaagcgaaagcattaagtggctcgctccctgtagccggagggttattttccaagggttgagtcgcaggacccccggttcgagtctcgggccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctccccttctcctaccgcgtcaggaggggcaacatcc

SEQ ID NO: 105 - protein, 735 aa, unidentified [sequence omitted]

SEQ ID NO: 106 - protein, 598 aa, unidentified
  source 1..598: note = Adeno-Associated Virus
TAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 107 - protein, 533 aa, unidentified
  source 1..533: note = Adeno-Associated Virus
MATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 108 - protein, 204 aa, unidentified
  source 1..204: note = Adeno-Associated Virus
LETQTQYLTPSLSDSHQQPPLVWELIRWLQAVAHQWQTITRAPTEWVIPREIGIAIPHGWATESSPPAPEPGPCPPTTTTSTNKFPANQEPRTTITTLATAPLGGILTSTDSTATFHHVTGKDSSTTTGDSDPRDSTSSSLTFKSKRSRRMTVRRRLPITLPARFRCLLTRSTSSRTSSARRIKDASRRSQQTSSWCHSMDTSP

SEQ ID NO: 109 - protein, 119 aa, unidentified
  source 1..119: note = Adeno-Associated Virus
LAHHHQSPQSGIRTTAGVLCFLGTSTSDPSTDSTRESRSTRQTPRPSSTTKPTTGSSTAETTRTSSTTTPTRSFRSALKKIRLLGATSDEQSSRRKRGFLNLWAWLRNLLRRLREKRGR

SEQ ID NO: 110 - DNA, 1932 bp, unidentified
  source 1..1932: mol_type = other DNA; note = Adeno-Associated Virus
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctga

SEQ ID NO: 111 - protein, 641 aa, unidentified
  source 1..641: note = Adeno-Associated Virus
MPGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQMIIRYGCRWLSSRLARGHSL

SEQ ID NO: 112 - protein, 244 aa, synthetic construct
MPGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQ

SEQ ID NO: 113 - protein, 397 aa, synthetic construct
EDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQMIIRYGCRWLSSRLARGHSL

SEQ ID NO: 114 - protein, 234 aa, synthetic construct
MPGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDK

SEQ ID NO: 115 - protein, 407 aa, synthetic construct
GITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQMIIRYGCRWLSSRLARGHSL

SEQ ID NO: 116 - protein, 247 aa, synthetic construct
MPGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQ

SEQ ID NO: 117 - protein, 374 aa, synthetic construct
ASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 118 - protein, 47 aa, synthetic construct
MASREEEQRETTPERGRGAARRPPTMEDVSSPSPSPPPPRAPPKKRL

SEQ ID NO: 119 - protein, 482 aa, synthetic construct
RRRLESEDEEDSSQDALVPRTPSPRPSTSTADLAIASKKKKKRPSPKPERPPSPEVIVDSEEEREDVALQMVGFSNPPVLIKHGKGGKRTVRRLNEDDPVARGMRTQEEKEESSEAESESTVINPLSLPIVSAWEKGMEAARALMDKYHVDNDLKANFKLLPDQVEALAAVCKTWLNEEHRGLQLTFTSNKTFVTMMGRFLQAYLQSFAEVTYKHHEPTGCALWLHRCAEIEGELKCLHGSIMINKEHVIEMDVTSENGQRALKEQSSKAKIVKNRWGRNVVQISNTDARCCVHDAACPANQFSGKSCGMFFSEGAKAQVAFKQIKAFMQALYPNAQTGHGHLLMPLRCECNSKPGHAPFLGRQLPKLTPFALSNAEDLDADLISDKSVLASVHHPALIVFQCCNPVYRNSRAQGGGPNCDFKISAPDLLNALVMVRSLWSENFTELPRMVVPEFKWSTKHQYRNVSLPVAHSDARQNPFDF

SEQ ID NO: 120 - protein, 54 aa, synthetic construct
MASREEEQRETTPERGRGAARRPPTMEDVSSPSPSPPPPRAPPKKRLRRRLESE

SEQ ID NO: 121 - protein, 475 aa, synthetic construct
DEEDSSQDALVPRTPSPRPSTSTADLAIASKKKKKRPSPKPERPPSPEVIVDSEEEREDVALQMVGFSNPPVLIKHGKGGKRTVRRLNEDDPVARGMRTQEEKEESSEAESESTVINPLSLPIVSAWEKGMEAARALMDKYHVDNDLKANFKLLPDQVEALAAVCKTWLNEEHRGLQLTFTSNKTFVTMMGRFLQAYLQSFAEVTYKHHEPTGCALWLHRCAEIEGELKCLHGSIMINKEHVIEMDVTSENGQRALKEQSSKAKIVKNRWGRNVVQISNTDARCCVHDAACPANQFSGKSCGMFFSEGAKAQVAFKQIKAFMQALYPNAQTGHGHLLMPLRCECNSKPGHAPFLGRQLPKLTPFALSNAEDLDADLISDKSVLASVHHPALIVFQCCNPVYRNSRAQGGGPNCDFKISAPDLLNALVMVRSLWSENFTELPRMVVPEFKWSTKHQYRNVSLPVAHSDARQNPFDF

SEQ ID NO: 122 - protein, 52 aa, synthetic construct
MASREEEQRETTPERGRGAARRPPTMEDVSSPSPSPPPPRAPPKKRLRRRLE

SEQ ID NO: 123 - protein, 477 aa, synthetic construct
SEDEEDSSQDALVPRTPSPRPSTSTADLAIASKKKKKRPSPKPERPPSPEVIVDSEEEREDVALQMVGFSNPPVLIKHGKGGKRTVRRLNEDDPVARGMRTQEEKEESSEAESESTVINPLSLPIVSAWEKGMEAARALMDKYHVDNDLKANFKLLPDQVEALAAVCKTWLNEEHRGLQLTFTSNKTFVTMMGRFLQAYLQSFAEVTYKHHEPTGCALWLHRCAEIEGELKCLHGSIMINKEHVIEMDVTSENGQRALKEQSSKAKIVKNRWGRNVVQISNTDARCCVHDAACPANQFSGKSCGMFFSEGAKAQVAFKQIKAFMQALYPNAQTGHGHLLMPLRCECNSKPGHAPFLGRQLPKLTPFALSNAEDLDADLISDKSVLASVHHPALIVFQCCNPVYRNSRAQGGGPNCDFKISAPDLLNALVMVRSLWSENFTELPRMVVPEFKWSTKHQYRNVSLPVAHSDARQNPFDF

SEQ ID NO: 124 - protein, 26 aa, synthetic construct
MTTSGVPFGMTLRPTRSRLSRRTPYS

SEQ ID NO: 125 - protein, 268 aa, synthetic construct
RDRLPPFETETRATILEDHPLLPECNTLTMHNVSYVRGLPCSVGFTLIQEWVVPWDMVLTREELVILRKCMHVCLCCANIDIMTSMMIHGYESWALHCHCSSPGSLQCIAGGQVLASWFRMVVDGAMFNQRFIWYREVVNYNMPKEVMFMSSVFMRGRHLIYLRLWYDGHVGSVVPAMSFGYSALHCGILNNIVVLCCSYCADLSEIRVRCCARRTRRLMLRAVRIIAEETTAMLYSCRTERRRQQFIRALLQHHRPILMHDYDSTPM

SEQ ID NO: 126 - protein, 43 aa, synthetic construct
MTTSGVPFGMTLRPTRSRLSRRTPYSRDRLPPFETETRATILE

SEQ ID NO: 127 - protein, 251 aa, synthetic construct
DHPLLPECNTLTMHNVSYVRGLPCSVGFTLIQEWVVPWDMVLTREELVILRKCMHVCLCCANIDIMTSMMIHGYESWALHCHCSSPGSLQCIAGGQVLASWFRMVVDGAMFNQRFIWYREVVNYNMPKEVMFMSSVFMRGRHLIYLRLWYDGHVGSVVPAMSFGYSALHCGILNNIVVLCCSYCADLSEIRVRCCARRTRRLMLRAVRIIAEETTAMLYSCRTERRRQQFIRALLQHHRPILMHDYDSTPM

SEQ ID NO: 128 - protein, 25 aa, synthetic construct
MTTSGVPFGMTLRPTRSRLSRRTPY

SEQ ID NO: 129 - protein, 269 aa, synthetic construct
SRDRLPPFETETRATILEDHPLLPECNTLTMHNVSYVRGLPCSVGFTLIQEWVVPWDMVLTREELVILRKCMHVCLCCANIDIMTSMMIHGYESWALHCHCSSPGSLQCIAGGQVLASWFRMVVDGAMFNQRFIWYREVVNYNMPKEVMFMSSVFMRGRHLIYLRLWYDGHVGSVVPAMSFGYSALHCGILNNIVVLCCSYCADLSEIRVRCCARRTRRLMLRAVRIIAEETTAMLYSCRTERRRQQFIRALLQHHRPILMHDYDSTPM

SEQ ID NO: 130 - DNA, 103 bp, synthetic construct
  source 1..103: mol_type = other DNA
ggttctggctaagttaaattaatatatctgggttgatggtactggagaaggactgcgctgcaaaacccgcaccctggactgtgagcccaagtgtgtagaagag

SEQ ID NO: 131 - DNA, 106 bp, synthetic construct
  source 1..106: mol_type = other DNA
ggctcaggttaagttaaattaattacctgagtggaattttgatggctctagtacctttcagtctgagggctccaacagtgacatctatctcagccctgtagaagag

SEQ ID NO: 132 - DNA, 325 bp, synthetic construct
  source 1..325: mol_type = other DNA
gtaagttaacttgaacggcactgataacttcgtatagcatacattatacgaagttatacgaatgacgctgacatggcctaacctatgaaaaccctgtcttttgcctttctctccacagggttctggctaagttaaattaatatatctgggttgatggtactggagaaggactgcgctgcaaaacccgcaccctggactgtgagcccaagtgtgtagaagaggtaagtccatgccataacttcgtatagcatacattatacgaagttattactttggccctcatatgatatatcgatacggtatgcctcttgccctcatccggtag

SEQ ID NO: 133 - DNA, 331 bp, synthetic construct
  source 1..331: mol_type = other DNA
gtaagctaacctgaaccctggtcataacttcgtataggatactttatacgaagttatctgcaacttcctgacatggactaacccacttgaaccttgtcttctgccttttcctctacagggctcaggttaagttaaattaattacctgagtggaattttgatggctctagtacctttcagtctgagggctccaacagtgacatctatctcagccctgtagaagaggtaagtaaaaacaataacttcgtataggatactttatacgaagttattcaaccaaatctgacacgatctatcgaacttgcgctacgcccgacacctcctatctgtag

SEQ ID NO: 134 - DNA, 9987 bp, synthetic construct
  source 1..9987: mol_type = other DNA
atggatccggtacccaactccatgcttaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactaggagacactttcaataaaggcaaatgtttttatttgtacactctcgggtgattatttaccccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggtgcatgcccaggctttgagttgcactcgcaccgtagtggcatcagaaggtgaccgtgcccggtctgggcgttaggatacagcgcctgcatgaaagccttgatctgcttaaaagccacctgagcctttgcgccttcagagaagaacatgccgcaagacttgccggaaaactgattggccggacaggccgcgtcatgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgctcccgtgtagacacttaagctcgccttcgatctcagcgcagcggtgcagccacaacgcgcagcccgtgggctcgtggtgcttgtaggttacctctgcaaacgactgcaggtacgcctgcaggaatcgccccatcatcgtcacaaaggtcttgttgctggtgaaggtcagctgcaacccgcggtgctcctcgtttagccaggtcttgcatacggccgccagagcttccacttggtcaggcagtagcttgaagtttgcctttagatcgttatccacgtggtacttgtccatcaacgcgcgcgcagcctccatgcccttctcccacgcagacacgatcggcaggctcagcgggtttatcaccgtgctttcactttccgcttcactggactcttccttttcctcttgcgtccgcataccccgcgccactgggtcgtcttcattcagccgccgcaccgtgcgcttacctcccttgccgtgcttgattagcaccggtgggttgctgaaacccaccatttgtagcgccacatcttctctttcttcctcgctgtccacgatcacctctggggatggcgggcgctcgggcttgggagaggggcgcttctttttctttttggacgcaatggccaaatccgccgtcgaggtcgatggccgcgggctgggtgtgcgcggcaccagcgcatcttgtgacgagtcttcttcgtcctcggactcgagacgccgcctaccggatgagggcaagaggcataccgtatcgatatatcatatgagggccaaagtaataacttcgtataatgtatgctatacgaagttatggcatggacttacctcttctacacacttgggctcacagtccagggtgcgggttttgcagcgcagtccttctccagtaccatcaacccagatatattaatttaacttagccagaaccctgtggagagaaaggcaaaagacagggttttcataggttaggccatgtcagcgtcattcgtataacttcgtataatgtatgctatacgaagttatcagtgccgttcaagttaacttacctcagccgcttttttgggggcgcgcggggaggcggcggcgacggcgacggggacgacacgtcctccatggttggtggacgtcgcgccgcaccgcgtccgcgctcgggggtggtttcgcgctgctcctcttcccgactggccatttccttctcctataggcagaaaaagatcatggagtcagtcgagaaggaggacagcctaaccgccccctttgagttcgccaccaccgcctccaccgatgccgccaacgcgcctaccaccttccccgtcgaggcacccccgcttgaggaggaggaagtgattatcgagcaggacccaggttttgtaagcgaagacgacgaggatcgctcagtaccaacagaggataaaaagcaagaccaggacgacgcagaggcaaacgaggaacaagtcgggcggggggaccaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctgttctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcgacgaagtgccaaaaatctttgagggtcttggacgcgacgagaaacgcgcggcaaacgctctgcaacaagaaaacagcgaaaatgaaagtcactgtggagtgctggtggaacttgagggtgacaacgcgcgcctagccgtgctgaaacgcagcatcgaggtcacccactttgcctacccggcacttaacctaccccccaaggttatgagcacagtcatgagcgagctgatcgtgcgccgtgcacgacccctggagagggatgcaaacttgcaagaacaaaccgaggagggcctacccgcagttggcgatgagcagctggcgcgctggcttgagacgcgcgagcctgccgacttggaggagcgacgcaagctaatgatggccgcagtgcttgttaccgtggagcttgagtgcatgcagcggttctttgctgacccggagatgcagcgcaagctagaggaaacgttgcactacacctttcgccagggctacgtgcgccaggcctgcaaaatttccaacgtggagctctgcaacctggtctcctaccttggaattttgcacgaaaaccgcctcgggcaaaacgtgcttcattccacgctcaagggcgaggcgcgccgcgactacgtccgcgactgcgtttacttatttctgtgctacacctggcaaacggccatgggcgtgtggcagcaatgcctggaggagcgcaacctaaaggagctgcagaagctgctaaagcaaaacttgaaggacctatggacggccttcaacgagcgctccgtggccgcgcacctggcggacattatcttccccgaacgcctgcttaaaaccctgcaacagggtctgccagacttcaccagtcaaagcatgttgcaaaactttaggaactttatcctagagcgttcaggaattctgcccgccacctgctgtgcgcttcctagcgactttgtgcccattaagtaccgtgaatgccctccgccgctttggggtcactgctaccttctgcagctagccaactaccttgcctaccactccgacatcatggaagacgtgagcggtgacggcctactggagtgtcactgtcgctgcaacctatgcaccccgcaccgctccctggtctgcaattcgcaactgcttagcgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacatccttggccaattgcaagccatcaacaaagcccgccaagagtttctgctacgaaagggacggggggtttacctggacccccagtccggcgaggagctcaacccaatccccccgccgccgcagccctatcagcagccgcgggcccttgcttcccaggatggcacccaaaaagaagctgcagctgccgccgccgccacccacggacgaggaggaatactgggacagtcaggcagaggaggttttggacgaggaggaggagatgatggaagactgggacagcctagacgaagcttccgaggccgaagaggtgtcagacgaaacaccgtcaccctcggtcgcattcccctcgccggcgccccagaaattggcaaccgttcccagcatcgctacaacctccgctcctcaggcgccgccggcactgcctgttcgccgacccaaccgtagatgggacaccactggaaccagggccggtaagtctaagcagccgccgccgttagcccaagagcaacaacagcgccaaggctaccgctcgtggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccctactgcaccggcggcagcggcagcggcagcaacagcagcggtcacacagaagcaaaggcgaccggatagcaagactctgacaaagcccaagaaatccacagcggcggcagcagcaggaggaggagcgctgcgtctggcgcccaacgaacccgtatcgacccgcgagcttagaaataggatttttcccactctgtatgctatatttcaacaaagcaggggccaagaacaagagctgaaaataaaaaacaggtctctgcgctccctcacccgcagctgcctgtatcacaaaagcgaagatcagcttcggcgcacgctggaagacgcggaggctctcttcagcaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcctcgaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcgttttagggcggagtaacttgcatgtattgggaattgtagtttttttaaaatgggaagtgacgtatcgtgggaaaacggaagtgaagatttgaggaagttgtgggttttttggctttcgtttctgggcgtaggttcgcgtgcggttttctgggtgttttttgtggactttaaccgttacgtcattttttagtcctatatatactcgctctgtacttggccctttttacactgtgactgattgagctggtgccgtgtcgagtggtgttttttaataggtttttttactggtaaggctgactgttatggctgccgctgtggaagcgctgtatgttgttctggagcgggagggtgctattttgcctaggcaggagggtttttcaggtgtttatgtgtttttctctcctattaattttgttatacctcctatgggggctgtaatgttgtctctacgcctgcgggtatgtattcccccgggctatttcggtcgctttttagcactgaccgatgttaaccaacctgatgtgtttaccgagtcttacattatgactccggacatgaccgaggaactgtcggtggtgctttttaatcacggtgaccagtttttttacggtcacgccggcatggccgtagtccgtcttatgcttataagggttgtttttcctgttgtaagacaggcttctaatgtttaaatgtttttttttttgttattttattttgtgtttaatgcaggaacccgcagacatgtttgagagaaaaatggtgtctttttctgtggtggttccggaacttacctgcctttatctgcatgagcatgactacgatgtgcttgcttttttgcgcgaggctttgcctgattttttgagcagcaccttgcattttatatcgccgcccatgcaacaagcttacataggggctacgctggttagcatagctccgagtatgcgtgtcataatcagtgtgggttcttttgtcatggttcctggcggggaagtggccgcgctggtccgtgcagacctgcacgattatgttcagctggccctgcgaagggacctacgggatcgcggtatttttgttaatgttccgcttttgaatcttatacaggtctgtgaggaacctgaatttttgcaatcatgattcgctgcttgaggctgaaggtggagggcgctctggagcagatttttacaatggccggacttaatattcgggatttgcttagagacatattgataaggtggcgagatgaaaattatttgggcatggttgaaggtgctggaatgtttatagaggagattcaccctgaagggtttagcctttacgtccacttggacgtgagggcagtttgccttttggaagccattgtgcaacatcttacaaatgccattatctgttctttggctgtagagtttgaccacgccaccggaggggagcgcgttcacttaatagatcttcattttgaggttttggataatcttttggaataaaaaaaaaaaaacatggttcttccagctcttcccgctcctcccgtgtgtgactcgcagaacgaatgtgtaggttggctgggtgtggcttattctgcggtggtggatgttatcagggcagcggcgcatgaaggagtttacatagaacccgaagccagggggcgcctggatgctttgagagagtggatatactacaactactacacagagcgagctaagcgacgagaccggagacgcagatctgtttgtcacgcccgcacctggttttgcttcaggaaatatgactacgtccggcgttccatttggcatgacactacgaccaacacgatctcggttgtctcggcgcactccgtacagtaggtaagctaacctgaaccctggtcataacttcgtataggatactttatacgaagttatctgcaacttcctgacatggactaacccacttgaaccttgtcttctgccttttcctctacagggctcaggttaagttaaattaattacctgagtggaattttgatggctctagtacctttcagtctgagggctccaacagtgacatctatctcagccctgtagaagaggtaagtaaaaacaataacttcgtataggatactttatacgaagttattcaaccaaatctgacacgatctatcgaacttgcgctacgcccgacacctcctatctgtagggatcgcctacctccttttgagacagagacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctgacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgcatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgtctcatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtaggcgtggacttccccttcgccgcccgttgagcaaccgcaagttggacagcagcctgtggctcagcagctggacagcgacatgaacttaagcgagctgcccggggagtttattaatatcactgatgagcgtttggctcgacaggaaaccgtgtggaatataacacctaagaatatgtctgttacccatgatatgatgctttttaaggccagccggggagaaaggactgtgtactctgtgtgttgggagggaggtggcaggttgaatactagggttctgtgagtttgattaaggtacggtgatcaatataagctatgtggtggtggggctatactactgaatgaaaaatgacttgaaattttctgcaattgaaaaataaacacgttgaaacataacatgcaacaggttcacgattctttattcctgggcaatgtaggagaaggtgtaagagttggtagcaaaagtttcagtggtgtattttccactttcccaggaccatgtaaaagacatagagtaagtgcttacctcgctagtttctgtggattcactagaatcgatgtaggatgttgcccctcctgacgcggtaggagaaggggagggtgccctgcatgtctgccgctgctcttgctcttgccgctgctgaggaggggggcgcatctgccgcagcaccggatgcatctgggaaaagcaaaaaaggggctcgtccctgtttccggaggaatttgcaagcggggtcttgcatgacggggaggcaaacccccgttcgccgcagtccggccggcccgagactcgaaccgggggtcctgcgactcaacccttggaaaataaccctccggctacagggagcgagccacttaatgctttcgctttccagcctaaccgcttacgccgcgcgcggccagtggccaaaaaagctagcgcagcagccgccgcgcctggaaggaagccaaaaggagcgctcccccgttgtctgacgtcgcacacctgggttcgacacgcgggcggtaaccgcatggatcacggcggacggccggatccggggttcgaaccccggtcgtccgccatgatacccttgcgaatttatccaccagaccacggaagagtgcccgcttacaggctctccttttgcacggtctagagcgtcaacgactgcgcacgcctcaccggccagagcgtcccgaccatggagcactttttgccgctgcgcaacatctggaaccgcgtccgcgactttccgcgcgcctccaccaccgccgccggcatcacctggatgtccaggtacatctacggattacgtcgacgtttaaaccatatgatcagctcactcaaaggcggtaat

SEQ ID NO: 135 - DNA, 9987 bp, synthetic construct
  source 1..9987: mol_type = other DNA
atggatccggtacccaactccatgcttaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactaggagacactttcaataaaggcaaatgtttttatttgtacactctcgggtgattatttaccccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggtgcatgcccaggctttgagttgcactcgcaccgtagtggcatcagaaggtgaccgtgcccggtctgggcgttaggatacagcgcctgcatgaaagccttgatctgcttaaaagccacctgagcctttgcgccttcagagaagaacatgccgcaagacttgccggaaaactgattggccggacaggccgcgtcatgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgctcccgtgtagacacttaagctcgccttcgatctcagcgcagcggtgcagccacaacgcgcagcccgtgggctcgtggtgcttgtaggttacctctgcaaacgactgcaggtacgcctgcaggaatcgccccatcatcgtcacaaaggtcttgttgctggtgaaggtcagctgcaacccgcggtgctcctcgtttagccaggtcttgcatacggccgccagagcttccacttggtcaggcagtagcttgaagtttgcctttagatcgttatccacgtggtacttgtccatcaacgcgcgcgcagcctccatgcccttctcccacgcagacacgatcggcaggctcagcgggtttatcaccgtgctttcactttccgcttcactggactcttccttttcctcttgcgtccgcataccccgcgccactgggtcgtcttcattcagccgccgcaccgtgcgcttacctcccttgccgtgcttgattagcaccggtgggttgctgaaacccaccatttgtagcgccacatcttctctttcttcctcgctgtccacgatcacctctggggatggcgggcgctcgggcttgggagaggggcgcttctttttctttttggacgcaatggccaaatccgccgtcgaggtcgatggccgcgggctgggtgtgcgcggcaccagcgcatcttgtgacgagtcttcttcgtcctaccggatgagggcaagaggcataccgtatcgatatatcatatgagggccaaagtaataacttcgtataatgtatgctatacgaagttatggcatggacttacctcttctacacacttgggctcacagtccagggtgcgggttttgcagcgcagtccttctccagtaccatcaacccagatatattaatttaacttagccagaaccctgtggagagaaaggcaaaagacagggttttcataggttaggccatgtcagcgtcattcgtataacttcgtataatgtatgctatacgaagttatcagtgccgttcaagttaacttacctcggactcgagacgccgcctcagccgcttttttgggggcgcgcggggaggcggcggcgacggcgacggggacgacacgtcctccatggttggtggacgtcgcgccgcaccgcgtccgcgctcgggggtggtttcgcgctgctcctcttcccgactggccatttccttctcctataggcagaaaaagatcatggagtcagtcgagaaggaggacagcctaaccgccccctttgagttcgccaccaccgcctccaccgatgccgccaacgcgcctaccaccttccccgtcgaggcacccccgcttgaggaggaggaagtgattatcgagcaggacccaggttttgtaagcgaagacgacgaggatcgctcagtaccaacagaggataaaaagcaagaccaggacgacgcagaggcaaacgaggaacaagtcgggcggggggaccaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctgttctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcgacgaagtgccaaaaatctttgagggtcttggacgcgacgagaaacgcgcggcaaacgctctgcaacaagaaaacagcgaaaatgaaagtcactgtggagtgctggtggaacttgagggtgacaacgcgcgcctagccgtgctgaaacgcagcatcgaggtcacccactttgcctacccggcacttaacctaccccccaaggttatgagcacagtcatgagcgagctgatcgtgcgccgtgcacgacccctggagagggatgcaaacttgcaagaacaaaccgaggagggcctacccgcagttggcgatgagcagctggcgcgctggcttgagacgcgcgagcctgccgacttggaggagcgacgcaagctaatgatggccgcagtgcttgttaccgtggagcttgagtgcatgcagcggttctttgctgacccggagatgcagcgcaagctagaggaaacgttgcactacacctttcgccagggctacgtgcgccaggcctgcaaaatttccaacgtggagctctgcaacctggtctcctaccttggaattttgcacgaaaaccgcctcgggcaaaacgtgcttcattccacgctcaagggcgaggcgcgccgcgactacgtccgcgactgcgtttacttatttctgtgctacacctggcaaacggccatgggcgtgtggcagcaatgcctggaggagcgcaacctaaaggagctgcagaagctgctaaagcaaaacttgaaggacctatggacggccttcaacgagcgctccgtggccgcgcacctggcggacattatcttccccgaacgcctgcttaaaaccctgcaacagggtctgccagacttcaccagtcaaagcatgttgcaaaactttaggaactttatcctagagcgttcaggaattctgcccgccacctgctgtgcgcttcctagcgactttgtgcccattaagtaccgtgaatgccctccgccgctttggggtcactgctaccttctgcagctagccaactaccttgcctaccactccgacatcatggaagacgtgagcggtgacggcctactggagtgtcactgtcgctgcaacctatgcaccccgcaccgctccctggtctgcaattcgcaactgcttagcgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacatccttggccaattgcaagccatcaacaaagcccgccaagagtttctgctacgaaagggacggggggtttacctggacccccagtccggcgaggagctcaacccaatccccccgccgccgcagccctatcagcagccgcgggcccttgcttcccaggatggcacccaaaaagaagctgcagctgccgccgccgccacccacggacgaggaggaatactgggacagtcaggcagaggaggttttggacgaggaggaggagatgatggaagactgggacagcctagacgaagcttccgaggccgaagaggtgtcagacgaaacaccgtcaccctcggtcgcattcccctcgccggcgccccagaaattggcaaccgttcccagcatcgctacaacctccgctcctcaggcgccgccggcactgcctgttcgccgacccaaccgtagatgggacaccactggaaccagggccggtaagtctaagcagccgccgccgttagcccaagagcaacaacagcgccaaggctaccgctcgtggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccctactgcaccggcggcagcggcagcggcagcaacagcagcggtcacacagaagcaaaggcgaccggatagcaagactctgacaaagcccaagaaatccacagcggcggcagcagcaggaggaggagcgctgcgtctggcgcccaacgaacccgtatcgacccgcgagcttagaaataggatttttcccactctgtatgctatatttcaacaaagcaggggccaagaacaagagctgaaaataaaaaacaggtctctgcgctccctcacccgcagctgcctgtatcacaaaagcgaagatcagcttcggcgcacgctggaagacgcggaggctctcttcagcaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcctcgaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcgttttagggcggagtaacttgcatgtattgggaattgtagtttttttaaaatgggaagtgacgtatcgtgggaaaacggaagtgaagatttgaggaagttgtgggttttttggctttcgtttctgggcgtaggttcgcgtgcggttttctgggtgttttttgtggactttaaccgttacgtcattttttagtcctatatatactcgctctgtacttggccctttttacactgtgactgattgagctggtgccgtgtcgagtggtgttttttaataggtttttttactggtaaggctgactgttatggctgccgctgtggaagcgctgtatgttgttctggagcgggagggtgctattttgcctaggcaggagggtttttcaggtgtttatgtgtttttctctcctattaattttgttatacctcctatgggggctgtaatgttgtctctacgcctgcgggtatgtattcccccgggctatttcggtcgctttttagcactgaccgatgttaaccaacctgatgtgtttaccgagtcttacattatgactccggacatgaccgaggaactgtcggtggtgctttttaatcacggtgaccagtttttttacggtcacgccggcatggccgtagtccgtcttatgcttataagggttgtttttcctgttgtaagacaggcttctaatgtttaaatgtttttttttttgttattttattttgtgtttaatgcaggaacccgcagacatgtttgagagaaaaatggtgtctttttctgtggtggttccggaacttacctgcctttatctgcatgagcatgactacgatgtgcttgcttttttgcgcgaggctttgcctgattttttgagcagcaccttgcattttatatcgccgcccatgcaacaagcttacataggggctacgctggttagcatagctccgagtatgcgtgtcataatcagtgtgggttcttttgtcatggttcctggcggggaagtggccgcgctggtccgtgcagacctgcacgattatgttcagctggccctgcgaagggacctacgggatcgcggtatttttgttaatgttccgcttttgaatcttatacaggtctgtgaggaacctgaatttttgcaatcatgattcgctgcttgaggctgaaggtggagggcgctctggagcagatttttacaatggccggacttaatattcgggatttgcttagagacatattgataaggtggcgagatgaaaattatttgggcatggttgaaggtgctggaatgtttatagaggagattcaccctgaagggtttagcctttacgtccacttggacgtgagggcagtttgccttttggaagccattgtgcaacatcttacaaatgccattatctgttctttggctgtagagtttgaccacgccaccggaggggagcgcgttcacttaatagatcttcattttgaggttttggataatcttttggaataaaaaaaaaaaaacatggttcttccagctcttcccgctcctcccgtgtgtgactcgcagaacgaatgtgtaggttggctgggtgtggcttattctgcggtggtggatgttatcagggcagcggcgcatgaaggagtttacatagaacccgaagccagggggcgcctggatgctttgagagagtggatatactacaactactacacagagcgagctaagcgacgagaccggagacgcagatctgtttgtcacgcccgcacctggttttgcttcaggaaatatgactacgtccggcgttccatttggcatgacactacgaccaacacgatctcggttgtctcggcgcactccgtacagtagggatcgcctacctccttttgagacagagacccgcgctaccatactggaggtaagctaacctgaaccctggtcataacttcgtataggatactttatacgaagttatctgcaacttcctgacatggactaacccacttgaaccttgtcttctgccttttcctctacagggctcaggttaagttaaattaattacctgagtggaattttgatggctctagtacctttcagtctgagggctccaacagtgacatctatctcagccctgtagaagaggtaagtaaaaacaataacttcgtataggatactttatacgaagttattcaaccaaatctgacacgatctatcgaacttgcgctacgcccgacacctcctatctgtaggatcatccgctgctgcccgaatgtaacactttgacaatgcacaacgtgagttacgtgcgaggtcttccctgcagtgtgggatttacgctgattcaggaatgggttgttccctgggatatggttctgacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgcatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgtctcatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtaggcgtggacttccccttcgccgcccgttgagcaaccgcaagttggacagcagcctgtggctcagcagctggacagcgacatgaacttaagcgagctgcccggggagtttattaatatcactgatgagcgtttggctcgacaggaaaccgtgtggaatataacacctaagaatatgtctgttacccatgatatgatgctttttaaggccagccggggagaaaggactgtgtactctgtgtgttgggagggaggtggcaggttgaatactagggttctgtgagtttgattaaggtacggtgatcaatataagctatgtggtggtggggctatactactgaatgaaaaatgacttgaaattttctgcaattgaaaaataaacacgttgaaacataacatgcaacaggttcacgattctttattcctgggcaatgtaggagaaggtgtaagagttggtagcaaaagtttcagtggtgtattttccactttcccaggaccatgtaaaagacatagagtaagtgcttacctcgctagtttctgtggattcactagaatcgatgtaggatgttgcccctcctgacgcggtaggagaaggggagggtgccctgcatgtctgccgctgctcttgctcttgccgctgctgaggaggggggcgcatctgccgcagcaccggatgcatctgggaaaagcaaaaaaggggctcgtccctgtttccggaggaatttgcaagcggggtcttgcatgacggggaggcaaacccccgttcgccgcagtccggccggcccgagactcgaaccgggggtcctgcgactcaacccttggaaaataaccctccggctacagggagcgagccacttaatgctttcgctttccagcctaaccgcttacgccgcgcgcggccagtggccaaaaaagctagcgcagcagccgccgcgcctggaaggaagccaaaaggagcgctcccccgttgtctgacgtcgcacacctgggttcgacacgcgggcggtaaccgcatggatcacggcggacggccggatccggggttcgaaccccggtcgtccgccatgatacccttgcgaatttatccaccagaccacggaagagtgcccgcttacaggctctccttttgcacggtctagagcgtcaacgactgcgcacgcctcaccggccagagcgtcccgaccatggagcactttttgccgctgcgcaacatctggaaccgcgtccgcgactttccgcgcgcctccaccaccgccgccggcatcacctggatgtccaggtacatctacggattacgtcgacgtttaaaccatatgatcagctcactcaaaggcggtaat

SEQ ID NO: 136 - DNA, 9987 bp, synthetic construct
  source 1..9987: mol_type = other DNA
atggatccggtacccaactccatgcttaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactaggagacactttcaataaaggcaaatgtttttatttgtacactctcgggtgattatttaccccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggtgcatgcccaggctttgagttgcactcgcaccgtagtggcatcagaaggtgaccgtgcccggtctgggcgttaggatacagcgcctgcatgaaagccttgatctgcttaaaagccacctgagcctttgcgccttcagagaagaacatgccgcaagacttgccggaaaactgattggccggacaggccgcgtcatgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgctcccgtgtagacacttaagctcgccttcgatctcagcgcagcggtgcagccacaacgcgcagcccgtgggctcgtggtgcttgtaggttacctctgcaaacgactgcaggtacgcctgcaggaatcgccccatcatcgtcacaaaggtcttgttgctggtgaaggtcagctgcaacccgcggtgctcctcgtttagccaggtcttgcatacggccgccagagcttccacttggtcaggcagtagcttgaagtttgcctttagatcgttatccacgtggtacttgtccatcaacgcgcgcgcagcctccatgcccttctcccacgcagacacgatcggcaggctcagcgggtttatcaccgtgctttcactttccgcttcactggactcttccttttcctcttgcgtccgcataccccgcgccactgggtcgtcttcattcagccgccgcaccgtgcgcttacctcccttgccgtgcttgattagcaccggtgggttgctgaaacccaccatttgtagcgccacatcttctctttcttcctcgctgtccacgatcacctctggggatggcgggcgctcgggcttgggagaggggcgcttctttttctttttggacgcaatggccaaatccgccgtcgaggtcgatggccgcgggctgggtgtgcgcggcaccagcgcatcttgtgacgagtcttcttcgtcctcggactaccggatgagggcaagaggcataccgtatcgatatatcatatgagggccaaagtaataacttcgtataatgtatgctatacgaagttatggcatggacttacctcttctacacacttgggctcacagtccagggtgcgggttttgcagcgcagtccttctccagtaccatcaacccagatatattaatttaacttagccagaaccctgtggagagaaaggcaaaagacagggttttcataggttaggccatgtcagcgtcattcgtataacttcgtataatgtatgctatacgaagttatcagtgccgttcaagttaacttacctcgagacgccgcctcagccgcttttttgggggcgcgcggggaggcggcggcgacggcgacggggacgacacgtcctccatggttggtggacgtcgcgccgcaccgcgtccgcgctcgggggtggtttcgcgctgctcctcttcccgactggccatttccttctcctataggcagaaaaagatcatggagtcagtcgagaaggaggacagcctaaccgccccctttgagttcgccaccaccgcctccaccgatgccgccaacgcgcctaccaccttccccgtcgaggcacccccgcttgaggaggaggaagtgattatcgagcaggacccaggttttgtaagcgaagacgacgaggatcgctcagtaccaacagaggataaaaagcaagaccaggacgacgcagaggcaaacgaggaacaagtcgggcggggggaccaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctgttctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcgacgaagtgccaaaaatctttgagggtcttggacgcgacgagaaacgcgcggcaaacgctctgcaacaagaaaacagcgaaaatgaaagtcactgtggagtgctggtggaacttgagggtgacaacgcgcgcctagccgtgctgaaacgcagcatcgaggtcacccactttgcctacccggcacttaacctaccccccaaggttatgagcacagtcatgagcgagctgatcgtgcgccgtgcacgacccctggagagggatgcaaacttgcaagaacaaaccgaggagggcctacccgcagttggcgatgagcagctggcgcgctggcttgagacgcgcgagcctgccgacttggaggagcgacgcaagctaatgatggccgcagtgcttgttaccgtggagcttgagtgcatgcagcggttctttgctgacccggagatgcagcgcaagctagaggaaacgttgcactacacctttcgccagggctacgtgcgccaggcctgcaaaatttccaacgtggagctctgcaacctggtctcctaccttggaattttgcacgaaaaccgcctcgggcaaaacgtgcttcattccacgctcaagggcgaggcgcgccgcgactacgtccgcgactgcgtttacttatttctgtgctacacctggcaaacggccatgggcgtgtggcagcaatgcctggaggagcgcaacctaaaggagctgcagaagctgctaaagcaaaacttgaaggacctatggacggccttcaacgagcgctccgtggccgcgcacctggcggacattatcttccccgaacgcctgcttaaaaccctgcaacagggtctgccagacttcaccagtcaaagcatgttgcaaaactttaggaactttatcctagagcgttcaggaattctgcccgccacctgctgtgcgcttcctagcgactttgtgcccattaagtaccgtgaatgccctccgccgctttggggtcactgctaccttctgcagctagccaactaccttgcctaccactccgacatcatggaagacgtgagcggtgacggcctactggagtgtcactgtcgctgcaacctatgcaccccgcaccgctccctggtctgcaattcgcaactgcttagcgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacatccttggccaattgcaagccatcaacaaagcccgccaagagtttctgctacgaaagggacggggggtttacctggacccccagtccggcgaggagctcaacccaatccccccgccgccgcagccctatcagcagccgcgggcccttgcttcccaggatggcacccaaaaagaagctgcagctgccgccgccgccacccacggacgaggaggaatactgggacagtcaggcagaggaggttttggacgaggaggaggagatgatggaagactgggacagcctagacgaagcttccgaggccgaagaggtgtcagacgaaacaccgtcaccctcggtcgcattcccctcgccggcgccccagaaattggcaaccgttcccagcatcgctacaacctccgctcctcaggcgccgccggcactgcctgttcgccgacccaaccgtagatgggacaccactggaaccagggccggtaagtctaagcagccgccgccgttagcccaagagcaacaacagcgccaaggctaccgctcgtggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccctactgcaccggcggcagcggcagcggcagcaacagcagcggtcacacagaagcaaaggcgaccggatagcaagactctgacaaagcccaagaaatccacagcggcggcagcagcaggaggaggagcgctgcgtctggcgcccaacgaacccgtatcgacccgcgagcttagaaataggatttttcccactctgtatgctatatttcaacaaagcaggggccaagaacaagagctgaaaataaaaaacaggtctctgcgctccctcacccgcagctgcctgtatcacaaaagcgaagatcagcttcggcgcacgctggaagacgcggaggctctcttcagcaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcctcgaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcgttttagggcggagtaacttgcatgtattgggaattgtagtttttttaaaatgggaagtgacgtatcgtgggaaaacggaagtgaagatttgaggaagttgtgggttttttggctttcgtttctgggcgtaggttcgcgtgcggttttctgggtgttttttgtggactttaaccgttacgtcattttttagtcctatatatactcgctctgtacttggccctttttacactgtgactgattgagctggtgccgtgtcgagtggtgttttttaataggtttttttactggtaaggctgactgttatggctgccgctgtggaagcgctgtatgttgttctggagcgggagggtgctattttgcctaggcaggagggtttttcaggtgtttatgtgtttttctctcctattaattttgttatacctcctatgggggctgtaatgttgtctctacgcctgcgggtatgtattcccccgggctatttcggtcgctttttagcactgaccgatgttaaccaacctgatgtgtttaccgagtcttacattatgactccggacatgaccgaggaactgtcggtggtgctttttaatcacggtgaccagtttttttacggtcacgccggcatggccgtagtccgtcttatgcttataagggttgtttttcctgttgtaagacaggcttctaatgtttaaatgtttttttttttgttattttattttgtgtttaatgcaggaacccgcagacatgtttgagagaaaaatggtgtctttttctgtggtggttccggaacttacctgcctttatctgcatgagcatgactacgatgtgcttgcttttttgcgcgaggctttgcctgattttttgagcagcaccttgcattttatatcgccgcccatgcaacaagcttacataggggctacgctggttagcatagctccgagtatgcgtgtcataatcagtgtgggttcttttgtcatggttcctggcggggaagtggccgcgctggtccgtgcagacctgcacgattatgttcagctggccctgcgaagggacctacgggatcgcggtatttttgttaatgttccgcttttgaatcttatacaggtctgtgaggaacctgaatttttgcaatcatgattcgctgcttgaggctgaaggtggagggcgctctggagcagatttttacaatggccggacttaatattcgggatttgcttagagacatattgataaggtggcgagatgaaaattatttgggcatggttgaaggtgctggaatgtttatagaggagattcaccctgaagggtttagcctttacgtccacttggacgtgagggcagtttgccttttggaagccattgtgcaacatcttacaaatgccattatctgttctttggctgtagagtttgaccacgccaccggaggggagcgcgttcacttaatagatcttcattttgaggttttggataatcttttggaataaaaaaaaaaaaacatggttcttccagctcttcccgctcctcccgtgtgtgactcgcagaacgaatgtgtaggttggctgggtgtggcttattctgcggtggtggatgttatcagggcagcggcgcatgaaggagtttacatagaacccgaagccagggggcgcctggatgctttgagagagtggatatactacaactactacacagagcgagctaagcgacgagaccggagacgcagatctgtttgtcacgcccgcacctggttttgcttcaggaaatatgactacgtccggcgttccatttggcatgacactacgaccaacacgatctcggttgtctcggcgcactccgtacaggtaagctaacctgaaccctggtcataacttcgtataggatactttatacgaagttatctgcaacttcctgacatggactaacccacttgaaccttgtcttctgccttttcctctacagggctcaggttaagttaaattaattacctgagtggaattttgatggctctagtacctttcagtctgagggctccaacagtgacatctatctcagccctgtagaagaggtaagtaaaaacaataacttcgtataggatactttatacgaagttattcaaccaaatctgacacgatctatcgaacttgcgctacgcccgacacctcctatctgtagtagggatcgcctacctccttttgagacagagacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctgacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgcatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgtctcatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtaggcgtggacttccccttcgccgcccgttgagcaaccgcaagttggacagcagcctgtggctcagcagctggacagcgacatgaacttaagcgagctgcccggggagtttattaatatcactgatgagcgtttggctcgacaggaaaccgtgtggaatataacacctaagaatatgtctgttacccatgatatgatgctttttaaggccagccggggagaaaggactgtgtactctgtgtgttgggagggaggtggcaggttgaatactagggttctgtgagtttgattaaggtacggtgatcaatataagctatgtggtggtggggctatactactgaatgaaaaatgacttgaaattttctgcaattgaaaaataaacacgttgaaacataacatgcaacaggttcacgattctttattcctgggcaatgtaggagaaggtgtaagagttggtagcaaaagtttcagtggtgtattttccactttcccaggaccatgtaaaagacatagagtaagtgcttacctcgctagtttctgtggattcactagaatcgatgtaggatgttgcccctcctgacgcggtaggagaaggggagggtgccctgcatgtctgccgctgctcttgctcttgccgctgctgaggaggggggcgcatctgccgcagcaccggatgcatctgggaaaagcaaaaaaggggctcgtccctgtttccggaggaatttgcaagcggggtcttgcatgacggggaggcaaacccccgttcgccgcagtccggccggcccgagactcgaaccgggggtcctgcgactcaacccttggaaaataaccctccggctacagggagcgagccacttaatgctttcgctttccagcctaaccgcttacgccgcgcgcggccagtggccaaaaaagctagcgcagcagccgccgcgcctggaaggaagccaaaaggagcgctcccccgttgtctgacgtcgcacacctgggttcgacacgcgggcggtaaccgcatggatcacggcggacggccggatccggggttcgaaccccggtcgtccgccatgatacccttgcgaatttatccaccagaccacggaagagtgcccgcttacaggctctccttttgcacggtctagagcgtcaacgactgcgcacgcctcaccggccagagcgtcccgaccatggagcactttttgccgctgcgcaacatctggaaccgcgtccgcgactttccgcgcgcctccaccaccgccgccggcatcacctggatgtccaggtacatctacggattacgtcgacgtttaaaccatatgatcagctcactcaaaggcggtaat

SEQ ID NO: 137 - DNA, 4514 bp, synthetic construct
  source 1..4514: mol_type = other DNA
gccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggtaagctaacctgaaccctggtcataacttcgtataggatactttatacgaagttatctgcaacttcctgacatggactaacccacttgaaccttgtcttctgccttttcctctacagggctcaggttaagttaaattaattacctgagtggaattttgatggctctagtacctttcagtctgagggctccaacagtgacatctatctcagccctgtagaagaggtaagtaaaaacaataacttcgtataggatactttatacgaagttattcaaccaaatctgacacgatctatcgaacttgcgctacgcccgacacctcctatctgtaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaattgcttgttaatcaataaaccgtttaattcgtttcagttgaactttggtctctgcgtatttctttcttatctagtttccatggctac

SEQ ID NO: 138 - DNA, 4514 bp, synthetic construct
  source 1..4514: mol_type = other DNA
gccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggtaagctaacctgaaccctggtcataacttcgtataggatactttatacgaagttatctgcaacttcctgacatggactaacccacttgaaccttgtcttctgccttttcctctacagggctcaggttaagttaaattaattacctgagtggaattttgatggctctagtacctttcagtctgagggctccaacagtgacatctatctcagccctgtagaagaggtaagtaaaaacaataacttcgtataggatactttatacgaagttattcaaccaaatctgacacgatctatcgaacttgcgctacgcccgacacctcctatctgtaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaattgcttgttaatcaataaaccgtttaattcgtttcagttgaactttggtctctgcgtatttctttcttatctagtttccatggctac

SEQ ID NO: 139 - DNA, 4514 bp, synthetic construct
  source 1..4514: mol_type = other DNA
gccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggtaagctaacctgaaccctggtcataacttcgtataggatactttatacgaagttatctgcaacttcctgacatggactaacccacttgaaccttgtcttctgccttttcctctacagggctcaggttaagttaaattaattacctgagtggaattttgatggctctagtacctttcagtctgagggctccaacagtgacatctatctcagccctgtagaagaggtaagtaaaaacaataacttcgtataggatactttatacgaagttattcaaccaaatctgacacgatctatcgaacttgcgctacgcccgacacctcctatctgtaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaattgcttgttaatcaataaaccgtttaattcgtttcagttgaactttggtctctgcgtatttctttcttatctagtttccatggctac

SEQ ID NO: 140 - protein, 612 aa, synthetic construct
MDTYAGAYDRQSRERENSSAASPATQRSANEDKAADLQREVERDGGRFRFVGHFSEAPGTSAFGTAERPEFERILNECRAGRLNMIIVYDVSRFSRLKVMDAIPIVSELLALGVTIVSTQEGVFRQGNVMDLIHLIMRLDASHKESSLKSAKILDTKNLQRELGGYVGGKAPYGFELVSETKEITRNGRMVNVVINKLAHSTTPLTGPFEFEPDVIRWWWREIKTHKHLPFKPGSQAAIHPGSITGLCKRMDADAVPTRGETIGKKTASSAWDPATVMRILRDPRIAGFAAEVIYKKKPDGTPTTKIEGYRIQRDPITLRPVELDCGPIIEPAEWYELQAWLDGRGRGKGLSRGQAILSAMDKLYCECGAVMTSKRGEESIKDSYRCRRRKVVDPSAPGQHEGTCNVSMAALDKFVAERIFNKIRHAEGDEETLALLWEAARRFGKLTEAPEKSGERANLVAERADALNALEELYEDRAAGAYDGPVGRKHFRKQQAALTLRQQGAEERLAELEAAEAPKLPLDQWFPEDADADPTGPKSWWGRASVDDKRVFVGLFVDKIVVTKSTTGRGQGTPIEKRASITWAKPPTDDDEDDAQDGTEDVAAPKKKRKV

SEQ ID NO: 141 - protein, 343 aa, synthetic construct
MSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 142 - protein, 387 aa, synthetic construct
MIENQLSLLGDFSGVRPDDVKTAIQAAQKKGINVAENEQFKAAFEHLLNEFKKREERYSPNTLRRLESAWTCFVDWCLANHRHSLPATPDTVEAFFIERAEELHRNTLSVYRWAISRVHRVAGCPDPCLDIYVEDRLKAIARKKVREGEAVKQASPFNEQHLLKLTSLWYRSDKLLLRRNLALLAVAYESMLRASELANIRVSDMELAGDGTAILTIPITKTNHSGEPDTCILSQDVVSLLMDYTEAGKLDMSSDGFLFVGVSKHNTCIKPKKDKQTGEVLHKPITTKTVEGVFYSAWETLDLGRQGVKPFTAHSARVGAAQDLLKKGYNTLQIQQSGRWSSGAMVARYGRAILARDGAMAHSRVKTRSAPMQWGKDEKDPKKKRKV

SEQ ID NO: 143 - protein, 430 aa, synthetic construct
MSQFDILCKTPPKVLVRQFVERFERPSGEKIASCAAELTYLCWMITHNGTAIKRATFMSYNTIISNSLSFDIVNKSLQFKYKTQKATILEASLKKLIPAWEFTIIPYNGQKHQSDITDIVSSLQLQFESSEEADKGNSHSKKMLKALLSEGESIWEITEKILNSFEYTSRFTKTKTLYQFLFLATFINCGRFSDIKNVDPKSFKLVQNKYLGVIIQCLVTETKTSVSRHIYFFSARGRIDPLVYLDEFLRNSEPVLKRVNRTGNSSSNKQEYQLLKDNLVRSYNKALKKNAPYPIFAIKNGPKSHIGRHLMTSFLSMKGLTELTNVVGNWSDKRASAVARTTYTHQITAIPDHYFALVSRYYAYDPISKEMIALKDETNPIEEWQHIEQLKGSAEGSIRYPAWNGIISQEVLDYLSSYINRRIPKKKRKV

SEQ ID NO: 144 - protein, 507 aa, synthetic construct
MRALVVIRLSRVTDATTSPERQLESCQQLCAQRGWDVVGVAEDLDVSGAVDPFDRKRRPNLARWLAFEEQPFDVIVAYRVDRLTRSIRHLQQLVHWAEDHKKLVVSATEAHFDTTTPFAAVVIALMGTVAQMELEAIKERNRSAAHFNIRAGKYRGSLPPWGYLPTRVDGEWRLVPDPVQRERILEVYHRVVDNHEPLHLVAHDLNRRGVLSPKDYFAQLQGREPQGREWSATALKRSMISEAMLGYATLNGKTVRDDDGAPLVRAEPILTREQLEALRAELVKTSRAKPAVSTPSLLLRVLFCAVCGEPAYKFAGGGRKHPRYRCRSMGFPKHCGNGTVAMAEWDAFCEEQVLDLLGDAERLEKVWVAGSDSAVELAEVNAELVDLTSLIGSPAYRAGSPQREALDARIAALAARQEELEGLEARPSGWEWRETGQRFGDWWREQDTAAKNTWLRSMNVRLTFDVRGGLTRTIDFGDLQEYEQHLRLGSVVERLHTGMSPKKKRKV

SEQ ID NO: 145 - protein, 485 aa, synthetic construct
MTKKVAIYTRVSTTNQAEEGFSIDEQIDRLTKYAEAMGWQVSDTYTDAGFSGAKLERPAMQRLINDIENKAFDTVLVYKLDRLSRSVRDTLYLVKDVFTKNKIDFISLNESIDTSSAMGSLFLTILSAINEFERENIKERMTMGKLGRAKSGKSMMWTKTAFGYYHNRKTGILEIVPLQATIVEQIFTDYLSGISLTKLRDKLNESGHIGKDIPWSYRTLRQTLDNPVYCGYIKFKDSLFEGMHKPIIPYETYLKVQKELEERQQQTYERNNNPRPFQAKYMLSGMARCGYCGAPLKIVLGHKRKDGSRTMKYHCANRFPRKTKGITVYNDNKKCDSGTYDLSNLENTVIDNLIGFQENNDSLLKIINGNNQPILDTSSFKKQISQIDKKIQKNSDLYLNDFITMDELKDRTDSLQAEKKLLKAKISENKFNDSTDVFELVKTQLGSIPINELSYDNKKKIVNNLVSKVDVTADNVDIIFKFQLA

SEQ ID NO: 146 - protein, 469 aa, synthetic construct
MNRGGPTVRADIYVRISLDRTGEELGVERQEESCRELCKSLGMEVGQVWVDNDLSATKKNVVRPDFEAMIASNPQAIVCWHTDRLIRVTRDLERVIDLGVNVHAVMAGHLDLSTPAGRAVARTVTAWATYEGEQKAERQKLANIQNARAGKPYTPGIRPFGYGDDHMTIVTAEADAIRDGAKMILDGWSLSAVARYWEELKLQSPRSMAAGGKGWSLRGVKKVLTSPRYVGRSSYLGEVVGDAQWPPILDPDVYYGVVAILNNPDRFSGGPRTGRTPGTLLAGIALCGECGKTVSGRGYRGVLVYGCKDTHTRTPRSIADGRASSSTLARLMFPDFLPGLLASGQAEDGQSAASKHSEAQTLRERLDGLATAYAEGAISLSQMTAGSEALRKKLEVIEADLVGSAGIPPFDPVAGVAGLISGWPTTPLPTRRAWVDFCLVVTLNTQKGRHASSMTVDDHVTIEWRDVAE

SEQ ID NO: 147 - protein, 351 aa, synthetic construct
MPKKKRKVGSSELIISGSSGGFLRNIGKEYQEAAENFMRFMNDQGAYAPNTLRDLRLVFHSWARWCHARQLAWFPISPEMAREYFLQLHDADLASTTIDKHYAMLNMLLSHCGLPPLSDDKSVSLAMRRIRREAATEKGERTGQAIPLRWDDLKLLDVLLSRSERLVDLRNRAFLFVAYNTLMRMSEISRIRVGDLDQTGDTVTLHISHTKTITTAAGLDKVLSRRTTAVLNDWLDVSGLREHPDAVLFPPIHRSNKARITTTPLTAPAMEKIFSDAWVLLNKRDATPNKGRYRTWTGHSARVGAAIDMAEKQVSMVEIMQEGTWKKPETLMRYLRRGGVSVGANSRLMDS

SEQ ID NO: 148 - protein, 582 aa, synthetic construct
MTNPASRPKAYSYIRMSSAIQIKGDSFRRQAEASAKYAAEHDLDLIDDYKLADLGVSAFKSDNLTTGALGRFVAECEAGEIEAGSFLLIESLDRLSRDKILDAFSLFARILKTGVKIVTLSDGQVYDGSSDQVGSIYYAISVMIRSNDESKIKSTRGLANWSQKRKLAAEHGVKMSSQCPAWLKLSVDRKSYLIDKERAKIVQRIFEASASGKGANLITKELNRDKVPTFGRGALWAEAFVSKTLRNRAVLGEFQPGQYVSGKRQPAGDPIPGYFPPVIEEELFDIVQASLRGRLLAGGRRGEGQSNIFTHVAFCGYCGSKMRHRSKGSRVKGNPPHRYLTCFNRFNGPGCDCKPLPYAAFERSFLTFVRDVDLRGLLEGAKRKSEAKTIADRITVNEEKVRKADERIRDYLIKIEGAPDLAEIFMERIRELKAEKDDLVRSIEESNDALSKIKSDNVTDEELASLISTFQNPCGENRIRLADRIKSIIERIDVYPNGEIRKDDPAIDLVRASGDPDAEKIIAAMNAGSRLKDDPYFIVTFRNGAVQTVVPNPSNPDDIRVSVYAGEKTRRVEGSAYEYESD

SEQ ID NO: 149 - protein, 509 aa, synthetic construct
MPIAPEFLSLAYPGQEFPAYLYGRASRDPKRKGRSVQSQLDEGRATCLDAGWPIAGEFKDVDRSASAYARRTRDEFEEMIAGIQAGECRILVAFEASRYYRDLEAYVRLRRVCREAGVLLCYNGQVYDLSKSADRKATAQDAVNAEGEADDIRERNLRTTRLNAKRGGAHGPVPDGYKRRYDPDSGDLVDQIPHPDRAGLITEIFRRAAAAEPLAAICRDLNERGETTHRGKAWQRHHLHAILRNPAYIGHRRHLGVDTGKGMWAPICDDEDFAETFQAVQEILSLPGRQLSPGPEAQHLQTGIALCGEHPDEPPLRSVTVRGRTNYNCSTRYDVAMREDRMDAFVEESVITWLASDEAVAAFEDNTDDERTRKARIRLKVLEEQLEAAQKQARTLRPDGMGMLLSIDSLAGLEAELTPQIDKARQESRSLHVPALLRDLLGKPRADVDRAWNEALTLPQRRMILRMVVTIRLFKAGSRGVRAIEPGRITLSYVGEPGFKPVGGNRAKQ

SEQ ID NO: 150 - protein, 471 aa, synthetic construct
MRKVAIYSRVSTINQAEEGYSIQGQIEALTKYCEAMEWKIYKNYSDAGFSGGKLERPAITELIEDGKNNKFDTILVYKLDRLSRNVKDTLYLVKDVFTANNIHFVSLKENIDTSSAMGNLFLTLLSAIAEFEREQIKERMQFGVMNRAKSGKTTAWKTPPYGYRYNKDEKTLSVNELEAANVRQMFDMIISGCSIMSITNYARDNFVGNTWTHVKVKRILENETYKGLVKYREQTFSGDHQAIIDEKTYNKAQIALAHRTDTKTNTRPFQGKYMLSHIAKCGYCGAPLKVCTGRAKNDGTRRQTYVCVNKTESLARRSVNNYNNQKICNTGRYEKKHIEKYVIDVLYKLQHDKEYLKKIKKDDNIIDITPLKKEIEIIDKKINRLNDLYINDLIDLPKLKKDIEELNHLKDDYNKAIKLNYLDKKNEDSLGMLMDNLDIRKSSYDVQSRIVKQLIDRVEVTMDNIDIIFKF

SEQ ID NO: 151 - protein, 477 aa, synthetic construct
MITTRKVAIYVRVSTTNQAEEGYSIQGQIDSLIKYCEAMGWIIYEEYTDAGFSGGKIDRPAMSKLITDAKHKRFDTILVYKLDRLSRSVRDTLYLVKDVFNQNNIHFVSLQENIDTSSAMGNLFLTLLSAIAEFEREQITERMTMGKIGRAKSGKTMAWTYTPFGYDYNKEKGELILDPAKAPIVKMIYTDYLKGMSIQKIVDKLNKMDYNGKDCTWFPHGVKHLLDNPVYYGMTRYNNKLFPGNHQPIITKELFDKTQRERQRRRLGIEENHYTIPFQAKYMLSKFLRCRQCGSRMGLELGRPRKKEGKRSKKYYCLNSRPKRTASCDTPLYDAETLEDYVLHEIAKIQKDPSIASRQKHIEDHELKYKRERIEANINKTVNQLSKLNNLYLNDLITLEDLKTQTNTLIAKKRLLENELDKTCDNDDELDRQETIADFLALPDVWTMDYEGQKYAVELLVQRVKVDRDNIDIHWTF

SEQ ID NO: 152 - protein, 552 aa, synthetic construct
MPGMTTETGPDPAGLIDLFCRKSKAVKSRANGAGQRRKQEISIAAQETLGRKVAALLGMQVRHVWKEVGSASRFRKGKARDDQSKALKALESGEVGALWCYRLDRWDRGGAGAILKIIEPEDGMPRRLLFGWDEDTGRPVLDSTNKRDRGELIRRAEEAREEAEKLSERVRDTKAHQRENGEWVNARAPYGLRVVLVTVSDEEGDEYDERKLAADDEDAGGPDGLTKAEAARLVFTLPVTDRLSYAGTAHAMNTREIPSPTGGPWIAVTVRDMIQNPAYAGWQTTGRQDGKQRRLTFYNGEGKRVSVMHGPPLVTDEEQEAAKAAVKGEDGVGVPLDGSDHDTRRKHLLSGRMRCPGCGGSCSYSGNGYRCWRSSVKGGCPAPTYVARKSVEEYVAFRWAAKLAASEPDDPFVIAVADRWAALTHPQASEDEKYAKAAVREAEKNLGRLLRDRQNGVYDGPAEQFFAPAYQEALSTLQAAKDAVSESSASAAVDVSWIVDSSDYEELWLRATPTMRNAIIDTCIDEIWVAKGQRGRPFDGDERVKIKWAART

SEQ ID NO: 153 - protein, 519 aa, synthetic construct
MQLDATLTLRDEGLSAFHQRHIKQGALGVFLRAIEDGRIQPGSVLIVEGLDRLSRAEPIQAQAQLAQIINAGITVVTASDGREYNRERLKAQPMDLVYSLLVMIRAHEESDTKSKRVKAAIRRQCEGWVAGTWRGIIRNGKDPHWVRLGEHGKFEHVPERVLAVRTMIDLFLEGHGAIEITRRLTEQNLYVSNAGNYSVHMYRIVRNQALIGEKRISVDGEEFRLDGYYPPILTREEFAELQQTMSERGRRKGKGEIPNIITGLSITVCGYCGRAMTTQNSKARAPKGKSVVRRLSCPMNSFNEGCPIGGSCESEIVERALMRYCSDQFNLSRLLEGDDGTARRTAQLAVARQRASDIEAQIQRVTDALLSDDGKAPAAFTRRARELETQLEEQRREIEALEHQIAASSAHGIPAAAEAWAQLVDGVLALDYDARMKARQLVADTFRKIVVYQRGFAPIDDAAADRWKRSGTIGLMLVTKRGGMRLLNVDRRTGCWQAEDDLDPSLIPSDGLPMLPLDA

SEQ ID NO: 154 - protein, 462 aa, synthetic construct
MKVAIYVRVSTDEQAKEGFSIPAQRERLRAFCASQGWEIVQEYIEEGWSAKDLDRPQMQRLLKDIKKGNIDIVLVYRLDRLTRSVLDLYLLLQTFEKYNVAFRSATEVYDTSTAMGRLFITLVAALAQWERENLAERVKFGIEQMIDEGKKPGGHSPYGYKFDKDFNCTIIEEEADVVRMIYRMYCDGYGYRSIADRLNELMVKPRIAKEWNHNSVRDILTNDIYIGTYRWGDKVVPNNHPPIISETLFKKAQKEKEKRGVDRKRVGKFLFTGLLQCGNCGGHKMQGHFDKREQKTYYRCTKCHRITNEKNILEPLLDEIQLLITSKEYFMSKFSDRYDQQEVVDVSALTKELEKIKRQKEKWYDLYMDDRNPIPKEELFAKINELNKKEEEIYSKLSEVEEDKEPVEEKYNRLSKMIDFKQQFEQANDFTKKELLFSIFEKIVIYREKGKLKKITLDYTLK

SEQ ID NO: 155 - protein, 461 aa, synthetic construct
MKVAVYCRVSTLEQKEHGHSIEEQERKLKSFCDINDWTVYDTYIDAGYSGAKRDRPELQRLMNDINKFDLVLVYKLDRLTRNVRDLLDLLEIFEKNDVSFRSATEVYDTTTAMGRLFVTLVGAMAEWERETIRERTQMGKLAALRKGIMLTTPPFYYDRVDNKFVPNKYKDVILWAYDEAMKGQSAKAIARKLNNSDIPPPNNTQWQGRTITHALRNPFTRGHFDWGGVHIENNHEPIITDEMYEKVKDRLNERVNTKKVRHTSIFRGKLVCPVCNARLTLNSHKKKSNSGYIFVKQYYCNNCKVTPNLKPVYIKEKEVIKVFYNYLKRFDLEKYEVTQKQNEPEITIDINKVMEQRKRYHKLYASGLMQEDELFDLIKETDQTIAEYEKQNENREVKQYDIEDIKQYKDLLLEMWDISSDEDKEDFIKMAIKNIYFEYIIGTGNTSRKRNSLKITSIEFY

SEQ ID NO: 156 - protein, 461 aa, synthetic construct
MKVAIYTRVSTLEQKEKGHSIEEQERKLRAYSDINDWKIHKVYTDAGYSGAKKDRPALQEMLNEIDNFDLVLVYKLDRLTRSVKDLLEILELFENKNVLFRSATEVYDTTSAMGRLFVTLVGAMAEWERTTIQERTAMGRRASARKGLAKTVPPFYYDRVNDKFVPNEYKKVLRFAVEEAKKGTSLREITIKLNNSKYKAPLGKNWHRSVIGNALTSPVARGHLVFGDIFVENTHEAIISEEEYEEIKLRISEKTNSTIVKHNAIFRSKLLCPNCNQKLTLNTVKHTPKNKEVWYSKLYFCSNCKNTKNKNACNIDEGEVLKQFYNYLKQFDLTSYKIENQPKEIEDVGIDIEKLRKERARCQTLFIEGMMDKDEAFPIISRIDKEIHEYEKRKDNDKGKTFNYEKIKNFKYSLLNGWELMEDELKTEFIKMAIKNIHFEYVKGIKGKRQNSLKITGIEFY

SEQ ID NO: 157 - protein, 481 aa, synthetic construct
MITTNKVAIYVRVSTTNQVEEGYSIDEQKDKLSSYCDIKDWNVYKVYTDGGFSGSNTDRPALESLIKDAKKRKFDTVLVYKLDRLSRSQKDTLHLIEDVFIKNGIEFLSLQENFDTSTPFGKAMIGLLSVFAQLEREQIKERMQLGKLGRAKSGKSMMWAKTSYGYDYHKETGTVTINPAQALTIKFIFESYLRGRSITKLRDDLNEKYPKHVPWSYRAVRTILDNPVYCGFNQYKGEIYPGNHEPIISKEEYDKTQSELKIRQRTAAENVNPRPFQAKYILSGIAQCGYCGAPLKIMLGVKRKDGSRLKKYECHQRHPRTLRGVTTYNDNKKCDSGFYYKDKLEAYVLKEISKLQDDADYLDKIFSGDNAETIDRESYKKQIEELSKKLSRLNDLYIDDRITLEELQSKSAEFISMRGTLETELENDPALRKNKRKADMRKLLNAEKVFSMDYESQKVLVRRLINKVKVTAEDIVINWKI

SEQ ID NO: 158 - protein, 447 aa, synthetic construct
MLRCAIYIRVSTEEQAMHGLSMDAQKADLTDYAKKHNYEIIDYYVDSGKTARKRLSKRKDLQRMIEDVKLNKIDIIIFTKLDRWFRNVRDYYKIQEVLEDHNVDWKTIFENYDTSTANGRLHINIMLSVAQDEADRTSERIKRVFENKLKNNEPTSGSLPIGYKIKEKSIIIDEEKAPIAKDVFDFYYYHQSQTKVFKEILNKYNLSLCEKTIRRMLENKLYIGIYREHENFCPPLIDKNKFDEVQLILKRRNIKYIPTKRIFLFTSLLICKECRHKMIGNAQIRNTKAGKIEYILYRCNQSYARHTCNHRKVIYENKIETYLLNNIESELKKFIYDYELEDIPKVKNKVNKTNIKRKLEKLKELYINDLIDIDMYKEDYKKYTEILNTKEEKIEQRNLQPLKDFLNSDFKSLYSSISREEKRLLWRGIISEIQIDCNNDITIIPHP

SEQ ID NO: 159 - protein, 460 aa, synthetic construct
MKVAIYTRVSSAEQANEGYSIHEQKKKLISYCEIHDWNEYKVFTDAGISGGSMKRPALQKLMKHLSSFDLVLVYKLDRLTRNVRDLLDMLEEFEQYNVSFKSATEVFDTTSAIGKLFITMVGAMAEWERETIRERSLFGSRAAVREGNYIREAPFCYDNIEGKLHPNEYAKVIDLIVSMFKKGISANEIARRLNSSKVHVPNKKSWNRNSLIRLMRSPVLRGHTKYGDMLIENTHEPVLSEHDYNAINNAISSKTHKSKVKHHAIFRGALVCPQCNRRLHLYAGTVKDRKGYKYDVRRYKCETCSKNKDVKNVSFNESEVENKFVNLLKSYELNKFHIRKVEPVKKIEYDIDKINKQKINYTRSWSLGYIEDDEYFELMEEINATKKMIEEQTTENKQSVSKEQIQSINNFILKGWEELTIKDKEELILSTVDKIEFNFIPKDKKHKTNTLDINNIHFKF

SEQ ID NO: 160 - protein, 474 aa, synthetic construct
MAVGIYIRVSTQEQASEGHSIESQKKKLASYCEIQGWDDYRFYIEEGISGKNTNRPKLKLLMEHIEKGKINILLVYRLDRLTRSVIDLHKLLNFLQEHGCAFKSATETYDTTTANGRMSMGIVSLLAQWETENMSERIKLNLEHKVLVEGERVGAIPYGFDLSDDEKLVKNEKSAILLDMVERVENGWSVNRIVNYLNLTNNDRNWSPNGVLRLLRNPALYGATRWNDKIAENTHEGIISKERFNRLQQILADRSIHHRRDVKGTYIFQGVLRCPVCDQTLSVNRFIKKRKDGTEYCGVLYRCQPCIKQNKYNLAIGEARFLKALNEYMSTVEFQTVEDEVIPKKSEREMLESQLQQIARKREKYQKAWASDLMSDDEFEKLMVETRETYDECKQKLESCEDPIKIDETYLKEIVYMFHQTFNDLESEKQKEFISKFIRTIRYTVKEQQPIRPDKSKTGKGKQKVIITEVEFYQ

SEQ ID NO: 161 - protein, 471 aa, synthetic construct
MTVGIYIRVSTEEQVKEGFSISAQKEKLKAYCTAQGWEDFKFYVDEGKSAKDMHRPLLQEMISHIKKGLIDTVLVYKLDRLTRSVVDLHNLLSIFDEFNCAFKSATEVYDTSSAMGRFFITIISSVAQFERENTSERVSFGMAEKVRQGEYIPLAPFGYTKGTDGKLIVNKIEKEIFLQVVEMVSTGYSLRQTCEYLTNIGLKTRRSNDVWKVSTLIWMLKNPAVYGAIKWNNEIYENTHEPLIDKATFNKVAKILSIRSKSTTSRRGHVHHIFKNRLICPACGKRLSGLRTKYINKNKETFYNNNYRCATCKEHRRPAVQISEQKIEKAFIDYISNYTLNKANISSKKLDNNLRKQEMIQKEIISLQRKREKFQKAWAADLMNDDEFSKLMIDTKMEIDAAEDRKKEYDVSLFVSPEDIAKRNNILRELKINWTSLSPTEKTDFISMFIEGIEYVKDDENKAVITKISFL

SEQ ID NO: 162 - protein, 452 aa, synthetic construct
MKAAIYIRVSTQEQIENYSIQAQTEKLTALCRSKDWDVYDIFIDGGYSGSNMNRPALNEMLSKLHEIDAVVVYRLDRLSRSQRDTITLIEEYFLKNNVEFVSLSETLDTSSPFGRAMIGILSVFAQLERETIRDRMVMGKIKRIEAGLPLTTAKGRTFGYDVIDTKLYINEEEAKQLQMIYDIFEEEKSITTLQKRLKKLGFKVKSYSSYNNWLTNDLYCGYVSYADKVHTKGVHEPIISEEQFYRVQEIFSRMGKNPNMNRDSASLLNNLVVCGKCGLGFVHRRKDTISRGKKYHYRYYSCKTYKHTHELEKCGNKIWRADKLEELIIDRVNNYSFASRNVDKEDELDNLNEKLKTEHKKKKRLFDLYISGSYEVSELDAMMADIDAQINYYEAQIEANEELKKNKKIQENLADLATVDFDSLEFREKQLYLKSLINKIYIDGEQVTIEWL

SEQ ID NO: 163 - protein, 470 aa, synthetic construct
MKGESELDKKAAIYIRVSTQEQATEGYSIQAQTDRLIKYVEAKDFILYKKYIDAGYSASKLERPAMQDLIQDVQSKKVDVVIVYKLDRLSRSQKDTMYLIEDIFRPNDVELISMQESFDTSTAFGSATVGMLSVFAQLERKSISERMITGRVERAKKGFYHTGGQDRPPAGYQFNSDNQLIINEYEAAAIKDLFRLYNDGLGKSSISEYLKKNYPGKNKWLPSSIDRMLKNSLYIGKVKFSGAEYDGIHEPIIDEVTFYKTQKEIARRKQTNTKRYNYVALLGGLCECGICGAKMANRRAVGRKGKVYRYYRCYSKKGSPKHMMKTDGCSSKAQQQFIIDEAVINNLKNIDVEAELKRRSAPQTNTSLISSQIESIDKQINKLIDLFQVDSMPLDVISEKIDKLNKEKQSMEKLLERKNKLDKTELQHRFDVLKSFDWDNSSIESKRVVIEMLVQKVIIHDNSIEIILVE

SEQ ID NO: 164 - protein, 512 aa, synthetic construct
MRTNEHNFHNIEEEIKHVAVYLRLSRGEDESELDNHKTRLLNRCELNNWSYELYKEIGSGSTIDDRPVMQKLLTDVEKNLYDAVLVVDLDRLSRGNGTDNDRILYSMKVSETLIVVESPYQVLDANNESDEEIILFKGFFARFEFKQINKRMREGKKLAQSRGQWVNSVTPYGYIVNKTTKKLTPSEEEAKVVIMIKDFFFEGKSTSDIAWELNKRKIKPRRATEWRSSSIANILQNEVYVGNIVYNKSVGNKKPSKSKTRVTTPYRRLPEEEWRRVYNAHQPLYSKEEFDRIKQYFECNVKSHKGSEVRTYALTGLCKTPDGKTMRVTQGKKGTDDDLYLFPKKNKHGDSSIYKGISYNVVYETLKEVILQVKDYLDSVLDQNENKDLVEELKEELMKKEDELETIQKAKNRIVQGFLIGLYDEQDSIELKVEKEKEIDEKEKEIEAIKMKIDNAKTVNNSIKKTKIERLLSDVQSAESEKEINRFYKTLIKEIIVDRTDENEAKIKVNFL

SEQ ID NO: 165 - protein, 475 aa, synthetic construct
MITTNKVAIYVRVSTTNQVEEGYSIDEQKDKLEAYCKIKDWKIYDVYVDGGFSGANTQRPELERLISDVKRKKVDIVLVYKLDRLSRSQKDTLFLIEDVFAKNDVAFISLQENFDTSTPFGKASIGMLSVFAQLEREQIKERMMLGKEGRAKNGKSMSWTTIAFGYDYSKETGVLSVNPTQALIVNRIFTEYLNGKPVVKIIRDLNAEGHVGRKRPWGETITKYLLKNETYLGKVKYKDKVYEGQHEPIITQELFDLVQLEVERRQISAYEKYNNPRPFRAKYMLSGLMKCGYCGASLGLRYTRKDKNGISHHKYQCRNRHSKDLEKRCESGWYSKEELERGVIKELERIKFDPKYKNETLAKKEETIKVEEIKKQLERINNQVSKLTELYLDEIITRKELDEKNDKIKTERQFLEEQLENQKSNVLSIRKRKLTRLLKDFDVEKLSYEDASKIVKNIIKEIIVTKDGMSITLDF

SEQ ID NO: 166 - protein, 462 aa, synthetic construct
MGKSITVIPAKKVQTSVLHQDRKKIKVAAYCRVSTDQEEQLSSYENQVNYYREFISKHEDYELVDIYADEGISATNTKKRDAFNRLIQDCRAGKVDRILVKSISRFARNTLDCIKYVRELKELGVGVTFEKENIDSLDSKGEVLLTILSSLAQDESRSISENATWGIRKKFERGEVRVNTTKFMGYDKDENGRLIINPQQAETVKFIYEKFLEGYSPESIAKYLNDNEIPGWTGKANWYPSAIQKMLQNEKYKGDALLQKTFTVDFLTKKRVQNDGQVNQYYVENSHEAIIDEETWETVQLEMARRKTYRDEHQLKSYIMQSEDNPFTTKVFCGACGSAFGRKNWATSRGKRKVWQCNNRYRIKGVEGCYSSHLDEATLEQIFLKALELLSENIDLLDGKWEKILAENRLLDKHYSMALSDLLRQEQIDFNPSDMCRVLDHIRIGLDGEITVCLLEGTEVDL

SEQ ID NO: 167 - protein, 411 aa, synthetic construct
MRTVRRIQPIKSPCKPRFKVAAYARVSDSRLHHSLSTQISYYNRLIQAHPDWELVGIYYDEGISGKEQSNRQGFLNLIKDCEDGKIDRIITKSIARFGRNTVELLTTVRQLRLKNIGVTFEKENIDSLSSEGELMLTLLASVAQEESQNLSENIRWRIQKKFEKGIPHTPQDMYGYRWDGEQYQIEPNEAKVIRKVFKWYLDGDSVQQIVDKLNQEQVLTRLGNPFTVASIREFFKQEAYFGRLVLQKTYREAFSRNPKRNKGQRNKYIIENAHEPIVTKEYFDLVLHEKERRNQLMHQESHLNKGIFRDKISCSECGCLMIVKVDSKQVNKTVRYYCRTRNRFGASSCSCRTLGEKRLLASFKSKLGIVPDKEWVENNIKHIEYDFGYRILRVTPVKGRKYLIEIREGRY

SEQ ID NO: 168 - protein, 473 aa, synthetic construct
MRNKVAIYVRVSTASQADEGYSIDEQKSKLEAYCEIKDWKIYDTYIDGGFSGANTQRPELERLISDAKRKKIDIVLVYKLDRLSRSQKDTLFLIEDVFAKNDVAFISLQENFDTSTPFGKASIGMLSVFAQLEREQIKERMMLGKEGRAKNGKSMSWTTIPFGYDYSKETGILSVNPTQALIVKRIFTEYLNGKSVVKIIRDLNAEGHVGRKRPWGETITKYLLKNETYLGKSKYKGKVFEGQHDAIISQELFDLVQLEVEKRQISAFEKYNNPRPFRAKYMLSGLMKCGYCGASLGLYVAPKNKNGVSKYKYQCRHRYHKDKAIRCNSGWYSKDELEKRVIKELERLKFDPKYKKETLAKKDETIKVEDIKKQLERINKQVSKLTELYLDEVITRKDLDEKNAKIKTERQYLEEQLENQKSNVMSIRKRKLSRLLKDFDIEKLSYEEASKIVKSVIKEIVVTKDDMTITLDF

SEQ ID NO: 169 - protein, 463 aa, synthetic construct
MKVATYVRVSTDEQAKEGFSIPAQRERLRAFCESQGWEIVEEYIEEGWSAKDLDRPQMQRLLKDIKKGNIDIVLVYRLDRLTRSVLDLYLLLQTFEKYNVAFRSATEVYDTSTAMGRLFITLVAALAQWERENLAERVKFGIEQMIDEGKKPGGHSPYGYKFDKDFNCTIIEDEANTVRMIYRMYCDGYGYHSIAKRLNELGIKPRIAKEWNHNSVRDILTNDIYIGTYRWGNKVVLNNHPPIISETLFRKVQKEKEKRRVDRTRVGKFLLTGLLYCGNCNGHKMQGTFDKREQKTYYRCLKCNRITNEKNILEPLLDEIQLLITSKEYFMSKFSDQYDQKEEVDVSALKKELEKIKRQKEKWYDLYMDDRNPIPKEDLFAKINELNKKEEEIYNKLNEVEPEDKEPVEEKYNRLSKMIDFKQQFEQANDFTKKELLFSIFEKIVIYREKGKLKKITLDYTLK

SEQ ID NO: 170 - protein, 452 aa, synthetic construct
MLRVALYIRVSTEEQALNGDSIRTQIEALEQYSKENDFNIVGKYIDEGCSATNLKRPNLQRLLRDVEKDKVDLVLMTKIDRLSRGVKNYYKIMETLEKHKCDWKTILENYDSSTAAGRLHINIMLSVAENEAAQTSERIKFVFQDKLRRKEVISGTIPIGYKIENKHLVIDKEKKYIVKAIFDEYEKSGSVRTLIETINNLHGELYSYNKIKNILRNELYIGIYNKRGFYVEDYCEPIISKKQFKQIQRILEKNKKTTPNKNIHYHIFSGLLKCKECGYTLKGNSSNVGEKLYLSYRCSTFYLNKNCVHNVTHNEKHIENYLLTNLKPQLHKHMVKLEAQNEKIRRNKKSNKKDEKKKIMKKLDKIKDLYLEDLIDKETYRKDYEKLQSQLDNITEEQESQIIDTSHIKKFLDIDINEMYSDLSRVERRRFWLSIIDYIEIDNNKNITINFI

SEQ ID NO: 171 - protein, 592 aa, synthetic construct
MKITLLYYIKKFNIYCNRYLSQQINISVDIIGFYQFKNVTNSVTDVLKRGDNLDRICIYLRKSRADEELEKTIGVGETLSKHRKALLKFAKEKKLNIMEIKEEIVSADSIFFRPKMIELLKEVENNQYTGVLVMDIQRLGRGDTEDQGIIARIFKESHTKIITPMKTYDLDDDLDEDYFEFESFMGRKEYKMIKKRMQGGRVRSVEDGNYIATNPPFGYDIHWINKSRTLKFNSKESEIVKLIFKLYTEGNGAGTISNYLNSLGYKTKFGNNFSNSSIIFILKNPVYIGKITWKKKDIRKSKDPHKVKDTRTRDKSEWIIADGKHEPIIDEKIWNKAQEILNNKYHIPYKIANGPANPLAGVVICSKCNSKMVMRKYGKKLPHLICNNKECNNKSARFDYIEKAVLEGLDEYLKNYKVNVKANNKTSDIEPYEQQSNALNKELILLNEQKLKLFDFLEREIYTEEIFLERSKNLDERINTTTLAINKIKKILDNEKKKNNKNDIVKFEKILEGYKKTNDIQKKNELMKSLVFKIEYKKEQHQRNDGLLYIYFLSFCVRCISYLTQFISFFVYPYRILEIYLTFSFFIISYEH

SEQ ID NO: 172 - protein, 504 aa, synthetic construct
MRICMYLRKSRADEELEKTLGEGETLSKHRKALLKFAKEKNLNIVEIKEEIVSGESLFFRPKMLELLKEIENKQYSGVLVMDMQRLGRGNMQDQGIILETFKKSNTKIITPMKTYDLSNDFDEEYSEFEAFMSRKELKMINRRMQGGRVRSVEDGNYIATNAPYGYDIHWINKARTLKPNQKESEIVKLIFKLYIEGNGAGTIAKHLNSLGYKTKFGNSFNNSSIIFILKNPVYIGKITWKKKDIRKSKDPNKVKDTRTRDKSEWIIVDGKHDPIIDQITWKQAQEILNNRYHVPYKLVNGPANPLAGLIICTTCKSKMVMRKLRGTDRILCKNNKCNNISNRFDAVEKSVVESLENYLKAYKVNLPELNKTSNLKLYEQQISTLKKELKILNEQKLKLFDFLERGIYDEDTFLKRSKNLDERIEITNESLSNLNQIIAKENKAIKKEDIIKFEKVLDSYKSTADIRLKNELMKTLIFKIEYTKNKKGNDFKIKVFPKLKPLNI

SEQ ID NO: 173 - protein, 545 aa, synthetic construct
MIAAIYSRKSKFTGKGESVENQIEMCKEYLKRNFNNIDDIEIYEDEGFSGKDTNRPKFKKMIKAAKNKKFNILICYRLDRISRNVADFSNTIEELQKYNIDFISIKEQFDTSTPMGRAMMNIAAVFAQLERETIAERIKDNMVELAKTGRWLGGTSPLGYKSEPIEYSNEDGKSKKMYKLTEVENEMNIVKLIYKLYLEKRGFSSVATYLCKNKYKGKNGGEFSRETARQIVINPVYCISDKTIFKWFKSKGATTYGTPDGIHGLMVYNKREGGKKDKPINEWIIAVGKHRGVISSDIWLKCQNLIQQNNAKSSPRSGTGEKFLLSGMVVCKECGSGMSSWSHFNKKTNFMERYYRCNLRNRASNRCSTKMLNAYKAEEYVANYLKELDINAIKKMYHSNKKNIIDYDAKYEVNKLNKSIEENKKIIQGIIKKIALFDDLDILGMLKNELERLKKENDEMKIKLKELKSILELEDEEEIFLSTMEENISNFKKFYDFVNITQKRILIKGLVESIVWDTGGEEKILEINLIGSNTKLPSGKVKRRE

SEQ ID NO: 174 - protein, 485 aa, synthetic construct
MSKKVAIYTRVSTTNQAEEGYSIDEQIDKLKMYCEAMDWKVSEIYTDAGFTGSKLTRPAMEKMITDIGLKKFDTVIVYKLDRLSRSVRDTLYLVKDVFTKNEIDFISLSESIDTSSAMGSLFLTILSAINEFERENIKERMTMGKIGRAKSGKSMMWAKTAFGYSHNQETGILEINPLEASIVEQIFNEYLKGTSITKLRDKLNEDGHIAKELPWSYRTIRQTLDNPVYCGYIKYKNNTFEGLHKPIISHETYLSVQKELEARQQQTYEKNNNPRPFQAKYLLSGIARCGYCGAPLRIVLGHRRKDGSRTMKYQCVNRFPRKTKGVTTYNDNKKCDSGAYDMQWIEDIVLKTLNGFQKSDKKLRKILNIKEESKVDTSGFQKQLKSINNKIQKNSDLYLNDFITMDDLKKRTEMLQGEKKLIQARINEVDKPSTSEIFDLVKSELGETTISKISYEDKKKIVNNLISKVDVTADNIDIIFKFQLA

SEQ ID NO: 175 - protein, 458 aa, synthetic construct
MNEQKDKLKKYCEIKDWTIVKEYVDPGRSGSNINRPSMQQLIKDADTGLYDAVLVYKLDRLSRSQKDTLYLIEDVFQKNNIHFISLSENFDTSTAFGKAMIGILSVFAQLEREQIKERMSMGRVGRAKSGKIMEFNNPAFGYEVDGDNYKVDPLRAEIVKRIYKMYLSGTSINKIKETLNLEGHIGNKKNWSDTRIRYILSNPTYLGKIRYDGKTYDGKFSPIIDEETFNKTQNELKERQTATYKRFNMKLRPFQSKYMLSGLLRCGYCGATLFVNSYVYNGKRKLRYNCPSTYKSKQKTRTYKIMDPNCPFKLVYAKDLEPAVINEIKNLALNPQSIQKPVKKKPDIDVEAIQKELAKVRKQQQRLIDLYVISDDVNIDNISKKSADLKLQEETLKKQLAPLEEPNDDDKIVAFNEILAQIKDIDSLDYDKQKFIVKKLIKKIDVWNDNKIKIHWNI

SEQ ID NO: 176 - protein, 515 aa, synthetic construct
MKTAIYLRKSRADLEAEARGEGETLAKHRSTLLKIAKEMNLNVLSVREEIVSGESLVKRPEMLALLEEIEDNKYDAVLCMDMDRLGRGGMKEQGIILETFKRSNTKIMTPRKTYDLNDEWDEEYSEFEAFMARKELKIITRRMQRGRIASVEAGNYLGTHAPFGYDIHRLNKRERTLTINSEEASVVRMIFDWYANEDMGASAIRNKLNDLGYKSKLGNDWNPYSILDILKNNIYIGKVTWQKRKEVKRPDAVKRSCARQDKSDWIIADGKHEPIIPESLFEQAQEKLNSRYHVPYNTNGIKNPLAGIIKCSKCGYSMVQRYPKNRKETMDCKHRGCENKSSYTELIEKRLLEALKEWYINYKADFEAHKQGDKLKETQVIQMNEAALRKLEKELVDVQKQKNNLHDLLERGVYTVDMFLERSQVISDRINEITSTMENLKKEIKTEIKKEKVKKDTIPQVEHVLDLYFKTDDPKKKNSLLKSVLEKAVYKKEKWQRLDDFELVLYPKLPQDGDI

SEQ ID NO: 177 - protein, 516 aa, synthetic construct
MYRPESLDVCIYLRKSRKDVEEERRAIEEGSSYNALERHRKRLFAIAKAENHNIIDIFEEVASGESIQERPQMQQLLRKLEGNEIDGVLVIDLDRLGRGDMLDAGMIDRAFRYSSTKIITPTDVYDPDDESWELVFGIKSLISRQELKSITKRLQNGRIDSVKEGKHIGKKPPYGYLKDENLRLYPDPEKAWIVKKIFELMCDGKGRQMIAAELDRLGIDPPVTKRGAWDSSTITSIIKNEVYTGVIVWGKFKHKKRNGKYTRHKNPQEKWIMYENAHEPIISKELFDAANEAHSSRHKPAVITSKKLTNPLAGILKCKLCGYTMLIQTRKDRPHNYLRCNNPACKGKQKQSVFNLVEEKLLYSLQQIVDEYQAQKVEEVEIDDSKLISFKEKAIISKEKELKELQAQKGNLHDLLEQGIYTVEIFLERQKNLVERITSIENDIEVLQKEIETEQIKEHNKTEFIPALKTVIESYHKTTNIELKNQLLKTILSTVTYYRHPDWKTNEFEIQVYFKI

SEQ ID NO: 178 - protein, 513 aa, synthetic construct
MKYLALHENSRIAVYSRKSREDRDSEDTLAKHRNELEYLIKRENFKNVQWFEKVVSGETIDERPMFSLLLPRIENGEFDAVCAVAMDRLSRGSQIDSGRILEAFKQSGTLFITPKKTYDLSIEGDEMLSEFESIIARSEYRAIKRRTINGKKNATREGRLHSGSVPYGYKWDKNLKAAVVVEEKKKIYRMMIKWFLEEEYSCTVIAEMLNELKVPSPSGRSIWYGEVVSEILSNDFHRGYVWFGKYKKSKSNNSIVQNKNLDEVLIAKGHHETMKTDEEHALILNRIEKLRTYKVAGRRLNMNTHRLSGIVRCPYCHKAQAIEQPKGRRKHVRKCLRKSAERTKECEETKGIHEEVLFQSIMKEIKKYNESLFSPTEQDVNDDSYTAQLIGLREKAVKKAKGRIERIKEMYLDGDISKTEYKEKLKISQETLQKAENELAELIASTEFQNALSAETKKEKWSHHKVQEMIESTDGMSNSEINLILKMLISHVTYTVEDLGDGTKNLNIKVYYN

SEQ ID NO: 179 - protein, 580 aa, synthetic construct
MDPQHKPTRALIVIRLSRLTDETTSPERQLEACERFCAARGWEVVGVAEDLDVSAGTTSPFERPSLSQWIGDGKDNPGRIGEFDTVVFYRVDRLVRRVRHLHDVIAWSERFDVNMVSATESHFDLSTTIGALIAQLVASFAEMELEGISQRATSAHRHNVQLGKFVGGSPPFGYMPEETPDGWRLVHDPDVVPIILEVVDRVLEGEPLRRITDDLNARGATTARDLVKQRKGKETEGHKWHSNVLKRRLMSPAMLGYALRREPLTDSKGKPKLSAKGAKLYGPEEIVRGPDGLPVQRAEPILPKPLFDRVVAELEARELQKEPTKRINSMLLRVLYCGVCGQPVYRAKGQGGRSDRYRCRSIQDGANCGNPSVLTYELDDLVEESILVLMGDSERLAHVWNPGEDNASELAEVEARLADRTGLIGVGAYKAGTPQRATLDTLIEADAKLYERLKAATPRPAGWTWEPTGETFAEWWAALDTGARNVYLRNMGVRVTYDKRPVPEQVSAGEKPRVHLELGEVRKMAEQVAVTGTIGTLTRNYTRLGEIGITHVDIDAGSGKAVFVTKSGERFELPLNIPEE

SEQ ID NO: 180 - protein, 540 aa, synthetic construct
MKAIAIYARKSLFTGKGDSIGAQVDTCKRFIDYKFANEDYEIRTFKDEGWSGKTTDRPDFTNMVNLIKSKKIDYVITYKLDRIGRTARDLHNFLYELDNLGIVYLSATEPYDTTTSAGRFMISILAAMAQMERERLAERVKSGMIQIAKKGRWLGGQCPLGFDSKREIYIDDMGKERQMMRLTPNKEEIKIVKLIYDKYLEMGSMSQVRKYCLENSIRGKNGGDFSTNTLKQLLTSPIYVKSSDNIFKYLESQNINVFGTPNGNGMLTFNKTKEIRIERDKSEWIAAVGKHKGIIDDNKWLQIQQQLQQQSEKQIKSSGRQGTTSTGLLSGIIKCSKCGNNLLIKTGHKSKKNPGTTYSYYVCGKKDNSYGHKCDNKNVRTDEADSAVITQLKLYNKELLIKNLKEALIQNEKTDTDNIEILESKLKEKEKAVSNLVKKLSLIDDESISNIILNEVTNINKEINDIKLQLSNETLKINEVTKATLDTEIYIKILENFNKKIDDITDPIEKMNLLKSALESVEWNGDSGEFKINLIGSKKK

SEQ ID NO: 181 - protein, 460 aa, synthetic construct
MKVAIYTRVSTLEQREKGHSIDEQERKLRSFCDINDWTVKDVYVDAGFSGAKRDRPELTRLLDDISEFDLVLVYKLDRLTRSVRDLLDLLEVFENNNVAFRSATEVYDTTTAIGRLFVTLVGAMAEWERETIRERSLMGKRAAIKKGMILTAPPFYYDRVNNTYIPNQYKDVVLDVYNKVKKGYSIAHIARLYNNSDVKPPNGNEEWTTRMLMHALRNPVTRGHYQWGEIYIEDSHEPIITDEMYNTIIDRLDKHTNTKVVAHTSVFRGKLICPNCGYALTLNSQKRKRKNDTIVYKTYYCNNCKITKGMKPHHITETETLRVFKDHLSKIDLKQYETQEKEKQSHVTIDLSKVMEQRKRYHKLYASGMMQENELFELIKETDEMIEEYEKQRKQVDVKEFDICKIKEIKDVLLKSWDIFTLEDKADFIQMSIKAINIEYTKLKRGKSSNSMKIKDIEFY

SEQ ID NO: 182 - DNA, 1839 bp, synthetic construct
  source 1..1839: mol_type = other DNA
atggatacctacgccggagcctacgacagacagagccgggagagagagaacagcagcgccgccagccccgccacccagagaagcgccaacgaggataaggccgccgatctgcagagagaggtggagagggacggcggcagattcagatttgtgggccacttcagcgaggcccctggcaccagcgccttcggcaccgccgagaggcccgagttcgagagaatcctgaacgagtgtagggccggcaggctgaacatgatcatcgtgtacgacgtgtcccggttcagcaggctgaaggtgatggacgccatccctatcgtgtccgagctgctggccctgggcgtgaccatcgtgtccacccaggaaggcgtctttagacagggcaacgtgatggacctgatccacctgatcatgaggctggacgccagccacaaggagagcagcctgaaaagcgccaagatcctggacaccaagaacctgcagagggagctgggcggctatgtgggcggcaaggccccctacggcttcgagctggtgtccgaaaccaaggagatcacccggaacggcaggatggtgaacgtggtgatcaacaagctggcccacagcaccacccccctgaccggccccttcgagtttgagcccgacgtgatcaggtggtggtggcgggagatcaagacccacaagcacctgcctttcaagcccggcagccaggccgccatccaccccggcagcatcaccggcctgtgtaagagaatggacgccgacgccgtgcccaccagaggcgaaaccatcggcaagaaaaccgccagcagcgcctgggaccccgccaccgtgatgagaatcctgagggaccctaggatcgccggcttcgccgccgaggtgatctacaagaagaagcccgacggcacccccaccaccaagatcgagggctacagaatccagagggaccccatcaccctgaggcctgtggagctggactgtggccctatcatcgagcctgccgagtggtacgagctgcaggcctggctggacggcagaggcagaggcaagggcctgagcagaggccaggccatcctgagcgccatggacaagctgtactgtgagtgtggcgccgtgatgaccagcaagagaggcgaggagagcatcaaggacagctaccggtgccggagaagaaaggtggtggaccccagcgcccctggccagcacgagggcacctgtaatgtgagcatggccgccctggacaagttcgtggccgagcggatcttcaacaagatccggcacgccgagggcgacgaggaaaccctggccctgctgtgggaggccgccagaagattcggcaagctgaccgaggcccccgaaaagagcggcgagagggccaacctggtggccgagagagccgacgccctgaacgccctggaggagctgtacgaggacagagccgccggagcctatgacggccctgtgggcaggaagcacttcagaaagcagcaggccgccctgaccctgagacagcagggcgccgaggaaagactggccgagctggaggccgccgaggcccctaagctgcccctggatcagtggttccccgaggatgccgacgccgaccccaccggccccaagtcctggtggggcagagccagcgtggacgacaagagggtgttcgtgggcctgttcgtggataagatcgtggtgaccaagagcaccaccggcaggggccagggcacccccatcgagaagagagccagcatcacctgggccaagcctcccaccgacgacgacgaggatgacgcccaggacggcaccgaggacgtggccgcccccaagaaaaagcggaaggtgtaa

SEQ ID NO: 183 - DNA, 1032 bp, synthetic construct
  source 1..1032: mol_type = other DNA
atgtccaatctgctgaccgtgcaccagaacctgcctgctctgcccgtggacgccaccagcgacgaggtgcgcaagaacctgatggacatgttccgcgaccgccaggccttcagcgagcacacctggaagatgctgctgagcgtgtgccgcagctgggccgcctggtgcaagctgaacaaccgcaagtggttccccgccgagcccgaggacgtgcgcgactacctgctgtacctgcaggcccgcggcctggccgtgaaaaccatccagcagcacctgggccagctgaacatgctgcaccgccgcagcggcctgcctaggccatctgactctaatgccgtgtctctggtcatgcggcggatccggaaagaaaacgtggacgccggcgagagagctaagcaggctctggctttcgagagaaccgacttcgaccaagtgcggtccctgatggaaaactccgaccggtgccaggatatccggaacctggcttttctgggaatcgcctacaacaccctgctgcggatcgctgagatcgcccggatcagagtgaaggacatctctagaaccgacggcggcagaatgctgatccacatcggcagaacaaagaccctggtgtccacagctggcgtggaaaaggctctgtctctgggcgtgaccaagctggtggaacggtggatttctgtgtccggcgtggccgacgatcccaacaactacctgttctgcagagtccggaagaacggcgtggcagccccttctgctacatcccagctgtctacaagagccctggaaggcatcttcgaggctacccacagactgatctacggcgccaaggacgatagcggccagagatatttggcttggagcggccactccgctagagtgggagctgctagagatatggctagagccggcgtgtccattcctgagatcatgcaagctggcggctggaccaacgtgaacatcgtgatgaactacatccgcaacctggactccgagacaggcgctatggttcgactgctggaagatggcgactga

SEQ ID NO: 184 - DNA, 1164 bp, synthetic construct
  source 1..1164: mol_type = other DNA
atgatcgagaaccagctgagcctgctgggcgacttttctggcgtgcggcccgacgatgtgaaaaccgccattcaggccgcccagaaaaagggcatcaacgtggccgagaacgagcagttcaaggccgccttcgagcatctgctgaacgagttcaagaagcgggaagagagatacagccccaacaccctgcggcggctggaaagcgcctggacctgcttcgtggattggtgcctggccaaccacagacacagcctgcctgccacccccgataccgtggaagccttcttcatcgagcgggccgaggaactgcaccggaacaccctgagcgtgtacagatgggccatcagccgggtgcacagagtggccggatgccctgatccctgcctggacatctacgtggaagatcggctgaaggccattgcccggaagaaagtgcgggaaggcgaggccgtgaagcaggccagccctttcaacgagcagcatctgctgaagctgaccagcctgtggtacagaagcgacaagctgctgctgcggcggaacctggctctgctggctgtggcctacgagagcatgctgagagccagcgagctggccaacatccgggtgtccgatatggaactggccggcgacggaaccgccatcctgaccatccctatcaccaagaccaaccactccggcgagcccgatacctgcatcctgtcccaggatgtggtgtccctgctgatggactacaccgaggccggcaagctggatatgagcagcgacggcttcctgttcgtgggcgtgtccaagcacaacacctgtatcaagcccaagaaggacaagcagaccggcgaggtgctgcacaagcccatcaccaccaagacagtggaaggcgtgttctacagcgcctgggagacactggacctgggcagacagggcgtgaagcctttcacagcccacagcgccagagtgggagccgctcaggacctgctgaagaagggctacaataccctgcagatccagcagtccggccggtggtctagcggagccatggtggccagatacggcagagccatcctggctagggatggcgctatggcccacagcagagtgaaaaccagatccgcccccatgcagtggggcaaggacgagaaggaccccaagaaaaagcggaaggtgtga

SEQ ID NO: 185 - DNA, 1293 bp, synthetic construct
  source 1..1293: mol_type = other DNA
atgagccagttcgacatcctgtgcaagaccccccccaaggtgctggtgcggcagttcgtggagagattcgagaggcccagcggcgagaagatcgccagctgtgccgccgagctgacctacctgtgctggatgatcacccacaacggcaccgccatcaagagggccaccttcatgagctacaacaccatcatcagcaacagcctgagcttcgacatcgtgaacaagagcctgcagttcaagtacaagacccagaaggccaccatcctggaggccagcctgaagaagctgatccccgcctgggagttcaccatcatcccttacaacggccagaagcaccagagcgacatcaccgacatcgtgtccagcctgcagctgcagttcgagagcagcgaggaggccgacaagggcaacagccacagcaagaagatgctgaaggccctgctgtccgagggcgagagcatctgggagatcaccgagaagatcctgaacagcttcgagtacaccagcaggttcaccaagaccaagaccctgtaccagttcctgttcctggccacattcatcaactgcggcaggttcagcgacatcaagaacgtggaccccaagagcttcaagctggtgcagaacaagtacctgggcgtgatcattcagtgcctggtgaccgaaaccaagacaagcgtgtccaggcacatctactttttcagcgccagaggcaggatcgaccccctggtgtacctggacgagttcctgaggaacagcgagcccgtgctgaagagagtgaacaggaccggcaacagcagcagcaacaagcaggagtaccagctgctgaaggacaacctggtgcgcagctacaacaaggccctgaagaagaacgccccctaccccatcttcgctatcaagaacggccctaagagccacatcggcaggcacctgatgaccagctttctgagcatgaagggcctgaccgagctgacaaacgtggtgggcaactggagcgacaagagggcctccgccgtggccaggaccacctacacccaccagatcaccgccatccccgaccactacttcgccctggtgtccaggtactacgcctacgaccccatcagcaaggagatgatcgccctgaaggacgaaaccaaccccatcgaggagtggcagcacatcgagcagctgaagggcagcgccgagggcagcatcagataccccgcctggaacggcatcatcagccaggaggtgctggactacctgagcagctacatcaacaggcggatccccaagaaaaagcggaaggtgtga

SEQ ID NO: 186 - DNA, 1524 bp, synthetic construct
  source 1..1524: mol_type = other DNA
atgcgagccctggtggtcattcgcctgagcagagtcacagacgctactacaagccctgagcggcagctggagtcctgtcagcagctgtgcgcacagcgaggatgggatgtggtcggagtggcagaggatctggacgtgagcggggctgtcgatccattcgaccgaaagcggaggcccaacctggcacgatggctggctttcgaggaacagccctttgatgtgatcgtcgcctacagagtggacaggctgacacgctcaattcgacatctgcagcagctggtgcattgggccgaggatcacaagaaactggtggtcagcgcaactgaagcccacttcgacaccacaactccttttgccgctgtggtcatcgcactgatgggcaccgtggcccagatggagctggaagctatcaaggagcgaaaccggagcgcagcccatttcaatattcgggccgggaaatacagaggcagcctgcccccttggggctatctgcctacccgggtggatggggagtggagactggtgccagaccccgtccagagagagaggattctggaagtgtaccacagagtggtggacaaccacgaaccactgcatctggtggcccacgatctgaataggcgcggagtcctgtctccaaaggactattttgctcagctgcagggaagggagccacagggacgagaatggagtgctaccgcactgaagcggtctatgatcagtgaggctatgctgggctatgcaactctgaatgggaaaaccgtgagagatgatgacggagcaccactggtgcgggctgagcctattctgacaagagagcagctggaagctctgagggcagaactggtgaaaaccagtagggccaagcctgctgtgtcaacaccaagcctgctgctgcgagtgctgttctgcgcagtctgtggcgagccagcatacaaatttgccggcgggggaaggaagcatccccgctatcgatgccggagcatggggttccctaagcactgtggaaacggcactgtggctatggccgaatgggacgccttttgtgaggaacaggtgctggatctgctgggggacgcagagcgcctggaaaaagtgtgggtcgctggaagcgattccgctgtggagctggcagaagtcaatgccgagctggtggacctgacctccctgatcggatctcctgcatacagggcaggctccccacagcgagaagctctggatgcacgaattgctgcactggcagctcgacaggaggaactggaggggctggaagccagaccctctggatgggagtggcgagaaacaggccagcggtttggggattggtggagggagcaggacacagcagccaagaacacttggctgagatccatgaatgtcaggctgactttcgacgtgcgaggaggactgacccgaacaatcgattttggcgacctgcaggagtatgaacagcatctgcgcctgggaagtgtggtcgagcgactgcacaccggcatgtcacccaagaaaaagcggaaggtgtga

SEQ ID NO: 187 - DNA, 1458 bp, synthetic construct
  source 1..1458: mol_type = other DNA
atgaccaagaaggtggccatctacaccagagtgtccaccaccaaccaggccgaggaaggcttcagcatcgacgagcagatcgaccggctgaccaaatacgccgaggccatgggatggcaggtgtccgatacctacaccgacgccggctttagcggcgccaagctggaaagacccgccatgcagcggctgatcaacgacatcgagaacaaggccttcgacaccgtgctggtgtacaagctggacaggctgagcagaagcgtgcgggacaccctgtacctcgtgaaggacgtgttcaccaagaacaagatcgacttcatcagcctgaacgagagcatcgacaccagcagcgctatgggcagcctgttcctgaccatcctgagcgccatcaacgagttcgagcgcgagaacatcaaagaacggatgaccatgggcaagctgggcagagccaagagcggcaagagcatgatgtggaccaagaccgccttcggctactaccacaacagaaagaccggcatcctggaaatagtgccactgcaggccaccatcgtggaacagatcttcaccgactacctgagcggcatctccctgaccaagctgagagacaagctgaacgagtccggccacatcggcaaggacatcccttggagctaccggaccctgcggcagaccctggacaaccctgtgtactgcggctacatcaagttcaaggactccctgttcgagggcatgcacaagcccatcatcccttacgagacatacctgaaggtgcagaaagagctggaagagagacagcagcagacctacgagcggaacaacaaccccagacccttccaggccaagtacatgctgtccggcatggccagatgcggctactgtggcgcccctctgaagatcgtgctgggccacaagagaaaggacggcagccggaccatgaagtaccactgcgccaaccggttccctagaaagaccaagggcatcaccgtgtacaacgacaacaagaagtgcgacagcggcacctacgacctgagcaacctggaaaacaccgtgatcgacaacctgatcggcttccaggaaaacaacgacagcctgctgaagatcatcaacggcaacaaccagcccatcctggacacctccagcttcaagaagcagatcagccagatcgacaagaagatccagaagaacagcgacctgtacctgaacgatttcatcaccatggacgagctgaaggaccggaccgactctctgcaggccgagaagaagctgctgaaggccaagatctctgagaacaagttcaacgatagcaccgacgtgttcgagctcgtgaaaacacagctgggctccatccccatcaatgagctgagctacgataacaagaaaaagattgtgaacaacctggtgtctaaggtggacgtgaccgccgacaacgtggacatcatcttcaagttccagctggcctga

SEQ ID NO: 188 - DNA, 1410 bp, synthetic construct
  source 1..1410: mol_type = other DNA
atgaacagaggaggccctaccgtgcgggccgacatctacgtgcggatcagcctggatagaaccggcgaggaactcggcgtggaaagacaggaggaatcttgtagagaactgtgcaagagcctgggcatggaagtcggacaagtgtgggtcgacaacgacctgtccgctactaagaagaacgtggtgcgccccgattttgaggccatgatcgccagcaacccccaggccatcgtgtgctggcacacagaccggctgatcagggtgacccgggacctcgagcgggtgatcgacctgggcgtgaacgtgcacgccgtgatggctggccacctggacctgagcacccctgccggcagagccgtcgcccggaccgtgacagcctgggccacctacgagggagagcagaaggccgagagacagaagctggccaacatccagaatgccagagcgggaaaaccttatacacctggcatccggcccttcggctatggcgatgaccacatgaccatcgtgacagccgaagccgatgccattagagatggcgccaagatgatcctggacggctggagcctgagcgccgtggcccggtactgggaggaactgaagctgcagagccctagaagcatggccgctggcggcaagggctggtccctgagaggagtgaagaaagtgctgaccagcccacggtacgtgggcagaagcagctacctgggcgaagtggtgggcgacgcccagtggcctccaatcctggatcctgacgtgtactacggcgtggtcgccatcctgaacaaccctgacagattcagcggcggccccagaaccggcaggacccctggtacactgctggctgggatcgccctgtgtggcgagtgcggcaaaaccgtgtccggcagaggctacagaggcgttctggtgtacggctgcaaggacacccacaccagaacaccaagaagcatcgccgatggcagagcctctagctcaacactggcaagactgatgttccccgacttcctgcctggcctgctggccagcggacaggccgaggacggacaatctgctgcttctaagcacagcgaggctcagaccctgagagagagactggacggcctggccaccgcctacgccgagggcgccatctccctgtctcagatgaccgccggcagcgaggctctgcggaagaagctggaagtgatcgaggctgacctggtgggaagcgccggaattcctcctttcgaccctgtggccggcgttgccggcctgatctctggatggcccaccacccctctgcccacacgtagagcttgggtggatttctgcctggtcgtgacccttaatacccagaaaggccggcacgccagcagcatgacagtggacgatcatgtgacgatcgagtggcgggacgtggctgaatga

SEQ ID NO: 189 - DNA, 1056 bp, synthetic construct
  source 1..1056: mol_type = other DNA
atgcctaagaagaagagaaaggtgggcagcagcgagctgatcatctctggctctagcggaggatttctgagaaacatcggcaaggaataccaggaggccgctgaaaacttcatgagattcatgaacgaccagggcgcttacgcccctaataccctgagagatctgcggctggtgttccacagctgggccagatggtgccacgccagacagctggcctggttccccatcagccccgagatggccagagagtacttcctgcagctgcacgacgctgatctggcatctaccaccatcgacaagcactacgccatgctgaacatgctgctgagtcactgtggactgcctcctctgtctgatgacaagagcgtgtctctggccatgcggcgcattagaagggaagccgccacagaaaagggcgagagaaccggccaggctatccccctgagatgggacgacctgaagctgctggacgtgctgctctcccggagcgagcggctggtcgacctgagaaatagggccttcctgttcgtggcctacaacaccctgatgagaatgagcgagatcagcagaatccgggtgggcgacctggatcaaacaggcgataccgtgaccctccatatcagccacaccaagaccatcaccacagccgccggcctggacaaagtgctgagccggcggaccacagccgtgctgaatgactggctggatgtgtccggactgcgggaacaccccgatgctgttctgtttcctccaatccacagaagcaacaaggccagaatcacaacaacacctctgaccgctcctgccatggaaaagatcttcagcgacgcctgggtcctgctgaacaagcgggacgccacccctaacaaaggcagatatagaacctggaccggccacagcgccagagtgggagccgctatcgacatggccgagaaacaggtgtccatggtggaaatcatgcaggagggcacctggaagaaaccagagacactcatgcggtacctgcggagaggcggcgtgtccgtgggcgccaacagcagactgatggacagctga

SEQ ID NO: 190 - DNA, 1746 bp, synthetic construct
  source 1..1746: mol_type = other DNA
atgacaaaccccgccagcaggcctaaggcctactcctacatcagaatgtcctccgccatccagatcaagggcgactccttccggcggcaggccgaggcttccgccaagtacgctgccgagcacgacctggatctgatcgacgattacaaactggccgatctgggggtgtccgccttcaagtccgacaacctgaccaccggcgctctggggcggttcgtggccgagtgcgaggcgggagaaatcgaggctggatcctttctgctgatcgaatccctggacaggctgtcgagagacaagatcctggacgccttcagcctgtttgccagaattctgaaaaccggtgttaagatcgtcaccctgtctgacggccaagtgtacgacggctccagcgaccaggtgggctctatctactacgctatcagcgtgatgatccggagcaacgacgagtctaaaatcaagtccaccagaggactggccaactggtcccagaagagaaagctggctgcagaacacggcgtgaagatgtcctcccagtgtcccgcctggctgaagctgtctgtggatagaaagtcctacctgatcgacaaggaaagggctaagatcgtgcagagaatcttcgaggcctctgcctctggcaaaggcgccaatctgatcaccaaggaactgaaccgggacaaggtgcctaccttcggcagaggcgccctgtgggccgaagcctttgtgtccaagaccctgcggaaccgggccgtgttaggagagttccagcctggccagtacgtgtctggtaagagacagcccgctggcgacccaatccctggctacttccctcctgtgatcgaagaggagctgttcgatatcgtgcaagcctccctgagaggccgcctcctcgctggcggcagaagaggcgagggccagtccaacatcttcacccatgtagccttctgcggctactgcggctccaagatgagacacagaagcaagggcagcagagtgaagggcaacccccctcacagatacctgacctgtttcaacagattcaacggcccaggctgcgactgcaagcccctgccttacgccgctttcgagcgctctttcctgactttcgtgcgggatgtggacctgagaggcctgctggaaggcgccaagagaaagtccgaggccaagaccatcgctgacagaatcaccgtgaacgaggaaaaagtcagaaaagctgatgagagaatccgcgactacctgatcaagatcgaaggagctcctgacctggccgagatcttcatggaacggatcagagagctgaaggctgagaaggacgacctggtcagatctatcgaagagtccaacgacgctctgtccaagatcaaatctgacaacgtgacagacgaggagctggctagcttgatctctacctttcagaacccttgcggagagaatcggatcagactggccgaccggataaagtccatcatcgagagaatcgacgtgtatcccaacggcgaaatccggaaggacgaccctgccatcgatctggtccgggcttctggcgatcctgacgctgagaagatcatcgccgccatgaacgccggctctagactgaaggacgacccttacttcatcgtgaccttccggaatggcgctgtgcagaccgtggtgcctaacccttccaaccctgatgatattcgggtttctgtgtacgcaggcgaaaagacccgacgggtggaaggctctgcctatgagtacgagtccgat

SEQ ID NO: 191 - DNA, 1527 bp, synthetic construct
  source 1..1527: mol_type = other DNA
atgcctatcgcccctgagttcctgtctctggcctaccccggacaagagttccctgcctacctgtacggcagagcctctagagatcctaagcggaagggcagatctgtgcagagccagctggacgaaggcagagccacatgcctggatgccggctggcctattgccggcgaatttaaggacgtggatcggtccgcttctgcttacgccagacggacacgggacgaattcgaggagatgatcgctggcatccaggccggagagtgcaggattctggtcgccttcgaggcaagcagatactaccgggacctggaggcttatgttcggctgcggagagtgtgcagagaggccggcgtcctcctgtgctacaacggccaggtgtacgacctgtccaagtccgccgacagaaaggccaccgctcaggacgctgtgaacgccgagggagaagctgacgacatcagagaacggaacctgagaaccaccagactgaatgctaagagaggcggcgcccacggccctgtgcctgatggctacaagagaagatacgaccccgactctggcgacctggtggaccagatccctcatcctgatagagcgggcctgatcaccgagatcttccggcgcgctgccgctgctgagcccctggctgctatctgtcgggatctgaacgagagaggcgagacaacccacaggggaaaagcttggcagagacaccacctgcacgccatcctgagaaatcccgcctacatcggccaccggaggcatctgggcgtggacaccggcaaaggtatgtgggctcctatctgcgacgacgaggacttcgccgaaaccttccaggccgtgcaggagatcttatctttgccaggcagacagctgtctcctggcccagaagctcagcacctgcagaccggaatcgccctgtgtggcgagcaccctgacgagcctcctctgagatccgtgaccgtgcgcggccggaccaactacaactgctccaccagatatgatgtggccatgagagaagatcggatggacgccttcgtggaagagtccgtgatcacctggctggcctccgacgaagccgtggctgcctttgaggacaacaccgacgatgagcggacacggaaggcccggatccggctgaaggtgctggaggaacagctggaagccgcccagaagcaggctagaaccctgcggcctgacggcatgggcatgctgctgtccatcgactccctggctggcctggaagccgagctgacccctcaaatcgacaaggccagacaagaatcccggagcctgcacgtgcccgctctgctgagagatctgctgggcaagcctagagccgacgtcgaccgggcctggaacgaggctctaaccctgccccagcggcggatgatcctaagaatggtggtgaccatcagactgttcaaagctggctctagaggcgtgcgggccatcgagcctggccggatcaccctgtcctacgtgggcgagccaggcttcaagcccgtgggcggcaaccgggccaagcag

SEQ ID NO: 192 - DNA, 1413 bp, synthetic construct
  source 1..1413: mol_type = other DNA
atgagaaaggtggccatctacagccgggtgtccaccatcaaccaggccgaagagggctattctatccagggccaaatcgaggccctgaccaagtactgcgaggctatggaatggaagatctacaaaaactactccgacgccggcttctccggaggcaagctcgaaagacccgctataaccgagctgattgaggacggcaagaacaacaagtttgacaccatcctggtgtacaagctggatcggctgtcccggaacgtgaaggacacactctacctggttaaagatgtgttcaccgctaacaacatccacttcgtgtctcttaaggagaacatcgatacttcctctgccatgggaaacctgttcctgaccctgctgtctgctatcgccgagttcgagagagaacagatcaaggagcggatgcagttcggtgtgatgaaccgggctaagtccggcaaaacaacagcttggaaaacccctccttacggctacagatacaacaaggacgaaaagaccctgtctgtcaacgagctggaagccgccaacgtcagacagatgttcgacatgatcatctccggctgtagcatcatgtccatcaccaactacgcccgggacaactttgtgggcaacacctggacccacgtgaaggtgaagcggatcctggaaaacgaaacctacaagggcctggtcaagtacagagagcagacattttctggcgaccaccaggcaatcatcgatgagaaaacctacaataaggcccagatcgctctggctcatagaaccgacaccaagacaaacaccagaccattccagggcaagtacatgctgtctcatatcgccaagtgcggctactgtggcgctcctctgaaagtgtgcaccggcagagccaagaacgatggcaccagacggcaaacctacgtgtgcgtgaacaagaccgagtccctggccagaaggagcgtgaataattataacaaccagaagatctgcaacaccggccgctacgagaagaagcacatcgagaagtatgtgatcgacgtgctgtacaagctgcagcacgacaaagagtacctgaaaaagatcaaaaaggacgataatatcatcgacatcacccctctgaagaaagaaatcgagatcatcgacaagaagatcaacagactgaacgacctgtacatcaacgatctgatcgatctgcccaagctgaaaaaggatatcgaggaactgaaccacctgaaggacgactacaacaaggccatcaagctgaactacctggacaagaagaatgaggattctctgggcatgctgatggacaacctggacatccggaaaagctcctacgacgtgcagtccagaatcgtgaagcagctgatcgacagagtggaagtgaccatggacaatatcgacattatcttcaagttc

SEQ ID NO: 193 - DNA, 1431 bp, synthetic construct
  source 1..1431: mol_type = other DNA
atgatcacaaccagaaaggttgccatctatgtgagagtgtccaccaccaaccaggctgaagaaggctactccatccagggccagatcgactccctgattaagtactgcgaggctatgggctggatcatctacgaggagtacaccgacgctggcttctccggcggaaaaatcgatcggcctgccatgagtaagctgatcaccgatgccaagcacaagagattcgatacaatcctggtgtacaagctggacagactgagcagatccgtgcgggacacactgtacctggtcaaggatgtgttcaaccagaacaacatccacttcgtgtccctgcaggagaatatcgacacctccagcgccatgggaaacctgttcctgaccctgctctctgctatcgccgagttcgagagagagcagatcaccgagcggatgaccatgggcaagatcggcagagccaagtctggcaagaccatggcctggacctacaccccttttggctacgactataacaaagagaagggcgagctgatcctggatcctgctaaggcccccatcgtgaagatgatctacaccgactacctgaagggtatgagcatccaaaagatcgtggacaaactaaacaagatggactacaacggcaaggactgcacctggttcccacacggcgtgaaacatctgctggacaatcctgtgtactacggcatgactagatataacaacaagctgtttcctggcaaccaccagccaatcatcaccaaggaactgtttgacaagacccagcgcgagagacagagaagaaggctgggcatcgaagagaatcactacaccatacctttccaggccaaatacatgctgtctaagttcctgagatgcagacagtgcggctctagaatgggcctggagctgggcagacctcggaagaaagagggaaagcggtccaagaagtactactgtctgaactccaggcccaagagaaccgcctcctgcgacacccctctgtacgatgctgaaaccctggaagattacgtgctgcacgagatcgccaaaatccagaaggacccttctatcgcttctcggcaaaaacacatcgaagatcatgaattgaaatacaagcgggaacggatcgaggccaacatcaacaagaccgtgaaccagctgtccaagctgaacaacctgtacctgaatgacctgatcaccctcgaggacctgaaaacccagaccaacaccctgattgctaagaagcgactgctggaaaacgagctggacaagacctgtgacaacgacgacgagctcgacagacaagagacaatcgccgacttcctggctctgcctgacgtgtggacaatggattacgagggccagaagtacgccgtggaactgctggtgcagagagtgaaggtggaccgggacaacatcgacatccactggaccttc

SEQ ID NO: 194 - DNA, 1656 bp, synthetic construct
  source 1..1656: mol_type = other DNA
atgcctggcatgaccaccgaaaccggccccgatcctgccggcctgatcgacctgttctgcagaaaaagcaaagctgtcaagtccagagccaatggcgctggacagcggagaaagcaagaaatctccatcgccgcccaggaaaccctgggccgaaaggtggctgccctgctcggcatgcaggtgcggcatgtgtggaaggaagtgggatctgcttctcggtttagaaagggcaaggctcgggacgaccagtccaaggccctgaaggccctggaatctggcgaggtgggcgctctgtggtgctaccggctggatagatgggacagaggcggcgctggagccatcctgaagatcatcgagcctgaggacggcatgccccggcggctgctgtttggctgggatgaggacaccggcagacctgtcctggactccaccaacaagcgggatcggggcgagctgattagacgggccgaggaggccagagaagaagccgaaaagctgtccgagagagtcagagatacaaaagcccaccagagagagaacggcgagtgggtgaacgccagagccccttacggcctgagagtggtgctggtgaccgtgtccgacgaggaaggcgacgagtacgacgagcggaagctggctgccgacgatgaggacgctggcggccctgacggtctgaccaaggctgaagccgctagactggtgttcaccctgcctgtgaccgacagactctcttacgccggcaccgctcacgccatgaacaccagagagatcccatctcccaccggcggaccctggatcgccgttaccgtgcgggacatgatccagaaccccgcctacgctggctggcagaccacaggcagacaggacggcaagcagcggagactgaccttctataacggcgaaggcaaacgcgtgtccgtgatgcacggccctcctctggtcacagacgaggagcaggaagccgccaaggcagccgtgaagggagaggatggcgtgggcgtgccactggacggctctgaccacgacacccggcggaagcacctgctgtctggccggatgcggtgtcctggctgtggcggcagctgctcctactccggcaacggctacagatgctggcggtcctccgtgaagggcggctgccctgctccaacctacgtggctcgcaagtctgtggaagagtatgtggccttccggtgggctgccaagctggccgcctccgagcctgacgatcctttcgtgatcgccgtggccgatcggtgggccgctctgacccaccctcaggcttccgaagatgagaagtacgccaaggccgcagtgagggaggccgagaagaacctgggcagactgctaagagacagacagaatggcgtgtacgatggacctgccgaacagttcttcgcccctgcttaccaggaggctctgtctacactgcaggccgctaaggacgccgtgtctgagtcctccgcctctgccgctgtggacgtgagctggatcgtggacagcagcgactacgaggaactgtggctgagagctacccctaccatgagaaacgctatcatcgacacatgcatcgacgagatctgggtcgcgaaaggccagagaggcagacctttcgacggggacgagagagtgaagatcaagtgggccgctaggact

SEQ ID NO: 195 - DNA, 1557 bp, synthetic construct
  source 1..1557: mol_type = other DNA
atgcagctggacgccaccctgacactgcgggacgagggcctgagcgctttccaccagagacacatcaagcagggtgctctgggagtgttcctgagagctatcgaggacggccggatccagcctggctccgtgctgatcgtggaaggcctggacagactctctagagccgagcccatccaagctcaggcccagctggcccagatcatcaacgccggcatcaccgtggtgaccgcctctgatggccgagagtacaaccgggaaagactgaaagcccaacctatggaccttgtgtactccctgctggtgatgatcagagctcacgaggaatccgacaccaagtccaagcgggtgaaggccgccatcaggcggcagtgcgagggctgggtcgctggcacatggcggggcatcatccggaacggcaaggaccctcactgggtcagactgggcgagcacggcaagttcgagcatgtgcctgagcgggtgctggctgtgcggacaatgatcgacctgttcctggaaggccacggcgccatcgagatcaccaggcggctgaccgagcagaacctgtacgtgtccaacgccggcaactactctgtgcacatgtacagaatcgtgagaaaccaggctctgatcggcgagaagagaatctccgtggatggagaagagttccggctggacggctactaccctccaatcctgaccagagaagaatttgccgaactgcagcagaccatgtccgagagaggcagacggaagggcaaaggcgagatccctaacatcatcacaggactgtccatcacagtgtgcggctattgtggcagagccatgaccacccagaactctaaggctcgcgcccctaagggaaaaagcgtggtcagacggctgtcctgccccatgaattccttcaacgagggatgtcctatcggcggctcttgcgagtctgagatcgtcgagagagccctcatgagatactgctccgaccagttcaatctgtctcggttgctggagggcgacgacggcaccgcccggcggaccgctcaactggctgtggctagacaaagagcatctgacatcgaagcccagatccagcgcgtgaccgacgccctcctgagcgacgacggcaaggctcctgccgcctttacccgcagagctcgcgagctggaaacccagctggaggaacagagaagagagatcgaggctctggaacaccagatcgccgctagctctgctcatggcatccccgccgccgctgaggcctgggctcagctggttgacggcgtgctggccctggactacgatgctcggatgaaggccagacagctggtggccgataccttcagaaagatcgtggtgtaccagaggggcttcgccccaatcgacgatgctgctgccgacagatggaagagatccggcaccatcggcctgatgctggtcaccaagagaggaggcatgcggctgctgaacgtggaccggagaaccggctgctggcaggccgaggatgacctggatccttctctgattccttccgatggcctgcccatgctgcctctggatgcc

SEQ ID NO: 196 - DNA, 1386 bp, synthetic construct
  source 1..1386: mol_type = other DNA
atgaaagtggccatctacgtgcgggtttccaccgacgagcaggccaaagaaggtttcagcatccctgctcaaagagagcggctgagagccttctgcgcctctcaaggctgggagatcgtgcaggagtacatcgaggagggctggtccgctaaggatctggacagacctcagatgcagcggctgctgaaggacatcaagaagggcaatatcgatatcgtgctggtgtacagactggataggctgaccagatctgtgctggatctgtacctgctgctccagaccttcgagaagtacaacgtggcctttcggtctgccaccgaggtgtacgatacaagcaccgccatgggcagactgtttatcactctggtcgctgctctggctcagtgggaaagagagaacctggccgagagagtgaagttcggcatcgaacagatgatcgacgagggcaagaagccaggcggccattctccttacggctacaagtttgacaaggatttcaactgtaccatcatcgaggaagaagctgatgtggtgcggatgatttacagaatgtactgcgacggctatggctatagatccatcgccgacagactgaacgagctgatggttaagcctagaatcgccaaggagtggaaccacaactccgtcagagatattctgaccaacgacatctacatcggcacctacagatggggcgacaaggtggtgcctaacaaccacccccccatcatctccgagacactgtttaagaaggcccagaaagagaaggagaagcggggagtggaccggaagagagtgggcaagttcctgttcaccggcctgctgcagtgtggcaactgcggcggacacaagatgcagggccacttcgacaagcgcgagcagaaaacctactaccggtgcaccaagtgccaccggatcaccaacgagaagaacatcttggaacctctgctggatgagatccagctgctgatcacctctaaggagtacttcatgtccaagttcagcgacagatacgaccagcaagaagtggtcgacgtgtccgctctcacaaaagagctcgagaagatcaagcggcagaaggaaaagtggtacgacctgtacatggacgaccggaatcctatccccaaagaggagctgttcgccaagatcaacgagctgaacaagaaagaagaggaaatctactccaagctgtctgaagtggaagaggacaaagagcctgtggaagaaaagtacaacagactgtccaagatgatcgacttcaagcagcagttcgagcaggctaatgacttcaccaaaaaggaactgctgttctctatcttcgagaagatcgtgatctatcgggagaagggaaagctgaaaaagattacactggactacaccctgaag

SEQ ID NO: 197 - DNA, 1383 bp, synthetic construct
  source 1..1383: mol_type = other DNA
atgaaagtggccgtgtactgcagagtgtccaccctcgagcagaaggagcacggccattctattgaggaacaagagcggaagctgaagtccttctgcgacatcaacgactggacagtgtacgacacctacatcgacgctggatactctggcgccaagcgggacagacctgagctgcagcggctgatgaacgatatcaacaagttcgacctggtgctggtctacaagctggaccggctgaccagaaacgtgcgggatctgctggacctgctggaaatcttcgagaagaacgacgtcagcttcagatccgccaccgaggtgtacgacaccaccaccgctatgggccggctgttcgtgaccctggtgggcgctatggccgagtgggagagagagacaatcagagaacggacccagatgggcaagctggccgctctgagaaagggcatcatgctgaccacaccacctttttactacgacagagtggacaacaagttcgtgcctaacaagtacaaggacgtgatcctgtgggcctacgacgaggccatgaagggccagtccgctaaggccatcgccaggaagctgaacaactccgacatccctccccctaacaatacccagtggcagggcagaaccattacccacgccctgcgcaaccctttcaccagaggccacttcgattggggcggcgtgcacatcgaaaataaccatgagcctatcatcaccgatgagatgtacgagaaagtcaaggatagactgaatgagagagtgaacaccaagaaggtccgacacacctccatcttcagaggaaagctcgtgtgtcctgtgtgcaacgccagactgacactgaattctcacaagaagaagtccaactccggctacatctttgtgaagcagtactactgtaacaactgcaaggtgacccctaacctgaaacctgtgtacatcaaagagaaagaagtgatcaaagtgttctacaactacctgaaaagattcgacctggaaaagtacgaagtgacacagaaacagaacgaacctgagatcaccatcgatatcaataaggtgatggaacagcggaagagataccacaagctgtacgcctctggactgatgcaagaagatgaactgtttgatctgatcaaggaaaccgaccagaccatcgctgagtacgagaagcagaacgagaaccgggaggtgaaacagtatgacatcgaagatatcaagcagtataaggacctgctgctggaaatgtgggacatctcctctgacgaggacaaggaggacttcatcaagatggctatcaagaacatctacttcgagtatatcatcggcaccggcaacacctctcggaagcggaacagcctaaagatcactagcatcgagttctac

SEQ ID NO: 198 - DNA, 1383 bp, synthetic construct
  source 1..1383: mol_type = other DNA
atgaaagtggctatctacaccagagtgtccacactggaacagaaagagaagggccactccatcgaggagcaggaaagaaagctgagagcctactccgacatcaacgactggaagatccacaaggtgtacacagatgctggctactctggcgctaagaaagatagacctgccctgcaagagatgctgaacgagatcgacaacttcgacctggtgctggtttataagctggaccggctgacaagatccgtgaaagatctgctggaaatcctggaactgttcgagaacaagaacgtgttgttcagatccgccaccgaggtgtacgacaccaccagcgctatgggcagactgtttgtgaccctggtcggcgccatggctgagtgggaacggaccaccatccaggagagaaccgccatgggcagacgggcctctgctagaaaaggcctggccaagaccgtgcctccattctactacgaccgggtgaacgataagttcgtgcccaacgagtacaagaaggtgctgcggttcgccgtggaagaggccaagaagggcacctctctgagagagatcaccatcaaacttaacaactctaagtacaaggcccctctgggtaagaactggcaccggtctgtgatcggcaacgctctgacctcccctgtggccaggggccatctggtgttcggcgacatcttcgtggaaaacacccacgaggctatcatctctgaggaagaatatgaagagatcaaactgcgcatctccgaaaagaccaacagcaccatcgtgaagcacaacgccatcttccggtccaagctcctgtgccccaattgtaaccagaagctcacactgaacaccgtgaagcacacccctaaaaacaaggaagtgtggtacagcaagctgtacttttgctccaactgcaagaataccaagaacaagaatgcctgcaatatcgatgagggcgaggtcctgaaacagttctacaactacctgaagcagtttgatctgacctcctacaagatcgagaaccagcctaaggagatcgaggacgtgggaatcgacattgaaaagctgcggaaagagcgggccagatgtcagactctgttcatcgaaggaatgatggacaaggacgaggccttccctatcatcagccggatcgacaaggaaatccatgagtacgagaagcggaaggataatgacaagggaaagacattcaactacgagaagatcaagaacttcaaatactctctgctgaacggctgggagctgatggaggacgagctgaaaaccgaatttatcaagatggccatcaagaacatccacttcgagtacgtcaagggcatcaagggcaagagacagaactccctgaagatcaccggcatcgagttctat

SEQ ID NO: 199 - DNA, 1443 bp, synthetic construct
  source 1..1443: mol_type = other DNA
atgatcacaaccaacaaggtggctatctacgtcagagtgtccaccacaaatcaagtggaagaaggctactccatcgacgagcagaaggacaagctctcctcctactgtgacatcaaggattggaacgtgtacaaggtgtacaccgacggcggcttttccggaagcaacaccgatagacctgccctggaatctctgatcaaggatgcaaagaagcggaagttcgacaccgtgctggtgtacaagctggacagactgtccagatcccagaaggacaccctgcacctgatcgaggacgtgttcatcaagaacggcatcgagtttctgtccctgcaagagaacttcgatacatctaccccattcggcaaggccatgatcggtctgctgtctgtgttcgcccagctggagagagaacagatcaaagagcggatgcagctcggcaagctgggcagagctaagtctggaaagtccatgatgtgggccaaaaccagctacggctacgactaccacaaggaaaccggcaccgtgacgatcaaccccgctcaggctctgacaatcaagtttatcttcgagtcttacctgagaggcagatccatcaccaagctgagagatgacctgaacgagaagtaccctaagcacgtgccttggtcctacagagccgtgagaaccatcctggacaatcctgtgtactgtggcttcaaccagtacaagggcgagatctaccccggcaaccacgagcctatcatctccaaagaggagtacgacaagacccagtccgagctgaagatccggcagcggaccgctgctgagaacgtgaaccctcgccccttccaggccaagtacatcctgtctggcattgcccagtgcggatattgcggcgctcctctgaaaatcatgctgggcgtcaagagaaaggacggatctcggctgaagaaatacgagtgccaccagagacatcctagaaccctgagaggcgtgaccacctacaacgacaataagaagtgcgactcgggcttctactacaaggacaagctcgaggcctatgtgctgaaggaaatctctaagctgcaggacgacgccgattacctggataagatcttcagcggcgacaacgccgagacaatcgaccgcgagagctataagaagcagatcgaagaactgtccaaaaaactgagcagactgaacgacctgtacatcgacgaccggatcaccctggaggaactgcagtctaagtctgccgaattcatctccatgcggggcaccctggaaaccgagttggaaaacgatcctgctctgcggaagaacaagcggaaagccgacatgagaaagctgctgaacgctgaaaaggtgttctctatggactacgagtcccagaaagttctggtgcggagactgatcaacaaagtgaaggtcaccgccgaggatatcgtgatcaactggaagatc

SEQ ID NO: 200 - DNA, 1341 bp, synthetic construct
  source 1..1341: mol_type = other DNA
atgctgagatgcgccatctacatcagagtgtccaccgaggagcaggccatgcacggcctgtccatggacgctcagaaagccgatctgaccgactacgctaagaagcacaactacgagatcatcgactactacgtggactccggcaagaccgccagaaagagactgtccaagcgcaaggacctgcagcggatgatcgaggacgtcaagctgaacaagatcgacatcatcatctttaccaagctggacaggtggttccggaacgtgcgggactactacaagatccaagaggtgctggaggaccacaacgtcgactggaaaaccatcttcgagaattacgatacctctaccgctaacggcagactgcacatcaacatcatgctgtccgtggctcaggacgaggccgacagaacctccgaaagaatcaaacgggtgttcgagaacaagctgaagaacaacgagcctacatctggctctctgcctatcggctacaagatcaaagagaagtccatcattatcgatgaggaaaaggcccctatcgccaaggatgtgttcgatttctactactaccaccagtcccagaccaaggtgttcaaagaaatcctcaacaaatacaacctgtctctgtgcgaaaagaccatccggagaatgctggagaataagctgtacatcggcatctacagagagcacgagaacttctgtcctcctctgatcgacaagaacaagttcgacgaagtgcagctgattctgaagaggcggaacatcaagtatatccctactaagcggatctttctgttcaccagcctgctgatctgcaaggagtgtagacataagatgatcggcaacgcccagatcagaaacacaaaggctggaaagatcgagtacatcttgtaccggtgcaaccaatcttacgctcggcacacctgcaaccacagaaaggtgatctatgaaaacaagatcgaaacctatctgctgaacaacatcgagtccgagctgaaaaagtttatctacgactacgagctggaagatatccccaaggtgaagaacaaagtgaacaaaacaaatatcaagcggaagctggaaaagctgaaagaactgtacatcaacgacctcatcgacatcgacatgtacaaagaggattacaagaagtacaccgagatcctgaataccaaagaagaaaagatcgaacagagaaacctgcagcctctgaaggacttcctgaactccgacttcaagtctctgtactcctccatctctagagaagagaagcggctgctgtggagaggcataatcagcgagatccagatcgactgcaataacgatatcaccatcatcccccatcca

SEQ ID NO: 201 - DNA, 1380 bp, synthetic construct
  source 1..1380: mol_type = other DNA
atgaaggtggccatctacactagagtgtcctcggctgagcaggccaacgagggatactccatccacgagcaaaagaagaagctcatctcctactgcgaaatccacgactggaacgagtacaaagtgttcaccgacgccggcatctctggcggctctatgaagcggcctgctctgcagaaactgatgaaacatctgtctagcttcgacctggtgctggtgtacaagctggacagactgaccagaaacgtgcgcgacctgctggatatgctcgaagaattcgagcagtacaacgtatctttcaagtccgccaccgaagtgttcgacaccacctctgctatcggcaagctgttcatcaccatggtgggcgctatggccgagtgggaaagagaaaccatcagagagcggagcctgtttggatctcgggccgctgtgcgggaaggcaactacatcagagaggctcctttctgctacgacaacatcgagggcaagctgcatccaaacgaatacgccaaggtgatcgatctgatcgtgtccatgttcaagaagggcatctccgccaatgagatcgccagacggctgaactcctccaaggtgcacgtgcctaacaaaaagtcctggaaccggaacagcctgatccggctcatgagatctcccgttctgcggggccacaccaagtacggcgacatgctgatcgagaacacccatgagcctgtgctgtccgaacacgactacaatgctatcaataatgccatctccagcaagacccacaagtccaaggtcaagcaccacgccatcttcagaggagccctggtgtgtcctcagtgcaacagaaggctgcacctgtacgctggcacagtgaaggaccggaagggctacaagtacgatgtcagaagatacaagtgcgagacatgttctaagaacaaggacgtgaagaacgtgtccttcaacgagtctgaggtggaaaacaagttcgtgaacctgctgaagtcttacgagctgaacaagttccacatccggaaagtggaacccgtgaaaaagatcgagtatgatatcgacaagatcaacaagcagaagatcaactacaccagatcttggtccctgggctatatcgaggacgacgagtacttcgagctgatggaggagatcaacgccacaaagaagatgatcgaggaacagacaaccgagaacaagcagtctgtcagcaaagagcagatccagtccatcaacaactttatcctgaaaggctgggaggaactgaccatcaaggataaagaggagctgatcctgtccaccgtggacaagatagagttcaatttcattcctaaggataagaagcacaaaaccaacaccctggacatcaacaacatccactttaagttt

SEQ ID NO: 202 - DNA, 1422 bp, synthetic construct
  source 1..1422: mol_type = other DNA
atggccgtgggcatctacatcagagtgtccacccaggagcaggcctctgaaggccattccatcgagtcccagaaaaagaaactggcttcctactgcgagatccagggctgggacgactaccggttctacatcgaggaaggcatctccggcaagaacacaaatcggcctaagctgaagctgctgatggaacacatcgagaagggaaagatcaacatcctgctggtgtacagactggatagactgacaagatctgtgatcgacctgcacaagctgctgaacttcctgcaagagcacggctgtgccttcaagtccgccaccgagacatacgacaccaccactgccaacggcagaatgtccatgggcatcgtgtccctgctggctcagtgggaaaccgagaacatgtccgagcggatcaagttgaatctggaacataaggtgctggtcgagggcgaaagagtgggagccatcccttacggcttcgacctgtctgatgatgaaaagctggtgaagaacgagaagtctgctatcctgctggacatggtcgaacgggtggaaaatggatggtccgtgaacagaatcgtgaactatctgaacctgaccaacaacgaccgcaactggagccctaacggcgtgctgaggctgctgcggaatcctgctctgtacggcgctaccagatggaacgataagatcgccgagaacacccacgagggaatcatcagcaaagagagattcaaccggctgcagcagatcctcgccgacagatccatccaccaccggcgggacgtgaagggcacctatatcttccaaggcgtgctgagatgtcctgtgtgcgaccagaccctgtccgtgaaccggtttattaagaagagaaaggacggcaccgagtattgtggtgtgctgtaccggtgccagccttgcatcaagcagaacaagtacaacctggccatcggcgaggccagatttctgaaggccctgaacgagtacatgtctaccgtggaattccagacggttgaagatgaggtgatacccaagaagtctgagagagagatgctggagtctcagctgcagcagatcgctcggaagcgggaaaagtaccagaaggcttgggccagtgatctgatgagcgatgacgagttcgagaagctgatggtggaaaccagagaaacctacgacgagtgcaagcagaagctcgagtcctgcgaggacccaatcaaaatcgacgaaacctacctgaaagaaatcgtgtacatgttccaccagacattcaacgacctggaatccgagaagcagaaagagttcatcagcaagttcatcagaaccatcagatacaccgtgaaggagcagcagcccatcagacctgacaagtctaagaccggcaagggcaaacaaaaagtgatcatcaccgaagtggaattttaccag

SEQ ID NO: 203 - DNA, 1413 bp, synthetic construct
  source 1..1413: mol_type = other DNA
atgacagtgggcatctatatcagagtgtccaccgaggaacaggtcaaggagggcttctccattagcgctcagaaagaaaagctgaaggcctactgcaccgctcaaggctgggaggacttcaagttctacgtggacgaaggcaagtctgccaaggacatgcaccggcccctgctccaagagatgatctctcatatcaagaagggactgatcgataccgtgctggtgtacaagctggacagactgacaagatccgtggtggatctgcacaacctgctgtccatcttcgacgaattcaactgcgccttcaagtccgccacagaagtgtacgacacctccagcgccatgggcagattcttcatcacaatcatctcctccgtggcccagttcgagcgcgaaaacacctccgaaagagtgagctttggcatggccgagaaggtcagacagggcgagtacatccctctggctcctttcggctataccaagggcaccgacggaaagctgatcgtcaacaagatcgagaaagaaatcttcctgcaggtggttgagatggtgtctaccggctactctctgcggcagacctgcgagtacctgaccaacatcggcctgaaaacccggagatctaatgatgtgtggaaggtgagcaccctgatctggatgctgaagaaccccgccgtgtacggcgccatcaagtggaataacgagatctacgagaacacccacgagcctctgatcgacaaggctaccttcaacaaagtggctaagatcctgtctatcagatccaagtccaccacctctagaagaggccacgtgcaccatatctttaagaaccggcttatctgcccagcatgtggaaagcggctgtctggcctgcggaccaagtacatcaacaagaataaggaaactttctacaacaacaactacagatgtgctacctgcaaggagcacagacggcctgctgtgcagatctccgagcagaagatcgagaaggcctttatcgactacatctccaactacaccctgaacaaggccaacatcagctctaagaagctggacaacaacttaaggaagcaggaaatgatccagaaagagatcatcagcctgcagcggaagagagagaagttccagaaagcctgggccgccgacctgatgaacgacgatgagttctccaaactgatgatcgatacaaagatggaaatcgacgctgctgaggaccggaagaaagaatacgacgtgtccctcttcgtgtctcctgaagatatcgccaagcggaacaacatcctgcgggagctgaagatcaactggacctctctgtcccctaccgagaaaaccgattttatttccatgttcatcgaaggcatcgagtacgtgaaggacgacgagaataaggctgtgatcaccaagatctctttcctg

SEQ ID NO: 204 - DNA, 1356 bp, synthetic construct
  source 1..1356: mol_type = other DNA
atgaaggccgccatctatatcagagtgtccacccaggaacagatcgagaattacagtatccaggctcagaccgagaaactgaccgctctgtgcagatccaaggactgggacgtgtacgatatcttcatcgacggaggctactctggctccaacatgaacagacccgccctgaatgagatgctgtctaagctgcacgaaatcgacgccgtggtggtgtacaggctggacagactgtccagatcccagagagataccatcacactgatcgaagagtacttcctgaagaacaacgtggaattcgtgtccctcagcgaaaccctggacactagctctccatttggcagagccatgatcggcatcctgtctgtgttcgcccagctggaaagagagacaatccgggacagaatggtcatgggcaagatcaagcggatcgaggctggcctgcctctgacaaccgccaagggcagaacattcggctatgatgtgatcgacaccaagctgtacatcaacgaggaagaagctaagcagctgcagatgatctacgacattttcgaggaagagaagtccatcaccaccctgcagaagagactcaaaaaactgggcttcaaggtgaagtcctactcctcctacaacaactggctgaccaacgacctgtactgcggctacgtgtcctacgccgacaaagtccataccaagggcgtgcacgagcctatcatctctgaagaacagttctacagagtgcaggagatcttcagccggatgggcaaaaatcctaacatgaaccgggattctgctagcctgctcaacaatctggtcgtttgtggcaagtgtggactgggatttgtgcacagaagaaaggacaccatctccagaggtaagaagtaccactaccggtactacagctgcaagacctacaagcacacccatgagctggagaagtgcggcaacaagatctggcgggctgacaagctggaagaattgatcatcgatcgcgtgaacaactattccttcgcttctcggaacgtggacaaagaggacgagctggacaacctgaacgagaagctgaaaaccgagcacaagaaaaagaagcggctgttcgacctgtacatctccggctcttacgaggtgtctgagctggatgctatgatggccgacatcgatgcccaaatcaactactacgaggcccagatcgaagccaacgaggaactgaagaagaacaagaaaattcaagagaatctggctgatctggccaccgtggactttgactccctagagttccgggaaaagcagctgtacctgaagtctctgatcaacaagatctacatcgacggcgagcaggtgaccatcgagtggctg

SEQ ID NO: 205 - DNA, 1410 bp, synthetic construct
  source 1..1410: mol_type = other DNA
atgaagggcgagtctgagctggacaagaaggccgccatctacatcagagtttctacacaagagcaggctacagagggctattcgatccaggcacaaaccgacagactgatcaagtacgtggaagccaaggactttatcctgtataagaagtatatcgacgccggctacagcgcttctaagctcgaaagacccgctatgcaggatctcatccaggacgtccaaagcaagaaagtggacgtggtcatcgtgtacaagctggatagactgtctagatctcagaaggataccatgtacctgatcgaggacatcttccggcctaacgacgtggaactgatctctatgcaggaaagctttgacacctccaccgccttcggctctgccaccgtgggcatgctgtccgtgttcgcccaactggagaggaagtccatctccgaaagaatgatcacaggcagagtggagcgggctaagaaaggcttctaccacaccggcggccaggacagacctccagctggctaccagttcaactccgacaaccagctgatcatcaacgagtacgaggccgctgctatcaaggacctgtttcggctgtacaacgacggcctgggaaagtctagcatctccgagtacctgaagaagaactaccccggaaaaaacaagtggctgccttcttctatcgatcggatgctgaagaactccctgtacatcggcaaggtgaagttctccggcgccgagtacgacggcatccatgagcctatcatagacgaagtgaccttctacaagacccagaaggagatcgccagacggaagcagaccaacaccaagagatacaactacgtggccctgctgggcggcctgtgcgagtgcggcatctgtggcgctaagatggccaacagacgggccgtgggacgcaagggtaaggtgtaccggtactacagatgctactccaagaaaggatctcctaagcacatgatgaaaaccgatggctgctcctccaaggcccagcagcagttcatcatcgacgaggctgtgattaacaacctgaagaacatcgacgtcgaagccgaactgaaacgcagatctgctcctcagaccaatacctctctgatctccagccagatcgagagcatcgataagcagattaacaagctgatcgacctgttccaggtggactccatgcctctggatgtgatcagcgagaagatcgataagctgaacaaagagaagcagtccatggaaaaactgctggaacggaagaataagctggacaaaaccgagctgcagcacagattcgatgtgctgaagtccttcgactgggacaattccagtatcgagtccaagcgggtggtgatcgagatgctggtgcagaaagtgatcattcacgacaactccatcgaaatcatcctggtggaa

SEQ ID NO: 206 - DNA, 1536 bp, synthetic construct
  source 1..1536: mol_type = other DNA
atgcggaccaacgagcacaacttccacaacatcgaggaggagattaagcacgtggccgtgtacctgagactgtcccggggtgaggatgagagcgagctggataaccacaagactcggctgctgaacagatgtgaactcaacaactggtcctacgagctgtataaggaaatcggatctggctctaccatcgatgatagacctgtgatgcagaaactgctgaccgatgtggaaaagaacctgtacgacgccgtgctggtggtggacctggataggctgtcgagaggcaacggcaccgacaacgacagaatcctgtattccatgaaagtgtccgaaaccctgatcgtggtggaatccccctaccaggtgctggacgctaacaacgagtccgacgaagagatcatcctgtttaagggcttcttcgcccggttcgagttcaagcagatcaataagcggatgagagagggcaagaagctggctcagagcagaggccagtgggtcaactccgtgacaccctacggctacatcgttaacaagaccaccaagaaactgaccccttctgaagaggaagccaaagtggtgatcatgatcaaggacttcttctttgaaggcaagagcacctccgacatcgcttgggagctgaacaagagaaagatcaagcctagacgggctacagaatggcggtcctcctctatcgccaatatcctgcagaatgaagtgtacgtgggcaacatcgtgtacaacaagtctgtcggaaacaagaagccctctaagtccaagaccagagtgaccaccccatacagacggctgcctgaggaggagtggcggcgcgtgtacaacgcccaccagcctctgtactctaaggaagagttcgaccggatcaagcagtacttcgagtgcaacgtcaagagccataagggatccgaggtgcgcacctacgccctgaccggcctgtgcaagacccctgacggcaagaccatgagagtgacccagggcaagaagggcaccgacgacgacctgtatctgttccctaagaagaacaagcacggcgacagcagtatctacaagggcatttcctacaacgtcgtgtacgagacactcaaagaggtgatcttgcaagtgaaagactacctggactctgtgctggaccagaacgaaaataaggacctggtggaagaactgaaagaggaactgatgaagaaggaggatgaactggaaacaatccagaaggccaagaatcggatcgtgcaaggctttctgatcggcctgtacgacgagcaggactccatcgagttgaaggtggagaaggagaaagagatcgacgaaaaggaaaaggagatcgaggctatcaagatgaagatcgacaatgcaaaaaccgtgaacaactccatcaaaaaaaccaagatcgagagactgctgtctgacgtgcagtctgccgagtctgagaaagaaatcaaccggttctacaagaccctgatcaaggagatcatcgtggatagaaccgatgaaaacgaggctaagatcaaggtcaacttcctg

SEQ ID NO: 207 - DNA, 1425 bp, synthetic construct
  source 1..1425: mol_type = other DNA
atgatcacaacaaacaaggtggccatctacgtgcgggtgtctaccaccaaccaagtggaggaaggctactccatcgacgagcagaaggacaagctggaggcttactgcaagatcaaagactggaagatctacgatgtgtacgtggatggcggcttcagcggcgccaacacccagcggcctgagctggaacggctgatctccgacgtgaagcggaagaaggtggacatcgtgctggtgtataagctggacagactgtctagatcccagaaggacacactgtttctgatcgaggatgtgttcgccaagaacgacgtggctttcatcagcctgcaggagaacttcgacacctccacccctttcggaaaggcctctataggcatgctgtctgtgtttgctcagctggagcgggagcagatcaaggaaagaatgatgctgggcaaagaaggcagagccaagaatggcaagtccatgtcttggaccaccatcgccttcggctacgactactctaaggaaaccggcgtgctgtccgtgaaccctacccaggctctgatcgtcaaccggatcttcaccgagtacctgaacggcaagcctgtggtgaaaatcatccgggacctgaacgccgagggccatgtgggcagaaagcggccttggggcgagacaatcaccaagtacctgctgaagaacgagacatacctgggcaaggttaagtataaagacaaggtgtacgagggccagcacgagcccatcatcacccaagagctgttcgatctggtgcagctggaagtggagcggagacagatctccgcctacgaaaagtacaacaaccccagaccattcagagctaagtacatgctgagcggcctgatgaagtgcggatactgtggcgcttctctgggcctgagatacaccagaaaggacaagaacggcatctctcaccacaagtaccagtgccggaatcggcactccaaggacctggaaaaaagatgcgagtctggctggtactccaaagaggaactcgagcgcggagtgatcaaggaactggaacgtatcaagttcgatcctaagtataagaatgaaaccctggccaagaaagaggaaaccatcaaagtggaagagatcaagaagcagctggagcggatcaacaaccaggtgtccaaactgaccgagctgtacctcgatgagatcatcaccaggaaggagcttgatgaaaagaacgacaagatcaagaccgaaagacaattcctggaggagcagctggagaaccagaagtccaacgtgctctccatcagaaagcggaaactgaccagactgctgaaggattttgacgtcgagaagctgtcctacgaggacgcctctaagattgtcaagaacatcatcaaagaaatcatcgtgactaaggacggcatgtccatcaccctggacttc

SEQ ID NO: 208 - DNA, 1386 bp, synthetic construct
  source 1..1386: mol_type = other DNA
atgggcaagtctatcaccgtgatcccagctaaaaaagtgcagacctctgtgctgcatcaagaccggaagaagatcaaggtggccgcctactgtcgggtgtccaccgaccaggaggagcagctgtcctcctatgaaaaccaggtgaactactacagagagttcatctccaagcacgaggactacgagctggtggacatctacgccgacgagggcatctccgcaaccaacaccaagaagcgggacgccttcaaccggctgatccaagactgtagggccggaaaggtcgacagaatactggtgaagtccatctcgagattcgccagaaacacactggattgcatcaagtacgtgcgggagctgaaggaactgggcgtgggcgtgaccttcgagaaagagaacatcgacagcctggatagtaagggcgaggttctgctgaccattctgagctctctggctcaggacgagtctcgatctatctctgagaacgccacctggggcatcagaaagaagttcgagagaggcgaagtgcgcgtcaatacaacaaagttcatgggctacgacaaggacgagaacggcagactgatcatcaaccctcaacaggctgaaaccgtcaagtttatctacgagaaatttctggagggctactcccccgagtccatcgccaagtacctgaacgacaatgagatccctggctggaccggcaaggccaactggtacccttctgccatccagaagatgctgcagaacgagaagtacaagggcgacgctctgctgcagaaaacctttaccgtggacttcctgaccaagaagagagtgcagaacgatggacaggtgaaccagtactacgtggaaaattctcacgaggccatcatcgacgaagagacatgggaaacagtgcagctcgagatggccagaagaaagacctacagagatgagcaccagctgaaatcctacatcatgcagtccgaggataaccccttcaccaccaaggtgttctgcggcgcttgtggctccgctttcggccggaagaactgggctacctccagaggaaagcggaaagtgtggcagtgcaacaacagataccggatcaagggagtcgaaggctgctacagctcccacctggacgaggctaccctcgaacagatcttcctgaaagccctggaactgctgtccgaaaacatcgacctgctggatggcaagtgggagaagatcctggccgagaacagactgcttgataagcactatagcatggctttatctgatctgctgcggcaggaacagatcgacttcaatccttccgacatgtgcagagtgctggaccacatccggatcggcctggatggcgaaatcaccgtgtgcctgctggaaggtaccgaggtggacctg

SEQ ID NO: 209 - DNA, 1233 bp, synthetic construct
  source 1..1233: mol_type = other DNA
atgagaacagtcagacgcatccagcctatcaagtctccttgcaagcctagattcaaagtggccgcctatgctagagtgtccgactcacgcctgcaccactctctgtccacccagatctcctactacaacagactgatccaggcccatcctgattgggagttggtcggaatctactacgacgagggaatttccggcaaagagcagtccaacagacagggcttcctgaatctgatcaaggactgcgaggacggcaagatcgatagaatcatcaccaagtccatcgccagatttggacggaacaccgtggaactgctgaccaccgtgcggcagctgagactgaagaacatcggcgtgaccttcgagaaggaaaacatcgacagcctgtcctctgaaggcgagctgatgctgacactgctggcttctgtggcccaggaagagtcccagaacctgtctgagaatatcagatggcggatccagaagaagttcgaaaagggaatccctcacacccctcaggacatgtacggctatcggtgggatggcgaacagtaccagatcgaacccaacgaggccaaggtgatccggaaggtgttcaagtggtacctggacggcgactccgtgcagcagatcgtggacaagctgaaccaggagcaggtgctgacccggctcggcaaccccttcaccgtggctagcatcagagagttcttcaagcaggaagcttactttggtagactcgtgctgcagaaaacctacagagaagccttctccagaaatccaaagaggaacaaaggccagagaaacaagtacatcatcgagaacgctcacgagcccatcgttacaaaggaatacttcgacctggtgctgcatgagaaagagcgaagaaaccaactgatgcaccaagagtctcacctgaacaagggcatcttccgggataagatctcttgctccgagtgcggctgtctgatgatcgtgaaagtcgattccaagcaagtgaacaagaccgtgcggtactactgcagaaccagaaaccggttcggcgcttcttcctgcagctgtcggaccctgggcgagaagcggctgctggccagctttaaatccaagctgggcatcgtgcctgacaaggagtgggtggaaaacaacatcaagcacatcgagtacgacttcggctaccggatcctgcgggtgacacctgtgaagggcagaaagtacctgatcgagatcagagagggcagatac

SEQ ID NO: 210 - DNA, 1419 bp, synthetic construct
  source 1..1419: mol_type = other DNA
atgcggaacaaggttgccatctacgtccgggtgtccacagctagccaggccgacgagggctactccatcgacgaacagaaaagcaagctggaggcctactgcgagatcaaggactggaagatctacgacacctacatcgatggcggcttctccggggccaacacccagaggcccgaactggaacggctgatttctgatgccaagcggaagaagattgatatcgtgctggtgtacaagctggacagactgtccagatctcaaaaggacacactgttcctgatcgaggatgtgttcgctaagaacgacgtggctttcatcagcctgcaggagaacttcgacacctctacccctttcggcaaggcctccatcggcatgctgtccgtgttcgcccagctggagcgcgaacagatcaaagagcggatgatgctgggcaaagagggcagagccaagaatggcaagtccatgtcttggaccaccatcccttttggctacgactactccaaagagacaggcatcctgagcgtgaaccccacccaagctctgatcgtgaagagaatcttcaccgagtacctgaacggcaaatctgtggtgaagatcatccgggacctgaatgccgagggccatgtgggccggaagcggccttggggcgaaaccatcaccaagtatctgctgaaaaacgaaacctacctcggaaagtctaagtataagggcaaggtattcgaaggccagcacgacgccatcatctctcaggaactgtttgatctggtgcagctggaagtggagaagagacagatctccgccttcgagaagtacaacaaccctagacctttccgggctaagtacatgctgtctggcctaatgaagtgcggctactgcggcgcttctctgggactctacgtggcccctaagaacaagaacggcgtgagcaagtacaagtaccagtgtagacaccggtaccacaaggacaaagccatcagatgcaactccggatggtactccaaggacgagctggagaaaagagtgatcaaagagctcgagcggctgaagttcgatcctaagtacaagaaagaaaccctggccaagaaagatgagacaattaaggtggaggacatcaagaagcagctggaaagaatcaataagcaggtgtccaagctgaccgagctgtacctggacgaggtgatcaccagaaaggacctggacgaaaagaacgccaagatcaagaccgaaagacagtacctggaggagcagctggagaaccagaagtccaacgtgatgtccatccgaaagcggaagctgtctagactgctgaaggacttcgacatcgagaagctgtcctacgaggaagcttctaagatcgtgaagtccgtcatcaaggaaatcgtcgtgaccaaggacgacatgaccatcactctggatttt

SEQ ID NO: 211 - DNA, 1389 bp, synthetic construct
  source 1..1389: mol_type = other DNA
atgaaggtggccacttacgtgcgcgtgtccaccgacgagcaggctaaggagggcttctccatccccgcccaaagagagcggctgagagccttctgcgagtctcagggatgggaaatcgtggaagagtacatcgaagagggctggtccgccaaagacctggacagacctcagatgcagcggctgctcaaggatatcaagaagggcaatatcgacatcgtgctggtgtacaggctggatagactgacccggtctgtgctggatctgtacctgctgctgcagacctttgagaagtacaacgtggctttcagatccgctaccgaggtgtacgacacctctaccgccatgggcagactgttcattacccttgtggccgccctggctcagtgggagcgggagaacctggccgagagagtgaagttcggcatcgagcagatgatcgacgagggaaagaagcctggcggccactctccatacggatacaagtttgacaaggacttcaactgcaccatcatcgaggatgaggccaacaccgtgcggatgatttacagaatgtactgcgacggctacggctaccactccatcgctaagcgcctgaatgagctgggcatcaagcctagaatcgccaaagagtggaaccacaacagcgtccgggacatcctgaccaacgacatctacatcggcacctatagatggggcaacaaggttgtgctgaacaaccatcctcctatcatctccgagacactgttcagaaaggtgcagaaagaaaaagaaaagcggcgggtggaccggaccagagtgggcaagtttctgctgacaggcctgctgtactgtggcaattgcaacggccacaagatgcagggcacctttgacaaaagagaacagaaaacctactaccggtgtctgaagtgcaaccggatcaccaacgagaagaacatcctggaacctctgctggatgagatccagctgctgatcacatctaaagagtacttcatgtccaagttctccgaccagtacgatcaaaaggaggaagtggacgtgtctgctctgaagaaggagctcgaaaagatcaagagacagaaggaaaagtggtacgacctgtacatggacgacagaaaccccatccctaaggaagatctgttcgccaagatcaacgagctgaacaagaaggaagaagagatctataacaagctgaacgaggtcgaacccgaggacaaggagcctgtcgaagaaaagtacaacagactgagcaagatgatcgacttcaagcagcagttcgagcaagctaatgatttcaccaagaaagaactgctgttcagcatcttcgaaaaaatcgtgatctatcgggagaagggcaagctgaaaaagatcaccctggactacaccctgaag

SEQ ID NO: 212 - DNA, 1356 bp, synthetic construct
  source 1..1356: mol_type = other DNA
atgctgcgcgtggctctgtatatcagagtgtctaccgaggagcaggccctgaacggcgacagcatccggacccagatcgaggccctggaacagtactccaaggagaacgacttcaacatcgtgggcaagtacatcgacgagggctgttctgccaccaacctgaagcggcctaatctgcaaagactgctgcgggacgtggaaaaagacaaagtggacctggtgctgatgactaagatcgatcggctgtctagaggagtcaagaactactacaagatcatggaaacactggagaagcacaagtgcgactggaaaaccatcctggaaaactacgactcctccaccgccgctggcagactccacatcaacatcatgctgtccgtggccgagaacgaggctgctcagacctccgagagaatcaagttcgtgttccaggacaagttgagaagaaaggaagtgatctctggtacaatccccatcggctacaaaatcgagaataagcatctggtgatcgataaagagaagaagtacattgtgaaggccatcttcgacgagtacgagaagtctggctccgttaggaccctgatcgaaaccatcaacaacctgcacggcgaactgtactcctataacaagatcaagaacatcctgagaaacgagctgtacatcggcatttacaataagagaggcttctacgtggaggactactgcgagcctatcatcagcaagaagcagttcaagcagatccagcggatcctggaaaagaataagaaaaccacaccaaacaagaacatccactaccacatcttcagcggcctgctcaagtgcaaggagtgtggctacaccctgaagggcaactcctccaacgtgggagagaagctgtacctgtcttacagatgctccaccttttacctgaacaagaactgcgtgcacaacgtgacccacaacgagaagcatattgagaactatctgctgaccaacctgaagcctcagctgcacaagcacatggtgaagctggaagcccagaacgaaaagatcagacggaacaaaaagtccaacaagaaggatgagaaaaagaaaatcatgaagaaactggataagatcaaggacctgtacctggaggacctgatcgataaagaaacctaccggaaggactacgagaagctgcagtcccagctggacaacatcaccgaggaacaagagtctcagatcatcgacacctctcacatcaagaagtttctggacatcgacatcaatgagatgtactctgatctgagcagagtcgagcggcggagattctggctgtccatcatagactacatcgagatcgataacaacaaaaacatcaccatcaacttcatc

SEQ ID NO: 213 - DNA, 1776 bp, synthetic construct
  source 1..1776: mol_type = other DNA
atgaagatcaccctgctgtactacatcaagaagttcaacatctactgcaacagatacctgagccagcagatcaacatctccgtggacatcatcggcttctaccagttcaagaacgtcaccaactctgtgaccgacgtgctgaagagaggtgataatctggacagaatctgtatctacctgcggaagtccagagccgatgaagaactggaaaagaccatcggagtgggcgaaaccctgagcaagcacagaaaggctctgctgaagttcgccaaggaaaagaagctgaatatcatggaaatcaaagaggaaatagtgtccgctgactccatcttcttcagacctaagatgatcgaactgctgaaggaggtggagaacaaccagtacaccggcgtgctggttatggacatccagagactgggcagaggcgacaccgaggaccagggcatcattgctagaatcttcaaggagtctcacaccaagatcatcacccctatgaaaacctacgacctggacgacgatttggacgaggactactttgagttcgagagtttcatgggccgcaaagagtacaagatgatcaagaagcggatgcagggcggcagagtgcggtccgtggaagatggcaactacatcgccaccaatcctccatttggctacgacatccactggatcaacaagtccaggacactgaagttcaactccaaggaatctgagatcgtgaaactgatctttaaactgtataccgagggaaatggcgctggcaccatctccaactacctgaactccctgggctataagaccaagttcggcaacaacttcagcaactcttctatcatcttcatcctgaagaaccctgtgtacatcggaaagatcacctggaagaagaaggacatcagaaagtccaaggatcctcacaaggtcaaagatacccggaccagagacaagtccgagtggatcatcgccgacggcaagcacgagcctatcatcgacgaaaagatctggaacaaggctcaagagatcctgaacaacaagtaccacatcccttacaagatcgccaacggccccgctaaccctctggccggagtggtgatctgctccaagtgcaactccaaaatggtgatgcggaagtacggcaagaagctgcctcatctgatctgcaataacaaggagtgtaacaataagtccgccagattcgactacatcgagaaggccgtgctggaaggcctggacgagtatctgaagaactacaaagtgaacgtgaaggccaacaacaaaaccagcgatatcgagccctacgagcagcagtctaacgccctgaacaaagagctgatcctcctgaacgagcagaaactgaagctattcgactttttggaaagagagatctacacagaagagatctttcttgagagatctaagaacctggatgagcggatcaacaccaccacactggctataaacaagatcaagaaaattctggacaacgagaaaaagaagaacaacaagaacgacatcgtcaagttcgagaaaatcctggaaggctacaagaaaaccaacgatatccagaagaaaaatgaactgatgaaatctctggtgttcaagatcgagtataagaaagaacagcaccagcggaacgacggcctgctgtacatctacttcctgagcttctgcgtgcggtgcatctcctacctgacacaattcatttccttcttcgtgtacccctaccggatcctggagatctacctgaccttctcttttttcatcatctcttacgagcat

SEQ ID NO: 214 - DNA, 1512 bp, synthetic construct
  source 1..1512: mol_type = other DNA
atgcggatctgcatgtacctgcggaagtccagagctgatgaggaactggaaaagaccctgggcgaaggcgagactctgagcaagcacagaaaggctctgctgaagttcgccaaggagaaaaatctgaatatcgtggagatcaaagaggaaatcgtgtctggtgagtccctgttcttcagacctaagatgctggaactgctgaaagaaatcgagaacaaacagtactccggcgtgctcgtgatggacatgcagagactgggaaggggaaacatgcaggaccagggcatcatcctcgagacatttaagaaatctaacaccaagatcatcacccctatgaaaacctacgacctgtctaacgacttcgacgaagagtactctgagttcgaggccttcatgtcccggaaggaacttaagatgatcaatcggcggatgcaaggcggcagagtgcggagcgtcgaggacggcaactacatcgctaccaacgccccctacggctacgacatccactggatcaacaaggccagaaccctgaagcccaaccagaaggaatctgaaatcgtcaagctgatcttcaagctctacatcgagggcaacggcgctggcaccatcgctaagcatctgaacagcctgggctataagaccaagttcggcaactccttcaacaactcctccatcatcttcattctgaaaaaccctgtgtatatcggcaagatcacctggaagaaaaaggacattcggaagtccaaggatcctaacaaagtgaaggacacccggaccagagacaagtctgagtggatcatcgtggacggcaagcacgaccctatcatcgaccagatcacctggaagcaggctcaagagatcctgaataaccggtaccacgtgccttacaagctggtcaacggccctgccaaccccctggccggcctgatcatctgtaccacctgcaagtccaagatggtgatgagaaagctgagaggcaccgacagaatcctgtgcaagaacaacaagtgcaacaacatctccaacagattcgatgccgtggaaaagtccgtggtggaatctctggaaaactacctgaaggcctacaaggtgaacctgcctgagctgaacaagacctccaacctgaaactgtacgagcagcagatcagcacactgaagaaagaactgaaaattttgaacgaacagaaactgaagctgttcgattttctggagcgcggaatctacgacgaggataccttcctgaagagatctaagaacctggacgagagaatcgagatcaccaacgagtctctgtctaatctgaatcagatcatcgccaaggagaacaaggccatcaagaaagaagatatcatcaagtttgagaaggtgctggatagctacaagtccaccgctgacatccggctgaaaaacgagctgatgaaaaccttaatcttcaagatcgagtacaccaagaacaagaagggcaatgacttcaagatcaaggtgttccctaagctgaagccactgaacatc

SEQ ID NO: 215 - DNA, 1635 bp, synthetic construct
  source 1..1635: mol_type = other DNA
atgatcgccgctatctactctagaaagtctaaattcaccggcaagggcgagtccgtggaaaaccagatcgaaatgtgcaaggaatacctgaagagaaacttcaataacatcgatgacatcgaaatctacgaggacgagggcttctctggcaaggacaccaaccggcccaagtttaagaagatgatcaaggccgctaaaaacaagaagttcaacatcctcatctgctaccggctggacagaatctctcgcaacgtggctgatttcagcaataccatcgaggagctgcagaaatacaacatcgactttatatccatcaaggagcagttcgataccagcaccccaatgggcagagccatgatgaacatcgctgctgtgttcgcccagctggagcgggaaaccatcgccgagcggatcaaggacaacatggtggaactggccaagaccggacggtggctgggcggcacctctcctctgggctacaagtccgaacccatcgagtactccaatgaggacggcaagtccaagaagatgtacaagctgaccgaggttgagaacgagatgaacatcgtgaagctgatctacaagctgtacctggagaagagaggctttagctctgtcgccacctacctgtgcaagaacaagtacaaaggcaagaacggcggcgagttctccagagagacagctaggcaaatcgtgatcaatcctgtgtactgtatctccgacaagacaatcttcaagtggttcaaatccaagggcgctaccacctacggcacacctgacggaattcacggcctgatggtgtacaacaagcgggaaggcggaaagaaggacaagcctatcaacgagtggatcatcgccgtgggcaagcatagaggagtcatctcctctgatatctggctgaagtgccaaaatctgatccagcagaacaacgctaagtcctcccctagatccggtactggagagaagtttctgctgtccggcatggtggtgtgtaaggagtgcggctccggcatgagctcctggagccacttcaacaaaaaaaccaacttcatggaaagatactacagatgcaacctgcggaatagagcctccaaccggtgttccaccaagatgctgaatgcctacaaggccgaggaatacgtggccaactacctcaaggaactagatatcaacgccattaaaaagatgtaccactctaacaagaagaacatcatcgactatgacgccaagtatgaggtgaacaagctgaacaagagcatcgaggagaacaagaagatcatccagggcatcatcaagaagatcgctctgttcgacgacctggatatcctgggcatgctgaagaacgaactggagagactgaaaaaagaaaacgacgagatgaagatcaaactgaaagaactgaagtccatcctggaattggaggatgaagaggagatcttcctgtctaccatggaggagaacatctctaacttcaaaaagttctacgacttcgtgaacatcacccagaagcggattctgatcaagggcctggtggaaagtatcgtgtgggacacaggcggtgaggaaaagatcctggagatcaacctgatcggctctaacaccaagctgccttccggcaaggtgaagcgaagagag

SEQ ID NO: 216 - DNA, 1455 bp, synthetic construct
  source 1..1455: mol_type = other DNA
atgtccaaaaaggtggccatctatacaagagtgtccaccaccaaccaggccgaggaaggctactccatcgacgagcagatcgacaagctgaaaatgtactgcgaggccatggactggaaggtgtctgagatctacaccgacgccggcttcactggctccaagctgaccagacctgccatggaaaagatgatcaccgacatcggcctgaagaagttcgataccgtgatcgtgtacaagctggacagactgtccaggtccgtgcgggataccctgtacctggtcaaggatgtgttcaccaagaatgagatcgactttatcagcctgtctgagtctattgacacctcctccgctatgggttctctgttcctgacaatcctgagcgctatcaacgagttcgagagggagaacataaaagaacggatgaccatgggcaagatcggcagagccaagtctggaaagtccatgatgtgggctaagaccgccttcggctactctcacaaccaagagacaggcatcctggaaatcaaccctctggaagcttccatcgtggaacagatcttcaacgagtacctgaagggcacctctatcacaaagctgcgggacaagctgaacgaggatggccacatcgccaaggagctgccttggtcctacagaaccatcagacagaccctggacaaccccgtgtactgtggatacatcaagtacaaaaacaacacctttgagggcctgcacaagcccatcatctcccacgaaacctacctcagcgtgcagaaagaactggaagccagacaacagcagacctatgagaagaacaataatcctagaccatttcaagccaagtatctgctgtctggcatcgctagatgcggatactgtggcgctcctctccggatcgtgctgggccatcgccggaaggacggcagtagaaccatgaagtaccagtgcgtgaacagattccctcgcaaaaccaagggcgtgaccacatacaacgataacaagaagtgcgactccggcgcttacgacatgcagtggatcgaggacatcgtgctgaaaaccctgaacggcttccagaagtccgacaaaaagctgcggaagatcctgaatatcaaggaagagtccaaggtggacaccagcggatttcagaagcagctgaagtccatcaacaataagatccagaagaactccgatctgtacctcaacgacttcatcaccatggacgacctgaaaaagcggaccgagatgctgcagggcgagaagaaactgatccaggccagaatcaacgaagtggataagccttccacatctgagatcttcgacctggtcaagtctgagctgggcgaaaccaccatctctaagatctcctacgaagataagaagaagatcgtcaacaacctgatctctaaagttgacgtgaccgccgacaacatcgatatcatcttcaagttccagctggct

SEQ ID NO: 217 - DNA, 1374 bp, synthetic construct
  source 1..1374: mol_type = other DNA
atgaacgagcaaaaggacaagctgaagaaatactgcgagattaaggactggaccatcgtcaaagagtacgtcgatcctggccggagcggctccaacatcaacagaccatccatgcagcagctcattaaggacgccgataccggcctgtacgacgctgtgctggtgtacaagctggaccggctgtctagatctcagaaggacaccctatatctgatcgaggacgtgttccagaagaacaacatccacttcatctctctgtccgagaacttcgacacctccaccgcctttggaaaggccatgatcggcatcctctccgtgttcgcccagctggaaagagagcagatcaaagagcggatgtctatgggcagagtgggcagagccaaatccggcaaaatcatggaattcaacaaccccgcctttggttacgaggtggatggcgacaactacaaagtggacccactgcgggccgagatcgtgaagagaatctacaagatgtacctgagcggcacctctatcaacaagatcaaggaaaccctgaacctggaaggccacatcggcaacaagaagaactggtccgacaccagaatcagatatatcctgtccaatcccacctacctgggaaagatccggtacgacggcaaaacctacgacggcaagttctcccctatcatcgacgaggaaaccttcaacaagacccagaacgaactgaaagagagacagaccgctacatacaagagattcaacatgaagctacgcccctttcagtctaagtacatgctgtctggcctgctgaggtgcggctactgcggcgctaccctgttcgtgaactcctatgtgtacaacggcaagcggaagctgcgatacaactgtccttctacctacaagtccaagcaaaaaacacggacatacaagatcatggaccccaactgccctttcaagctggtgtacgccaaggatctggaacctgctgtgatcaacgagatcaagaatctggctctgaaccctcagtccatccagaagcctgtgaagaagaaacctgatatcgatgtggaagccatccagaaagagctggccaaggtgcggaagcagcagcagagactgatcgatctgtacgtgatcagcgacgacgtgaatatcgacaatatcagcaagaagtctgccgacctgaagctgcaagaggagacactgaagaagcagctggctcctctggaggagcctaacgacgacgataagatcgtggccttcaatgagattctggctcagatcaaggatatcgactccctggactacgataagcagaagttcatcgtcaagaagctgatcaagaaaatcgacgtgtggaacgacaacaagatcaagatccattggaacatc

SEQ ID NO: 218 - DNA, 1545 bp, synthetic construct
  source 1..1545: mol_type = other DNA
atgaaaaccgccatctacctgagaaagtctagagccgatctggaggccgaggctagaggcgagggcgagacactggccaagcaccggtcgacactcctgaagatcgccaaggagatgaacctgaacgtgctgtctgtgagagaagaaatcgtgtccggcgagtctctggtcaagcggcccgagatgctggctctgctggaagagatcgaggacaacaagtacgacgccgtgctgtgcatggatatggacagactgggaaggggcggcatgaaggaacagggaatcatcctggaaaccttcaagcggtccaacaccaagatcatgacccctagaaagacctacgacctgaacgacgagtgggacgaggagtactctgagtttgaggccttcatggccagaaaagaacttaagatcatcaccagaagaatgcagagaggccggatcgccagcgtggaagctggcaactatctcggcacccacgctccattcggctatgatatccaccggctgaacaaaagagagagaaccctgacaatcaactccgaggaggcctccgtggtgcggatgatcttcgactggtacgccaacgaggacatgggcgccagtgctatccggaacaagctgaacgacttgggctacaagtccaagctgggcaatgactggaacccctactccatcctggatatcctgaagaacaacatctacatcggcaaagtcacctggcagaaacgtaaggaagtgaagcggcctgatgctgtcaagagatcctgtgccagacaggacaagtccgattggatcatcgctgacggcaagcacgagcctatcatccctgagtccctgttcgagcaggcccaagagaagctgaattctcggtaccacgtgccatacaataccaacggcattaagaaccctctggctggaatcattaagtgtagcaagtgcggctactccatggtgcagagataccctaagaatcggaaggaaaccatggactgcaagcatagaggctgcgagaacaagtctagctacaccgagctgatcgagaagcgcctcctggaagctctgaaggaatggtacatcaactacaaggctgactttgaagctcacaagcagggcgacaagctgaaggagacacaagtgatccagatgaacgaggctgccctgcggaagctggaaaaagaactggtggacgtgcagaagcagaagaacaacctgcacgacctgctggagcggggcgtgtacaccgtggacatgttcctggaaagatctcaggtgatctccgaccggatcaacgagatcacctctaccatggaaaacctgaaaaaggagatcaagaccgaaatcaagaaggagaaagtgaagaaggacaccatcccccaggtggagcatgtgctggacctgtacttcaagactgacgatcctaagaaaaagaattctctgctgaagtccgtgctggaaaaggccgtgtacaagaaagaaaaatggcagagactggacgacttcgagctggttctgtaccctaagctgcctcaggatggagacatc

SEQ ID NO: 219 - DNA, 1548 bp, synthetic construct
  source 1..1548: mol_type = other DNA
atgtaccggccagagagcctggacgtgtgcatctatctgcgcaagtctcggaaagatgtggaagaagaacggcgggctattgaagagggctcctcctacaacgccctggaaagacacagaaagagactgttcgccatcgctaaggccgagaaccacaacatcatcgacatcttcgaggaagtggcctctggggagtctatccaagagcggcctcagatgcagcagctgctgcggaagttggaaggcaacgagattgacggagtgctggtcatcgatctggataggctgggcagaggcgatatgctggacgctggcatgattgacagagccttcagatactcctctaccaagatcatcacccctaccgacgtgtacgaccccgacgacgagtcctgggagctcgtgttcggcatcaagagcctgatctccagacaagaactgaagtccatcaccaagaggctgcagaacggccggatcgattccgtgaaagaaggcaagcacatcggtaagaaaccaccttacggctacctgaaggatgagaacctgagactgtaccctgatcctgagaaagcttggatcgtgaagaagatcttcgagctgatgtgcgacggcaaaggcagacagatgatcgccgctgagctggacagactgggcatcgaccctcctgtgaccaagcggggcgcctgggactcttctacaattacctctatcatcaagaacgaggtgtacaccggcgtgatcgtgtggggaaagttcaagcacaagaagcggaacggcaagtacaccagacataagaatcctcaagagaagtggatcatgtacgagaacgctcacgagcctatcatctctaaggaactgttcgacgccgccaacgaggcccattcttcgagacacaagcccgccgtgatcacttccaagaaactgaccaaccccctggccggcatcctgaagtgcaagctgtgtggctacaccatgctgatccagacccggaaggaccggcctcacaactacctgagatgcaacaaccccgcctgtaaaggcaagcagaagcagtctgtgttcaacctggttgaggaaaagctcctgtatagcctgcagcagatcgtggacgagtaccaggctcagaaggtggaagaagtggagatcgacgattccaagctgatctccttcaaggagaaggctatcatctccaaggagaaggaactcaaagaactgcaggcccagaagggcaacctgcacgacctgctggaacagggcatctacacagtcgagatctttctggaaagacagaagaatctggtcgaaagaatcacctccatcgagaacgacatcgaggtgctgcagaaggagatcgagacagagcagatcaaggagcacaacaagaccgagtttatccctgctctgaaaacagtgatcgagagctaccataagaccaccaatatcgagctgaagaatcagctgctgaaaaccatcctgtccaccgtgacctactacagacaccctgactggaaaaccaacgagttcgaaatccaggtgtactttaaaatc

SEQ ID NO: 220 - DNA, 1539 bp, synthetic construct
  source 1..1539: mol_type = other DNA
atgaagtacctggctctgcatgagaactcccggatcgccgtgtacagccggaagtccagagaggacagagactccgaggataccctggccaagcaccggaacgagctggaatacctgatcaagagagaaaacttcaaaaacgtgcagtggttcgagaaggtggtgtccggcgaaaccatcgacgagcggcctatgttctccctgctgctgcctagaattgaaaacggcgagttcgacgctgtgtgtgccgtggccatggaccggctgtctagaggctcccagatcgattctggaagaatcctggaggcctttaagcagtccggcaccctgttcatcacccctaagaaaacctacgacctgtccatcgagggcgacgagatgctgtccgagttcgaatccatcatcgccagatctgagtacagagctatcaagcggagaaccatcaacggcaagaagaatgccacccgcgaaggccggctgcacagcggatccgtgccttatggttacaagtgggacaagaacctgaaagctgctgtcgtggtggaagagaagaagaagatctatcggatgatgattaagtggtttctggaagaagagtactcctgcaccgtgatcgctgagatgctgaatgaactcaaggtgccctccccctcaggcagatctatctggtacggcgaggtggtgtctgagatcctgtccaacgacttccacagaggatacgtgtggttcggcaagtataagaagtccaagagcaacaacagcatcgtgcagaacaagaacctggatgaggttctcatcgccaagggccaccatgaaaccatgaaaaccgatgaggagcacgccctgatcctgaaccggatcgagaagctgcggacctacaaggtggctggcagacggctgaacatgaacacccatagactgtccggcatcgtgcggtgtccttactgccacaaggctcaggccatcgagcaaccaaagggcagacggaagcacgtgagaaagtgcctgagaaagtccgctgagaggaccaaagagtgcgaggaaacaaaaggcatccacgaggaagtgctgtttcagtctatcatgaaagagatcaagaaatacaatgagtctctgttctctcctaccgagcaggacgtgaacgacgactcctacactgcccagctgatcggcctgagggagaaggccgtgaagaaggctaagggccgcatcgagcggatcaaagagatgtacctggacggagacatctccaaaaccgagtacaaggaaaagctgaagatcagccaagagacactgcagaaggctgagaacgaacttgccgaactgatagcctctacagagttccagaacgccctgtctgccgagacaaagaaagagaagtggtcccaccacaaggtgcaggaaatgatcgagagcaccgacggcatgtccaactctgaaatcaacttgatcctgaagatgctgatctctcacgtgacctacaccgtcgaagatctgggcgatggcaccaagaatctgaacatcaaggtgtactacaac

SEQ ID NO: 221 - DNA, 1740 bp, synthetic construct
  source 1..1740: mol_type = other DNA
atggaccctcagcacaagcctacccgggctctgatcgtgatccggctgtcccggctgacagacgaaaccacctctcctgagcggcagctggaggcctgcgagagattctgcgccgcaagaggctgggaggtcgtgggcgtggctgaagatctggacgtgtctgctggaaccaccagccccttcgagcggccttctctgagccagtggatcggcgatggtaaggacaacccaggaagaatcggcgagttcgacaccgtggttttctacagagtggatcggctcgtgcggagagtgcggcacctgcacgacgtgatcgcctggagcgagcgcttcgatgtgaacatggtgtccgccaccgagtctcacttcgacctgtccacaaccattggcgctctgatcgctcagctggtggcctccttcgccgagatggaactggagggcatctctcagagagctacctctgctcacagacacaacgtgcagctgggcaagttcgtgggcggctcccctcctttcggctacatgcctgaagaaacccctgatggctggcggctggtgcacgatcccgacgtcgtgcccatcatcctcgaggtggtggacagagtcctggaaggcgaacccctgagaagaatcaccgacgatctgaacgcccggggcgctacaaccgcccgggacctggtgaagcagagaaagggcaaagaaaccgagggccacaagtggcactccaacgtgctgaagcggcggctgatgtcccctgccatgctgggctacgccctgagaagagaacctctgaccgactccaagggcaagcccaaactgtctgccaagggcgccaagctgtacggccctgaggaaatcgtgagaggacctgacggcctgcctgtgcaacgcgctgagcctatcctgcctaagcctctgttcgaccgggtggtggctgagctagaagctagagagctacagaaagagcctaccaagcggatcaactccatgctgttgagagtgctgtactgcggcgtgtgtggccagcctgtctaccgggcaaaaggacagggcggtagatccgacagataccggtgcagatccatccaggatggcgccaactgtggcaacccctccgtgctgacctatgagctggacgacctggtggaagagtctatcctggtgctgatgggcgactctgagagactggcccatgtgtggaaccctggcgaggacaatgctagcgagctggctgaagtggaagcccggctggccgacagaaccggcctgatcggagtgggagcctacaaggctggcaccccccagagagccaccctggataccctgatcgaggctgatgccaagctgtacgagaggctgaaggccgccacccctagacctgctggctggacctgggaaccaacaggcgaaaccttcgccgagtggtgggctgctctggacaccggcgccagaaatgtgtacctgcggaacatgggggtcagagtcacctacgacaagcggcctgtgccagagcaggtgtccgccggcgagaagcctagagtgcatctggaactgggcgaagtgcggaagatggccgaacaagtggctgtgaccggcaccatcggaacactgaccagaaactacacaagactgggagagatcggcatcacccacgtggacatcgacgccggatctggcaaggccgtgtttgtgacaaagtccggcgagcggttcgagctccctctgaacatccctgaggaa

SEQ ID NO: 222 - DNA, 1620 bp, synthetic construct
  source 1..1620: mol_type = other DNA
atgaaggctatcgccatctacgccagaaagtctctgttcaccggcaagggcgactccattggcgcccaggtggacacctgcaagcggttcatcgactacaagttcgccaatgaggactatgagatccggacatttaaggacgaaggctggtccggcaagaccactgacagaccagattttaccaacatggtgaacctgatcaagtctaagaagatcgactatgtcatcacctacaagctggaccggatcggccggacagctcgggacctgcacaacttcctgtacgagctggataatctgggaatcgtgtacctgagcgccaccgagccttacgacacaaccacatctgccggaagattcatgatcagcattctggctgctatggctcagatggaacgcgaaagactggccgagagagtgaagtccggcatgatccagatcgccaagaagggaagatggctgggcggccagtgtcctctgggcttcgactctaagagagagatctacatcgatgacatggggaaagagcggcagatgatgcggctgacccctaacaaggaggaaatcaagatcgtgaagctgatctacgacaagtacctggagatgggatccatgtcccaagtgcggaagtactgcctggaaaactccatcagaggcaagaacggcggcgacttctccacaaacaccctgaagcagctgctgacctctcctatctacgtcaagtcctccgacaacatcttcaagtacctggagtctcagaatatcaatgtgttcggcacccccaacggcaacggcatgctgaccttcaacaagaccaaagagatcaggatcgagcgggacaagtccgagtggattgctgctgtgggcaagcacaagggcatcatcgacgataacaagtggctgcagatccagcagcagctgcagcagcagtctgaaaagcagatcaagagctctggcagacagggcacgacctccaccggcctgctgtccggcatcatcaagtgctccaagtgcggcaacaacctgctgatcaagaccggacacaagtccaagaaaaaccctggcaccacctactcctactacgtgtgtggcaagaaggataactcttacggccataagtgcgacaacaaaaacgtgagaaccgacgaggccgactccgccgtgatcacccagctgaaactgtacaacaaagaactgctcatcaaaaatctcaaggaagccctgatccaaaacgaaaagaccgataccgacaacatcgagatcctggagtccaaattaaaagaaaaagagaaggccgtgtccaacctggtgaaaaagctgtctctgatcgacgacgagtccatcagcaatatcatcctgaacgaggttaccaatatcaacaaggaaatcaacgacatcaagctgcaattgtctaacgagacactgaagatcaacgaagtgaccaaggccacactggataccgagatctacatcaagatcctggagaactttaacaagaagatcgacgatatcaccgaccccatcgaaaagatgaacttgctgaagtctgctctggaatccgtggaatggaacggcgattctggcgagttcaagatcaacctgatcggcagcaaaaagaaa

SEQ ID NO: 223 - DNA, 1380 bp, synthetic construct
  source 1..1380: mol_type = other DNA
atgaaggttgctatctacaccagagtgtccaccctggagcagcgggaaaagggacactctatcgacgagcaagagcggaaactgagatctttctgcgacattaacgactggaccgtgaaagatgtgtacgtggatgctggcttctccggagccaagcgggacagacctgagctgaccagactcctggacgacatctccgagttcgacctggtgctggtctacaagctggaccggctgacaagaagcgtcagagatctgctggacctgctggaagtgttcgagaacaataacgtggccttcagatctgctaccgaggtgtacgacaccaccaccgccatcggcagactgttcgtgacactcgtgggcgccatggccgagtgggagagagagacaatccgggaaagaagcctgatgggcaagagagccgctattaagaagggcatgatcctgaccgctccacccttctactacgacagggtgaacaacacctacatccctaaccagtataaggatgtggtcctcgatgtgtacaacaaggtcaagaaaggctactccatcgctcatatcgccagactgtacaacaactccgacgtgaagcctcctaacggcaacgaggaatggaccacccggatgctgatgcacgccctgagaaaccctgtgacccggggccactaccagtggggcgagatctacatcgaggactctcatgagcctatcatcacagacgagatgtacaatacaatcatcgaccgcctggacaagcacaccaacaccaaggtggtggcccacacctccgtgtttcggggcaagctgatctgccccaactgtggctacgctctgaccctgaacagccagaagagaaagcggaagaacgacaccatcgtgtacaagacctattactgcaataactgcaagatcaccaagggcatgaagcctcaccacatcaccgagacagaaaccctgcgggtgttcaaggaccacctgtccaagatcgacctgaaacagtacgaaacccaagagaaagagaagcagtctcacgtgaccatcgatctgtctaaagtgatggaacagagaaagagataccacaagctgtacgcctctggcatgatgcaggaaaacgagctgtttgaactgatcaaggaaaccgacgaaatgatcgaagagtacgagaagcagcgcaagcaggtggacgtgaaagagttcgacatctgtaagatcaaagaaatcaaggatgtgctgctgaagtcctgggacatcttcaccctggaagataaggccgacttcatccagatgtccatcaaggctatcaacatcgagtataccaagctgaagcggggaaagagctctaattccatgaagatcaaggatatcgagttttac

SEQ ID NO: 224 - protein, 248 aa, synthetic construct
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGGPADALDDFDLDMLPADALDDFDLDMLPADALDDFDLDMLPG

SEQ ID NO: 225 - protein, 298 aa, synthetic construct
MDMPRIKPGQRVMMALRKMIASGEIKSGERIAEIPTAAALGVSRMPVRIALRSLEQEGLVVRLGARGYAARGVSSDQIRDAIEVRGVLEGFAARRLAERGMTAETHARFVVLIAEGEALFAAGRLNGEDLDRYAAYNQAFHDTLVSAAGNGAVESALARNGFEPFAAAGALALDLMDLSAEYEHLLAAHRQHQAVLDAVSCGDAEGAERIMRDHALAAIRNAKVFEAAASAGAPLGAAWSIRADSGGGGPTDALDDFDLDMLPADALDDFDLDMLPADALDDFDLDMLPGPPKKKRKV

SEQ ID NO: 226 - protein, 264 aa, synthetic construct
MVRRTKEEAQETRAQIIEAAERAFYKRGVARTTLADIAELAGVTRGAIYWHFNNKAELVQALLDSLHETHDHLARASESEDEVDPLGCMRKLLLQVFNELVLDARTRRINEILHHKCEFTDDMCEIRQQHQSAVLDCHKGITLTLANVVRRGQLPGELDAERAAVAMFAYVDGLIRRWLLLPDSVDLLGDVEKWVDTGLDMLRLSPALRKSGGGGPTDALDDFDLDMLPADALDDFDLDMLPADALDDFDLDMLPGPPKKKRKV

SEQ ID NO: 227 - protein, 254 aa, synthetic construct
MARTPSRSSIGSLRSPHTHKAILTSTIEILKECGYSGLSIESVARRAGAGKPTIYRWWTNKAALIAEVYENEIEQVRKFPDLGSFKADLDFLLHNLWKVWRETICGEAFRCVIAEAQLDPVTLTQLKDQFMERRREIPKKLVEDAISNGELPKDINRELLLDMIFGFCWYRLLTEQLTVEQDIEEFTFLLINGVCPGTQCSGGGGPTDALDDFDLDMLPADALDDFDLDMLPADALDDFDLDMLPGPPKKKRKV

SEQ ID NO: 228 - protein, 257 aa, synthetic construct
MSPKRRTQAERAMETQGKLIAAALGVLREKGYAGFRIADVPGAAGVSRGAQSHHFPTKLELLLATFEWLYEQITERSRARLAKLKPEDDVIQQMLDDAAEFFLDDDFSIGLDLIVAADRDPALREGIQRTVERNRFVVEDMWLGVLVSRGLSRDDAEDILWLIFNSVRGLVVRSLWQKDKERFERVRNSTLEIARERYAKFKRSGGGGPTDALDDFDLDMLPADALDDFDLDMLPADALDDFDLDMLPGPPKKKRKV